(19) United States
(12) Patent Application Publication
(10) Pub. No.: US 2025/0115558 A1
(43) Pub. Date: Apr. 10, 2025

(54) PYRIDAZINE DERIVATIVES AND RELATED USES

(71) Applicant: NodThera Limited, Little Chesterford (GB)

(72) Inventors: Mark G. BOCK, Lexington, MA (US); Samuel BROOKES, Little Chesterford (GB); David HARRISON, Little Chesterford (GB); M. Katharine HOLLOWAY, Austin, TX (US)

(21) Appl. No.: 18/906,310

(22) Filed: Oct. 4, 2024

Related U.S. Application Data
(60) Provisional application No. 63/543,223, filed on Oct. 9, 2023.

(51) Int. Cl. C07D 237/08; C07D 401/12; C07D 403/06; C07D 403/08; C07D 405/12; C07D 409/12; C07D 413/12; C07D 417/12

(52) U.S. Cl. CPC C07D 237/08; C07D 401/12; C07D 403/06; C07D 403/08; C07D 405/12; C07D 409/12; C07D 413/12; C07D 417/12

(57) ABSTRACT

The present disclosure relates to compounds of Formula (I):

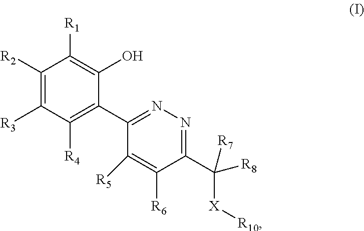

and to their pharmaceutically acceptable salts, pharmaceutical compositions, methods of use, and methods for their preparation. The compounds disclosed herein are useful for inhibiting the maturation of cytokines of the IL-1 family by inhibiting inflammasomes and may be used in the treatment of disorders in which inflammasome activity is implicated, such as inflammatory, autoinflammatory and autoimmune diseases and cancers.

### RELATED APPLICATIONS

This application claims priority to, and the benefit of, U.S. Provisional Application No. 63/543,223, filed on Oct. 9, 2023, the entire contents of which are herein incorporated by reference.

### BACKGROUND

Autoimmune diseases are associated with the overproduction of proinflammatory factors. One of them is interleukin-1 (IL-1), produced by activated macrophages, monocytes, fibroblasts, and other components of the innate immune system like dendritic cells. IL-1 is involved in a variety of cellular activities, including cell proliferation, differentiation and apoptosis (Masters, S. L., et. al., Annu. Rev. Immunol. 2009. 27:621-68).

In humans, 22 NLR proteins are divided into four NLR subfamilies according to their N-terminal domains. NLRA contains a CARD-AT domain, NLRB (NAIP) contains a BIR domain, NLRC (including NOD1 and NOD2) contains a CARD domain, and NLRP contains a pyrin domain. Multiple NLR family members are associated with inflammasome formation.

Although inflammasome activation appears to have evolved as an important component of host immunity to pathogens, the NLRP3 inflammasome is unique in its ability to activate in response to endogenous sterile danger signals. Many such sterile signals have been elucidated, and their formation is associated with specific disease states. For example, uric acid crystals found in gout patients are effective triggers of NLRP3 activation. Similarly, cholesterol crystals found in atherosclerotic patients can also promote NLRP3 activation. Recognition of the role of sterile danger signals as NLRP3 activators led to IL-1 and IL-18 being implicated in a diverse range of pathophysiological indications including metabolic, physiologic, inflammatory, hematologic and immunologic disorders.

The disclosure arises from a need to provide further compounds for the specific modulation of NLRP3-dependent cellular processes. In particular, compounds with improved physicochemical, pharmacological and pharmaceutical properties to existing compounds are desirable.

### SUMMARY

In some aspects, the present disclosure relates to a compound of Formula (I):

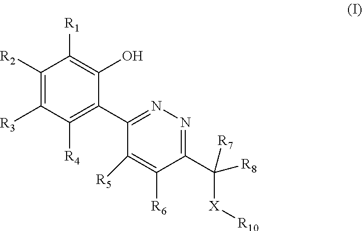

or a prodrug, solvate, or pharmaceutically acceptable salt thereof, wherein:
X is NR₉, O, S, S(O), or SO₂;
each R₁, R₂, R₃, and R₄ are independently H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{a};
R^{a} is halo, —CN, —OH, C₁-C₆ alkyl, C₂-C₆ alkenyl, C₂-C₆ alkynyl, C₁-C₆ haloalkyl, or C₁-C₆ alkoxyl;
each R₅ and R₆ are independently H, halo, —CN, —OH, —NO₂, —NH₂, —NH(C₁-C₆ alkyl), —N(C₁-C₆ alkyl)₂, C₁-C₃ alkyl, C₂-C₄ alkenyl, C₂-C₄ alkynyl, —C₁-C₃ alkoxyl, or C₁-C₃ haloalkyl;
each R₇ and R₈ are independently H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, or C₁-C₆ haloalkyl,
or R₇ and R₈, together with the atoms to which they are attached, form C₃-C₇ cycloalkyl;
R₉ is H, C₁-C₆ alkyl, C₂-C₆ alkenyl, C₂-C₆ alkynyl, or C₁-C₆ haloalkyl;
R₁₀ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{b}; and
R^{b} is halo, oxo, =NH, ═S, —CN, —OH, —NH₂, —NH(C₁-C₆ alkyl), —C(O)(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxyl, C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more C₁-C₆ alkyl, C₂-C₆ alkenyl, C₂-C₆ alkynyl, C₁-C₆ haloalkyl, or C₁-C₆ alkoxyl, or
R₉ and R₁₀, together with the atoms to which they are attached, form 5- to 10-membered heteroaryl or 3- to 10-membered heterocyclyl, wherein the heteroaryl or heterocyclyl is optionally substituted with one or more R^{b}.

In some aspects, the present disclosure provides a compound obtainable by, or obtained by, a method for preparing a compound as described herein.

In some embodiments, the present disclosure provides a pharmaceutical composition comprising a compound described herein and one or more pharmaceutically acceptable carriers or excipients.

In some aspects, the present disclosure provides an intermediate as described herein, being suitable for use in a method for preparing a compound as described herein.

In some aspects, the present disclosure provides a method of inhibiting inflammasome (e.g., the NLRP3 inflammasome) activity (e.g., in vitro or in vivo), comprising contacting a cell with an effective amount of a compound of the present disclosure.

In some aspects, the present disclosure provides a method of treating or preventing a disease or disorder disclosed herein in a subject in need thereof, comprising administering to the subject a therapeutically effective amount of a compound of the present disclosure or a pharmaceutical composition of the present disclosure.

In some aspects, the present disclosure provides a compound of the present disclosure for use in inhibiting inflammasome (e.g., the NLRP3 inflammasome) activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides use of a compound of the present disclosure in the manufacture of a medicament for inhibiting inflammasome (e.g., the NLRP3 inflammasome) activity (e.g., in vitro or in vivo).

In some aspects, the present disclosure provides use of a compound of the present disclosure in the manufacture of a medicament for treating or preventing a disease or disorder disclosed herein.

In some aspects, the present disclosure provides a method of preparing a compound of the present disclosure.

In some aspects, the present disclosure provides a method of a compound, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods, and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

Autoimmune diseases are associated with the overproduction of proinflammatory factors. One of them is interleukin-1 (IL-1), produced by activated macrophages, monocytes, fibroblasts, and other components of the innate immune system like dendritic cells, involved in a variety of cellular activities, including cell proliferation, differentiation and apoptosis (Masters, S. L. et al., Annu. Rev. Immunol. 2009. 27:621-68).

Autoimmune diseases are associated with the overproduction of proinflammatory factors. One of them is interleukin-1 (IL-1), produced by activated macrophages, monocytes, fibroblasts, and other components of the innate immune system like dendritic cells, involved in a variety of cellular activities, including cell proliferation, differentiation, and apoptosis (Masters, S. L., et al. Annu. Rev. Immunol. 2009. 27:621-68).

Cytokines from the IL-1 family are highly active and, as important mediators of inflammation, primarily associated with acute and chronic inflammation (Sims, J. et al., Nature Reviews Immunology 10, 89-102 (February 2010)). The overproduction of IL-1 is considered to be a mediator of some autoimmune and autoinflammatory diseases. Autoinflammatory diseases are characterised by recurrent and unprovoked inflammation in the absence of autoantibodies, infection, or antigen-specific T lymphocytes.

Proinflammatory cytokines of the IL-1 superfamily include IL-1α, IL-1β, IL-18, and IL-36α, β, λ and are produced in response to pathogens and other cellular stressors as part of a host innate immune response. Unlike many other secreted cytokines, which are processed and released via the standard cellular secretory apparatus consisting of the endoplasmic reticulum and Golgi apparatus, IL-1 family members lack leader sequences required for endoplasmic reticulum entry and thus are retained intracellularly following translation. In addition, IL-1β, IL-18, and IL-36α, β, λ are synthesised as procytokines that require proteolytic activation to become optimal ligands for binding to their cognate receptors on target cells.

In the case of IL-1α, IL-1β and IL-18, it is now appreciated that a multimeric protein complex known as an inflammasome is responsible for activating the proforms of IL-1β and IL-18 and for release of these cytokines extracellularly. An inflammasome complex typically consists of a sensor molecule, such as an NLR (Nucleotide-Oligerimisation Domain (NOD)-like receptor), an adaptor molecule ASC (Apoptosis-associated speck-like protein containing a CARD (Caspase Recruitment Domain)) and procaspase-1. In response to a variety of “danger signals”, including pathogen-associated molecule patterns (PAMPs) and danger associated molecular patterns (DAMPs), subunits of an inflammasome oligomerise to form a supramolecular structure within the cell. PAMPs include molecules such as peptidoglycan, viral DNA or RNA and bacterial DNA or RNA. DAMPs, on the other hand, consist of a wide range of endogenous or exogenous sterile triggers including monosodium urate crystals, silica, alum, asbestos, fatty acids, ceramides, cholesterol crystals and aggregates of beta-amyloid peptide. Assembly of an inflammasome platform facilitates autocatalysis of procaspase-1 yielding a highly active cysteine protease responsible for activation and release of pro-IL-1β and pro-IL-18. Thus, release of these highly inflammatory cytokines is achieved only in response to inflammasome sensors detecting and responding to specific molecular danger signals.

In humans, 22 NLR proteins are divided into four NLR subfamilies according to their N-terminal domains. NLRA contains a CARD-AT domain, NLRB (NAIP) contains a BIR domain, NLRC (including NOD1 and NOD2) contains a CARD domain, and NLRP contains a pyrin domain. Multiple NLR family members are associated with inflammasome formation including NLRP1, NLRP3, NLRP6, NLRP7, NLRP12 and NLRC4 (IPAF).

Two other structurally distinct inflammasome structures containing a PYHIN domain (pyrin and HIN domain containing protein) namely Absent in Melanoma 2 (AIM2) and IFNλ-inducible protein 16 (IFI16) (Latz et al., Nat Rev Immunol 2013 13(6) 397-311) serve as intracellular DNA sensors. Pyrin (encoded by the MEFV gene) represents another type of inflammasome platform associated with proIL-1β activation (Chae et al., Immunity 34, 755-768, 2011).

Requiring assembly of an inflammasome platform to achieve activation and release of IL-1β and IL-18 from monocytes and macrophages ensures their production is carefully orchestrated via a 2-step process. First, the cell must encounter a priming ligand (such as the TLR4 receptor ligand LPS, or an inflammatory cytokine such as TNFα) which leads to NFkB dependent transcription of NLRP3, pro-IL-1β and pro-IL-18. The newly translated procytokines remain intracellular and inactive unless producing cells encounter a second signal leading to activation of an inflammasome scaffold and maturation of procaspase-1.

In addition to proteolytic activation of pro-IL-1β and pro-IL-18, active caspase-1 also triggers a form of inflammatory cell death known as pyroptosis through cleavage of gasdermin-D. Pyroptosis allows the mature forms of IL-1β and IL-18 to be externalised along with release of alarmin molecules (compounds that promote inflammation and activate innate and adaptive immunity) such as high mobility group box 1 protein (HMGB1), IL-33, and IL-1α.

Although inflammasome activation appears to have evolved as an important component of host immunity to pathogens, the NLRP3 inflammasome is unique in its ability activate in response to endogenous and exogenous sterile danger signals. Many such sterile signals have been elucidated, and their formation is associated with specific disease states. For example, uric acid crystals found in gout patients are effective triggers of NLRP3 activation. Similarly, cholesterol crystals found in atherosclerotic patients can also promote NLRP3 activation. Recognition of the role of sterile danger signals as NLRP3 activators led to IL-1β and IL-18 being implicated in a diverse range of pathophysiological indications including metabolic, physiologic, inflammatory, hematologic and immunologic disorders.

A link to human disease is best exemplified by discovery that mutations in the NLRP3 gene which lead to gain-of-function confer a range of autoinflammatory conditions collectively known as cryopyrin-associated periodic syndromes (CAPS) including familial cold autoinflammatory syndrome (FCAS), Muckle-Wells syndrome (MWS) and Neonatal onset multisystem inflammatory disease (NOMID) (Hoffman et al., Nat. Genet. 29(3) (2001) 301-305). Likewise, sterile mediator-induced activation of NLRP3 has been implicated in a wide range of disorders including joint degeneration (gout, rheumatoid arthritis, osteoarthritis), cardiometabolic (type 2 diabetes, atherosclerosis, hypertension), Central Nervous System (Parkinson's disease, Alzheimer's disease, multiple sclerosis, amyotrophic lateral sclerosis, or Huntington's disease), gastrointestinal (Crohn's disease, ulcerative colitis, Irritable Bowel Disease (IBD)), lung (chronic obstructive pulmonary disease (COPD), asthma, idiopathic pulmonary fibrosis) and liver (fibrosis, non-alcoholic fatty liver disease, non-alcoholic steatohepatitis (NASH)). It is further believed that NLRP3 activation promotes kidney inflammation and thus contributes to chronic kidney disease (CKD).

Current treatment options for diseases where IL-1 is implicated as a contributor to pathogenesis include the IL-1 receptor antagonist anakinra, an Fc-containing fusion construct of the extracellular domains of the IL-1 receptor and IL-1 receptor accessory protein (rilonacept) and the anti-IL-1β monoclonal antibody canakinumab. For example, canakinumab is licensed for CAPS, Tumor Necrosis Factor Receptor Associated Periodic Syndrome (TRAPS), Hyperimmunoglobulin D Syndrome (HIDS)/Mevalonate Kinase Deficiency (MKD), Familial Mediterranean Fever (FMF) and gout.

Some small molecules have been reported to inhibit function of the NLRP3 inflammasome. Glyburide, for example, is a specific inhibitor of NLRP3 activation, albeit at micromolar concentrations which are unlikely attainable in vivo. Non-specific agents such as parthenolide, Bay 11-7082, and 3,4-methylenedioxy-p-nitrostyrene are reported to impair NLRP3 activation but are expected to possess limited therapeutic utility due to their sharing of a common structural feature consisting of an olefin activated by substitution with an electron withdrawing group; this can lead to undesirable formation of covalent adducts with protein-bearing thiol groups. A number of natural products, for example p-hydroxybutyrate, sulforaphane, quercetin, and salvianolic acid, also are reported to suppress NLRP3 activation. Likewise, numerous effectors/modulators of other molecular targets have been reported to impair NLRP3 activation including agonists of the G-protein coupled receptor TGR5, an inhibitor of sodium-glucose co-transport epigliflozin, the dopamine receptor antagonist A-68930, the serotonin reuptake inhibitor fluoxetine, fenamate non-steroidal anti-inflammatory drugs, and the β-adrenergic receptor blocker nebivolol. Utility of these molecules as therapeutics for the chronic treatment of NLRP3-dependent inflammatory disorders remains to be established.

The disclosure relates to compounds useful for the modulation of NLRP3-dependent cellular processes. In particular, compounds with improved physicochemical, pharmacological and pharmaceutical properties to existing NLRP3-modulating compounds are desired.

### Compounds of the Present Disclosure

In some aspects, the present disclosure relates to a compound of Formula (I):

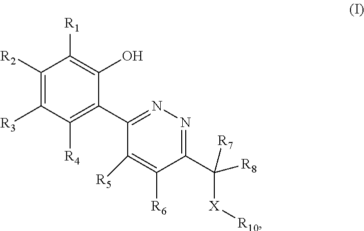

or a prodrug, solvate, or pharmaceutically acceptable salt thereof, wherein:
X is NR₉, O, S, S(O), or SO₂;
each R₁, R₂, R₃, and R₄ are independently H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{a};
R^{a} is halo, —CN, —OH, C₁-C₆ alkyl, C₂-C₆ alkenyl, C₂-C₆ alkynyl, C₁-C₆ haloalkyl, or C₁-C₆ alkoxyl;
each R₅ and R₆ are independently H, halo, —CN, —OH, —NO₂, —NH₂, —NH(C₁-C₆ alkyl), —N(C₁-C₆ alkyl)₂, C₁-C₃ alkyl, C₂-C₄ alkenyl, C₂-C₄ alkynyl, —C₁-C₃ alkoxyl, or C₁-C₃ haloalkyl;
each R₇ and R₈ are independently H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, or C₁-C₆ haloalkyl, or R₇ and R₈, together with the atoms to which they are attached, form C₃-C₇ cycloalkyl;
R₉ is H, C₁-C₆ alkyl, C₂-C₆ alkenyl, C₂-C₆ alkynyl, or C₁-C₆ haloalkyl;
R₁₀ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{b}; and
R^{b} is halo, oxo, =NH, ═S, —CN, —OH, —NH₂, —NH(C₁-C₆ alkyl), —C(O)(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxyl, C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more C₁-C₆ alkyl, C₂-C₆ alkenyl, C₂-C₆ alkynyl, C₁-C₆ haloalkyl, or C₁-C₆ alkoxyl, or
R₉ and R₁₀, together with the atoms to which they are attached, form 5- to 10-membered heteroaryl or 3- to 10-membered heterocyclyl, wherein the heteroaryl or heterocyclyl is optionally substituted with one or more R^{b}.

In some aspects, the present disclosure relates to a compound of Formula (I):

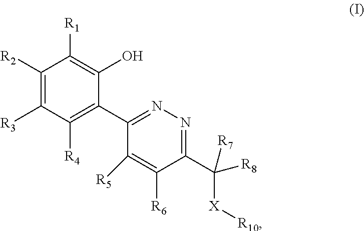

or a prodrug, solvate, or pharmaceutically acceptable salt thereof, wherein:
X is NR₉ or O;
each R₁, R₂, R₃, and R₄ are independently H, halo, —NO₂, C₁-C₆ alkyl, C₁-C₆ alkoxyl, or C₃-C₆ cycloalkyl, wherein the alkyl, alkoxyl, or cycloalkyl is optionally substituted with one or more R^{a};
R^{a} is halo;
each R₅ and R₆ are independently H or C₁-C₃ alkyl;
each R₇ and R₈ are independently H, or C₁-C₆ alkyl,
or R₇ and R₈, together with the atoms to which they are attached, form C₃-C₇ cycloalkyl;
R₉ is H or C₁-C₆ alkyl; R₁₀ is H, C₁-C₆ alkyl, C₁-C₆ alkoxyl, C₃-C₆ cycloalkyl, 5- to 6-membered heteroaryl, or 3- to 10-membered heterocyclyl, wherein the alkyl, alkoxyl, cycloalkyl, heteroaryl, or heterocyclyl is optionally substituted with one or more R^{b}; and
R^{b} is halo, oxo, =NH, ═S, —OH, —NH₂, —N(C₁-C₆ alkyl)₂, —C(O)(C₁-C₆ alkyl), C₁-C₆ alkyl, C₁-C₆ alkoxyl, C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more C₁-C₆ alkyl, or
R₉ and R₁₀, together with the atoms to which they are attached, form 5- to 10-membered heteroaryl or 3- to 10-membered heterocyclyl, wherein the heteroaryl or heterocyclyl is optionally substituted with one or more R^{b}.

It is understood that, for a compound of Formula (I), X, R₁, R₂, R₃, R₄, R₅, R₆, R₇, R₈, R₉, R₁₀, R^{a}, and R^{b} can each be, where applicable, selected from the groups described herein, and any group described herein for any of X, R₁, R₂, R₃, R₄, R₅, R₆, R₇, R₈, R₉, R₁₀, R^{a}, and R^{b} can be combined, where applicable, with any group described herein for one or more of the remainder of X, R₁, R₂, R₃, R₄, R₅, R₆, R₇, R₈, R₉, R₁₀, R^{a}, and R^{b}.

In some embodiments, X is NR₉, O, S, S(O), or SO₂.

In some embodiments, X is NR₉.

In some embodiments, X is NH.

In some embodiments, X is O.

In some embodiments, X is S, S(O), or SO₂.

In some embodiments, X is S. In some embodiments, X is S(O). In some embodiments, X is SO₂.

In some embodiments, each R₁, R₂, R₃, and R₄ are independently H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{a}.

In some embodiments, each R₁, R₂, R₃, and R₄ are independently H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl.

In some embodiments, R₁ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{a}.

In some embodiments, R₁ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl.

In some embodiments, R₁ is halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{a}.

In some embodiments, R₁ is halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl.

In some embodiments, R₁ is H, halo, —CN, —OH, —NO₂, or —NH₂.

In some embodiments, R₁ is H.

In some embodiments, R₁ is halo.

In some embodiments, R₁ is F, Cl, Br, or I. In some embodiments, R₁ is F. In some embodiments, R₁ is C₁. In some embodiments, R₁ is Br. In some embodiments, R₁ is I.

In some embodiments, R₁ is —CN. In some embodiments, R₁ is —OH. In some embodiments, R₁ is —NO₂. In some embodiments, R₁ is —NH₂.

In some embodiments, R₁ is —NH(C₁-C₆ alkyl) or —N(C₁-C₆ alkyl)₂.

In some embodiments, R₁ is —NH(C₁-C₆ alkyl). In some embodiments, R₁ is —N(C₁-C₆ alkyl)₂.

In some embodiments, R₁ is C₁-C₆ alkyl, C₂-C₆ alkenyl, C₂-C₆ alkynyl, or C₁-C₆ alkoxyl, wherein the alkyl, alkenyl, alkynyl, or alkoxyl is optionally substituted with one or more R^{a}.

In some embodiments, R₁ is C₁-C₆ alkyl, C₂-C₆ alkenyl, C₂-C₆ alkynyl, or C₁-C₆ alkoxyl.

In some embodiments, R₁ is C₁-C₆ alkyl optionally substituted with one or more R^{a}.

In some embodiments, R₁ is C₁-C₆ alkyl substituted with one or more R^{a}.

In some embodiments, R₁ is C₁-C₆ alkyl.

In some embodiments, R₁ is methyl optionally substituted with one or more R^{a}.

In some embodiments, R₁ is methyl substituted with one or more R^{a}.

In some embodiments, R₁ is methyl.

In some embodiments, R₁ is C₂-C₆ alkenyl optionally substituted with one or more R^{a}.

In some embodiments, R₁ is C₂-C₆ alkenyl substituted with one or more R^{a}.

In some embodiments, R₁ is C₂-C₆ alkenyl.

In some embodiments, R₁ is C₂-C₆ alkynyl optionally substituted with one or more R^{a}.

In some embodiments, R₁ is C₂-C₆ alkynyl substituted with one or more R^{a}.

In some embodiments, R₁ is C₂-C₆ alkynyl.

In some embodiments, R₁ is C₁-C₆ alkoxyl optionally substituted with one or more R^{a}.

In some embodiments, R₁ is C₁-C₆ alkoxyl substituted with one or more R^{a}.

In some embodiments, R₁ is C₁-C₆ alkoxyl.

In some embodiments, R₁ is C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more R^{a}.

In some embodiments, R₁ is C₃-C₆ cycloalkyl, C₆-C₁₀ aryl, 5- to 6-membered heteroaryl, or 3- to 10-membered heterocyclyl.

In some embodiments, R₁ is C₃-C₆ cycloalkyl optionally substituted with one or more R^{a}.

In some embodiments, R₁ is C₃-C₆ cycloalkyl substituted with one or more R^{a}.

In some embodiments, R₁ is C₃-C₆ cycloalkyl.

In some embodiments, R₁ is C₆-C₁₀ aryl optionally substituted with one or more R^{a}.

In some embodiments, R₁ is C₆-C₁₀ aryl substituted with one or more R^{a}.

In some embodiments, R₁ is C₆-C₁₀ aryl.

In some embodiments, R₁ is 5- to 6-membered heteroaryl optionally substituted with one or more R^{a}.

In some embodiments, R₁ is 5- to 6-membered heteroaryl substituted with one or more R^{a}.

In some embodiments, R₁ is 5- to 6-membered heteroaryl.

In some embodiments, R₁ is 3- to 10-membered heterocyclyl optionally substituted with one or more R^{a}.

In some embodiments, R₁ is 3- to 10-membered heterocyclyl substituted with one or more R^{a}.

In some embodiments, R₁ is 3- to 10-membered heterocyclyl.

In some embodiments, R₁ is H or C₁-C₆ alkyl.

In some embodiments, R₁ is H or —CH₃.

In some embodiments, R₂ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{a}.

In some embodiments, R₂ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl.

In some embodiments, R₂ is halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{a}.

In some embodiments, R₂ is halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl.

In some embodiments, R₂ is halo, —NO₂, C₁-C₆ alkyl, C₁-C₆ alkoxyl, or C₃-C₆ cycloalkyl, wherein the alkyl, alkoxyl or cycloalkyl is optionally substituted with one or more R^{a}.

In some embodiments, R₂ is halo, —NO₂, C₁-C₆ alkyl, C₁-C₆ alkoxyl, or C₃-C₆ cycloalkyl.

In some embodiments, R₂ is H, halo, —CN, —OH, —NO₂, or —NH₂.

In some embodiments, R₂ is H.

In some embodiments, R₂ is halo.

In some embodiments, R₂ is F, Cl, Br, or I.

In some embodiments, R₂ is Cl, F, or Br.

In some embodiments, R₂ is F. In some embodiments, R₂ is CL. In some embodiments, R₂ is Br. In some embodiments, R₂ is I.

In some embodiments, R₂ is —CN. In some embodiments, R₂ is —OH. In some embodiments, R₂ is —NO₂. In some embodiments, R₂ is —NH₂.

In some embodiments, R₂ is —NH(C₁-C₆ alkyl) or —N(C₁-C₆ alkyl)₂.

In some embodiments, R₂ is —NH(C₁-C₆ alkyl). In some embodiments, R₂ is —N(C₁-C₆ alkyl)₂.

In some embodiments, R₂ is C₁-C₆ alkyl, C₂-C₆ alkenyl, C₂-C₆ alkynyl, or C₁-C₆ alkoxyl, wherein the alkyl, alkenyl, alkynyl, or alkoxyl is optionally substituted with one or more R^{a}.

In some embodiments, R₂ is C₁-C₆ alkyl, C₂-C₆ alkenyl, C₂-C₆ alkynyl, or C₁-C₆ alkoxyl.

In some embodiments, R₂ is C₁-C₆ alkyl optionally substituted with one or more R^{a}.

In some embodiments, R₂ is C₁-C₆ alkyl substituted with one or more R^{a}.

In some embodiments, R₂ is C₁-C₆ alkyl.

In some embodiments, R₂ is methyl optionally substituted with one or more R^{a}.

In some embodiments, R₂ is methyl substituted with one or more R^{a}.

In some embodiments, R₂ is methyl.

In some embodiments, R₂ is methyl optionally substituted with one or more halo.

In some embodiments, R₂ is methyl substituted with one or more halo.

In some embodiments, R₂ is C₂-C₆ alkenyl optionally substituted with one or more R^{a}.

In some embodiments, R₂ is C₂-C₆ alkenyl substituted with one or more R^{a}.

In some embodiments, R₂ is C₂-C₆ alkenyl.

In some embodiments, R₂ is C₂-C₆ alkynyl optionally substituted with one or more R^{a}.

In some embodiments, R₂ is C₂-C₆ alkynyl substituted with one or more R^{a}.

In some embodiments, R₂ is C₂-C₆ alkynyl.

In some embodiments, R₂ is C₁-C₆ alkoxyl optionally substituted with one or more R^{a}.

In some embodiments, R₂ is C₁-C₆ alkoxyl substituted with one or more R^{a}.

In some embodiments, R₂ is C₁-C₆ alkoxyl.

In some embodiments, R₂ is C₁-C₆ alkoxyl optionally substituted with one or more halo.

In some embodiments, R₂ is C₁-C₆ alkoxyl substituted with one or more halo.

In some embodiments, R₂ is C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more R^{a}.

In some embodiments, R₂ is C₃-C₆ cycloalkyl, C₆-C₁₀ aryl, 5- to 6-membered heteroaryl, or 3- to 10-membered heterocyclyl.

In some embodiments, R₂ is C₃-C₆ cycloalkyl optionally substituted with one or more R^{a}.

In some embodiments, R₂ is C₃-C₆ cycloalkyl substituted with one or more R^{a}.

In some embodiments, R₂ is C₃-C₆ cycloalkyl.

In some embodiments, R₂ is cyclopropyl optionally substituted with one or more R^{a}.

In some embodiments, R₂ is cyclopropyl substituted with one or more R^{a}.

In some embodiments, R₂ is cyclopropyl.

In some embodiments, R₂ is C₆-C₁₀ aryl optionally substituted with one or more R^{a}.

In some embodiments, R₂ is C₆-C₁₀ aryl substituted with one or more R^{a}.

In some embodiments, R₂ is C₆-C₁₀ aryl.

In some embodiments, R₂ is 5- to 6-membered heteroaryl optionally substituted with one or more R^{a}.

In some embodiments, R₂ is 5- to 6-membered heteroaryl substituted with one or more R^{a}.

In some embodiments, R₂ is 5- to 6-membered heteroaryl.

In some embodiments, R₂ is 3- to 10-membered heterocyclyl optionally substituted with one or more R^{a}.

In some embodiments, R₂ is 3- to 10-membered heterocyclyl substituted with one or more R^{a}.

In some embodiments, R₂ is 3- to 10-membered heterocyclyl.

In some embodiments, R₂ is Cl, F, Br, —NO₂, —CF₃, —OCF₂H, —OCF₃, or cyclopropyl.

In some embodiments, R₃ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{a}.

In some embodiments, R₃ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl.

In some embodiments, R₃ is halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{a}.

In some embodiments, R₃ is halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl.

In some embodiments, R₃ is H, halo, —CN, —OH, —NO₂, or —NH₂.

In some embodiments, R₃ is H.

In some embodiments, R₃ is halo.

In some embodiments, R₃ is F, Cl, Br, or I. In some embodiments, R₃ is F. In some embodiments, R₃ is CL. In some embodiments, R₃ is Br. In some embodiments, R₃ is I.

In some embodiments, R₃ is —CN. In some embodiments, R₃ is —OH. In some embodiments, R₃ is —NO₂. In some embodiments, R₃ is —NH₂.

In some embodiments, R₃ is —NH(C₁-C₆ alkyl) or —N(C₁-C₆ alkyl)₂.

In some embodiments, R₃ is —NH(C₁-C₆ alkyl). In some embodiments, R₃ is —N(C₁-C₆ alkyl)₂.

In some embodiments, R₃ is C₁-C₆ alkyl, C₂-C₆ alkenyl, C₂-C₆ alkynyl, or C₁-C₆ alkoxyl, wherein the alkyl, alkenyl, alkynyl, or alkoxyl is optionally substituted with one or more R^{a}.

In some embodiments, R₃ is C₁-C₆ alkyl, C₂-C₆ alkenyl, C₂-C₆ alkynyl, or C₁-C₆ alkoxyl.

In some embodiments, R₃ is C₁-C₆ alkyl optionally substituted with one or more R^{a}.

In some embodiments, R₃ is C₁-C₆ alkyl substituted with one or more R^{a}.

In some embodiments, R₃ is C₁-C₆ alkyl.

In some embodiments, R₃ is methyl optionally substituted with one or more R^{a}.

In some embodiments, R₃ is methyl substituted with one or more R^{a}.

In some embodiments, R₃ is methyl.

In some embodiments, R₃ is C₂-C₆ alkenyl optionally substituted with one or more R^{a}.

In some embodiments, R₃ is C₂-C₆ alkenyl substituted with one or more R^{a}.

In some embodiments, R₃ is C₂-C₆ alkenyl.

In some embodiments, R₃ is C₂-C₆ alkynyl optionally substituted with one or more R^{a}.

In some embodiments, R₃ is C₂-C₆ alkynyl substituted with one or more R^{a}.

In some embodiments, R₃ is C₂-C₆ alkynyl.

In some embodiments, R₃ is C₁-C₆ alkoxyl optionally substituted with one or more R^{a}.

In some embodiments, R₃ is C₁-C₆ alkoxyl substituted with one or more R^{a}.

In some embodiments, R₃ is C₁-C₆ alkoxyl.

In some embodiments, R₃ is C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more R^{a}.

In some embodiments, R₃ is C₃-C₆ cycloalkyl, C₆-C₁₀ aryl, 5- to 6-membered heteroaryl, or 3- to 10-membered heterocyclyl.

In some embodiments, R₃ is C₃-C₆ cycloalkyl optionally substituted with one or more R^{a}.

In some embodiments, R₃ is C₃-C₆ cycloalkyl substituted with one or more R^{a}.

In some embodiments, R₃ is C₃-C₆ cycloalkyl.

In some embodiments, R₃ is C₆-C₁₀ aryl optionally substituted with one or more R^{a}.

In some embodiments, R₃ is C₆-C₁₀ aryl substituted with one or more R^{a}.

In some embodiments, R₃ is C₆-C₁₀ aryl.

In some embodiments, R₃ is 5- to 6-membered heteroaryl optionally substituted with one or more R^{a}.

In some embodiments, R₃ is 5- to 6-membered heteroaryl substituted with one or more R^{a}.

In some embodiments, R₃ is 5- to 6-membered heteroaryl.

In some embodiments, R₃ is 3- to 10-membered heterocyclyl optionally substituted with one or more R^{a}.

In some embodiments, R₃ is 3- to 10-membered heterocyclyl substituted with one or more R^{a}.

In some embodiments, R₃ is 3- to 10-membered heterocyclyl.

In some embodiments, R₃ is H or C₁-C₆ alkyl.

In some embodiments, R₃ is H or —CH₃.

In some embodiments, R₄ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{a}.

In some embodiments, R₄ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl.

In some embodiments, R₄ is halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{a}.

In some embodiments, R₄ is halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl.

In some embodiments, R₄ is H, halo, —CN, —OH, —NO₂, or —NH₂.

In some embodiments, R₄ is H.

In some embodiments, R₄ is halo.

In some embodiments, R₄ is F, Cl, Br, or I.

In some embodiments, R₄ is F or CL.

In some embodiments, R₄ is F. In some embodiments, R₄ is CL. In some embodiments, R₄ is Br. In some embodiments, R₄ is I.

In some embodiments, R₄ is —CN. In some embodiments, R₄ is —OH. In some embodiments, R₄ is —NO₂. In some embodiments, R₄ is —NH₂.

In some embodiments, R₄ is —NH(C₁-C₆ alkyl) or —N(C₁-C₆ alkyl)₂.

In some embodiments, R₄ is —NH(C₁-C₆ alkyl). In some embodiments, R₄ is —N(C₁-C₆ alkyl)₂.

In some embodiments, R₄ is C₁-C₆ alkyl, C₂-C₆ alkenyl, C₂-C₆ alkynyl, or C₁-C₆ alkoxyl, wherein the alkyl, alkenyl, alkynyl, or alkoxyl is optionally substituted with one or more R^{a}.

In some embodiments, R₄ is C₁-C₆ alkyl, C₂-C₆ alkenyl, C₂-C₆ alkynyl, or C₁-C₆ alkoxyl.

In some embodiments, R₄ is C₁-C₆ alkyl optionally substituted with one or more R^{a}.

In some embodiments, R₄ is C₁-C₆ alkyl substituted with one or more R^{a}.

In some embodiments, R₄ is C₁-C₆ alkyl.

In some embodiments, R₄ is methyl optionally substituted with one or more R^{a}.

In some embodiments, R₄ is methyl substituted with one or more R^{a}.

In some embodiments, R₄ is methyl.

In some embodiments, R₄ is C₂-C₆ alkenyl optionally substituted with one or more R^{a}.

In some embodiments, R₄ is C₂-C₆ alkenyl substituted with one or more R^{a}.

In some embodiments, R₄ is C₂-C₆ alkenyl.

In some embodiments, R₄ is C₂-C₆ alkynyl optionally substituted with one or more R^{a}.

In some embodiments, R₄ is C₂-C₆ alkynyl substituted with one or more R^{a}.

In some embodiments, R₄ is C₂-C₆ alkynyl.

In some embodiments, R₄ is C₁-C₆ alkoxyl optionally substituted with one or more R^{a}.

In some embodiments, R₄ is C₁-C₆ alkoxyl substituted with one or more R^{a}.

In some embodiments, R₄ is C₁-C₆ alkoxyl.

In some embodiments, R₄ is C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more R^{a}.

In some embodiments, R₄ is C₃-C₆ cycloalkyl, C₆-C₁₀ aryl, 5- to 6-membered heteroaryl, or 3- to 10-membered heterocyclyl.

In some embodiments, R₄ is C₃-C₆ cycloalkyl optionally substituted with one or more R^{a}.

In some embodiments, R₄ is C₃-C₆ cycloalkyl substituted with one or more R^{a}.

In some embodiments, R₄ is C₃-C₆ cycloalkyl.

In some embodiments, R₄ is C₆-C₁₀ aryl optionally substituted with one or more R^{a}.

In some embodiments, R₄ is C₆-C₁₀ aryl substituted with one or more R^{a}.

In some embodiments, R₄ is C₆-C₁₀ aryl.

In some embodiments, R₄ is 5- to 6-membered heteroaryl optionally substituted with one or more R^{a}.

In some embodiments, R₄ is 5- to 6-membered heteroaryl substituted with one or more R^{a}.

In some embodiments, R₄ is 5- to 6-membered heteroaryl.

In some embodiments, R₄ is 3- to 10-membered heterocyclyl optionally substituted with one or more R^{a}.

In some embodiments, R₄ is 3- to 10-membered heterocyclyl substituted with one or more R^{a}.

In some embodiments, R₄ is 3- to 10-membered heterocyclyl.

In some embodiments, R₄ is H, halo, or C₁-C₆ alkyl.

In some embodiments, R₄ is H, F, CL, or —CH₃.

In some embodiments, R^{a} is halo, —CN, —OH, C₁-C₆ alkyl, C₂-C₆ alkenyl, C₂-C₆ alkynyl, C₁-C₆ haloalkyl, or C₁-C₆ alkoxyl.

In some embodiments, R^{a} is halo, —CN, or —OH.

In some embodiments, R^{a} is halo.

In some embodiments, R^{a} is F, Cl, Br, or I. In some embodiments, R^{a} is F. In some embodiments, R^{a} is CL. In some embodiments, R^{a} is Br. In some embodiments, R^{a} is I.

In some embodiments, R^{a} is —CN. In some embodiments, R^{a} is —OH.

In some embodiments, R^{a} is C₁-C₆ alkyl, C₂-C₆ alkenyl, C₂-C₆ alkynyl, C₁-C₆ alkoxyl, or C₁-C₆ haloalkyl.

In some embodiments, R^{a} is C₁-C₆ alkyl.

In some embodiments, R^{a} is C₂-C₆ alkenyl.

In some embodiments, R^{a} is C₂-C₆ alkynyl.

In some embodiments, R^{a} is C₁-C₆ alkoxyl.

In some embodiments, R^{a} is C₁-C₆ haloalkyl.

In some embodiments, each R₅ and R₆ are independently H, halo, —CN, —OH, —NO₂, —NH₂, —NH(C₁-C₆ alkyl), —N(C₁-C₆ alkyl)₂, C₁-C₃ alkyl, C₂-C₄ alkenyl, C₂-C₄ alkynyl, —C₁-C₃ alkoxyl, or C₁-C₃ haloalkyl.

In some embodiments, R₅ is H, halo, —CN, —OH, —NO₂, —NH₂, —NH(C₁-C₆ alkyl), —N(C₁-C₆ alkyl)₂, C₁-C₃ alkyl, C₂-C₄ alkenyl, C₂-C₄ alkynyl, C₁-C₃ alkoxyl, or C₁-C₃ haloalkyl.

In some embodiments, R₅ is H, halo, —CN, —OH, —NO₂, or —NH₂.

In some embodiments, R₅ is H.

In some embodiments, R₅ is halo.

In some embodiments, R₅ is F, Cl, Br, or I. In some embodiments, R₅ is F. In some embodiments, R₅ is CL. In some embodiments, R₅ is Br. In some embodiments, R₅ is I.

In some embodiments, R₅ is —CN. In some embodiments, R₅ is —OH. In some embodiments, R₅ is —NO₂. In some embodiments, R₅ is —NH₂.

In some embodiments, R₅ is —NH(C₁-C₆ alkyl) or —N(C₁-C₆ alkyl)₂.

In some embodiments, R₅ is —NH(C₁-C₆ alkyl). In some embodiments, R₅ is —N(C₁-C₆ alkyl)₂.

In some embodiments, R₅ is C₁-C₃ alkyl, C₂-C₄ alkenyl, C₂-C₄ alkynyl, C₁-C₃ alkoxyl, or C₁-C₃ haloalkyl.

In some embodiments, R₅ is C₁-C₃ alkyl.

In some embodiments, R₅ is methyl.

In some embodiments, R₅ is C₂-C₄ alkenyl.

In some embodiments, R₅ is C₂-C₄ alkynyl.

In some embodiments, R₅ is C₁-C₃ alkoxyl.

In some embodiments, R₅ is C₁-C₃ haloalkyl.

In some embodiments, R₅ is H or C₁-C₃ alkyl.

In some embodiments, R₅ is H or —CH₃.

In some embodiments, R₆ is H, halo, —CN, —OH, —NO₂, —NH₂, —NH(C₁-C₆ alkyl), —N(C₁-C₆ alkyl)₂, C₁-C₃ alkyl, C₂-C₄ alkenyl, C₂-C₄ alkynyl, C₁-C₃ alkoxyl, or C₁-C₃ haloalkyl.

In some embodiments, R₆ is H, halo, —CN, —OH, —NO₂, or —NH₂.

In some embodiments, R₆ is H.

In some embodiments, R₆ is halo.

In some embodiments, R₆ is F, Cl, Br, or I. In some embodiments, R₆ is F. In some embodiments, R₆ is C₁. In some embodiments, R₆ is Br. In some embodiments, R₆ is I.

In some embodiments, R₆ is —CN. In some embodiments, R₆ is —OH. In some embodiments, R₆ is —NO₂. In some embodiments, R₆ is —NH₂.

In some embodiments, R₆ is —NH(C₁-C₆ alkyl) or —N(C₁-C₆ alkyl)₂.

In some embodiments, R₆ is —NH(C₁-C₆ alkyl). In some embodiments, R₆ is —N(C₁-C₆ alkyl)₂.

In some embodiments, R₆ is C₁-C₃ alkyl, C₂-C₄ alkenyl, C₂-C₄ alkynyl, C₁-C₃ alkoxyl, or C₁-C₃ haloalkyl.

In some embodiments, R₆ is C₁-C₃ alkyl.

In some embodiments, R₆ is methyl.

In some embodiments, R₆ is C₂-C₄ alkenyl.

In some embodiments, R₆ is C₂-C₄ alkynyl.

In some embodiments, R₆ is C₁-C₃ alkoxyl.

In some embodiments, R₆ is C₁-C₃ haloalkyl.

In some embodiments, each R₇ and R₈ are independently H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, or C₁-C₆ haloalkyl.

In some embodiments, R₇ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, or C₁-C₆ haloalkyl, or R₇ and R₈, together with the atoms to which they are attached, form C₃-C₇ cycloalkyl.

In some embodiments, R₇ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, or C₁-C₆ haloalkyl.

In some embodiments, R₇ is H, halo, —CN, —OH, —NO₂, or —NH₂.

In some embodiments, R₇ is H.

In some embodiments, R₇ is halo.

In some embodiments, R₇ is F, Cl, Br, or I. In some embodiments, R₇ is F. In some embodiments, R₇ is C₁. In some embodiments, R₇ is Br. In some embodiments, R₇ is I.

In some embodiments, R₇ is —CN. In some embodiments, R₇ is —OH. In some embodiments, R₇ is —NO₂. In some embodiments, R₇ is —NH₂.

In some embodiments, R₇ is —NH(C₁-C₆ alkyl) or —N(C₁-C₆ alkyl)₂.

In some embodiments, R₇ is —NH(C₁-C₆ alkyl). In some embodiments, R₇ is —N(C₁-C₆ alkyl)₂.

In some embodiments, R₇ is C₁-C₆ alkyl, C₂-C₆ alkenyl, C₂-C₆ alkynyl, C₁-C₆ alkoxyl, or C₁-C₆ haloalkyl.

In some embodiments, R₇ is C₁-C₆ alkyl.

In some embodiments, R₇ is methyl.

In some embodiments, R₇ is C₂-C₆ alkenyl.

In some embodiments, R₇ is C₂-C₆ alkynyl.

In some embodiments, R₇ is C₁-C₆ alkoxyl.

In some embodiments, R₇ is C₁-C₆ haloalkyl.

In some embodiments, R₇ is H or C₁-C₆ alkyl.

In some embodiments, R₇ is H or —CH₃.

In some embodiments, R₈ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, or C₁-C₆ haloalkyl, or R₇ and R₈, together with the atoms to which they are attached, form C₃-C₇ cycloalkyl.

In some embodiments, R₈ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, or C₁-C₆ haloalkyl.

In some embodiments, R₈ is H, halo, —CN, —OH, —NO₂, or —NH₂.

In some embodiments, R₈ is H.

In some embodiments, R₈ is halo.

In some embodiments, R₈ is F, Cl, Br, or I. In some embodiments, R₈ is F. In some embodiments, R₈ is CL. In some embodiments, R₈ is Br. In some embodiments, R₈ is I.

In some embodiments, R₈ is —CN. In some embodiments, R₈ is —OH. In some embodiments, R₈ is —NO₂. In some embodiments, R₈ is —NH₂.

In some embodiments, R₈ is —NH(C₁-C₆ alkyl) or —N(C₁-C₆ alkyl)₂.

In some embodiments, R₈ is —NH(C₁-C₆ alkyl). In some embodiments, R₈ is —N(C₁-C₆ alkyl)₂.

In some embodiments, R₈ is C₁-C₆ alkyl, C₂-C₆ alkenyl, C₂-C₆ alkynyl, C₁-C₆ alkoxyl, or C₁-C₆ haloalkyl.

In some embodiments, R₈ is C₁-C₆ alkyl.

In some embodiments, R₈ is methyl.

In some embodiments, R₈ is C₂-C₆ alkenyl.

In some embodiments, R₈ is C₂-C₆ alkynyl.

In some embodiments, R₈ is C₁-C₆ alkoxyl.

In some embodiments, R₈ is C₁-C₆ haloalkyl.

In some embodiments, R₈ is H or C₁-C₆ alkyl.

In some embodiments, R₈ is H or —CH₃.

In some embodiments, R₇ and R₈, together with the atoms to which they are attached, form C₃-C₇ cycloalkyl.

In some embodiments, R₇ and R₈, together with the atoms to which they are attached, form cyclopropyl.

In some embodiments, R₉ is H, C₁-C₆ alkyl, C₂-C₆ alkenyl, C₂-C₆ alkynyl, or C₁-C₆ haloalkyl.

In some embodiments, R₉ is H, C₁-C₆ alkyl, C₂-C₆ alkenyl, C₂-C₆ alkynyl, or C₁-C₆ haloalkyl.

In some embodiments, R₉ is H.

In some embodiments, R₉ is C₁-C₆ alkyl, C₂-C₆ alkenyl, C₂-C₆ alkynyl, or C₁-C₆ haloalkyl.

In some embodiments, R₉ is C₁-C₆ alkyl.

In some embodiments, R₉ is methyl.

In some embodiments, R₉ is C₂-C₆ alkenyl.

In some embodiments, R₉ is C₂-C₆ alkynyl.

In some embodiments, R₉ is C₁-C₆ haloalkyl.

In some embodiments, R₉ is H or C₁-C₆ alkyl.

In some embodiments, R₉ is H or —CH₃.

In some embodiments, R₁₀ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{b}; and R^{b} is halo, oxo, =NH, ═S, —CN, —OH, —NH₂, —NH(C₁-C₆ alkyl), —C(O)(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxyl, C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more C₁-C₆ alkyl, C₂-C₆ alkenyl, C₂-C₆ alkynyl, C₁-C₆ haloalkyl, or C₁-C₆ alkoxyl.

In some embodiments, R₁₀ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{b}.

In some embodiments, R₁₀ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl.

In some embodiments, R₁₀ is halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{b}.

In some embodiments, R₁₀ is halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl.

In some embodiments, R₁₀ is H, halo, —CN, —OH, —NO₂, or —NH₂.

In some embodiments, R₁₀ is H.

In some embodiments, R₁₀ is halo.

In some embodiments, R₁₀ is F, Cl, Br, or I.

In some embodiments, R₁₀ is F or CL.

In some embodiments, R₁₀ is F. In some embodiments, R₁₀ is C₁. In some embodiments, R₁₀ is Br. In some embodiments, R₁₀ is I.

In some embodiments, R₁₀ is —CN. In some embodiments, R₁₀ is —OH. In some embodiments, R₁₀ is —NO₂. In some embodiments, R₁₀ is —NH₂.

In some embodiments, R₁₀ is —NH(C₁-C₆ alkyl) or —N(C₁-C₆ alkyl)₂.

In some embodiments, R₁₀ is —NH(C₁-C₆ alkyl). In some embodiments, R₁₀ is —N(C₁-C₆ alkyl)₂.

In some embodiments, R₁₀ is C₁-C₆ alkyl, C₂-C₆ alkenyl, C₂-C₆ alkynyl, or C₁-C₆ alkoxyl, wherein the alkyl, alkenyl, alkynyl, or alkoxyl is optionally substituted with one or more R^{b}.

In some embodiments, R₁₀ is C₁-C₆ alkyl, C₂-C₆ alkenyl, C₂-C₆ alkynyl, or C₁-C₆ alkoxyl.

In some embodiments, R₁₀ is C₁-C₆ alkyl optionally substituted with one or more R^{b}.

In some embodiments, R₁₀ is C₁-C₆ alkyl substituted with one or more R^{b}.

In some embodiments, R₁₀ is C₁-C₆ alkyl.

In some embodiments, R₁₀ is C₂-C₆ alkenyl optionally substituted with one or more R^{b}.

In some embodiments, R₁₀ is C₂-C₆ alkenyl substituted with one or more R^{b}.

In some embodiments, R₁₀ is C₂-C₆ alkenyl.

In some embodiments, R₁₀ is C₂-C₆ alkynyl optionally substituted with one or more R^{b}.

In some embodiments, R₁₀ is C₂-C₆ alkynyl substituted with one or more R^{b}.

In some embodiments, R₁₀ is C₂-C₆ alkynyl.

In some embodiments, R₁₀ is C₁-C₆ alkoxyl optionally substituted with one or more R^{b}.

In some embodiments, R₁₀ is C₁-C₆ alkoxyl substituted with one or more R^{b}.

In some embodiments, R₁₀ is C₁-C₆ alkoxyl.

In some embodiments, R₁₀ is C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more R^{b}.

In some embodiments, R₁₀ is C₃-C₆ cycloalkyl, C₆-C₁₀ aryl, 5- to 6-membered heteroaryl, or 3- to 10-membered heterocyclyl.

In some embodiments, R₁₀ is C₃-C₆ cycloalkyl optionally substituted with one or more R^{b}.

In some embodiments, R₁₀ is C₃-C₆ cycloalkyl substituted with one or more R^{b}.

In some embodiments, R₁₀ is C₃-C₆ cycloalkyl.

In some embodiments, R₁₀ is C₆-C₁₀ aryl optionally substituted with one or more R^{b}.

In some embodiments, R₁₀ is C₆-C₁₀ aryl substituted with one or more R^{b}.

In some embodiments, R₁₀ is C₆-C₁₀ aryl.

In some embodiments, R₁₀ is 5- to 6-membered heteroaryl optionally substituted with one or more R^{b}.

In some embodiments, R₁₀ is 5- to 6-membered heteroaryl substituted with one or more R^{b}.

In some embodiments, R₁₀ is 5- to 6-membered heteroaryl.

In some embodiments, R₁₀ is 3- to 10-membered heterocyclyl optionally substituted with one or more R^{b}.

In some embodiments, R₁₀ is 3- to 10-membered heterocyclyl substituted with one or more R^{b}.

In some embodiments, R₁₀ is 3- to 10-membered heterocyclyl.

In some embodiments, R₁₀ is H, —CH₃, CH₂CH₃, isopropyl, CH₂CF₃, CH₂CF₂H,

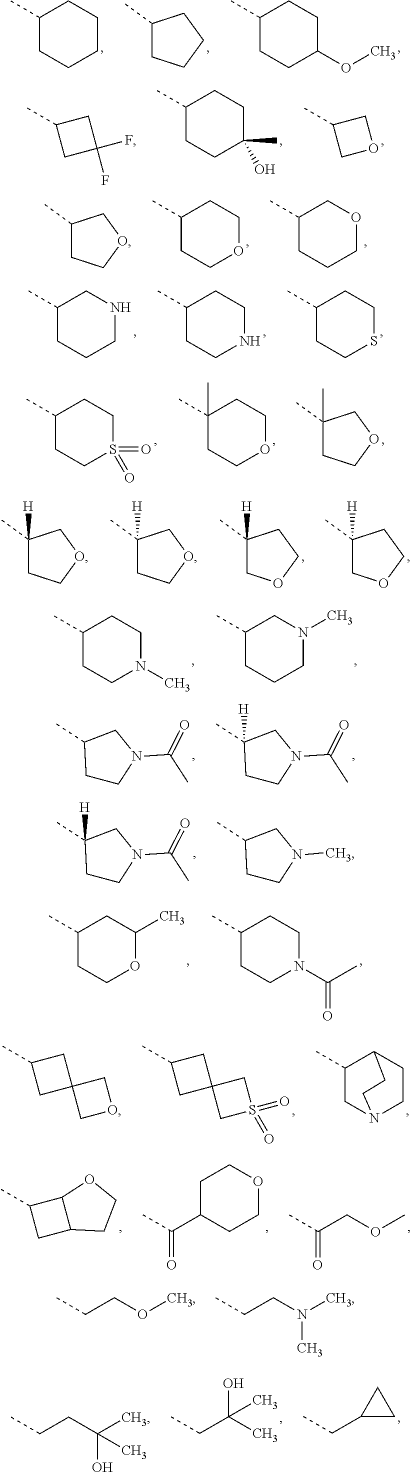

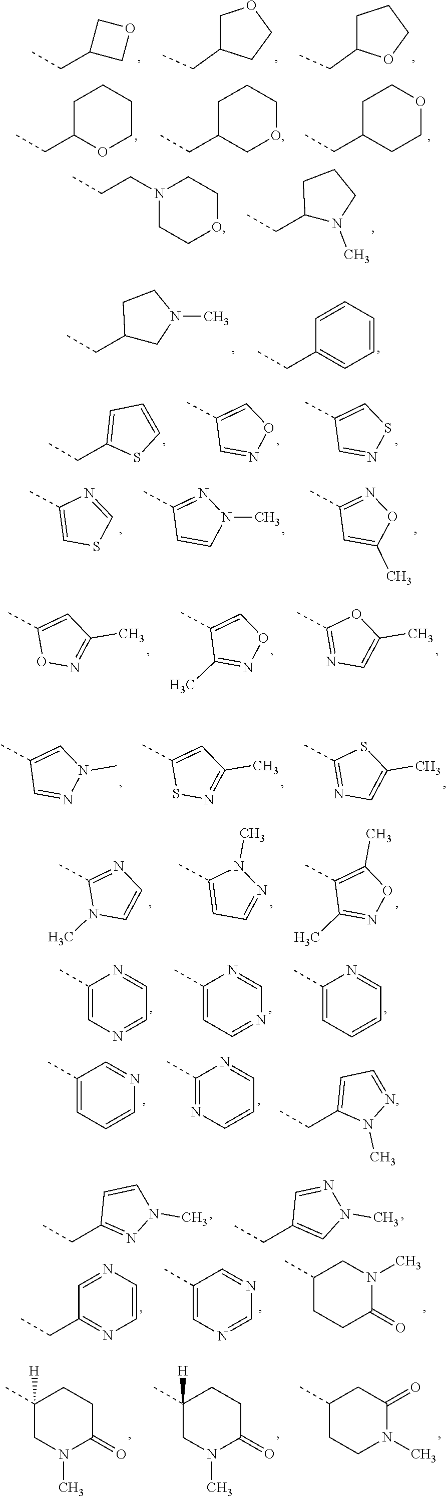

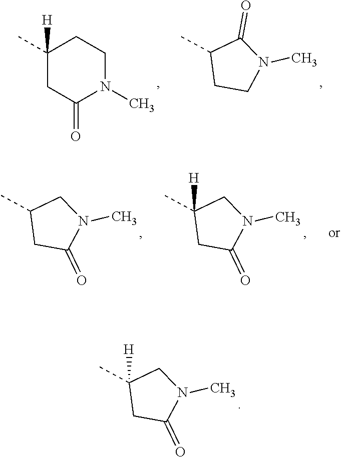

In some embodiments, R₉ and R₁₀, together with the atoms to which they are attached, form 5- to 10-membered heteroaryl or 3- to 10-membered heterocyclyl, wherein the heteroaryl or heterocyclyl is optionally substituted with one or more R^{b}.

In some embodiments, R₉ and R₁₀, together with the atoms to which they are attached, form 5- to 10-membered heteroaryl optionally substituted with one or more R^{b}.

In some embodiments, R₉ and R₁₀, together with the atoms to which they are attached, form 5- to 10-membered heteroaryl substituted with one or more R^{b}.

In some embodiments, R₉ and R₁₀, together with the atoms to which they are attached, form 5- to 10-membered heteroaryl.

In some embodiments, R₉ and R₁₀, together with the atoms to which they are attached, form 3- to 10-membered heterocyclyl optionally substituted with one or more R^{b}.

In some embodiments, R₉ and R₁₀, together with the atoms to which they are attached, form 3- to 10-membered heterocyclyl substituted with one or more R^{b}.

In some embodiments, R₉ and R₁₀, together with the atoms to which they are attached, form 3- to 10-membered heterocyclyl.

In some embodiments, R₉ and R₁₀, together with the atoms to which they are attached, form

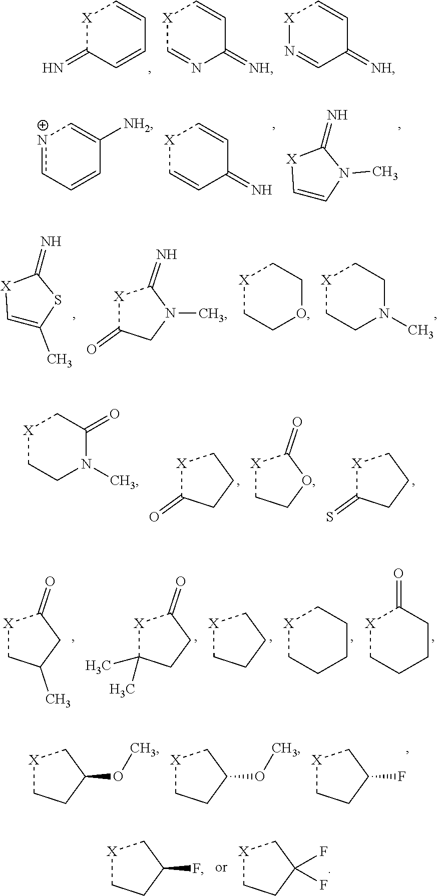

In some embodiments, R^{b} is halo, oxo, =NH, ═S, —CN, —OH, —NH₂, —NH(C₁-C₆ alkyl), —C(O)(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxyl, C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more C₁-C₆ alkyl, C₂-C₆ alkenyl, C₂-C₆ alkynyl, C₁-C₆ haloalkyl, or C₁-C₆ alkoxyl.

In some embodiments, R^{b} is halo, oxo, =NH, ═S, —CN, —OH, —NH₂, —NH(C₁-C₆ alkyl), —C(O)(C₁-C₆ alkyl), —N(C₁-C₆ alkyl)₂.

In some embodiments, R^{b} is halo.

In some embodiments, R^{b} is F, Cl, Br, or I. In some embodiments, R^{b} is F. In some embodiments, R^{b} is Cl. In some embodiments, R^{b} is Br. In some embodiments, R^{b} is I.

In some embodiments, R^{b} is oxo.

In some embodiments, R^{b} is =NH or =S.

In some embodiments, R^{b} is =NH. In some embodiments, R^{b} is =S.

In some embodiments, R^{b} is —CN.

In some embodiments, R^{b} is —OH.

In some embodiments, R^{b} is —NH₂.

In some embodiments, R^{b} is —NH(C₁-C₆ alkyl), —C(O)(C₁-C₆ alkyl), or —N(C₁-C₆ alkyl)₂.

In some embodiments, R^{b} is —NH(C₁-C₆ alkyl). In some embodiments, R^{b} is —C(O)(C₁-C₆ alkyl). In some embodiments, R^{b} is —N(C₁-C₆ alkyl)₂.

In some embodiments, R^{b} is C₁-C₆ alkyl, C₂-C₆ alkenyl, C₂-C₆ alkynyl, C₁-C₆ haloalkyl, C₁-C₆ alkoxyl, C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more C₁-C₆ alkyl, C₂-C₆ alkenyl, C₂-C₆ alkynyl, C₁-C₆ haloalkyl, or C₁-C₆ alkoxyl.

In some embodiments, R^{b} is C₁-C₆ alkyl, C₂-C₆ alkenyl, C₂-C₆ alkynyl, C₁-C₆ haloalkyl, C₁-C₆ alkoxyl, C₃-C₆ cycloalkyl, C₆-C₁₀ aryl, 5- to 6-membered heteroaryl, or 3- to 10-membered heterocyclyl.

In some embodiments, R^{b} is C₁-C₆ alkyl, C₂-C₆ alkenyl, C₂-C₆ alkynyl, C₁-C₆ haloalkyl, or C₁-C₆ alkoxyl.

In some embodiments, R^{b} is C₁-C₆ alkyl, C₂-C₆ alkenyl, or C₂-C₆ alkynyl.

In some embodiments, R^{b} is C₁-C₆ alkyl.

In some embodiments, R^{b} is C₂-C₆ alkenyl.

In some embodiments, R^{b} is C₂-C₆ alkynyl.

In some embodiments, R^{b} is C₁-C₆ haloalkyl.

In some embodiments, R^{b} is C₁-C₆ alkoxyl.

In some embodiments, R^{b} is C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more C₁-C₆ alkyl, C₂-C₆ alkenyl, C₂-C₆ alkynyl, C₁-C₆ haloalkyl, or C₁-C₆ alkoxyl.

In some embodiments, R^{b} is C₃-C₆ cycloalkyl, C₆-C₁₀ aryl, 5- to 6-membered heteroaryl, or 3- to 10-membered heterocyclyl.

In some embodiments, R^{b} is C₃-C₆ cycloalkyl optionally substituted with one or more C₁-C₆ alkyl, C₂-C₆ alkenyl, C₂-C₆ alkynyl, C₁-C₆ haloalkyl, or C₁-C₆ alkoxyl.

In some embodiments, R^{b} is C₃-C₆ cycloalkyl substituted with one or more C₁-C₆ alkyl, C₂-C₆ alkenyl, C₂-C₆ alkynyl, C₁-C₆ haloalkyl, or C₁-C₆ alkoxyl.

In some embodiments, R^{b} is C₃-C₆ cycloalkyl.

In some embodiments, R^{b} is C₆-C₁₀ aryl optionally substituted with one or more C₁-C₆ alkyl, C₂-C₆ alkenyl, C₂-C₆ alkynyl, C₁-C₆ haloalkyl, or C₁-C₆ alkoxyl.

In some embodiments, R^{b} is C₆-C₁₀ aryl substituted with one or more C₁-C₆ alkyl, C₂-C₆ alkenyl, C₂-C₆ alkynyl, C₁-C₆ haloalkyl, or C₁-C₆ alkoxyl.

In some embodiments, R^{b} is C₆-C₁₀ aryl.

In some embodiments, R^{b} is 5- to 6-membered heteroaryl optionally substituted with one or more C₁-C₆ alkyl, C₂-C₆ alkenyl, C₂-C₆ alkynyl, C₁-C₆ haloalkyl, or C₁-C₆ alkoxyl.

In some embodiments, R^{b} is 5- to 6-membered heteroaryl substituted with one or more C₁-C₆ alkyl, C₂-C₆ alkenyl, C₂-C₆ alkynyl, C₁-C₆ haloalkyl, or C₁-C₆ alkoxyl.

In some embodiments, R^{b} is 5- to 6-membered heteroaryl.

In some embodiments, R^{b} is 3- to 10-membered heterocyclyl optionally substituted with one or more C₁-C₆ alkyl, C₂-C₆ alkenyl, C₂-C₆ alkynyl, C₁-C₆ haloalkyl, or C₁-C₆ alkoxyl.

In some embodiments, R^{b} is 3- to 10-membered heterocyclyl substituted with one or more C₁-C₆ alkyl, C₂-C₆ alkenyl, C₂-C₆ alkynyl, C₁-C₆ haloalkyl, or C₁-C₆ alkoxyl.

In some embodiments, R^{b} is 3- to 10-membered heterocyclyl.

In some embodiments, R^{b} is halo, oxo, =NH, ═S, —OH, —NH₂, —C(O)(C₁-C₆ alkyl), C₁-C₆ alkyl, C₁-C₆ haloalkyl, C₁-C₆ alkoxyl, C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more C₁-C₆ alkyl.

In some embodiments, R₁ is H; R₂ is C₁-C₆ alkyl optionally substituted with one or more R^{a}; R₃ is H; and R₄ is C₁-C₆ alkyl.

In some embodiments, X is NR₉; R₁ is H; R₂ is halo; R₃ is H; R₄ is C₁-C₆ alkyl; R₅ is H; R₆ is H; R₇ is H; R₈ is H; R₉ is H; and R₁₀ is 3- to 10-membered heterocyclyl optionally substituted with one or more R^{b}.

In some embodiments, X is NR₉; R₁ is H; R₂ is halo; R₃ is H; R₄ is C₁-C₆ alkyl; R₅ is H; R₆ is H; R₇ is H; R₈ is H; R₉ is C₁-C₆ alkyl; and R₁₀ is 3- to 10-membered heterocyclyl optionally substituted with one or more R^{b}.

In some embodiments, X is NR₉; R₁ is H; R₂ is halo; R₃ is H; R₄ is C₁-C₆ alkyl; R₅ is H; R₆ is H; R₇ is H; R₈ is H; R₉ is H; and R₁₀ is 5- to 6-membered heteroaryl optionally substituted with one or more R^{b}.

In some embodiments, X is NR₉; R₁ is H; R₂ is halo; R₃ is H; R₄ is H; R₅ is H; R₆ is H; R₇ is H; R₈ is H; R₉ is H; and R₁₀ is 3- to 10-membered heterocyclyl optionally substituted with one or more R^{b}.

In some embodiments, X is NR₉; R₁ is H; R₂ is halo; R₃ is H; R₄ is halo; R₅ is H; R₆ is H; R₇ is H; R₈ is H; R₉ is H; and R₁₀ is 3- to 10-membered heterocyclyl optionally substituted with one or more R^{b}.

In some embodiments, X is NR₉; R₁ is H; R₂ is —NO₂; R₃ is H; R₄ is C₁-C₆ alkyl; R₅ is H; R₆ is H; R₇ is H; R₈ is H; R₉ is H; and R₁₀ is 3- to 10-membered heterocyclyl optionally substituted with one or more R^{b}.

In some embodiments, X is NR₉; R₁ is H; R₂ is C₁-C₆ alkyl optionally substituted with one or more R^{a}; R₃ is H; R₄ is H; R₅ is C₁-C₃ alkyl; R₆ is H; R₇ is H; R₈ is H; R₉ is H; and R₁₀ is 3- to 10-membered heterocyclyl optionally substituted with one or more R^{b}.

In some embodiments, X is NR₉; R₁ is H; R₂ is C₁-C₆ alkyl optionally substituted with one or more R^{a}; R₃ is H; R₄ is C₁-C₆ alkyl; R₅ is H; R₆ is H; R₇ is H; R₈ is H; R₉ is H; and R₁₀ is H.

In some embodiments, X is NR₉; R₁ is H; R₂ is C₁-C₆ alkyl optionally substituted with one or more R^{a}; R₃ is H; R₄ is C₁-C₆ alkyl; R₅ is H; R₆ is H; R₇ is H; R₈ is H; R₉ is H; and R₁₀ is C₁-C₆ alkyl.

In some embodiments, X is NR₉; R₁ is H; R₂ is C₁-C₆ alkyl optionally substituted with one or more R^{a}; R₃ is H; R₄ is C₁-C₆ alkyl; R₅ is H; R₆ is H; R₇ is H; R₈ is H; R₉ is C₁-C₆ alkyl; and R₁₀ is C₁-C₆ alkyl optionally substituted with one or more R^{b}.

In some embodiments, X is NR₉; R₁ is H; R₂ is C₁-C₆ alkyl optionally substituted with one or more R^{a}; R₃ is H; R₄ is C₁-C₆ alkyl; R₅ is H; R₆ is H; R₇ is H; R₈ is H; R₉ is H; and R₁₀ is C₁-C₆ alkyl optionally substituted with one or more R^{b}.

In some embodiments, X is NR₉; R₁ is H; R₂ is C₁-C₆ alkyl optionally substituted with one or more R^{a}; R₃ is H; R₄ is C₁-C₆ alkyl; R₅ is H; R₆ is H; R₇ is H; R₈ is H; R₉ is H; and R₁₀ is C₃-C₆ cycloalkyl optionally substituted with one or more R^{b}.

In some embodiments, X is NR₉; R₁ is H; R₂ is C₁-C₆ alkyl optionally substituted with one or more R^{a}; R₃ is H; R₄ is C₁-C₆ alkyl; R₅ is H; R₆ is H; R₇ is H; R₈ is H; R₉ is H; and R₁₀ is 3- to 10-membered heterocyclyl optionally substituted with one or more R^{b}.

In some embodiments, X is NR₉; R₁ is H; R₂ is C₁-C₆ alkyl optionally substituted with one or more R^{a}; R₃ is H; R₄ is C₁-C₆ alkyl; R₅ is H; R₆ is H; R₇ is H; R₈ is H; R₉ is H; and R₁₀ is 5- to 6-membered heteroaryl optionally substituted with one or more R^{b}.

In some embodiments, X is NR₉; R₁ is H; R₂ is C₁-C₆ alkyl optionally substituted with one or more R^{a}; R₃ is H; R₄ is C₁-C₆ alkyl; R₅ is C₁-C₃ alkyl; R₆ is H; R₇ is H; R₈ is H; R₉ is H; and R₁₀ is 3- to 10-membered heterocyclyl optionally substituted with one or more R^{b}.

In some embodiments, X is NR₉; R₁ is H; R₂ is C₁-C₆ alkyl optionally substituted with one or more R^{a}; R₃ is H; R₄ is halo; R₅ is H; R₆ is H; R₇ is H; R₈ is H; R₉ is H; and R₁₀ is 3- to 10-membered heterocyclyl optionally substituted with one or more R^{b}.

In some embodiments, X is NR₉; R₁ is H; R₂ is C₁-C₆ alkyl optionally substituted with one or more R^{a}; R₃ is C₁-C₆ alkyl; R₄ is C₁-C₆ alkyl; R₅ is H; R₆ is H; R₇ is H; R₈ is H; R₉ is H; and R₁₀ is 3- to 10-membered heterocyclyl optionally substituted with one or more R^{b}.

In some embodiments, X is NR₉; R₁ is C₁-C₆ alkyl; R₂ is C₁-C₆ alkyl optionally substituted with one or more R^{a}; R₃ is H; R₄ is H; R₅ is H; R₆ is H; R₇ is H; R₈ is H; R₉ is H; and R₁₀ is 3- to 10-membered heterocyclyl optionally substituted with one or more R^{b}.

In some embodiments, X is NR₉; R₁ is H; R₂ is C₁-C₆ alkoxyl optionally substituted with one or more R^{a}; R₃ is H; R₄ is C₁-C₆ alkyl; R₅ is H; R₆ is H; R₇ is H; R₈ is H; R₉ is H; and R₁₀ is 3- to 10-membered heterocyclyl optionally substituted with one or more R^{b}.

In some embodiments, X is NR₉; R₁ is H; R₂ is C₃-C₆ cyclopropyl optionally substituted with one or more R^{a}; R₃ is H; R₄ is C₁-C₆ alkyl; R₅ is H; R₆ is H; R₇ is H; R₈ is H; R₉ is H; and R₁₀ is 3- to 10-membered heterocyclyl optionally substituted with one or more R^{b}.

In some embodiments, X is NR₉; R₁ is H; R₂ is C₁-C₆ alkyl optionally substituted with one or more R^{a}; R₃ is H; R₄ is C₁-C₆ alkyl; R₅ is H; R₆ is H; R₇ is H; R₈ is H; and R₉ and R₁₀ together with the atoms to which they are attached, form 5- to 10-membered heteroaryl or 3- to 10-membered heterocyclyl, wherein the heteroaryl or heterocyclyl is optionally substituted with one or more R^{b}.

In some embodiments, X is NR₉; R₁ is H; R₂ is C₁-C₆ alkyl optionally substituted with one or more R^{a}; R₃ is H; R₄ is C₁-C₆ alkyl; R₅ is H; R₆ is H; R₇ is C₁-C₆ alkyl; R₈ is H; and R₉ and R₁₀ together with the atoms to which they are attached, form 5- to 10-membered heteroaryl or 3- to 10-membered heterocyclyl, wherein the heteroaryl or heterocyclyl is optionally substituted with one or more R^{b}.

In some embodiments, X is NR₉; R₁ is H; R₂ is C₁-C₆ alkyl optionally substituted with one or more R^{a}; R₃ is H; R₄ is C₁-C₆ alkyl; R₅ is H; R₆ is H; R₇ is C₁-C₆ alkyl; R₈ is C₁-C₆ alkyl; and R₉ and R₁₀ together with the atoms to which they are attached, form 5- to 10-membered heteroaryl or 3- to 10-membered heterocyclyl, wherein the heteroaryl or heterocyclyl is optionally substituted with one or more R^{b}.

In some embodiments, X is NR₉; R₁ is H; R₂ is halo; R₃ is H; R₄ is C₁-C₆ alkyl; R₅ is H; R₆ is H; R₇ is H; R₈ is H; and R₉ and R₁₀ together with the atoms to which they are attached, form 5- to 10-membered heteroaryl or 3- to 10-membered heterocyclyl, wherein the heteroaryl or heterocyclyl is optionally substituted with one or more R^{b}.

In some embodiments, X is NR₉; R₁ is H; R₂ is C₁-C₆ alkyl optionally substituted with one or more R^{a}; R₃ is H; R₄ is C₁-C₆ alkyl; R₅ is H; R₆ is H; R₇ and R₈ together with the atoms to which they are attached, form C₃-C₇ cycloalkyl; and R₉ and R₁₀ together with the atoms to which they are attached, form 5- to 10-membered heteroaryl or 3- to 10-membered heterocyclyl, wherein the heteroaryl or heterocyclyl is optionally substituted with one or more R^{b}.

In some embodiments, X is NR₉; R₁ is H; R₂ is halo; R₃ is H; R₄ is C₁-C₆ alkyl; R₅ is H; R₆ is H; R₇ is H; R₈ is H; R₉ is H; and R₁₀ is 3- to 10-membered heterocyclyl optionally substituted with one or more oxo, —C(O)C₁-C₆ alkyl, or C₁-C₆ alkyl.

In some embodiments, X is NR₉; R₁ is H; R₂ is halo; R₃ is H; R₄ is C₁-C₆ alkyl; R₅ is H; R₆ is H; R₇ is H; R₈ is H; R₉ is C₁-C₆ alkyl; and R₁₀ is 3- to 10-membered heterocyclyl optionally substituted with one or more oxo or C₁-C₆ alkyl.

In some embodiments, X is NR₉; R₁ is H; R₂ is halo; R₃ is H; R₄ is C₁-C₆ alkyl; R₅ is H; R₆ is H; R₇ is H; R₈ is H; R₉ is H; and R₁₀ is 5- to 6-membered heteroaryl optionally substituted with one or more oxo or C₁-C₆ alkyl.

In some embodiments, X is NR₉; R₁ is H; R₂ is halo; R₃ is H; R₄ is H; R₅ is H; R₆ is H; R₇ is H; R₈ is H; R₉ is H; and R₁₀ is 3- to 10-membered heterocyclyl optionally substituted with one or more oxo or C₁-C₆ alkyl.

In some embodiments, X is NR₉; R₁ is H; R₂ is halo; R₃ is H; R₄ is halo; R₅ is H; R₆ is H; R₇ is H; R₈ is H; R₉ is H; and R₁₀ is 3- to 10-membered heterocyclyl optionally substituted with one or more oxo or C₁-C₆ alkyl.

In some embodiments, X is NR₉; R₁ is H; R₂ is —NO₂; R₃ is H; R₄ is C₁-C₆ alkyl; R₅ is H; R₆ is H; R₇ is H; R₈ is H; R₉ is H; and R₁₀ is 3- to 10-membered heterocyclyl optionally substituted with one or more oxo or C₁-C₆ alkyl.

In some embodiments, X is NR₉; R₁ is H; R₂ is C₁-C₆ alkyl optionally substituted with one or more halo; R₃ is H; R₄ is H; R₅ is C₁-C₃ alkyl; R₆ is H; R₇ is H; R₈ is H; R₉ is H; and R₁₀ is 3- to 10-membered heterocyclyl optionally substituted with one or more oxo or C₁-C₆ alkyl.

In some embodiments, X is NR₉; R₁ is H; R₂ is C₁-C₆ alkyl optionally substituted with one or more halo; R₃ is H; R₄ is C₁-C₆ alkyl; R₅ is H; R₆ is H; R₇ is H; R₈ is H; R₉ is H; and R₁₀ is H.

In some embodiments, X is NR₉; R₁ is H; R₂ is C₁-C₆ alkyl optionally substituted with one or more halo; R₃ is H; R₄ is C₁-C₆ alkyl; R₅ is H; R₆ is H; R₇ is H; R₈ is H; R₉ is H; and R₁₀ is C₁-C₆ alkyl.

In some embodiments, X is NR₉; R₁ is H; R₂ is C₁-C₆ alkyl optionally substituted with one or more halo; R₃ is H; R₄ is C₁-C₆ alkyl; R₅ is H; R₆ is H; R₇ is H; R₈ is H; R₉ is C₁-C₆ alkyl; and R₁₀ is C₁-C₆ alkyl optionally substituted with one or more halo.

In some embodiments, X is NR₉; R₁ is H; R₂ is C₁-C₆ alkyl optionally substituted with one or more halo; R₃ is H; R₄ is C₁-C₆ alkyl; R₅ is H; R₆ is H; R₇ is H; R₈ is H; R₉ is H; and R₁₀ is C₁-C₆ alkyl optionally substituted with one or more oxo, —OH, —NH₂, C₁-C₆ alkoxyl, C₃-C₆ cycloalkyl, C₆-C₁₀ aryl, 5- to 6-membered heteroaryl, or 3- to 10-membered heterocyclyl, wherein the heterocyclyl or heteroaryl is optionally substituted with one or more C₁-C₆ alkyl.

In some embodiments, X is NR₉; R₁ is H; R₂ is C₁-C₆ alkyl optionally substituted with one or more halo; R₃ is H; R₄ is C₁-C₆ alkyl; R₅ is H; R₆ is H; R₇ is H; R₈ is H; R₉ is H; and R₁₀ is C₃-C₆ cycloalkyl optionally substituted with one or more oxo, halo, —OH, C₁-C₆ alkyl, or C₁-C₆ alkoxyl.

In some embodiments, X is NR₉; R₁ is H; R₂ is C₁-C₆ alkyl optionally substituted with one or more halo; R₃ is H; R₄ is C₁-C₆ alkyl; R₅ is H; R₆ is H; R₇ is H; R₈ is H; R₉ is H; and R₁₀ is 3- to 10-membered heterocyclyl optionally substituted with one or more oxo, —C(O)(C₁-C₆ alkyl), or C₁-C₆ alkyl.

In some embodiments, X is NR₉; R₁ is H; R₂ is C₁-C₆ alkyl optionally substituted with one or more halo; R₃ is H; R₄ is C₁-C₆ alkyl; R₅ is H; R₆ is H; R₇ is H; R₈ is H; R₉ is H; and R₁₀ is 5- to 6-membered heteroaryl optionally substituted with one or more C₁-C₆ alkyl.

In some embodiments, X is NR₉; R₁ is H; R₂ is C₁-C₆ alkyl optionally substituted with one or more halo; R₃ is H; R₄ is C₁-C₆ alkyl; R₅ is C₁-C₃ alkyl; R₆ is H; R₇ is H; R₈ is H; R₉ is H; and R₁₀ is 3- to 10-membered heterocyclyl optionally substituted with one or more oxo or C₁-C₆ alkyl.

In some embodiments, X is NR₉; R₁ is H; R₂ is C₁-C₆ alkyl optionally substituted with one or more halo; R₃ is H; R₄ is halo; R₅ is H; R₆ is H; R₇ is H; R₈ is H; R₉ is H; and R₁₀ is 3- to 10-membered heterocyclyl optionally substituted with one or more oxo or C₁-C₆ alkyl.

In some embodiments, X is NR₉; R₁ is H; R₂ is C₁-C₆ alkyl optionally substituted with one or more halo; R₃ is C₁-C₆ alkyl; R₄ is C₁-C₆ alkyl; R₅ is H; R₆ is H; R₇ is H; R₈ is H; R₉ is H; and R₁₀ is 3- to 10-membered heterocyclyl optionally substituted with one or more oxo or C₁-C₆ alkyl.

In some embodiments, X is NR₉; R₁ is C₁-C₆ alkyl; R₂ is C₁-C₆ alkyl optionally substituted with one or more halo; R₃ is H; R₄ is H; R₅ is H; R₆ is H; R₇ is H; R₈ is H; R₉ is H; and R₁₀ is 3- to 10-membered heterocyclyl optionally substituted with one or more oxo or C₁-C₆ alkyl.

In some embodiments, X is NR₉; R₁ is H; R₂ is C₁-C₆ alkoxyl optionally substituted with one or more halo; R₃ is H; R₄ is C₁-C₆ alkyl; R₅ is H; R₆ is H; R₇ is H; R₈ is H; R₉ is H; and R₁₀ is 3- to 10-membered heterocyclyl optionally substituted with one or more oxo or C₁-C₆ alkyl.

In some embodiments, X is NR₉; R₁ is H; R₂ is C₃-C₆ cyclopropyl optionally substituted with one or more halo; R₃ is H; R₄ is C₁-C₆ alkyl; R₅ is H; R₆ is H; R₇ is H; R₈ is H; R₉ is H; and R₁₀ is 3- to 10-membered heterocyclyl optionally substituted with one or more oxo, —C(O)(C₁-C₆ alkyl), or C₁-C₆ alkyl.

In some embodiments, X is NR₉; R₁ is H; R₂ is C₁-C₆ alkyl optionally substituted with one or more halo; R₃ is H; R₄ is C₁-C₆ alkyl; R₅ is H; R₆ is H; R₇ is H; R₈ is H; and R₉ and R₁₀ together with the atoms to which they are attached, form 5- to 10-membered heteroaryl or 3- to 10-membered heterocyclyl, wherein the heteroaryl or heterocyclyl is optionally substituted with one or more oxo, halo, =NH, —NH₂, C₁-C₆ alkyl, C₁-C₆ alkoxyl.

In some embodiments, X is NR₉; R₁ is H; R₂ is C₁-C₆ alkyl optionally substituted with one or more halo; R₃ is H; R₄ is C₁-C₆ alkyl; R₅ is H; R₆ is H; R₇ is C₁-C₆ alkyl; R₈ is H; and R₉ and R₁₀ together with the atoms to which they are attached, form 5- to 10-membered heteroaryl or 3- to 10-membered heterocyclyl, wherein the heteroaryl or heterocyclyl is optionally substituted with one or more oxo, halo, =NH, —NH₂, C₁-C₆ alkyl, C₁-C₆ alkoxyl.

In some embodiments, X is NR₉; R₁ is H; R₂ is C₁-C₆ alkyl optionally substituted with one or more halo; R₃ is H; R₄ is C₁-C₆ alkyl; R₅ is H; R₆ is H; R₇ is C₁-C₆ alkyl; R₈ is C₁-C₆ alkyl; and R₉ and R₁₀ together with the atoms to which they are attached, form 5- to 10-membered heteroaryl or 3- to 10-membered heterocyclyl, wherein the heteroaryl or heterocyclyl is optionally substituted with one or more oxo, halo, =NH, —NH₂, C₁-C₆ alkyl, C₁-C₆ alkoxyl.

In some embodiments, X is NR₉; R₁ is H; R₂ is halo; R₃ is H; R₄ is C₁-C₆ alkyl; R₅ is H; R₆ is H; R₇ is H; R₈ is H; and R₉ and R₁₀ together with the atoms to which they are attached, form 5- to 10-membered heteroaryl or 3- to 10-membered heterocyclyl, wherein the heteroaryl or heterocyclyl is optionally substituted with one or more oxo, halo, =NH, ═S, —NH₂, C₁-C₆ alkyl, C₁-C₆ alkoxyl.

In some embodiments, X is NR₉; R₁ is H; R₂ is C₁-C₆ alkyl optionally substituted with one or more halo; R₃ is H; R₄ is C₁-C₆ alkyl; R₅ is H; R₆ is H; R₇ and R₈ together with the atoms to which they are attached, form C₃-C₇ cycloalkyl; and R₉ and R₁₀ together with the atoms to which they are attached, form 5- to 10-membered heteroaryl or 3- to 10-membered heterocyclyl, wherein the heteroaryl or heterocyclyl is optionally substituted with one or more oxo, halo, =NH, —NH₂, C₁-C₆ alkyl, C₁-C₆ alkoxyl.

In some embodiments, X is O; R₁ is H; R₂ is C₁-C₆ alkyl optionally substituted with one or more R^{a}; R₃ is H; R₄ is C₁-C₆ alkyl; R₅ is H; R₆ is H; R₇ is H; R₈ is H; and R₁₀ is 3- to 10-membered heterocyclyl optionally substituted with one or more R^{b}.

In some embodiments, X is O; R₁ is H; R₂ is halo; R₃ is H; R₄ is C₁-C₆ alkyl; R₅ is H; R₆ is H; R₇ is H; R₈ is H; and R₁₀ is 3- to 10-membered heterocyclyl optionally substituted with one or more R^{b}.

In some embodiments, X is O; R₁ is H; R₂ is C₁-C₆ alkyl optionally substituted with one or more halo; R₃ is H; R₄ is C₁-C₆ alkyl; R₅ is H; R₆ is H; R₇ is H; R₈ is H; and R₁₀ is 3- to 10-membered heterocyclyl optionally substituted with one or more oxo or C₁-C₆ alkyl.

In some embodiments, X is O; R₁ is H; R₂ is halo; R₃ is H; R₄ is C₁-C₆ alkyl; R₅ is H; R₆ is H; R₇ is H; R₈ is H; and R₁₀ is 3- to 10-membered heterocyclyl optionally substituted with one or more oxo. —C(O)C₁-C₆ alkyl, or C₁-C₆ alkyl.

In some embodiments, the compound of Formula (I) is of Formula (I-a):

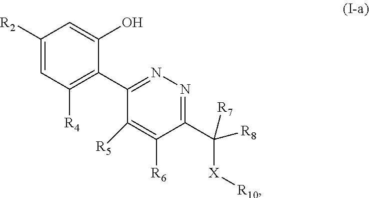

or a prodrug, solvate, or pharmaceutically acceptable salt thereof.

In some embodiments, the compound of Formula (I) is of Formula (I-b):

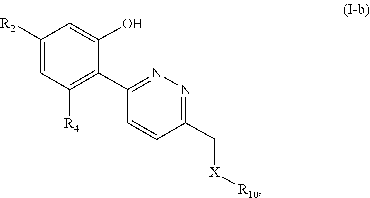

or a prodrug, solvate, or pharmaceutically acceptable salt thereof.

In some embodiments, the compound of Formula (I) is of Formula (I-c):

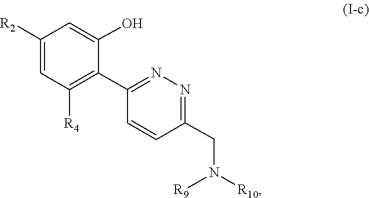

or a prodrug, solvate, or pharmaceutically acceptable salt thereof.

In some embodiments, the compound of Formula (I) is of Formula (I-d):

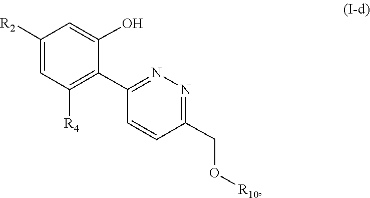

or a prodrug, solvate, or pharmaceutically acceptable salt thereof.

In some embodiments, the compound of Formula (I) is of Formula (I-e):

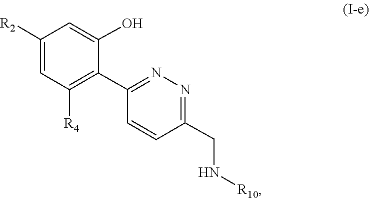

or a prodrug, solvate, or pharmaceutically acceptable salt thereof.

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound is selected from the compounds described in Table 1.

TABLE 1

| Compound | |
|---|---|
| Number | Structure |
| 1 | 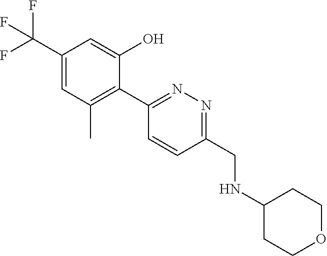 [SMILES: Cc1cc(C(F)(F)F)cc(O)c1-c1ccc(CNC2CCOCC2)nn1] |
| 2 | 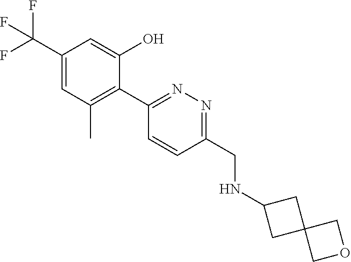 [SMILES: Cc1cc(C(F)(F)F)cc(O)c1-c1ccc(CNC2CC3(COC3)C2)nn1] |
| 3 | 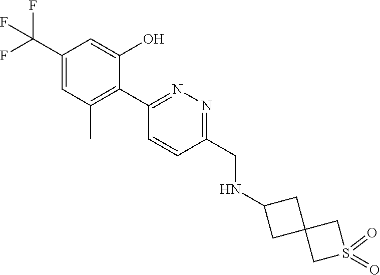 [SMILES: Cc1cc(C(F)(F)F)cc(O)c1-c1ccc(CNC2CC3(C2)CS(=O)(=O)C3)nn1] |
| 4 | 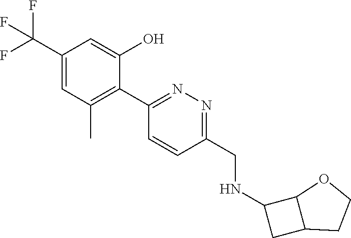 [SMILES: Cc1cc(C(F)(F)F)cc(O)c1-c1ccc(CNC2CC3CCOC32)nn1] |
| 5 | 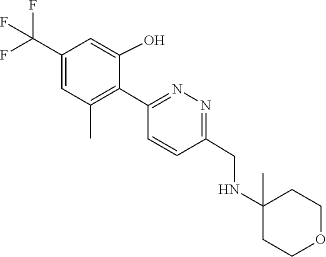 [SMILES: Cc1cc(C(F)(F)F)cc(O)c1-c1ccc(CNC2(C)CCOCC2)nn1] |
| 6 | 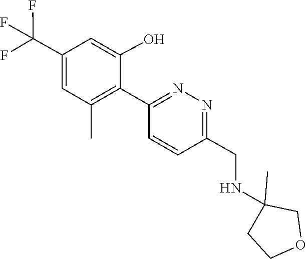 [SMILES: Cc1cc(C(F)(F)F)cc(O)c1-c1ccc(CNC2(C)CCOC2)nn1] |
| 7 | 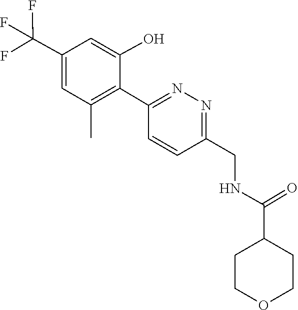 [SMILES: Cc1cc(C(F)(F)F)cc(O)c1-c1ccc(CNC(=O)C2CCOCC2)nn1] |
| 8 | 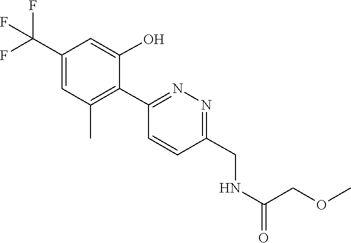 [SMILES: COCC(=O)NCc1ccc(-c2c(C)cc(C(F)(F)F)cc2O)nn1] |
| 9 | 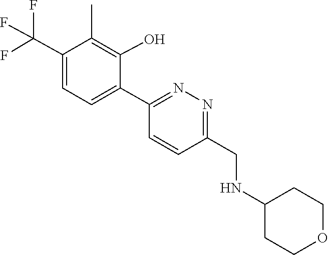 [SMILES: Cc1c(C(F)(F)F)ccc(-c2ccc(CNC3CCOCC3)nn2)c1O] |
| 10 | 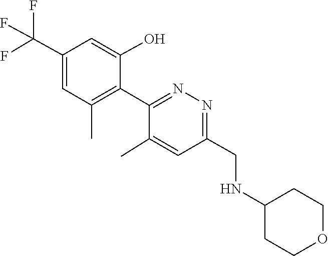 [SMILES: Cc1cc(CNC2CCOCC2)nnc1-c1c(C)cc(C(F)(F)F)cc1O] |
| 11 | 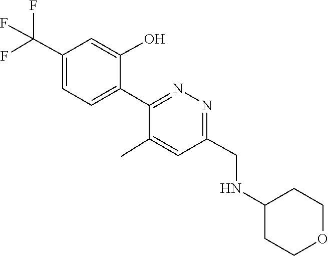 [SMILES: Cc1cc(CNC2CCOCC2)nnc1-c1ccc(C(F)(F)F)cc1O] |
| 12 | 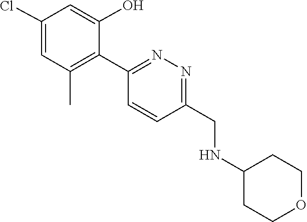 [SMILES: Cc1cc(Cl)cc(O)c1-c1ccc(CNC2CCOCC2)nn1] |
| 13 | 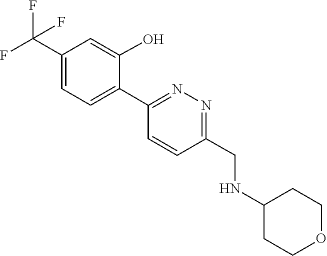 [SMILES: Oc1cc(C(F)(F)F)ccc1-c1ccc(CNC2CCOCC2)nn1] |
| 14 | 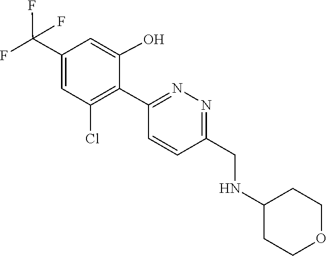 [SMILES: Oc1cc(C(F)(F)F)cc(Cl)c1-c1ccc(CNC2CCOCC2)nn1] |
| 15 | 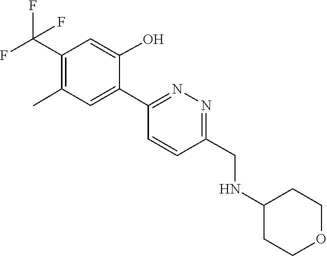 [SMILES: Cc1cc(-c2ccc(CNC3CCOCC3)nn2)c(O)cc1C(F)(F)F] |
| 16 | 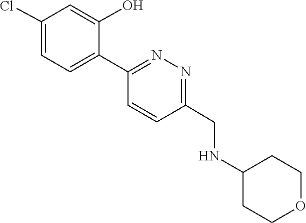 [SMILES: Oc1cc(Cl)ccc1-c1ccc(CNC2CCOCC2)nn1] |
| 17 | 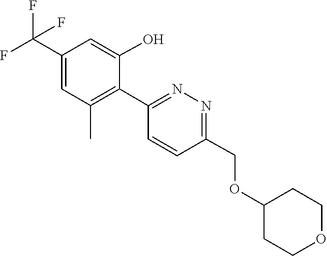 [SMILES: Cc1cc(C(F)(F)F)cc(O)c1-c1ccc(COC2CCOCC2)nn1] |
| 18 | 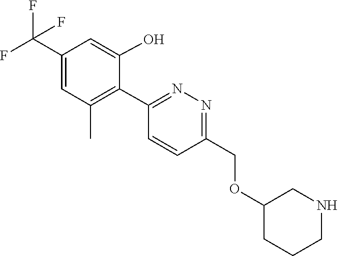 [SMILES: Cc1cc(C(F)(F)F)cc(O)c1-c1ccc(COC2CCCNC2)nn1] |
| 19 | 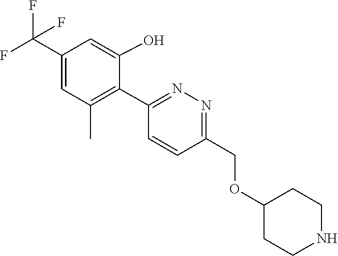 [SMILES: Cc1cc(C(F)(F)F)cc(O)c1-c1ccc(COC2CCNCC2)nn1] |
| 20 | 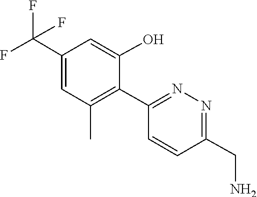 [SMILES: Cc1cc(C(F)(F)F)cc(O)c1-c1ccc(CN)nn1] |
| 21 | 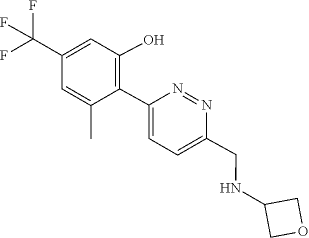 [SMILES: Cc1cc(C(F)(F)F)cc(O)c1-c1ccc(CNC2COC2)nn1] |
| 22 | 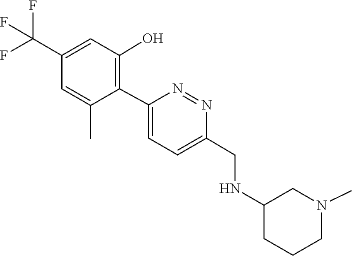 [SMILES: Cc1cc(C(F)(F)F)cc(O)c1-c1ccc(CNC2CCCN(C)C2)nn1] |
| 23 | 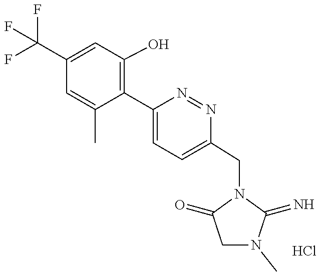 [SMILES: Cc1cc(C(F)(F)F)cc(O)c1-c1ccc(CN2C(=N)N(C)CC2=O)nn1 ; Cl] |
| 24 | 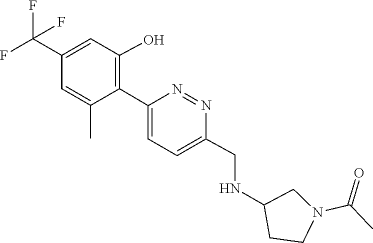 [SMILES: CC(=O)N1CCC(NCc2ccc(-c3c(C)cc(C(F)(F)F)cc3O)nn2)C1] |
| 25 | 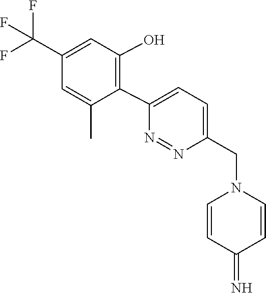 [SMILES: Cc1cc(C(F)(F)F)cc(O)c1-c1ccc(Cn2ccc(=N)cc2)nn1] |
| 26 | 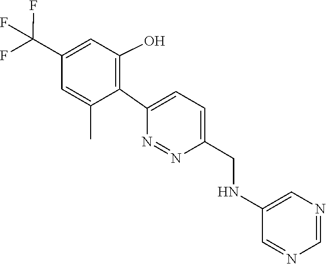 [SMILES: Cc1cc(C(F)(F)F)cc(O)c1-c1ccc(CNc2cncnc2)nn1] |
| 27 | 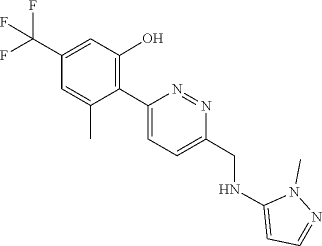 [SMILES: Cc1cc(C(F)(F)F)cc(O)c1-c1ccc(CNc2ccnn2C)nn1] |
| 28 | 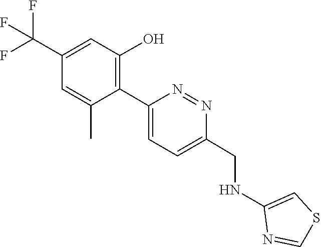 [SMILES: Cc1cc(C(F)(F)F)cc(O)c1-c1ccc(CNc2cscn2)nn1] |
| 29 | 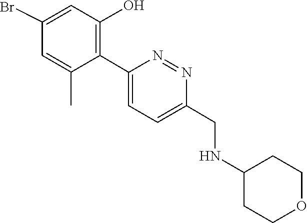 [SMILES: Cc1cc(Br)cc(O)c1-c1ccc(CNC2CCOCC2)nn1] |
| 30 | 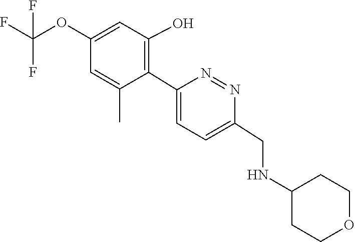 [SMILES: Cc1cc(OC(F)(F)F)cc(O)c1-c1ccc(CNC2CCOCC2)nn1] |
| 31 | 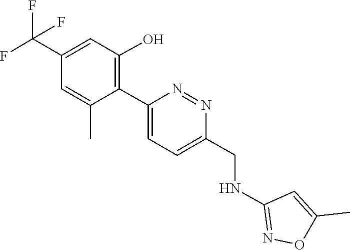 [SMILES: Cc1cc(NCc2ccc(-c3c(C)cc(C(F)(F)F)cc3O)nn2)no1] |
| 32 | 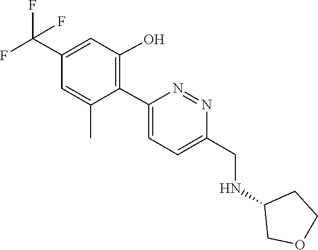 [SMILES: Cc1cc(C(F)(F)F)cc(O)c1-c1ccc(CN[C@@H]2CCOC2)nn1] |
| 33 | 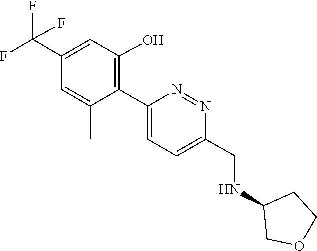 [SMILES: Cc1cc(C(F)(F)F)cc(O)c1-c1ccc(CN[C@H]2CCOC2)nn1] |
| 34 | 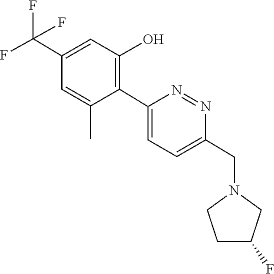 [SMILES: Cc1cc(C(F)(F)F)cc(O)c1-c1ccc(CN2CC[C@@H](F)C2)nn1] |
| 35 | 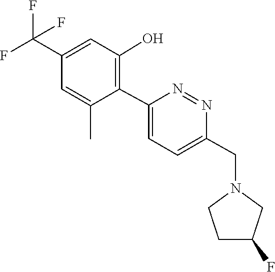 [SMILES: Cc1cc(C(F)(F)F)cc(O)c1-c1ccc(CN2CC[C@H](F)C2)nn1] |
| 36 | 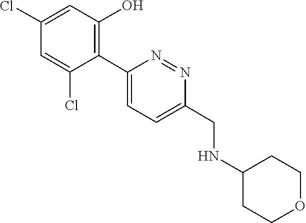 [SMILES: Oc1cc(Cl)cc(Cl)c1-c1ccc(CNC2CCOCC2)nn1] |
| 37 | 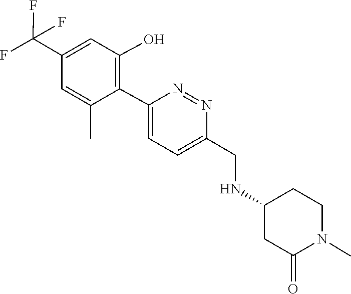 [SMILES: Cc1cc(C(F)(F)F)cc(O)c1-c1ccc(CN[C@@H]2CCN(C)C(=O)C2)nn1] |
| 38 | 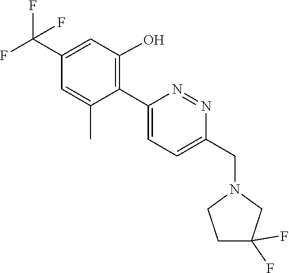 [SMILES: Cc1cc(C(F)(F)F)cc(O)c1-c1ccc(CN2CCC(F)(F)C2)nn1] |
| 39 | 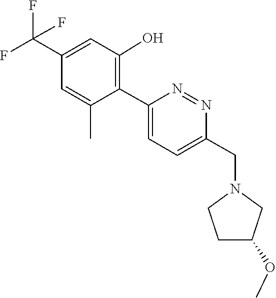 [SMILES: CO[C@@H]1CCN(Cc2ccc(-c3c(C)cc(C(F)(F)F)cc3O)nn2)C1] |
| 40 | 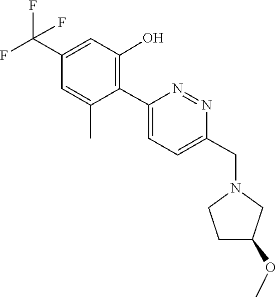 [SMILES: CO[C@H]1CCN(Cc2ccc(-c3c(C)cc(C(F)(F)F)cc3O)nn2)C1] |
| 41 | 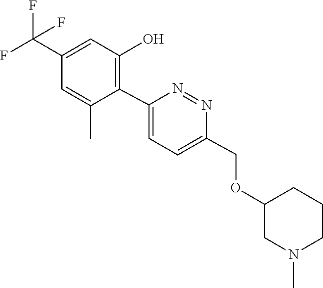 [SMILES: Cc1cc(C(F)(F)F)cc(O)c1-c1ccc(COC2CCCN(C)C2)nn1] |
| 42 | 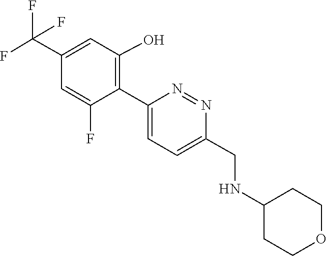 [SMILES: Oc1cc(C(F)(F)F)cc(F)c1-c1ccc(CNC2CCOCC2)nn1] |
| 43 | 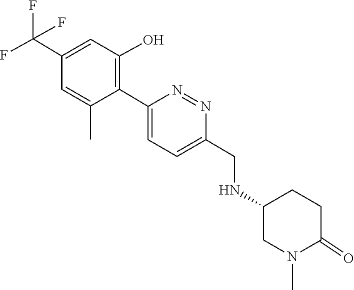 [SMILES: Cc1cc(C(F)(F)F)cc(O)c1-c1ccc(CN[C@@H]2CCC(=O)N(C)C2)nn1] |
| 44 | 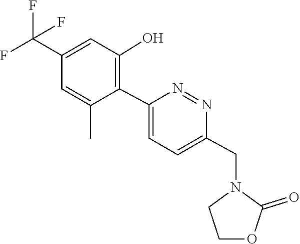 [SMILES: Cc1cc(C(F)(F)F)cc(O)c1-c1ccc(CN2CCOC2=O)nn1] |
| 45 | 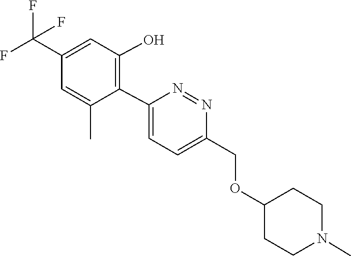 [SMILES: Cc1cc(C(F)(F)F)cc(O)c1-c1ccc(COC2CCN(C)CC2)nn1] |
| 46 | 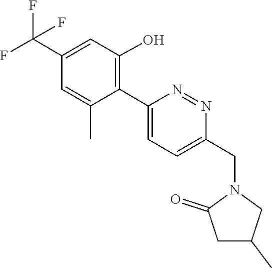 [SMILES: Cc1cc(C(F)(F)F)cc(O)c1-c1ccc(CN2CC(C)CC2=O)nn1] |
| 47 | 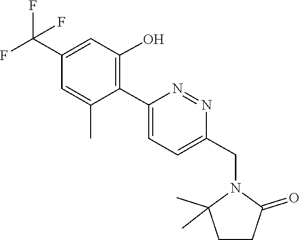 [SMILES: Cc1cc(C(F)(F)F)cc(O)c1-c1ccc(CN2C(=O)CCC2(C)C)nn1] |
| 48 | 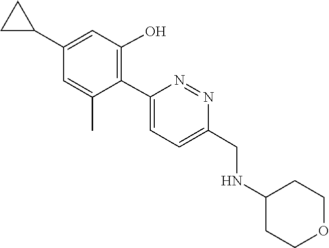 [SMILES: Cc1cc(C2CC2)cc(O)c1-c1ccc(CNC2CCOCC2)nn1] |
| 49 | 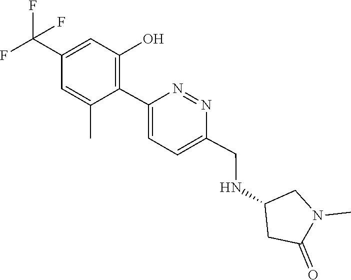 [SMILES: Cc1cc(C(F)(F)F)cc(O)c1-c1ccc(CN[C@H]2CC(=O)N(C)C2)nn1] |
| 50 | 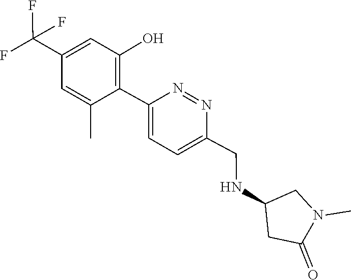 [SMILES: Cc1cc(C(F)(F)F)cc(O)c1-c1ccc(CN[C@@H]2CC(=O)N(C)C2)nn1] |
| 51 | 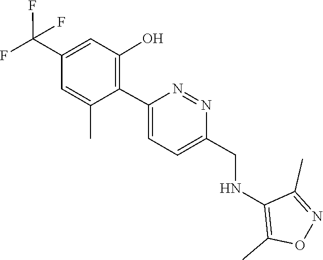 [SMILES: Cc1cc(C(F)(F)F)cc(O)c1-c1ccc(CNc2c(C)noc2C)nn1] |
| 52 | 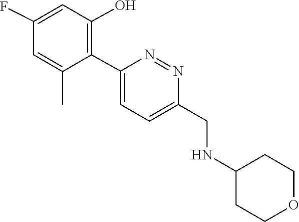 [SMILES: Cc1cc(F)cc(O)c1-c1ccc(CNC2CCOCC2)nn1] |
| 53 | 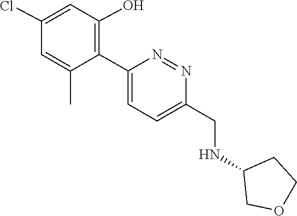 [SMILES: Cc1cc(Cl)cc(O)c1-c1ccc(CN[C@@H]2CCOC2)nn1] |
| 54 | 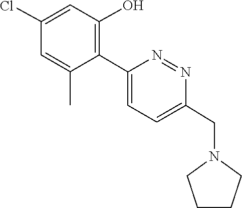 [SMILES: Cc1cc(Cl)cc(O)c1-c1ccc(CN2CCCC2)nn1] |
| 55 | 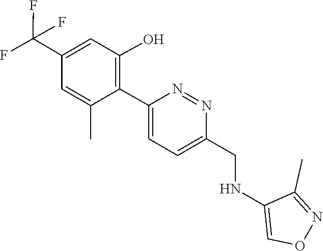 [SMILES: Cc1cc(C(F)(F)F)cc(O)c1-c1ccc(CNc2conc2C)nn1] |
| 56 | 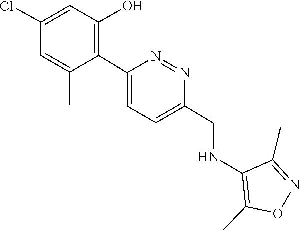 [SMILES: Cc1cc(Cl)cc(O)c1-c1ccc(CNc2c(C)noc2C)nn1] |
| 57 | 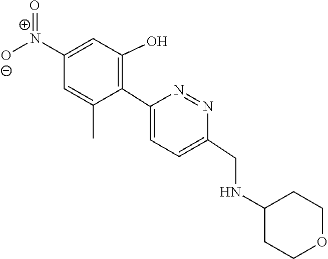 [SMILES: Cc1cc([N+](=O)[O-])cc(O)c1-c1ccc(CNC2CCOCC2)nn1] |
| 58 | 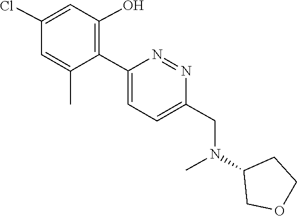 [SMILES: Cc1cc(Cl)cc(O)c1-c1ccc(CN(C)[C@@H]2CCOC2)nn1] |
| 59 | 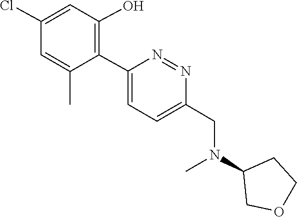 [SMILES: Cc1cc(Cl)cc(O)c1-c1ccc(CN(C)[C@H]2CCOC2)nn1] |
| 60 | 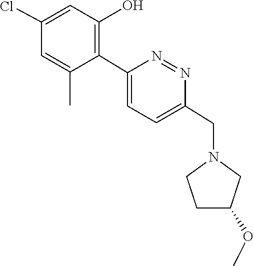 [SMILES: CO[C@@H]1CCN(Cc2ccc(-c3c(C)cc(Cl)cc3O)nn2)C1] |
| 61 | 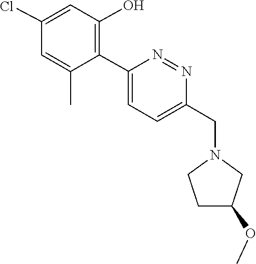 [SMILES: CO[C@H]1CCN(Cc2ccc(-c3c(C)cc(Cl)cc3O)nn2)C1] |
| 62 | 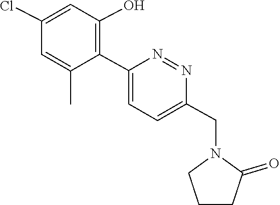 [SMILES: Cc1cc(Cl)cc(O)c1-c1ccc(CN2CCCC2=O)nn1] |
| 63 | 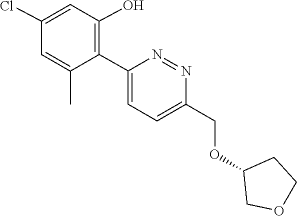 [SMILES: Cc1cc(Cl)cc(O)c1-c1ccc(CO[C@@H]2CCOC2)nn1] |
| 64 | 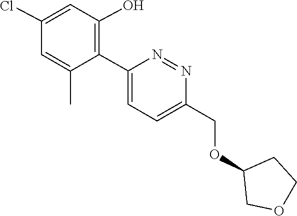 [SMILES: Cc1cc(Cl)cc(O)c1-c1ccc(CO[C@H]2CCOC2)nn1] |
| 65 | 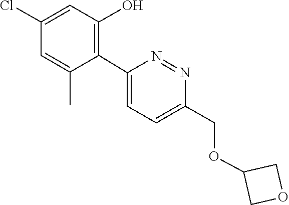 [SMILES: Cc1cc(Cl)cc(O)c1-c1ccc(COC2COC2)nn1] |
| 66 | 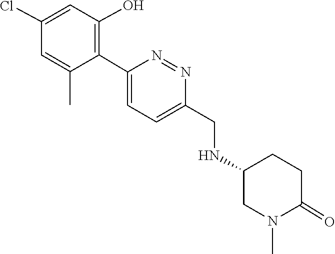 [SMILES: Cc1cc(Cl)cc(O)c1-c1ccc(CN[C@@H]2CCC(=O)N(C)C2)nn1] |
| 67 | 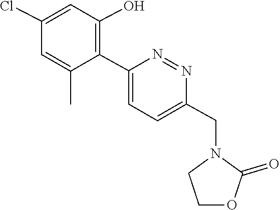 [SMILES: Cc1cc(Cl)cc(O)c1-c1ccc(CN2CCOC2=O)nn1] |
| 68 | 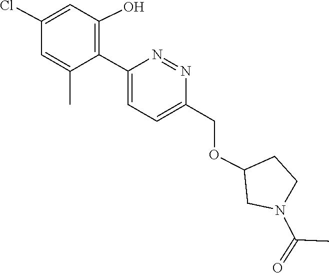 [SMILES: CC(=O)N1CCC(OCc2ccc(-c3c(C)cc(Cl)cc3O)nn2)C1] |
| 69 | 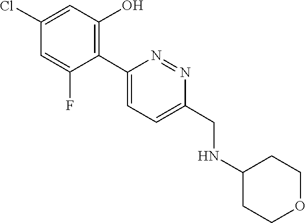 [SMILES: Oc1cc(Cl)cc(F)c1-c1ccc(CNC2CCOCC2)nn1] |
| 70 | 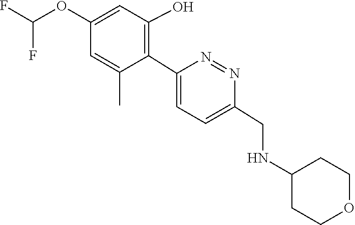 [SMILES: Cc1cc(OC(F)F)cc(O)c1-c1ccc(CNC2CCOCC2)nn1] |
| 71 | 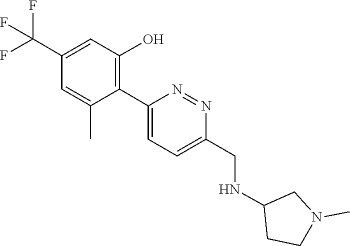 [SMILES: Cc1cc(C(F)(F)F)cc(O)c1-c1ccc(CNC2CCN(C)C2)nn1] |
| 72 | 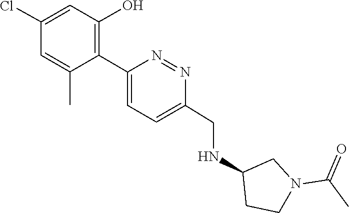 [SMILES: CC(=O)N1CC[C@@H](NCc2ccc(-c3c(C)cc(Cl)cc3O)nn2)C1] |
| 73 | 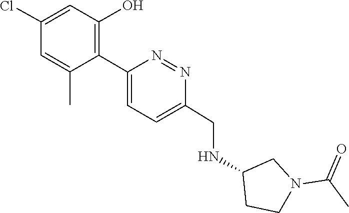 [SMILES: CC(=O)N1CC[C@H](NCc2ccc(-c3c(C)cc(Cl)cc3O)nn2)C1] |
| 74 | 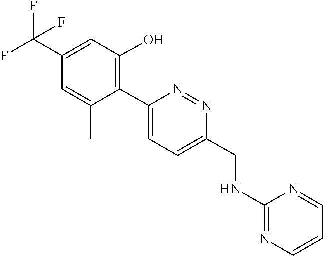 [SMILES: Cc1cc(C(F)(F)F)cc(O)c1-c1ccc(CNc2ncccn2)nn1] |
| 75 | 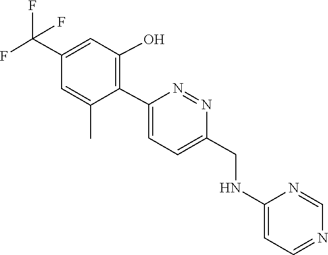 [SMILES: Cc1cc(C(F)(F)F)cc(O)c1-c1ccc(CNc2ccncn2)nn1] |
| 76 | 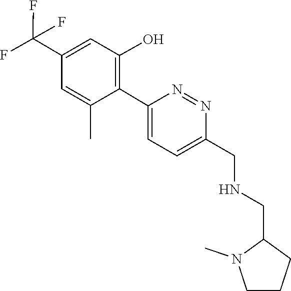 [SMILES: Cc1cc(C(F)(F)F)cc(O)c1-c1ccc(CNCC2CCCN2C)nn1] |
| 77 | 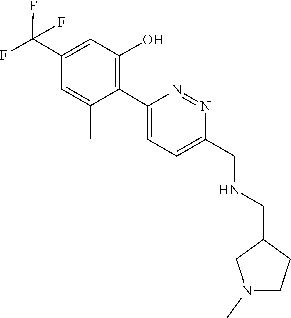 [SMILES: Cc1cc(C(F)(F)F)cc(O)c1-c1ccc(CNCC2CCN(C)C2)nn1] |
| 78 | 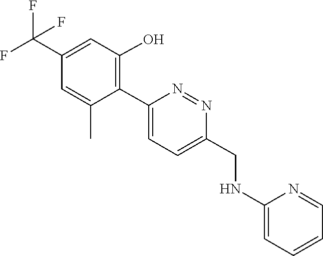 [SMILES: Cc1cc(C(F)(F)F)cc(O)c1-c1ccc(CNc2ccccn2)nn1] |
| 79 | 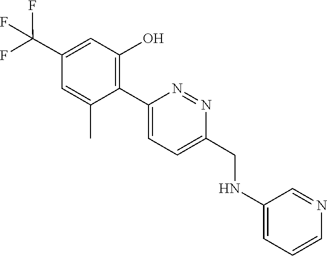 [SMILES: Cc1cc(C(F)(F)F)cc(O)c1-c1ccc(CNc2cccnc2)nn1] |
| 80 | 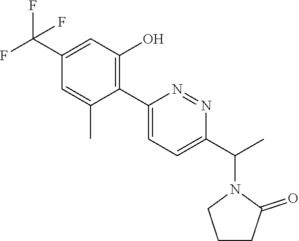 [SMILES: Cc1cc(C(F)(F)F)cc(O)c1-c1ccc(C(C)N2CCCC2=O)nn1] |
| 81 | 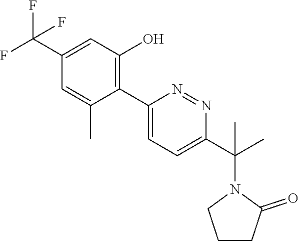 [SMILES: Cc1cc(C(F)(F)F)cc(O)c1-c1ccc(C(C)(C)N2CCCC2=O)nn1] |
| 82 | 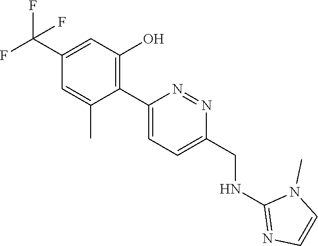 [SMILES: Cc1cc(C(F)(F)F)cc(O)c1-c1ccc(CNc2nccn2C)nn1] |
| 83 | 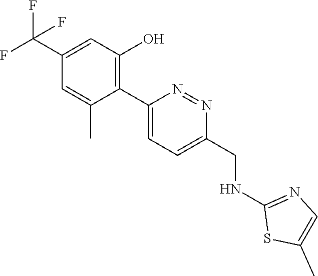 [SMILES: Cc1cnc(NCc2ccc(-c3c(C)cc(C(F)(F)F)cc3O)nn2)s1] |
| 84 | 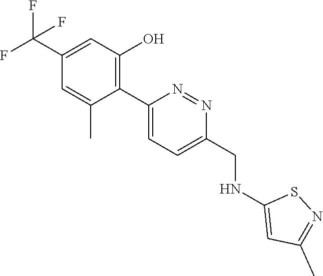 [SMILES: Cc1cc(NCc2ccc(-c3c(C)cc(C(F)(F)F)cc3O)nn2)sn1] |
| 85 | 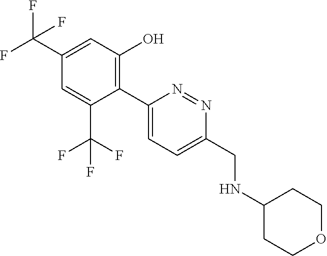 [SMILES: Oc1cc(C(F)(F)F)cc(C(F)(F)F)c1-c1ccc(CNC2CCOCC2)nn1] |
| 86 | 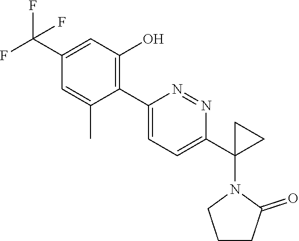 [SMILES: Cc1cc(C(F)(F)F)cc(O)c1-c1ccc(C2(N3CCCC3=O)CC2)nn1] |
| 87 | 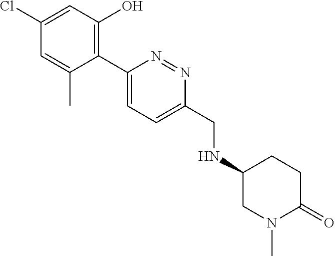 [SMILES: Cc1cc(Cl)cc(O)c1-c1ccc(CN[C@H]2CCC(=O)N(C)C2)nn1] |
| 88 | 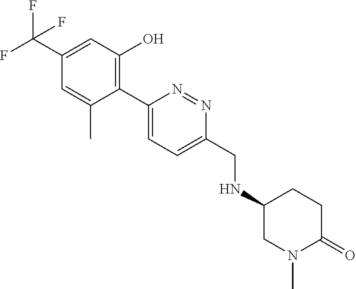 [SMILES: Cc1cc(C(F)(F)F)cc(O)c1-c1ccc(CN[C@H]2CCC(=O)N(C)C2)nn1] |
| 89 | 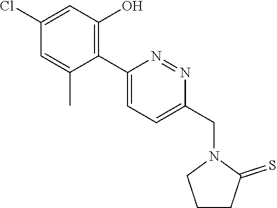 [SMILES: Cc1cc(Cl)cc(O)c1-c1ccc(CN2CCCC2=S)nn1] |
| 90 | 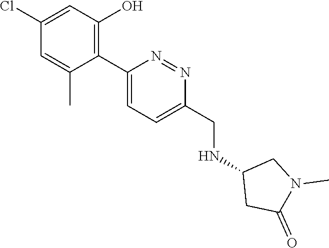 [SMILES: Cc1cc(Cl)cc(O)c1-c1ccc(CN[C@H]2CC(=O)N(C)C2)nn1] |
| 91 | 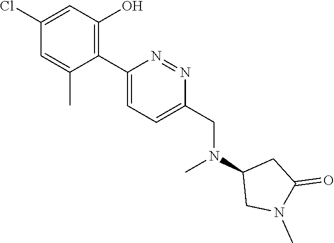 [SMILES: Cc1cc(Cl)cc(O)c1-c1ccc(CN(C)[C@H]2CC(=O)N(C)C2)nn1] |
| 92 | 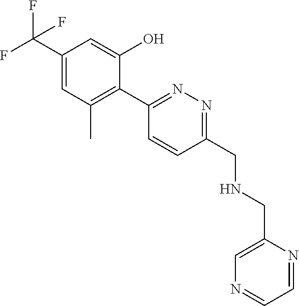 [SMILES: Cc1cc(C(F)(F)F)cc(O)c1-c1ccc(CNCc2cnccn2)nn1] |
| 93 | 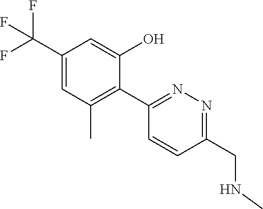 [SMILES: CNCc1ccc(-c2c(C)cc(C(F)(F)F)cc2O)nn1] |
| 94 | 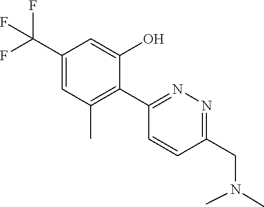 [SMILES: Cc1cc(C(F)(F)F)cc(O)c1-c1ccc(CN(C)C)nn1] |
| 95 | 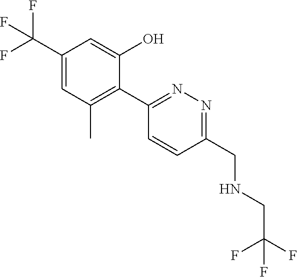 [SMILES: Cc1cc(C(F)(F)F)cc(O)c1-c1ccc(CNCC(F)(F)F)nn1] |
| 96 | 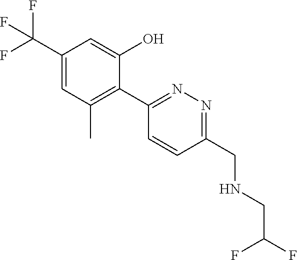 [SMILES: Cc1cc(C(F)(F)F)cc(O)c1-c1ccc(CNCC(F)F)nn1] |
| 97 | 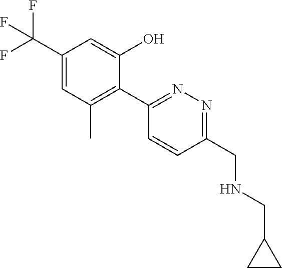 [SMILES: Cc1cc(C(F)(F)F)cc(O)c1-c1ccc(CNCC2CC2)nn1] |
| 98 | 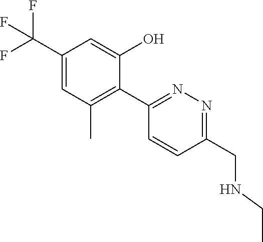 [SMILES: CCNCc1ccc(-c2c(C)cc(C(F)(F)F)cc2O)nn1] |
| 99 | 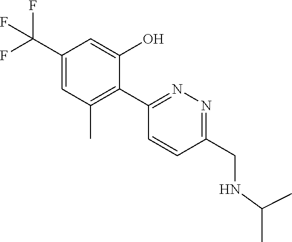 [SMILES: Cc1cc(C(F)(F)F)cc(O)c1-c1ccc(CNC(C)C)nn1] |
| 100 | 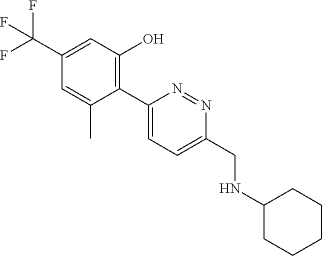 [SMILES: Cc1cc(C(F)(F)F)cc(O)c1-c1ccc(CNC2CCCCC2)nn1] |
| 101 | 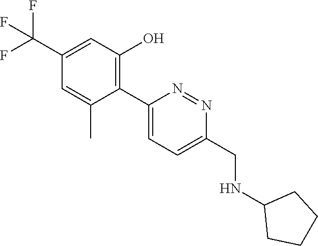 [SMILES: Cc1cc(C(F)(F)F)cc(O)c1-c1ccc(CNC2CCCC2)nn1] |
| 102 | 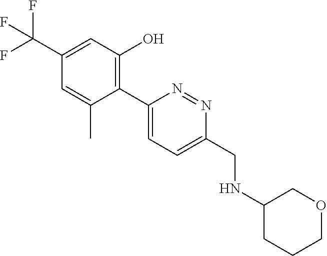 [SMILES: Cc1cc(C(F)(F)F)cc(O)c1-c1ccc(CNC2CCCOC2)nn1] |
| 103 | 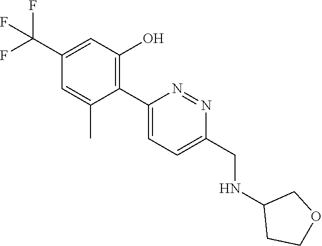 [SMILES: Cc1cc(C(F)(F)F)cc(O)c1-c1ccc(CNC2CCOC2)nn1] |
| 104 | 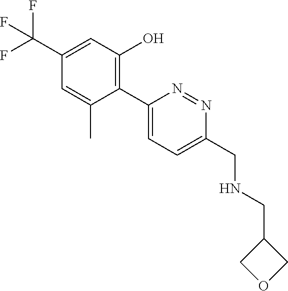 [SMILES: Cc1cc(C(F)(F)F)cc(O)c1-c1ccc(CNCC2COC2)nn1] |
| 105 | 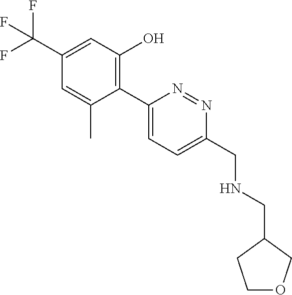 [SMILES: Cc1cc(C(F)(F)F)cc(O)c1-c1ccc(CNCC2CCOC2)nn1] |
| 106 | 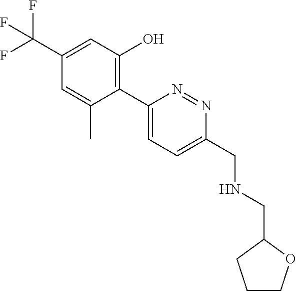 [SMILES: Cc1cc(C(F)(F)F)cc(O)c1-c1ccc(CNCC2CCCO2)nn1] |
| 107 | 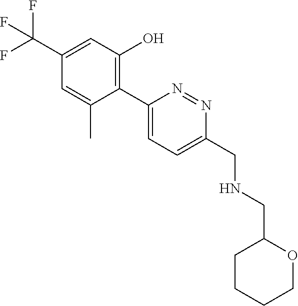 [SMILES: Cc1cc(C(F)(F)F)cc(O)c1-c1ccc(CNCC2CCCCO2)nn1] |
| 108 | 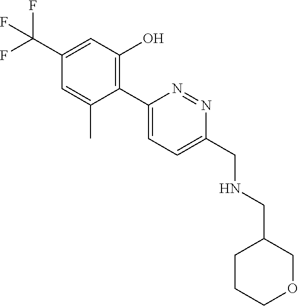 [SMILES: Cc1cc(C(F)(F)F)cc(O)c1-c1ccc(CNCC2CCCOC2)nn1] |
| 109 | 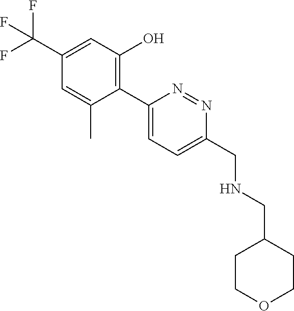 [SMILES: Cc1cc(C(F)(F)F)cc(O)c1-c1ccc(CNCC2CCOCC2)nn1] |
| 110 | 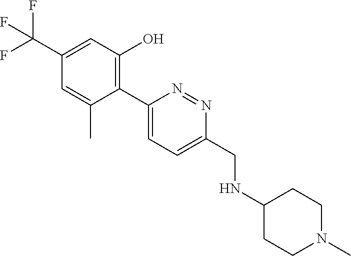 [SMILES: Cc1cc(C(F)(F)F)cc(O)c1-c1ccc(CNC2CCN(C)CC2)nn1] |
| 111 | 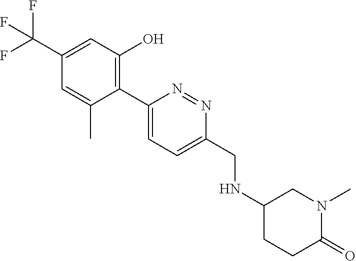 [SMILES: Cc1cc(C(F)(F)F)cc(O)c1-c1ccc(CNC2CCC(=O)N(C)C2)nn1] |
| 112 | 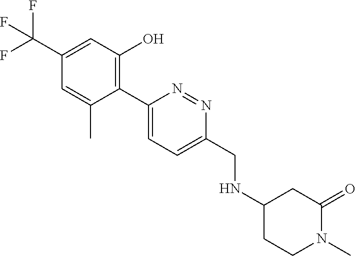 [SMILES: Cc1cc(C(F)(F)F)cc(O)c1-c1ccc(CNC2CCN(C)C(=O)C2)nn1] |
| 113 | 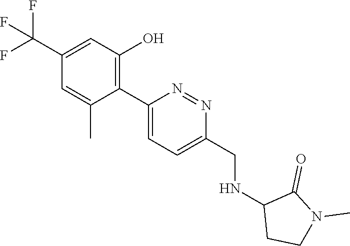 [SMILES: Cc1cc(C(F)(F)F)cc(O)c1-c1ccc(CNC2CCN(C)C2=O)nn1] |
| 114 | 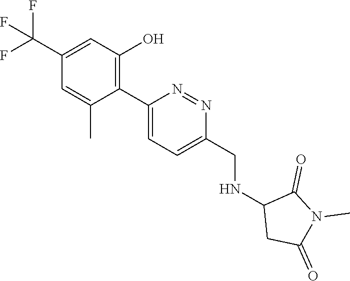 [SMILES: Cc1cc(C(F)(F)F)cc(O)c1-c1ccc(CNC2CC(=O)N(C)C2=O)nn1] |
| 115 | 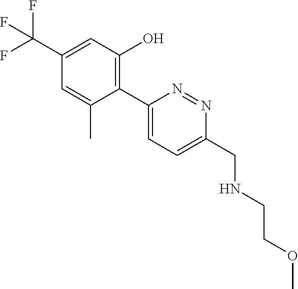 [SMILES: COCCNCc1ccc(-c2c(C)cc(C(F)(F)F)cc2O)nn1] |
| 116 | 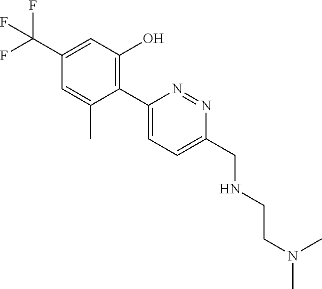 [SMILES: Cc1cc(C(F)(F)F)cc(O)c1-c1ccc(CNCCN(C)C)nn1] |
| 117 | 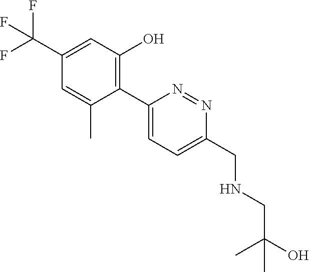 [SMILES: Cc1cc(C(F)(F)F)cc(O)c1-c1ccc(CNCC(C)(C)O)nn1] |
| 118 | 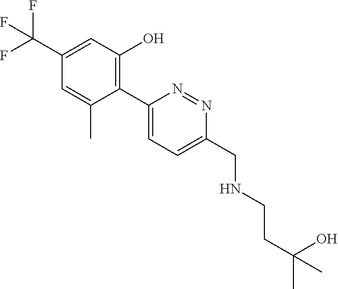 [SMILES: Cc1cc(C(F)(F)F)cc(O)c1-c1ccc(CNCCC(C)(C)O)nn1] |
| 119 | 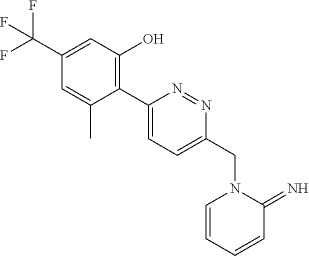 [SMILES: Cc1cc(C(F)(F)F)cc(O)c1-c1ccc(Cn2ccccc2=N)nn1] |
| 120 | 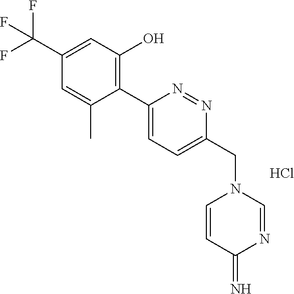 [SMILES: Cc1cc(C(F)(F)F)cc(O)c1-c1ccc(Cn2ccc(=N)nc2)nn1 ; Cl] |
| 121 | 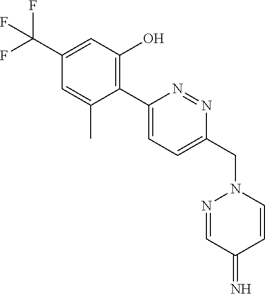 [SMILES: Cc1cc(C(F)(F)F)cc(O)c1-c1ccc(Cn2ccc(=N)cn2)nn1] |
| 122 | 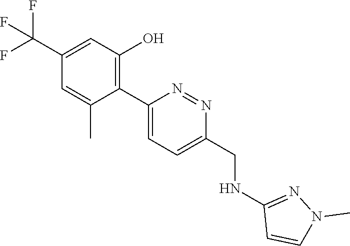 [SMILES: Cc1cc(C(F)(F)F)cc(O)c1-c1ccc(CNc2ccn(C)n2)nn1] |
| 123 | 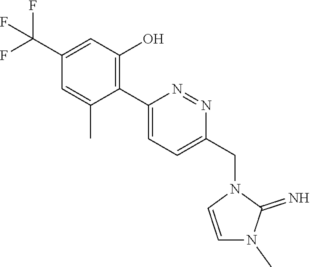 [SMILES: Cc1cc(C(F)(F)F)cc(O)c1-c1ccc(Cn2ccn(C)c2=N)nn1] |
| 124 | 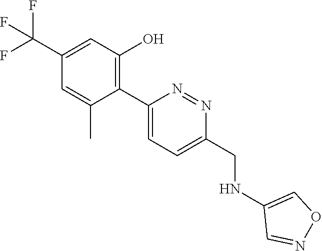 [SMILES: Cc1cc(C(F)(F)F)cc(O)c1-c1ccc(CNc2cnoc2)nn1] |
| 125 | 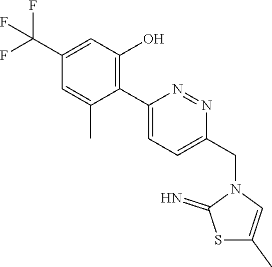 [SMILES: Cc1cn(Cc2ccc(-c3c(C)cc(C(F)(F)F)cc3O)nn2)c(=N)s1] |
| 126 | 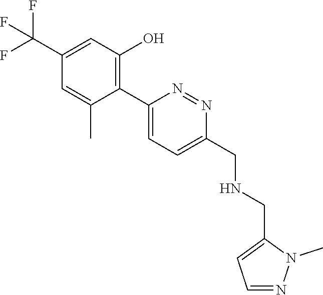 [SMILES: Cc1cc(C(F)(F)F)cc(O)c1-c1ccc(CNCc2ccnn2C)nn1] |
| 127 | 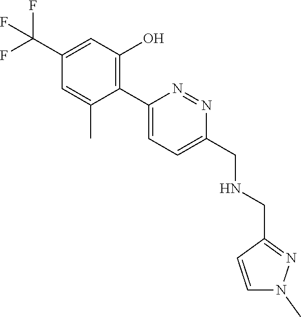 [SMILES: Cc1cc(C(F)(F)F)cc(O)c1-c1ccc(CNCc2ccn(C)n2)nn1] |
| 128 | 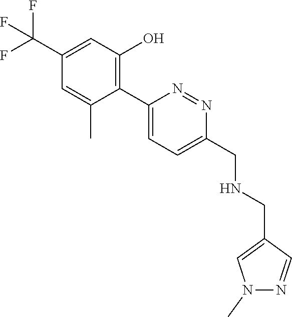 [SMILES: Cc1cc(C(F)(F)F)cc(O)c1-c1ccc(CNCc2cnn(C)c2)nn1] |
| 129 | 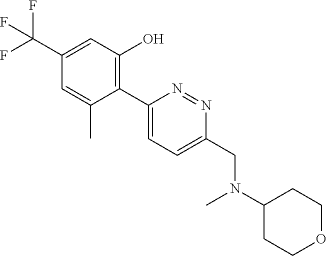 [SMILES: Cc1cc(C(F)(F)F)cc(O)c1-c1ccc(CN(C)C2CCOCC2)nn1] |
| 130 | 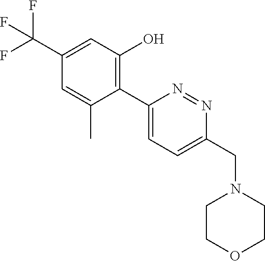 [SMILES: Cc1cc(C(F)(F)F)cc(O)c1-c1ccc(CN2CCOCC2)nn1] |
| 131 | 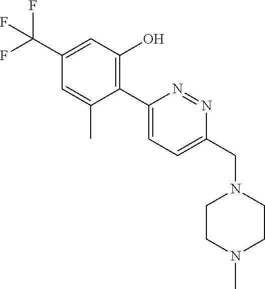 [SMILES: Cc1cc(C(F)(F)F)cc(O)c1-c1ccc(CN2CCN(C)CC2)nn1] |
| 132 | 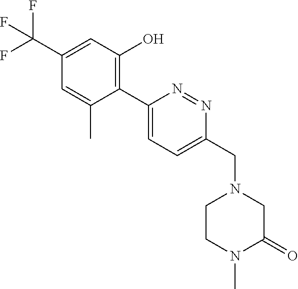 [SMILES: Cc1cc(C(F)(F)F)cc(O)c1-c1ccc(CN2CCN(C)C(=O)C2)nn1] |
| 133 | 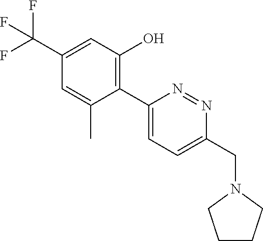 [SMILES: Cc1cc(C(F)(F)F)cc(O)c1-c1ccc(CN2CCCC2)nn1] |
| 134 | 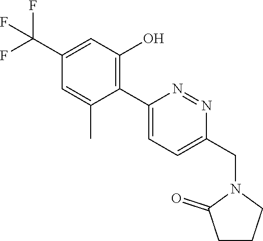 [SMILES: Cc1cc(C(F)(F)F)cc(O)c1-c1ccc(CN2CCCC2=O)nn1] |
| 135 | 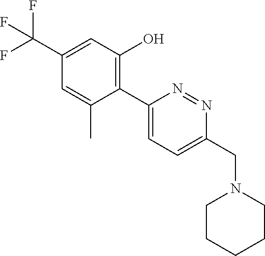 [SMILES: Cc1cc(C(F)(F)F)cc(O)c1-c1ccc(CN2CCCCC2)nn1] |
| 136 | 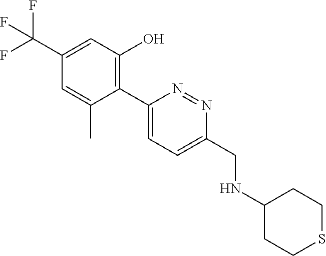 [SMILES: Cc1cc(C(F)(F)F)cc(O)c1-c1ccc(CNC2CCSCC2)nn1] |
| 137 | 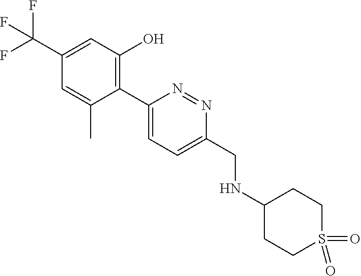 [SMILES: Cc1cc(C(F)(F)F)cc(O)c1-c1ccc(CNC2CCS(=O)(=O)CC2)nn1] |
| 138 | 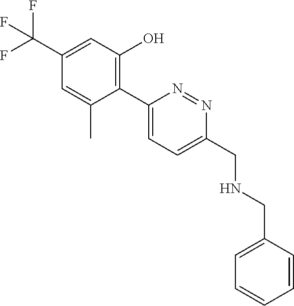 [SMILES: Cc1cc(C(F)(F)F)cc(O)c1-c1ccc(CNCc2ccccc2)nn1] |
| 139 | 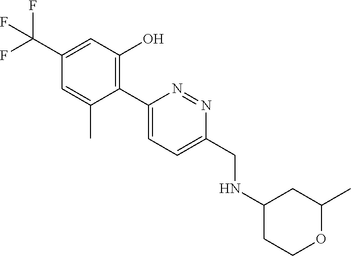 [SMILES: Cc1cc(C(F)(F)F)cc(O)c1-c1ccc(CNC2CCOC(C)C2)nn1] |
| 140 | 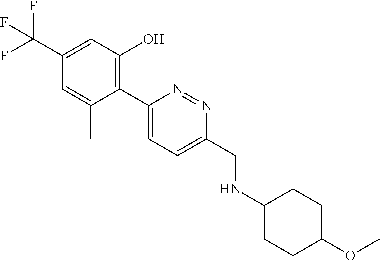 [SMILES: COC1CCC(NCc2ccc(-c3c(C)cc(C(F)(F)F)cc3O)nn2)CC1] |
| 141 | 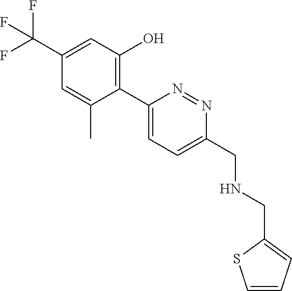 [SMILES: Cc1cc(C(F)(F)F)cc(O)c1-c1ccc(CNCc2cccs2)nn1] |
| 142 | 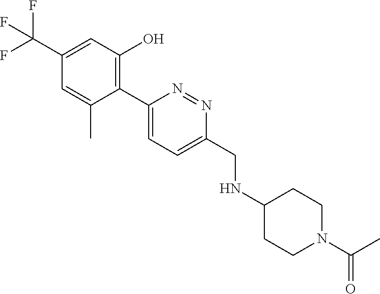 [SMILES: CC(=O)N1CCC(NCc2ccc(-c3c(C)cc(C(F)(F)F)cc3O)nn2)CC1] |
| 143 | 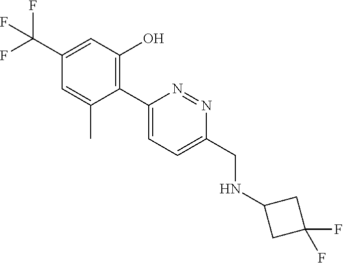 [SMILES: Cc1cc(C(F)(F)F)cc(O)c1-c1ccc(CNC2CC(F)(F)C2)nn1] |
| 144 | 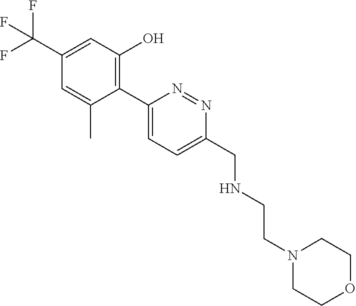 [SMILES: Cc1cc(C(F)(F)F)cc(O)c1-c1ccc(CNCCN2CCOCC2)nn1] |
| 145 | 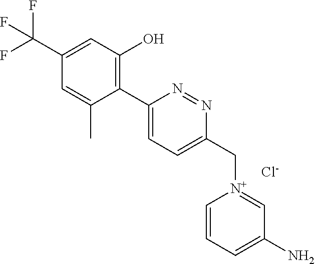 [SMILES: Cc1cc(C(F)(F)F)cc(O)c1-c1ccc(C[n+]2cccc(N)c2)nn1 ; [Cl-]] |
| 146 | 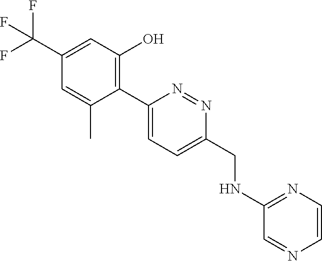 [SMILES: Cc1cc(C(F)(F)F)cc(O)c1-c1ccc(CNc2cnccn2)nn1] |
| 147 | 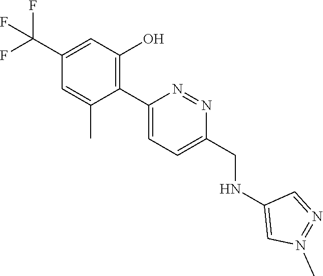 |
| 148 | 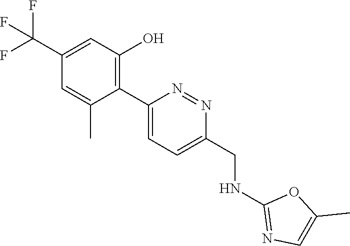 [SMILES: Cc1cnc(NCc2ccc(-c3c(C)cc(C(F)(F)F)cc3O)nn2)o1] |
| 149 | 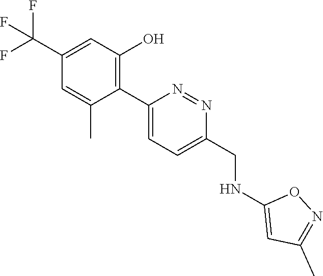 [SMILES: Cc1cc(NCc2ccc(-c3c(C)cc(C(F)(F)F)cc3O)nn2)on1] |
| 150 | 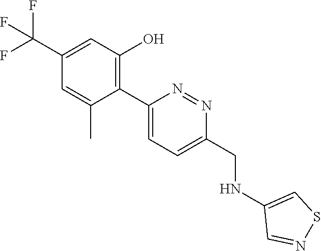 [SMILES: Cc1cc(C(F)(F)F)cc(O)c1-c1ccc(CNc2cnsc2)nn1] |
| 151 | 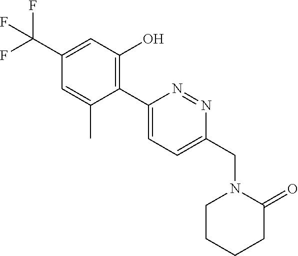 [SMILES: Cc1cc(C(F)(F)F)cc(O)c1-c1ccc(CN2CCCCC2=O)nn1] |
| 152 | 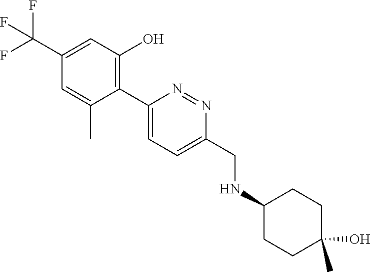 [SMILES: Cc1cc(C(F)(F)F)cc(O)c1-c1ccc(CN[C@H]2CC[C@](C)(O)CC2)nn1] |
| 153 | 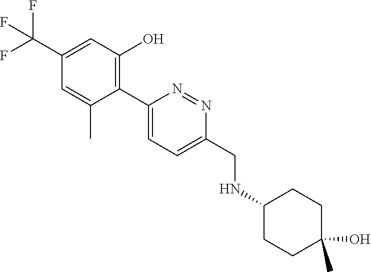 [SMILES: Cc1cc(C(F)(F)F)cc(O)c1-c1ccc(CN[C@H]2CC[C@@](C)(O)CC2)nn1] |
| 154 | 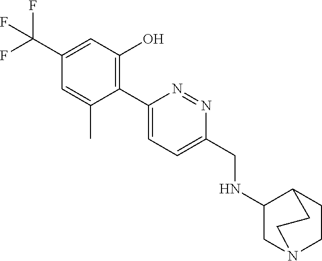 [SMILES: Cc1cc(C(F)(F)F)cc(O)c1-c1ccc(CNC2CN3CCC2CC3)nn1] |

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognised techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

In some embodiments, the compound is a deuterium labeled compound of any one of the compounds described in Table 1 and prodrugs and pharmaceutically acceptable salts thereof.

In some embodiments, the compound is a deuterium labeled compound of any one of the compounds described in Table 1 and pharmaceutically acceptable salts thereof.

In some embodiments, the compound is a deuterium labeled compound of any one of the compounds described in Table 1.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognised techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the invention or a pharmaceutically acceptable salt or solvate thereof that contains the aforementioned deuterium atom(s) is within the scope of the invention. Further, substitution with deuterium (i.e., ²H) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

A suitable pharmaceutically acceptable salt of a compound of the disclosure is, for example, an acid-addition salt of a compound of the disclosure, which is sufficiently basic, for example, an acid-addition salt with, for example, an inorganic or organic acid, for example hydrochloric, hydrobromic, sulphuric, phosphoric, formic, citric methane sulphonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral centre” refers to a carbon atom bonded to four nonidentical substituents.

As used herein, the term “chiral isomer” means a compound with at least one chiral centre. Compounds with more than one chiral centre may exist either as an individual diastereomer or as a mixture of diastereomers, termed “diastereomeric mixture.” When one chiral centre is present, a stereoisomer may be characterised by the absolute configuration (R or S) of that chiral centre. Absolute configuration refers to the arrangement in space of the substituents attached to the chiral centre. The substituents attached to the chiral centre under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulphate, bisulphate, sulphamate, nitrate, phosphate, citrate, methanesulphonate, glutamate, glucuronate, glutarate, malate, maleate, succinate, fumarate, tartrate, tosylate, salicylate, lactate, naphthalenesulphonate, and acetate.

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulphonamides, tetrazoles, sulphonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NTR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

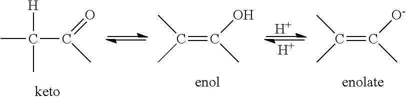

Compounds of any one of the Formulae disclosed herein containing an amine function may also form N-oxides. A reference herein to a compound of Formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents in a compound of the any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; H. E. Roche (editor), “Bioreversible Carriers in Drug Design”, Pergamon Press, 1987. A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

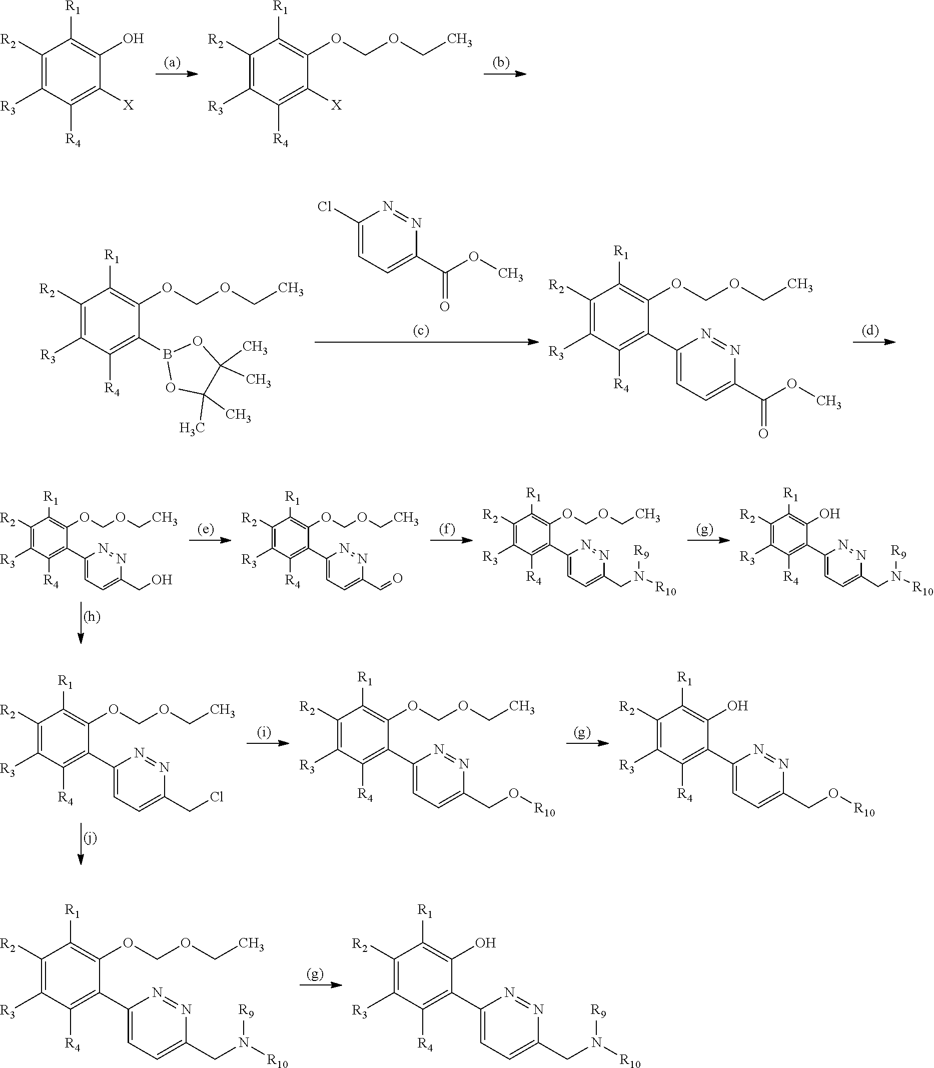

Selected compounds of Formula (I) may be synthesized according to the generic scheme shown above, wherein X is a leaving group (e.g., chloro, bromo, iodo, mesylate, or triflate). All reactions are conducted in an appropriate solvent. Step (a) is an alkylation using chloromethyl ethyl ether and a base. Step (b) is a palladium-catalysed borylation using bis(pinacolato)diboron, a Pd catalyst, and a base. Step (c) is a Suzuki-Miyaura coupling using methyl 6-chloropyridazine-3-carboxylate, a Pd catalyst, and a base. Step (d) is a reduction using a reducing agent (e.g., sodium borohydride). Step (e) is an oxidation using an oxidising agent (e.g., Dess-Martin periodinane). Step (f) is a reductive amination using an amine and reducing agent (e.g., sodium triacetoxyborohydride). Step (g) is a dealkylation using an acid (e.g., hydrochloric acid). Step (h) is a chlorination using a chlorination agent (e.g., thionyl chloride). Step (i) is an ether-forming reaction using an alcohol and a base. Step (j) is an amine-forming reaction using an amine and a base.

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I) has been synthesised by any one of the processes defined herein, the processes may then further comprise the additional steps of: (i) removing any protecting groups present; (ii) converting the compound Formula (I) into another compound of Formula (I); (iii) forming a pharmaceutically acceptable salt, hydrate or solvate thereof; and/or (iv) forming a prodrug thereof.

The resultant compounds of Formula (I) can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulphoxides, such as dimethyl sulphoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilising the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognise which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesised by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons). Compounds of Formula (I) can be prepared as described in the Examples.

### Biological Assays

Compounds designed, selected and/or optimised by methods described above, once produced, can be characterised using a variety of assays known to those skilled in the art to determine whether the compounds have biological activity. For example, the molecules can be characterised by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological away is a biological away testing inhibitory activity against IL-1β release upon NLRP3 activation in peripheral blood mononuclear cells (PBMC).

In some embodiments, the biological assay is a PBMC IC₅₀ Determination Assay. In some embodiments, the biological assay is a PBMC IC₅₀ Determination Assay described in Example 13.

In some embodiments, the compounds of the present disclosure may be tested for their inhibitory activity against IL-1β release upon NLRP3 activation in blood cells (e.g., peripheral blood mononuclear cells (PBMC)).

In some embodiments, PBMC may be isolated and seeded into the wells of a plate and incubated for a period of time (e.g., for 3 hours with a lipopolysaccharide). Following incubation, the medium may be exchanged and a compound added to the well (e.g., a compound of the present disclosure) and the cells may be incubated. Next, the cells may be stimulated (e.g., with ATP or nigericin) and the cell culture media collected for further analysis.

In some embodiments, the release of IL-1β into the media may be determined by a quantitative detection of IL-1β in the media (e.g., using ELISA).

In some embodiments, PBMC may be isolated (e.g., from buffy coats). Isolated cells may be seeded into wells and incubated (e.g., for 3 hours with lipopolysaccharide). The compounds of the present disclosure may then be added and the cells incubated. Next, the cells may be stimulated and the media from the wells collected for further analysis.

In some embodiments, the release of IL-1β into the media may be determined by quantitative detection (e.g., of IL-1β in media using HTRF®).

### Pharmaceutical Compositions

In some aspects, the present disclosure provides a pharmaceutical composition comprising a compound of the present disclosure as an active ingredient.

In some embodiments, the present disclosure provides a pharmaceutical composition comprising a compound described herein and one or more pharmaceutically acceptable carriers or excipients. In some embodiments, the present disclosure provides a pharmaceutical composition comprising at least one compound selected from Tables 1-3.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed-release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intra-muscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulphated β-cyclodextrin (S-β-CD), maltosyl-β-cyclodextrin, β-cyclodextrin sulphobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilise the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular closing or as a suppository for rectal dosing).

The compositions of the disclosure may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present disclosure for use in therapy is an amount sufficient to treat or prevent an inflammasome related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or patient and the route of administration, according to well-known principles of medicine.

### Methods of Use

In some aspects, the present disclosure provides a method of inhibiting inflammasome (e.g., the NLRP3 inflammasome) activity (e.g., in vitro or in vivo), comprising contacting a cell with an effective amount of a compound of the present disclosure or a pharmaceutically acceptable salt thereof.

In some aspects, the present disclosure provides a method of inhibiting inflammasome (e.g., the NLRP3 inflammasome) activity (e.g., in vitro or in vivo), comprising contacting a cell with a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a disease or disorder disclosed herein in a subject in need thereof, comprising administering to the subject a compound of the present disclosure or a pharmaceutically acceptable salt thereof, or a pharmaceutical composition of the present disclosure.

In some embodiments, the disease or disorder is associated with an implicated inflammasome activity. In some embodiments, the disease or disorder is a disease or disorder in which inflammasome activity is implicated.

In some embodiments, the disease or disorder is an inflammatory disorder, autoinflammatory disorder, an autoimmune disorder, a neurodegenerative disease, or cancer.

In some embodiments, the disease or disorder is an inflammatory disorder, autoinflammatory disorder and/or an autoimmune disorder.

In some embodiments, the disease or disorder is cytokine release syndrome (CRS).

In some embodiments, the disease or disorder is selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Irritable Bowel Disease (IBD), Crohn's disease, chronic obstructive pulmonary disease (COPD), chronic kidney disease (CKD), fibrosis, obesity, type 2 diabetes, multiple sclerosis, dermatological disease (e.g. acne) and neuroinflammation occurring in protein misfolding diseases (e.g., Prion diseases).

In some embodiments, the disease or disorder is a neurodegenerative disease.

In some embodiments, the disease or disorder is Parkinson's disease, Alzheimer's disease, multiple sclerosis, amyotrophic lateral sclerosis, or Huntington's disease.

In some embodiments, the disease or disorder is a dermatological disease.

In some embodiments, the dermatological disease is acne.

In some embodiments, the disease or disorder is cancer.

In some embodiments, the cancer is metastasising cancer, gastrointestinal cancer, skin cancer, non-small-cell lung carcinoma, brain cancer (e.g. glioblastoma) or colorectal adenocarcinoma.

In some aspects, the present disclosure provides a method of treating or preventing an autoinflammatory disorder, an autoimmune disorder, a neurodegenerative disease or cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing an autoinflammatory disorder, an autoimmune disorder, a neurodegenerative disease or cancer in a subject in need thereof, comprising administering to the subject a compound of the present disclosure or a pharmaceutically acceptable salt thereof, or a pharmaceutical composition of the present disclosure.

In some aspects, the present disclosure provides a method of treating an autoinflammatory disorder, an autoimmune disorder, a neurodegenerative disease or cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating an autoinflammatory disorder, an autoimmune disorder, a neurodegenerative disease or cancer in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing an inflammatory disorder, autoinflammatory disorder and/or an autoimmune disorder selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Irritable Bowel Disease (IBD), Crohn's disease, chronic obstructive pulmonary disease (COPD), chronic kidney disease (CKD), fibrosis, obesity, type 2 diabetes, multiple sclerosis, dermatological disease (e.g. acne) and neuroinflammation occurring in protein misfolding diseases (e.g., Prion diseases) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing an inflammatory disorder, autoinflammatory disorder and/or an autoimmune disorder selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Irritable Bowel Disease (IBD), Crohn's disease, chronic obstructive pulmonary disease (COPD), chronic kidney disease (CKD), fibrosis, obesity, type 2 diabetes, multiple sclerosis, dermatological disease (e.g. acne) and neuroinflammation occurring in protein misfolding diseases (e.g., Prion diseases) in a subject in need thereof, comprising administering to the subject a compound of the present disclosure or a pharmaceutically acceptable salt thereof, or a pharmaceutical composition of the present disclosure.

In some aspects, the present disclosure provides a method of treating an inflammatory disorder, autoinflammatory disorder and/or an autoimmune disorder selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Irritable Bowel Disease (IBD), Crohn's disease, chronic obstructive pulmonary disease (COPD), chronic kidney disease (CKD), fibrosis, obesity, type 2 diabetes, multiple sclerosis, dermatological disease (e.g. acne) and neuroinflammation occurring in protein misfolding diseases (e.g., Prion diseases) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating an inflammatory disorder, autoinflammatory disorder and/or an autoimmune disorder selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Irritable Bowel Disease (IBD), Crohn's disease, chronic obstructive pulmonary disease (COPD), chronic kidney disease (CKD), fibrosis, obesity, type 2 diabetes, multiple sclerosis, dermatological disease (e.g. acne) and neuroinflammation occurring in protein misfolding diseases (e.g., Prion diseases) in a subject in need thereof, comprising administering to the subject a compound of the present disclosure or a pharmaceutically acceptable salt thereof, or a pharmaceutical composition of the present disclosure.

In some aspects, the present disclosure provides a method of treating or preventing cytokine release syndrome (CRS) in a subject in need thereof, comprising administering to the subject a therapeutically effective amount of a compound of the present disclosure or a pharmaceutically acceptable salt thereof, or a pharmaceutical composition of the present disclosure. In some embodiments, the CRS is associated with COVID-19. In some embodiments, the CRS is associated with adoptive cell therapy.

In some aspects, the present disclosure provides a method of treating or preventing cytokine release syndrome (CRS) in a subject in need thereof, comprising administering to the subject a compound of the present disclosure or a pharmaceutically acceptable salt thereof, or a pharmaceutical composition of the present disclosure. In some embodiments, the CRS is associated with COVID-19. In some embodiments, the CRS is associated with adoptive cell therapy.

In some aspects, the present disclosure provides a method of treating cytokine release syndrome (CRS) in a subject in need thereof, comprising administering to the subject a therapeutically effective amount of a compound of the present disclosure or a pharmaceutically acceptable salt thereof, or a pharmaceutical composition of the present disclosure. In some embodiments, the CRS is associated with COVID-19. In some embodiments, the CRS is associated with adoptive cell therapy.

In some aspects, the present disclosure provides a method of treating cytokine release syndrome (CRS) in a subject in need thereof, comprising administering to the subject a compound of the present disclosure or a pharmaceutically acceptable salt thereof, or a pharmaceutical composition of the present disclosure. In some embodiments, the CRS is associated with COVID-19. In some embodiments, the CRS is associated with adoptive cell therapy.

In some aspects, the present disclosure provides a method of treating or preventing a neurodegenerative disease (e.g., Parkinson's disease, Alzheimer's disease, multiple sclerosis, amyotrophic lateral sclerosis, or Huntington's disease) in a subject in need thereof, said method comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a neurodegenerative disease (e.g., Parkinson's disease, Alzheimer's disease, multiple sclerosis, amyotrophic lateral sclerosis, or Huntington's disease) in a subject in need thereof, said method comprising administering to the subject a compound of the present disclosure or a pharmaceutically acceptable salt thereof, or a pharmaceutical composition of the present disclosure.

In some aspects, the present disclosure provides a method of treating a neurodegenerative disease (e.g., Parkinson's disease, Alzheimer's disease, multiple sclerosis, amyotrophic lateral sclerosis, or Huntington's disease) in a subject in need thereof, said method comprising administering to the subject a therapeutically effective amount of a compound of the present disclosure or a pharmaceutically acceptable salt thereof, or a pharmaceutical composition of the present disclosure.

In some aspects, the present disclosure provides a method of treating a neurodegenerative disease (e.g., Parkinson's disease, Alzheimer's disease, multiple sclerosis, amyotrophic lateral sclerosis, or Huntington's disease) in a subject in need thereof, said method comprising administering to the subject a compound of the present disclosure or a pharmaceutically acceptable salt thereof, or a pharmaceutical composition of the present disclosure.

In some aspects, the present disclosure provides a method of treating or preventing cancer in a subject in need thereof, said method comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing cancer in a subject in need thereof, said method comprising administering to the subject a compound of the present disclosure or a pharmaceutically acceptable salt thereof, or a pharmaceutical composition of the present disclosure.

In some aspects, the present disclosure provides a method of treating cancer in a subject in need thereof, said method comprising administering to the subject a therapeutically effective amount of a compound of the present disclosure or a pharmaceutically acceptable salt thereof, or a pharmaceutical composition of the present disclosure.

In some aspects, the present disclosure provides a method of treating cancer in a subject in need thereof, said method comprising administering to the subject a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in inhibiting inflammasome (e.g., the NLRP3 inflammasome) activity (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing an inflammatory disorder, an autoinflammatory disorder, an autoimmune disorder, a neurodegenerative disease or cancer in a subject in need thereof.

In some aspects, the present disclosure provides a compound of the present disclosure or a pharmaceutically acceptable salt thereof for use in treating an inflammatory disorder, an autoinflammatory disorder, an autoimmune disorder, a neurodegenerative disease or cancer in a subject in need thereof.

In some aspects, the present disclosure provides a compound of the present disclosure or a pharmaceutically acceptable salt thereof for use in treating or preventing an inflammatory disorder, an autoinflammatory disorder and/or an autoimmune disorder selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Irritable Bowel Disease (IBD), Crohn's disease, chronic obstructive pulmonary disease (COPD), chronic kidney disease (CKD), fibrosis, obesity, type 2 diabetes, multiple sclerosis and neuroinflammation occurring in protein misfolding diseases (e.g., Prion diseases) in a subject in need thereof.

In some aspects, the present disclosure provides a compound of the present disclosure or a pharmaceutically acceptable salt thereof for use in treating an inflammatory disorder, an autoinflammatory disorder and/or an autoimmune disorder selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Irritable Bowel Disease (IBD), Crohn's disease, chronic obstructive pulmonary disease (COPD), chronic kidney disease (CKD), fibrosis, obesity, type 2 diabetes, multiple sclerosis and neuroinflammation occurring in protein misfolding diseases (e.g., Prion diseases) in a subject in need thereof.

In some aspects, the present disclosure provides a compound of the present disclosure or a pharmaceutically acceptable salt thereof for use in treating or preventing CRS in a subject in need thereof.

In some aspects, the present disclosure provides a compound of the present disclosure or a pharmaceutically acceptable salt thereof for use in treating CRS in a subject in need thereof.

In some aspects, the present disclosure provides a compound of the present disclosure or a pharmaceutically acceptable salt thereof for use in treating or preventing a neurodegenerative disease (e.g., Parkinson's disease, Alzheimer's disease, multiple sclerosis, amyotrophic lateral sclerosis, or Huntington's disease) in a subject in need thereof.

In some aspects, the present disclosure provides a compound of the present disclosure or a pharmaceutically acceptable salt thereof for use in treating a neurodegenerative disease (e.g., Parkinson's disease, Alzheimer's disease, multiple sclerosis, amyotrophic lateral sclerosis, or Huntington's disease) in a subject in need thereof.

In some aspects, the present disclosure provides a compound of the present disclosure or a pharmaceutically acceptable salt thereof for use in treating or preventing cancer in a subject in need thereof.

In some aspects, the present disclosure provides a compound of the present disclosure or a pharmaceutically acceptable salt thereof for use in treating cancer in a subject in need thereof.

In some aspects, the present disclosure provides use of a compound of the present disclosure or a pharmaceutically acceptable salt thereof in the manufacture of a medicament for inhibiting inflammasome (e.g., the NLRP3 inflammasome) activity (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing an inflammatory disorder, an autoinflammatory disorder, an autoimmune disorder, a neurodegenerative disease or cancer in a subject in need thereof.

In some aspects, the present disclosure provides use of a compound of the present disclosure or a pharmaceutically acceptable salt thereof in the manufacture of a medicament for treating an inflammatory disorder, an autoinflammatory disorder, an autoimmune disorder, a neurodegenerative disease or cancer in a subject in need thereof.

In some aspects, the present disclosure provides use of a compound of the present disclosure or a pharmaceutically acceptable salt thereof in the manufacture of a medicament for treating or preventing an inflammatory disorder, an autoinflammatory disorder and/or an autoimmune disorder selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Irritable Bowel Disease (IBD), Crohn's disease, chronic obstructive pulmonary disease (COPD), chronic kidney disease (CKD), fibrosis, obesity, type 2 diabetes, multiple sclerosis, dermatological disorders (e.g., acne) and neuroinflammation occurring in protein misfolding diseases (e.g., Prion diseases) in a subject in need thereof.

In some aspects, the present disclosure provides use of a compound of the present disclosure or a pharmaceutically acceptable salt thereof in the manufacture of a medicament for treating an inflammatory disorder, an autoinflammatory disorder and/or an autoimmune disorder selected from cryopyrin-associated autoinflammatory syndrome (CAPS; e.g., familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), gout, rheumatoid arthritis, osteoarthritis, Irritable Bowel Disease (IBD), Crohn's disease, chronic obstructive pulmonary disease (COPD), chronic kidney disease (CKD), fibrosis, obesity, type 2 diabetes, multiple sclerosis, dermatological disorders (e.g., acne) and neuroinflammation occurring in protein misfolding diseases (e.g., Prion diseases) in a subject in need thereof.

In some aspects, the present disclosure provides use of a compound of the present disclosure or a pharmaceutically acceptable salt thereof in the manufacture of a medicament for treating or preventing CRS in a subject in need thereof.

In some aspects, the present disclosure provides use of a compound of the present disclosure or a pharmaceutically acceptable salt thereof in the manufacture of a medicament for treating CRS in a subject in need thereof.

In some aspects, the present disclosure provides use of a compound of the present disclosure or a pharmaceutically acceptable salt thereof in the manufacture of a medicament for treating or preventing a neurodegenerative disease (e.g., Parkinson's disease, Alzheimer's disease, multiple sclerosis, amyotrophic lateral sclerosis, or Huntington's disease) in a subject in need thereof.

In some aspects, the present disclosure provides use of a compound of the present disclosure or a pharmaceutically acceptable salt thereof in the manufacture of a medicament for treating a neurodegenerative disease (e.g., Parkinson's disease, Alzheimer's disease, multiple sclerosis, amyotrophic lateral sclerosis, or Huntington's disease) in a subject in need thereof.

In some aspects, the present disclosure provides use of a compound of the present disclosure or a pharmaceutically acceptable salt thereof in the manufacture of a medicament for treating or preventing cancer in a subject in need thereof.

In some aspects, the present disclosure provides use of a compound of the present disclosure or a pharmaceutically acceptable salt thereof in the manufacture of a medicament for treating cancer in a subject in need thereof.

The present disclosure provides compounds that function as inhibitors of inflammasome activity. The present disclosure therefore provides a method of inhibiting inflammasome activity in vitro or in vivo, said method comprising contacting a cell with an effective amount of a compound, or a pharmaceutically acceptable salt thereof, as defined herein.

Effectiveness of compounds of the disclosure can be determined by industry-accepted assays/disease models according to standard practices of elucidating the same as described in the art and are found in the current general knowledge.

The present disclosure also provides a method of treating a disease or disorder in which inflammasome activity is implicated in a patient in need of such treatment, said method comprising administering to said patient a therapeutically effective amount of a compound, or a pharmaceutically acceptable salt thereof, or a pharmaceutical composition as defined herein.

On a general level, the compounds of the present disclosure, which inhibit the maturation of cytokines of the IL-1 family, are effective in all therapeutic indications that are mediated or associated with elevated levels of active forms of cytokines belonging to IL-1 family of cytokines (Sims J. et al. Nature Reviews Immunology 10, 89-102 (February 2010).

Exemplary diseases and the corresponding references will be given in the following: inflammatory, autoinflammatory and autoimmune diseases like CAPS (Dinarello, C. A. Immunity. 2004 March; 20(3):243-4; Hoffman, H. M. et al. Reumatologia 2005; 21(3)), gout, rheumatoid arthritis (Gabay, C. et al. Arthritis Research & Therapy 2009, 11:230; Schett, G. et al. Nat Rev Rheumatol. 2016 January; 12(1):14-24), Crohn's disease (Jung Mogg Kim Korean J Gastroenterol. Vol. 58 No. 6, 300-310), COPD (Mortaz, E. et al. Tanaffos. 2011; 10(2): 9-14), fibrosis (Gasse, P. et al. Am. J. Respir. Crit. Care Med. 2009 May 15; 179(10):903-13), obesity, type 2 diabetes ((Dinarello, C. A. et al. Curr. Opin. Endocrinol. Diabetes Obes. 2010 August; 17(4):314-21)) multiple sclerosis (see EAE-model in Coll, R. C. et al. Nat. Med. 2015 March; 21(3):248-55) and many others (Martinon, F. et al. Immunol. 2009. 27:229-65) like Parkinson's disease or Alzheimer's disease (Michael, T. et al. Nature 493, 674-678 (31 Jan. 2013); Halle, A. et al., Nat. Immunol. 2008 August; 9(8):857-65; Saresella, M. et al. Mol. Neurodegener. 2016 Mar. 3; 11:23) and some oncological disorders.

Suitably, the compounds according to the present disclosure can be used for the treatment of a disease selected from the group consisting of cytokine release syndrome (CRS), an inflammatory disease, an autoinflammatory disease, an autoimmune disease, a neurodegenerative disease and cancer. Said inflammatory, autoinflammatory and autoimmune disease is suitably selected from the group consisting of a cryopyrin-associated autoinflammatory syndrome (CAPS, such as for example familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), chronic kidney disease (CKD), gout, rheumatoid arthritis, osteoarthritis, Irritable Bowel Disease (IBD), Crohn's disease, COPD, fibrosis, obesity, type 2 diabetes, multiple sclerosis, dermatological diseases (e.g., acne) and neuroinflammation occurring in protein misfolding diseases, such as Prion diseases. Said neurodegenerative disease includes, but is not limited, to Parkinson's disease and Alzheimer's disease.

Accordingly, the compounds of the present disclosure can be used for the treatment of a disease selected from the group consisting of cryopyrin-associated autoinflammatory syndrome (CAPS, such as for example familial cold autoinflammatory syndrome (FCAS), Muckle-Wells syndrome (MWS), chronic infantile neurological cutaneous and articular (CINCA) syndrome/neonatal-onset multisystem inflammatory disease (NOMID)), familial Mediterranean fever (FMF), nonalcoholic fatty liver disease (NAFLD), non-alcoholic steatohepatitis (NASH), chronic kidney disease (CKD), gout, rheumatoid arthritis, osteoarthritis, Irritable Bowel Disease (IBD), Crohn's disease, COPD, fibrosis, obesity, type 2 diabetes, multiple sclerosis, dermatological diseases (e.g., acne) neuroinflammation occurring in protein misfolding diseases, such as Prion diseases, neurogenerative diseases (e.g., Parkinson's disease, Alzheimer's disease, multiple sclerosis, amyotrophic lateral sclerosis, or Huntington's disease) and oncological disorders.

Inflammatory Disease Associated with Infection

In some embodiments, the disease or disorder is an inflammatory disease.

In some embodiments, the inflammatory disease is associated with an infection.

In some embodiments, the inflammatory disease is associated with an infection by a virus.

In some embodiments, the inflammatory disease is associated with an infection by an RNA virus. In some embodiments, the RNA virus is a single stranded RNA virus. Single stranded RNA viruses include group IV (positive strand) and group V (negative strand) single stranded RNA viruses. Group IV viruses include coronaviruses.

In some embodiments, the inflammatory disease is associated with an infection by a coronavirus. In some embodiments, the coronavirus is Severe Acute Respiratory Syndrome Coronavirus 2 (SARS-CoV 2), SARS coronavirus (SARS CoV) or Middle East respiratory syndrome-related coronavirus (MERS).

In some embodiments, the inflammatory disease is associated with an infection by SARS-CoV 2. In some embodiments, SARS-CoV 2 infection leads to 2019 novel coronavirus disease (COVID-19).

In some embodiments, the inflammatory disease is an inflammatory disease of lung.

In some embodiments, the inflammatory disease of lung is associated with an infection by SARS-CoV 2.

In some embodiments, the inflammatory disease comprises cytokine release syndrome (CRS).

In some embodiments, the cytokine release syndrome (CRS) is associated with an infection by SARS-CoV 2.

### Cytokine Release Syndrome and Immunotherapy

In some embodiments, the disease or disorder is an inflammatory disease.

In some embodiments, the inflammatory disease is associated with an immunotherapy.

In some embodiments, the immunotherapy causes cytokine release syndrome (CRS).

The effectiveness of immunotherapies, such as CAR-T, are hampered by the frequency with which such therapies induce cytokine release syndrome. Without wishing to be bound by theory, it is thought that the severity of CRS induced by immunotherapy is mediated by IL-6, IL-1 and NO production (Giavridis et al. Nature Medicine; doi.org/10.1038/s41591-018-0041-7). Alternatively, or in addition, CRS may occur when cells targeted by the adoptive cell therapy undergo pyroptosis, a highly inflammatory form of programmed cell death. Pyroptosis leads to release of factors that stimulate macrophages to produce pro-inflammatory cytokines, leading to CRS (Liu et al. Science Immunology (2020) V: eeax7969).

In some embodiments, the immunotherapy comprises an antibody or an adoptive cell therapy.

In some embodiments, the adoptive cell therapy comprises a CAR-T or TCR-T cell therapy.

In some embodiments, the adoptive cell therapy comprises a cancer therapy. For example, the cancer therapy can be to treat B cell lymphoma or B cell acute lymphoblastic leukemia. For example, the adoptive cells may express a CAR targeting CD19+ B cell acute lymphoblastic leukemia cells.

In some embodiments, the adoptive cell therapy comprises administration of T cells, B cells or NK cells.

In some embodiments, the adoptive cell therapy is autologous.

In some embodiments, the adoptive therapy is allogeneic.

Treatment in Cancer; Links with Inflammasome

Chronic inflammation responses have long been observed to be associated with various types of cancer. During malignant transformation or cancer therapy inflammasomes may become activated in response to danger signals and this activation may be both beneficial and detrimental in cancer.

IL-1β expression is elevated in a variety of cancers (including breast, prostate, colon, lung, head and neck cancers and melanomas) and patients with IL-1β producing tumours generally have a worse prognosis (Lewis, Anne M., et al. “Interleukin-1 and cancer progression: the emerging role of interleukin-1 receptor antagonist as a novel therapeutic agent in cancer treatment.” Journal of translational medicine 4.1 (2006): 48).

Cancers derived from epithelial cells (carcinoma) or epithelium in glands (adenocarcinoma) are heterogeneous; consisting of many different cell types. This may include fibroblasts, immune cells, adipocytes, endothelial cells and pericytes amongst others, all of which may be cytokine/chemokine secreting (Grivennikov, Sergei I., Florian R. Greten, and Michael Karin. “Immunity, inflammation, and cancer.” Cell 140.6 (2010): 883-899). This can lead to cancer-associated inflammation through the immune cell infiltration. The presence of leukocytes in tumours is known but it has only recently become evident that an inflammatory microenvironment is an essential component of all tumours. Most tumours (>90%) are the result of somatic mutations or environmental factors rather than germline mutations and many environmental causes of cancer are associated with chronic inflammation (20% of cancers are related to chronic infection, 30% to smoking/inhaled pollutants and 35% to dietary factors (20% of all cancers are linked to obesity) (Aggarwal, Bharat B., R. V. Vijayalekshmi, and Bokyung Sung. “Targeting inflammatory pathways for prevention and therapy of cancer: short-term friend, long-term foe.” Clinical Cancer Research 15.2 (2009): 425-430).

### GI Cancers

Cancers of the gastrointestinal (GI) tract are frequently associated with chronic inflammation. For example, H. pylori infection is associated with gastric cancer (Amieva, Manuel, and Richard M. Peek. “Pathobiology of Helicobacter pylori-Induced Gastric Cancer.” Gastroenterology 150.1 (2016): 64-78). Colorectal cancer is associated with inflammatory bowel disease (Bernstein, Charles N., et al. “Cancer risk in patients with inflammatory bowel disease.” Cancer 91.4 (2001): 854-862). Chronic inflammation in stomach leads to the upregulation of IL-1 and other cytokines (Basso, D. et al., (1996) Helicobacter pylori infection enhances mucosal interleukin-1 beta, interleukin-6, and the soluble receptor of interleukin-2. Int J Clin Lab Res 26:207-210) and polymorphisms in IL-1β gene can increase risk of gastric cancer (Wang, P. et al., (2007) Association of interleukin-1 gene polymorphisms with gastric cancer: a meta-analysis. Int J Cancer 120:552-562).

In 19% of gastric cancer cases, caspase-1 expression is decreased which correlates with stage, lymph node metastasis and survival (Jee et al., 2005). Mycoplasma hyorhinis is associated with the development of gastric cancer its activation of the NLRP3 inflammasome may be associated with its promotion of gastric cancer metastasis (Xu et al., 2013).

### Skin Cancers

Ultraviolet radiation is the greatest environmental risk for skin cancer which is promoted by causing DNA damage, immunosuppression and inflammation. The most malignant skin cancer, melanoma, is characterised by the upregulation of inflammatory cytokines, all of which can be regulated by IL-1β (Lázár-Molnár, Eszter, et al. “Autocrine and paracrine regulation by cytokines and growth factors in melanoma.” Cytokine 12.6 (2000): 547-554). Systemic inflammation induces an enhancement of melanoma cell metastasis and growth by IL-1-dependent mechanisms in vivo. Using thymoquinone inhibition of metastasis in a B16F10 mouse melanoma model was shown to be dependent on inhibition of the NLRP3 inflammasome (Ahmad, Israr, et al. “Thymoquinone suppresses metastasis of melanoma cells by inhibition of NLRP3 inflammasome.” Toxicology and applied pharmacology 270.1 (2013): 70-76).

### Glioblastoma

NLRP3 contributes to radiotherapy resistance in glioma. Ionising radiation can induce NLRP3 expression whereas NLRP3 inhibition reduced tumour growth and prolonged mouse survival following radiation therapy. NLRP3 inflammasome inhibition can therefore provide a therapeutic strategy for radiation-resistant glioma (Li, Lianling, and Yuguang Liu. “Aging-related gene signature regulated by Nlrp3 predicts glioma progression.” American journal of cancer research 5.1 (2015): 442).

### Metastasis

More widely, NLRP3 is considered by the applicants to be involved in the promotion of metastasis and consequently modulation of NLRP3 should plausibly block this. IL-1 is involved in tumour genesis, tumour invasiveness, metastasis, tumour host interactions (Apte, Ron N., et al. “The involvement of IL-1 in tumorigenesis, tumour invasiveness, metastasis and tumour-host interactions.” Cancer and Metastasis Reviews 25.3 (2006): 387-408) and angiogenesis (Voronov, Elena, et al. “IL-1 is required for tumor invasiveness and angiogenesis.” Proceedings of the National Academy of Sciences 100.5 (2003): 2645-2650).

The IL-1 gene is frequently expressed in metastases from patients with several types of human cancers. For example, IL-1mRNA was highly expressed in more than half of all tested metastatic human tumour specimens including specifically non-small-cell lung carcinoma, colorectal adenocarcinoma, and melanoma tumour samples (Elaraj, Dina M., et al. “The role of interleukin 1 in growth and metastasis of human cancer xenografts.” Clinical Cancer Research 12.4 (2006): 1088-1096) and IL-1RA inhibits xenograft growth in IL-1 producing tumours but without anti-proliferative effects in vitro.

Further, IL-1 signalling is a biomarker for predicting breast cancer patients at increased risk for developing bone metastasis. In mouse models IL-1β and its receptor are upregulated in breast cancer cells that metastasise to bone compared with cells that do not. In a mouse model the IL-1 receptor antagonist anakinra reduced proliferation and angiogenesis in addition to exerting significant effects on the tumour environment reducing bone turnover markers, IL-1β and TNF alpha (Holen, Ingunn, et al. “IL-1 drives breast cancer growth and bone metastasis in vivo.” Oncotarget (2016).

IL-18 induced the production of MMP-9 in the human leukaemia cell line HL-60, thus favouring degradation of the extracellular matrix and the migration and invasiveness of cancer cells (Zhang, Bin, et al. “IL-18 increases invasiveness of HL-60 myeloid leukemia cells: up-regulation of matrix metalloproteinases-9 (MMP-9) expression.” Leukemia research 28.1 (2004): 91-95). Additionally IL-18 can support the development of tumour metastasis in the liver by inducing expression of VCAM-1 on hepatic sinusoidal endothelium (Carrascal, Maria Teresa, et al. “Interleukin-18 binding protein reduces b16 melanoma hepatic metastasis by neutralizing adhesiveness and growth factors of sinusoidal endothelium.” Cancer Research 63.2 (2003): 491-497).

CD36

The fatty acid scavenger receptor CD36 serves a dual role in priming gene transcription of pro-IL-1β and inducing assembly of the NLRP3 inflammasome complex. CD36 and the TLR4-TLR6 heterodimer recognise oxLDL, which initiates a signalling pathway leading to transcriptional upregulation of NLRP3 and pro-IL-1β (signal 1). CD36 also mediates the internalisation of oxLDL into the lysosomal compartment, where crystals are formed that induce lysosomal rupture and activation of the NLRP3 inflammasome (signal 2) (Kagan, J and Horng T., “NLRP3 inflammasome activation: CD36 serves double duty.” Nature immunology 14.8 (2013): 772-774).

A subpopulation of human oral carcinoma cells express high levels of the fatty acid scavenger receptor CD36 and are unique in their ability to initiate metastasis. Palmitic acid or a high fat diet boosted the metastatic potential of the CD36+ cells. Neutralising anti-CD36 antibodies blocked metastasis in orthotopic mouse models of human oral cancer. The presence of CD36+ metastasis-initiating cells correlates with a poor prognosis for numerous types of carcinomas. It is suggested that dietary lipids may promote metastasis (Pasqual, G, Avgustinova, A., Mejetta, S, Martin, M, Castellanos, A, Attolini, CS—O, Berenguer, A., Prats, N, Toll, A, Hueto, JA, Bescos, C, Di Croce, L, and Benitah, S A. 2017 “Targeting metastasis-initiating cells through the fatty acid receptor CD36” Nature 541:41-45).

In hepatocellular carcinoma exogenous palmitic acid activated an epithelial-mesenchymal transition (EMT)-like program and induced migration that was decreased by the CD36 inhibitor, sulpho-N-succinimidyl oleate (Nath, Aritro, et al. “Elevated free fatty acid uptake via CD36 promotes epithelial-mesenchymal transition in hepatocellular carcinoma.” Scientific reports 5 (2015). Body mass index was not associated with the degree of EMT highlighting that it is actually CD36 and free fatty acids that are important.

Cancer stems cells (CSCs) use CD36 to promote their maintenance. Oxidised phospholipids, ligands of CD36, were present in glioblastoma and the proliferation of CSCs but not non-CSCs increased with exposure to oxidised LDL. CD36 also correlated with patient prognosis.

### Chemotherapy Resistance

In addition to direct cytotoxic effects, chemotherapeutic agents harness the host immune system which contributes to anti-tumour activity. However, gemcitabine and 5-FU were shown to activate NLRP3 in myeloid-derived suppressor cells leading to production of IL-1β which curtails anti-tumour efficacy. Mechanistically these agents destabilised the lysosome to release cathepsin B to activate NLRP3. IL-1β drove the production of IL-17 from CD4+ T cells, which in turn blunted the efficacy of the chemotherapy. Higher anti-tumoral effects for both gemcitabine and 5-FU were observed when tumours were established in NLRP3−/− or Caps1−/− mice, or WT mice treated with IL-1RA. Myeloid-derived suppressor cell NLRP3 activation therefore limits the anti-tumour efficacy of gemcitabine and 5-FU (Bruchard, Melanie, et al. “Chemotherapy-triggered cathepsin B release in myeloid-derived suppressor cells activates the Nlrp3 inflammasome and promotes tumour growth.” Nature medicine 19.1 (2013): 57-64). Compounds of the present disclosure may therefore be useful in chemotherapy to treat a range of cancers.

Compounds of the present disclosure, or pharmaceutically acceptable salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced). Alternatively, by way of example only, the benefit experienced by an individual may be increased by administering the compound of Formula (I) with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which inflammasome activity is implicated comprising a compound of the disclosure as defined hereinbefore, or a pharmaceutically acceptable salt thereof, and another suitable agent.

According to a further aspect of the disclosure there is provided a pharmaceutical composition which comprises a compound of the disclosure, or a pharmaceutically acceptable salt thereof, in combination with a suitable, in association with a pharmaceutically acceptable diluent or carrier.

In addition to its use in therapeutic medicine, compounds of Formula (I) and pharmaceutically acceptable salts thereof are also useful as pharmacological tools in the development and standardisation of in vitro and in vivo test systems for the evaluation of the effects of inhibitors of inflammasome in laboratory animals such as dogs, rabbits, monkeys, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options.

It is to be understood that a compound of the present disclosure may be depicted in a neutral form, a cationic form (e.g., carrying one or more positive charges), or an anionic form (e.g., carrying one or more negative charges), all of which are intended to be included in the scope of the present disclosure. For example, when a compound of the present disclosure is depicted in an anionic form, such depiction also refers to the various neutral forms, cationic forms, and anionic forms of the compound. For another example, when a compound the present disclosure is depicted in an anionic form, such depiction also refers to various salts (e.g., sodium salt) of the anionic form of the compound.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, alkylsulphinyl, sulphonato, sulphamoyl, sulphonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 6-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic.

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur. The nitrogen atom may be substituted or unsubstituted (i.e., N or NR wherein R is H or other substituents, as defined). The nitrogen and sulphur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthyridine, indole, benzofuran, purine, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system.

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O—.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

As used herein “Severe Acute Respiratory Syndrome Coronavirus 2 (SARS-CoV 2)” refers to the coronavirus that causes 2019 novel coronavirus disease (COVID-19). COVID-19 was first identified in 2019 in Wuhan, China, and has resulted in an ongoing global pandemic. As of August 2020, more than 25 million cases have been reported globally, resulting in an estimated 848,000 deaths. Common symptoms of COVID-19 include fever, cough, fatigue, shortness of breath, and loss of smell and taste. While many people have mild symptoms, some people develop acute respiratory distress syndrome, possibly caused by cytokine release syndrome (CRS), multi-organ failure, septic shock, and blood clots. Time from exposure to the virus to symptom onset is typically around 5 days but may range from 2 to 14 days.

As used herein “cytokine release syndrome (CRS)” refers to a systemic inflammatory response that can be triggered by a variety of factors, including but not limited to drugs, infections such as SARS-CoV 2, and immunotherapies such as chimeric antigen receptor T cell (CAR-T) therapies. In CRS, large numbers of immune cells (e.g. T cells) are activated and release inflammatory cytokines, which in turn activate additional immune cells. Symptoms include fever, fatigue, loss of appetite, muscle and joint pain, nausea, vomiting, diarrhea, rashes, respiratory insufficiency, low blood pressure, seizures, headache and confusion. CRS may respond to IL-6 receptor inhibition, and high doses of steroids.

As used herein, “adoptive cell therapy” refers to a form of treatment that uses immune cells to treat diseases such as cancer. Immune cells, for example T cells are collected from the subject or another source, grown in large numbers, and implanted into the subject to help the immune system fight the disease. Types of adoptive cell therapy include chimerica antigen receptor T cell (CAR-T) therapy, tumor infiltrating lymphocyte (TIL) therapy, and T cell receptor T cell (TCR-T) therapy.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. For example, CARs may direct specificity of the cell expressing the CAR to a tumor associated antigen. In some embodiments, CARs comprise an intracellular activation domain, a transmembrane domain, and an extracellular domain comprising an antigen binding domain, and optionally an extracellular hinge. The antigen binding domain can be any antigen binding domain known in the art, including antigen binding domains derived from antibodies, Fab, F(ab′)2, nanobodies, single domain antigen binding domains, scFv, VHH, and the like. In particular aspects, CARs comprise fusions of single-chain variable fragments (scFv) derived from monoclonal antibodies, fused to a CD3 transmembrane domain and endodomain. In certain cases, CARs comprise domains for additional co-stimulatory signaling, such as CD3, FcR, CD27, CD28, CD137, DAP10, and/or 0X40.

A “T cell receptor (TCR)” is a protein complex found on the surface of T cells, or T lymphocytes, that is responsible for recognizing fragments of antigen as peptides bound to major histocompatibility complex (MHC) molecules. T cell receptors can be engineered to express antigen binding domains specific to particular antigens and used in the adoptive cell therapies described herein.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated those compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognised reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches, and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulphite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount,” refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulphonic, acetic, ascorbic, benzene sulphonic, benzoic, bicarbonic, carbonic, citric, edetic, ethane disulphonic, 1,2-ethane sulphonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulphonic, maleic, malic, mandelic, methane sulphonic, napsylic, nitric, oxalic, pamoic, pantothenic, phenylacetic, phosphoric, polygalacturonic, propionic, salicylic, stearic, subacetic, succinic, sulphamic, sulphanilic, sulphuric, tannic, tartaric, toluene sulphonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulphonic acid, 2-naphthalenesulphonic acid, 4-toluenesulphonic acid, camphorsulphonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally, and parenterally. In one embodiment, the compound is administered orally. One skilled in the art will recognise the advantages of certain routes of administration.

The dosage regimen utilising the compounds is selected in accordance with a variety of factors including type, species, age, weight, sex and medical condition of the patient; the severity of the condition to be treated; the route of administration; the renal and hepatic function of the patient; and the particular compound or salt thereof employed.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier, excipient, adjuvant, or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognise that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Examples

For exemplary purpose, salts of the compounds of Formula (I) are synthesised and tested in the examples. It is understood that neutral compounds of Formula (I) may be similarly synthesised and tested using the exemplary procedures described in the examples. Further, it is understood that the salts (e.g., sodium salt) of the compounds of Formula (I) may be converted to the corresponding neutral compounds using routine techniques in the art (e.g., pH adjustment and, optionally, extraction (e.g., into an aqueous phase)).

### Synthesis of Compounds

Compounds of Formula (I) can be prepared using the methods detailed herein. Those skilled in the art may be able to envisage alternative synthetic routes, using a variety of starting materials and reagents to prepare the disclosed compounds of Formula (I) and to make further modifications. For exemplary purpose, salts of some of the compounds of Formula (I) are synthesised and tested in the examples. It is understood that neutral compounds of Formula (I) may be similarly synthesised and tested using the exemplary procedures described in the examples. Further, it is understood that the salts (e.g., hydrochloride salt) of the compounds of Formula (I) may be converted to the corresponding neutral compounds using routine techniques in the art (e.g., pH adjustment and, optionally, extraction (e.g., into an aqueous phase)).

¹H, ¹³C and ¹⁹F Nuclear magnetic resonance (NMR) spectra were recorded at 400 MHz or 300 MHz as stated and at 300.3 K unless otherwise stated; the chemical shifts (6) are reported in parts per million (ppm), relative to the residual solvent peak and the multiplicity reported together with the associated coupling constant (J), where applicable. Spectra were recorded using a Bruker or Varian instrument with 8, 16, 32 or 64 scans.

LC-MS chromatograms and spectra were recorded using an Agilent 1200 or Shimadzu LC-20 AD&MS 2020 instrument using a C-18 column such as a Luna-C18 2.0×30 mm or Xbridge Shield RPC18 2.1×50 mm. Injection volumes were 0.7-8.0 μL and the flow rates were typically 0.8 or 1.2 mL/min. Detection methods were diode array (DAD) or evaporative light scattering (ELSD) as well as positive ion electrospray ionisation. MS range was 100-1000 Da. Solvents were gradients of water and acetonitrile both containing a modifier (typically 0.01-0.04%) such as trifluoroacetic acid or ammonium carbonate.

UPLC-MS analysis was carried out on a Waters Acquity UPLC system consisting of an Acquity I-Class Sample Manager-FL, Acquity I-Class Binary Solvent Manager and an Acquity UPLC Column Manager. UV detection was afforded using an Acquity UPLC PDA detector (scanning from 210 to 400 nm), whilst mass detection was achieved using an Acquity QDa detector (mass scanning from 100-1250 Da; positive and negative modes simultaneously), and ELS detection was achieved using an Acquity UPLC ELS Detector. A Waters Acquity UPLC BEH C18 column (2.1×50 mm, 1.7 mm) was used to separate the analytes.

Samples were typically prepared by dissolution (with or without sonication) into 1 mL of 50% (v/v) MeCN in water. The resulting solutions were then filtered through a 0.2 mm syringe filter before submitting for analysis. All of the solvents, including formic acid and 36% ammonia solution, were purchased as HPLC grade. Solvents were gradients of water and acetonitrile both containing a modifier (typically 0.01-0.04%) such as formic acid or ammonia.

### Abbreviations

Ac Acetate
AcOH Acetic acid
CDCl₃ Chloroform-d
COMU 1-Cyano-2-ethoxy-2-oxoethylidenaminooxy)dimethylamino-morpholino-carbenium hexafluorophosphate
DCM Dichloromethane
DIPEA Diisopropylethylamine
DMF N,N-Dimethylformamide
DMSO-d₆ Dimethyl sulfoxide-d6
EDCI 1-Ethyl-3-(3-dimethylaminopropyl)carbodiimide hydrochloride
eq. Equivalents
ESI Electrospray Ionisation
EtOAc Ethyl Acetate
EtOH Ethanol
FCC Flash column chromatography
h Hour(s)
HATU (1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium
HOBt 1-Hydroxybenzotriazole
HPLC High-performance Liquid Chromatography
LCMS Liquid Chromatography-Mass Spectrometry
LiHMDS Lithium bis(trimethylsilyl)amide
MeCN Acetonitrile
MeOH-d₄ Deuterated methanol
MeOH Methanol
min Minute(s)
MTBE Methyl tert-butylether
NBS N-bromosuccinimide
NCS N-chlorosuccinimide
NMM N-methylmorpholine
NMR Nuclear Magnetic Resonance
pet. Petroleum
RM Reaction mixture
RP Reverse phase
Rt Room temperature
TEA Triethylamine
TFA Trifluoroacetic acid
THF Tetrahydrofuran
TMS Trimethylsilyl
Y Yield

### Example 1. Synthesis of Compound of Formula (I) Synthesis of Intermediates

### Intermediate A1. N-((6-Chloropyridazin-3-yl)methyl)tetrahydro-2H-pyran-4-amine

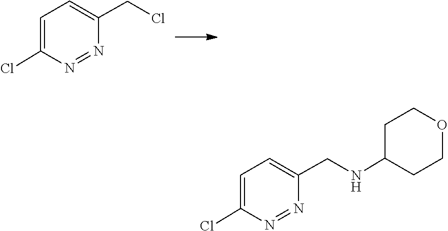 [SMILES: ClCc1ccc(Cl)nn1 ; Clc1ccc(CNC2CCOCC2)nn1]

To a solution of 3-chloro-6-(chloromethyl)pyridazine (800 mg, 4.91 mmol) in DMSO (8 mL) was added tetrahydropyran-4-amine (496 mg, 4.91 mmol) and DIPEA (634 mg, 4.91 mmol, 855 μL) at 25° C. under N₂. The mixture was stirred at 60° C. for 3 h then allowed to cool to room temperature. The crude mixture was purified directly by prep-HPLC (column: Waters Xbridge Prep OBD C18 150×40 mm, 10 μm; mobile phase: [(A) water (NH₄HCO₃)—(B) ACN]; Gradient: 5-35% B, 8 min) to give the title compound as a white solid, Y=72%. ¹H NMR (400 MHz, DMSO-d₆) δ 7.89-7.83 (m, 2H), 4.01 (s, 2H), 3.83-3.79 (m, 2H), 3.28-3.21 (m, 2H), 2.58-2.55 (m, 2H), 1.81-1.75 (m, 2H), 1.27-1.23 (m, 2H).

### Intermediate A2. N-((6-Chloro-5-methylpyridazin-3-yl)methyl)tetrahydro-2H-pyran-4-amine

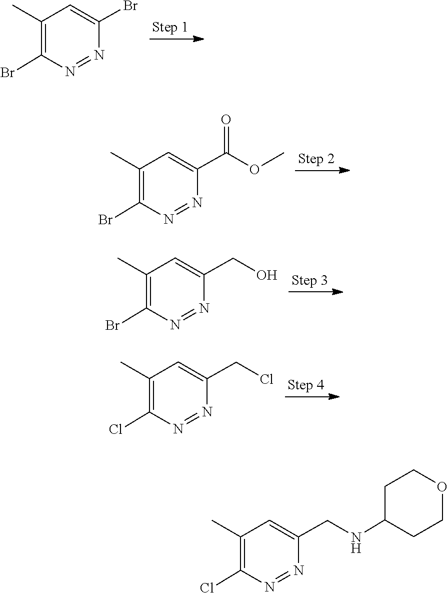

### Step 1. Methyl 6-bromo-5-methylpyridazine-3-carboxylate

To a solution of 3,6-dibromo-4-methyl-pyridazine (2.40 g, 9.53 mmol) in MeOH (50 mL) was added TEA (3.98 mL, 28.6 mmol) and Pd(dppf)Cl₂ (349 mg, 476 mol) at 25° C. under N₂. The suspension was vacuum purged and backfilled with CO three times, then the mixture was stirred under CO (50 Psi) at 60° C. for 8 h. After cooling to room temperature, the reaction mixture was concentrated under reduced pressure, diluted with H₂O (30 mL) and extracted with EtOAc (3×30 mL). The combined organic layers were washed with brine (90 mL), dried over Na₂SO₄, filtered and concentrated under reduced pressure. The crude material was purified by column chromatography (SiO₂, petroleum ether:ethyl acetate=10:1 to 1:1) to give the title compound as a white solid, Y=39%. ¹H NMR (400 MHz, DMSO-d₆) δ 8.23 (s, 1H), 3.96 (s, 3H), 2.46 (s, 3H).

### Step 2. (6-Bromo-5-methylpyridazin-3-yl)methanol

To a solution of methyl 6-bromo-5-methyl-pyridazine-3-carboxylate (0.87 g, 3.77 mmol) in DCM (2 mL) and MeOH (8 mL), at 0° C. under N₂ was added NaBH₄ (285 mg, 7.53 mmol). The mixture was stirred at 25° C. for 3 h then was cooled to 0° C. and quenched with 1 M HCl (8 mL). The mixture was extracted with EtOAc (3×8 mL) and the combined organic layers were washed with brine (20 mL), dried over Na₂SO₄, filtered and concentrated under reduced pressure. The residue was purified column chromatography (SiO₂, petroleum ether:ethyl acetate=10:1 to 0:1) to give the title compound as a yellow solid, Y=26%. ¹H NMR (400 MHz, DMSO-d₆) δ 7.72 (s, 1H), 4.68 (s, 2H), 2.39 (s, 3H).

### Step 3. 3-Chloro-6-(chloromethyl)-4-methylpyridazine

To a solution of (6-bromo-5-methyl-pyridazin-3-yl)methanol (0.20 g, 985 mol) in DCM (2.7 mL), under N₂, was added SOCl₂ (1.43 mL, 19.7 mmol) at 25° C. The mixture was stirred at 25° C. for 2 h then cooled to 0° C. and quenched with sat. NaHCO₃ solution (3 mL). The mixture was extracted with DCM (3×3 mL) and the combined organic layers were washed with brine (9 mL), dried over Na₂SO₄, filtered and concentrated under reduced pressure to give the title compound as a yellow solid, Y=86%.

### Step 4. N-((6-Chloro-5-methylpyridazin-3-yl)methyl)tetrahydro-2H-pyran-4-amine

To a solution of 3-chloro-6-(chloromethyl)-4-methyl-pyridazine (50 mg, 282 mol) in DMSO (1 mL) was added tetrahydropyran-4-amine (29 mg, 282 mol) and DIPEA (98 μL) at 25° C. The mixture was stirred at 120° C. for 15 min under N₂ then allowed to cool to room temperature and diluted with H₂O (1 mL). The mixture was extracted with EtOAc (3×1 mL).

The combined organic layers were washed with brine (3 mL), dried over Na₂SO₄, filtered and concentrated under reduced pressure. The residue was purified by prep-HPLC (column: Waters Xbridge C18 150×50 mm, 10 μm; mobile phase: [(A) H₂O (10 mM NH₄HCO₃)—(B) ACN]; gradient: 10-30% B, 8 min) and lyophilised to give the title compound as a white solid, Y=59%. ¹H NMR (400 MHz, DMSO-d₆) δ 7.78 (s, 1H), 3.96 (s, 2H), 3.85-3.78 (m, 2H), 3.29-3.20 (m, 2H), 2.63-2.54 (m, 1H), 2.37 (s, 3H), 1.85-1.70 (m, 2H), 1.32-1.20 (m, 2H).

### Intermediate B1. 3-Methyl-2-(4,4,5,5-tetramethyl-1,3,2-dioxaborolan-2-yl)-5-(trifluoromethyl)phenol

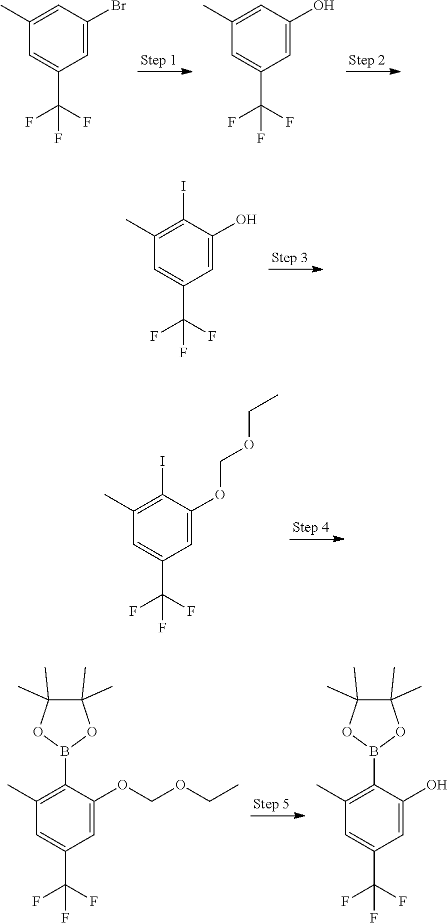 [SMILES: Cc1cc(Br)cc(C(F)(F)F)c1 ; Cc1cc(O)cc(C(F)(F)F)c1 ; Cc1cc(C(F)(F)F)cc(O)c1I ; CCOCOc1cc(C(F)(F)F)cc(C)c1I ; CCOCOc1cc(C(F)(F)F)cc(C)c1B1OC(C)(C)C(C)(C)O1 ; Cc1cc(C(F)(F)F)cc(O)c1B1OC(C)(C)C(C)(C)O1]

### Step 1. 3-Methyl-5-(trifluoromethyl)phenol

To a solution of 1-bromo-3-methyl-5-(trifluoromethyl)benzene (9.67 g, 40.5 mmol) in dioxane (100 mL) and H₂O (10 mL), at 25° C. under N₂, was added Pd₂(dba)₃ (741 mg, 809 mol), di-tert-butyl-[2-(1,3,5-triphenylpyrazol-4-yl)pyrazol-3-yl]phosphane (820 mg, 1.62 mmol) and LiOH·H₂O (5.09 g, 121 mmol). The mixture was stirred at 100° C. for 16 h then allowed to cool to room temperature. The mixture was filtered through a pad of celite then the filtrate was acidified by 2 M HCl solution (50 mL) and extracted with EtOAc (3×100 mL). The combined organic layers were washed with brine (100 mL), dried over anhydrous Na₂SO₄, filtered and concentrated under reduced pressure to give the title compound as a yellow oil, Y=84%. ¹H NMR (400 MHz, CDCl₃) δ 7.02 (s, 1H), 6.89 (s, 1H), 6.83 (s, 1H), 2.37 (s, 3H).

### Step 2. 2-Iodo-3-methyl-5-(trifluoromethyl)phenol

To a solution of 3-methyl-5-(trifluoromethyl)phenol (10.0 g, 56.8 mmol) in toluene (250 mL) was added NaH (3.63 g, 90.8 mmol, 60% in mineral oil) at 0° C. The mixture was stirred at 0° C. for 30 min then was treated with a solution of I₂ (9.15 mL, 45.4 mmol) in toluene (50 mL). The mixture was stirred at 25° C. for 2 h then quenched by addition of H₂O (100 mL) at 0° C. and adjusted to pH=4 by 2 M HCl aqueous solution. The mixture was extracted with EtOAc (3×300 mL) and the combined organic layers were washed with brine (300 mL), dried over anhydrous Na₂SO₄, filtered and concentrated under reduced pressure. The residue was purified by column chromatography (SiO₂, petroleum ether:ethyl acetate=20:1 to 10:1) to give as a yellow oil, Y=47%. ¹H NMR (400 MHz, DMSO-d₆) δ 10.96 (s, 1H), 7.13 (s, 1H), 6.93 (s, 1H), 2.46-2.39 (s, 3H).

### Step 3. 1-(Ethoxymethoxy)-2-iodo-3-methyl-5-(trifluoromethyl)benzene

To a solution of 2-iodo-3-methyl-5-(trifluoromethyl)phenol (6.48 g, 21.5 mmol) in DMF (65 mL) was added Cs₂CO₃ (6.99 g, 21.5 mmol) and chloromethoxyethane (3.58 mL 38.6 mmol) at 25° C. The mixture was stirred for 16 h then diluted with H₂O (20 mL) and extracted with EtOAc (3×20 mL). The combined organic layers were washed with brine (20 mL), dried over anhydrous Na₂SO₄, filtered and concentrated under reduced pressure. The crude material was purified by column chromatography (SiO₂, petroleum ether:ethyl acetate=20:1 to 10:1) to give the title compound as a yellow oil, Y=78. ¹H NMR (400 MHz, DMSO-d₆) δ 7.36 (s, 1H), 7.18 (s, 1H), 5.41 (s, 2H), 3.74 (q, J=7 Hz, 2H), 2.48 (s, 3H), 1.13 (t, J=7 Hz, 3H).

### Step 4. 2-(2-(Ethoxymethoxy)-6-methyl-4-(trifluoromethyl)phenyl)-4,4,5,5-tetramethyl-1,3,2-dioxaborolane

To a solution of 1-(ethoxymethoxy)-2-iodo-3-methyl-5-(trifluoromethyl)benzene (6.00 g, 16.7 mmol) in dioxane (60 mL), at 25° C. under N₂ were added TEA (17.2 mL, 123 mmol) then 4,4,5,5-tetramethyl-1,3,2-dioxaborolane (12.1 mL, 83.3 mmol), Pd(OAc)₂ (486 mg, 2.17 mmol) and dicyclohexyl-(2-phenylphenyl)phosphane (1.40 g, 4.00 mmol). The mixture was stirred at 80° C. for 18 h then allowed to cool to room temperature. The mixture was diluted with H₂O (60 mL) and extracted with EtOAc (3×60 mL). The combined organic layers were washed with brine (60 mL), dried over Na₂SO₄, filtered and concentrated under reduced pressure to give a residue. The residue was purified by column chromatography (SiO₂, petroleum ether:ethyl acetate=20:1 to 10:1) to give the title compound as a yellow oil, Y=83%. ¹H NMR (400 MHz, DMSO-d₆) δ 7.15 (s, 1H), 7.12 (s, 1H), 5.26 (s, 2H), 3.66 (q, J=7 Hz, 2H), 2.32 (s, 3H), 1.32 (s, 12H), 1.12 (t, J=7 Hz, 3H).

### Step 5. 3-Methyl-2-(4,4,5,5-tetramethyl-1,3,2-dioxaborolan-2-yl)-5-(trifluoromethyl)phenol

To a solution of 2-[2-(ethoxymethoxy)-6-methyl-4-(trifluoromethyl)phenyl]-4,4,5,5-tetramethyl-1,3,2-dioxaborolane (5.00 g, 13.9 mmol) in DCM (150 mL) was added TFA (30.8 mL, 416 mmol) at 0° C. The mixture was stirred at 0° C. for 1 h then concentrated under reduced pressure. The crude material was purified by column chromatography (SiO₂, petroleum ether:ethyl acetate=20:1 to 10:1) to give the title compound as a yellow oil, Y=62%. ¹H NMR (400 MHz, DMSO-d₆) δ 9.79 (s, 1H), 6.92 (s, 1H), 6.83 (s, 1H), 2.30 (s, 3H), 1.31 (s, 12H).

### Intermediate B2. 2-Methyl-6-(4,4,5,5-tetramethyl-1,3,2-dioxaborolan-2-yl)-3-(trifluoromethyl)phenol

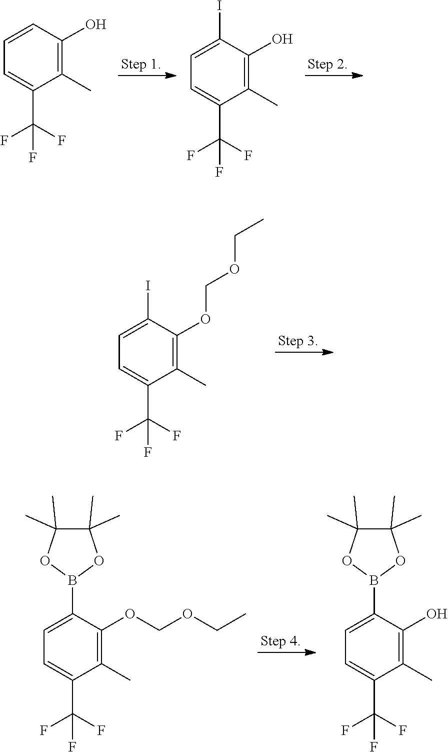

### Step 1. 6-Iodo-2-methyl-3-(trifluoromethyl)phenol

To a solution of 2-methyl-3-(trifluoromethyl) phenol (2.00 g, 11.4 mmol) in toluene (40 mL) at 0° C. and under N₂ was added NaH (545 mg, 13.6 mmol, 60% in mineral oil). The reaction mixture was stirred at 0° C. for 1 h then I₂ (2.29 mL, 11.4 mmol) was added. The reaction mixture was stirred at 0° C. for a further 2 h then sat. ammonium chloride solution (40 mL) was added. The resulting mixture was extracted with ethyl acetate (3×40 mL). The combined organic layers were washed with brine (40 mL), dried over Na₂SO₄, filtered and concentrated under reduced pressure. The crude material was purified by column chromatography (SiO₂, petroleum ether:ethyl acetate=10:1) to give the title compound as a white solid, Y=92%. ¹H NMR (400 MHz, DMSO-d₆) δ 9.61 (s, 1H), 7.76 (d, J=8 Hz, 1H), 6.96 (d, J=8 Hz, 1H), 2.31 (s, 3H).

### Step 2. 2-(Ethoxymethoxy)-1-iodo-3-methyl-4-(trifluoromethyl)benzene

To a mixture of 6-iodo-2-methyl-3-(trifluoromethyl)phenol (3.16 g, 10.5 mmol) and Cs₂CO₃ (6.82 g, 20.9 mmol) in DMF (32 mL) was added chloromethoxyethane (971 μL, 10.5 mmol) at 25° C. The mixture was stirred at 25° C. for 2 h then H₂O (32 mL), was added. The mixture was extracted with ethyl acetate (3×32 mL) and the combined organic layers were washed with brine (32 mL), dried over Na₂SO₄, filtered and concentrated under reduced pressure. The crude material was purified by column chromatography (SiO₂, petroleum ether:ethyl acetate=10:1) to give the title compound as a white solid, Y=98%. ¹H NMR (400 MHz, DMSO-d₆) δ 7.89 (d, J=8 Hz, 1H), 7.23 (d, J=8 Hz, 1H), 5.08 (s, 2H), 3.85 (q, J=7 Hz, 2H), 2.38 (s, 3H), 1.18 (t, J=7 Hz, 3H).

### Step 3. 2-(2-(Ethoxymethoxy)-3-methyl-4-(trifluoromethyl)phenyl)-4,4,5,5-tetramethyl-1,3,2-dioxaborolane

To a solution of 2-(ethoxymethoxy)-1-iodo-3-methyl-4-(trifluoromethyl)benzene (500 mg, 1.39 mmol) in dioxane (5 mL) was added TEA (1.43 mL, 10.3 mmol), 4,4,5,5-tetramethyl-1,3,2-dioxaborolane (1.01 mL, 6.94 mmol), Pd(OAc)₂ (40.5 mg, 181 mol), dicyclohexyl-(2-phenylphenyl)phosphane (117 mg, 333 mol) at 25° C. The mixture was stirred at 80° C. for 12 h under N₂ then allowed to cool to room temperature. The mixture was diluted with H₂O (5 mL) and extracted with ethyl acetate (3×5 mL). The combined organic layers were washed with brine (5 mL), dried over Na₂SO₄, filtered and concentrated under reduced pressure. The crude material was purified by column chromatography (SiO₂, petroleum ether:ethyl acetate=10:1) to give the title compound as a white solid, Y=60%. ¹H NMR (400 MHz, DMSO-d₆) δ 7.62 (d, J=8 Hz, 1H), 7.45 (d, J=8 Hz, 1H), 5.07 (s, 2H), 3.73 (q, J=7 Hz, 2H), 2.35 (s, 3H), 1.30 (s, 12H), 1.14 (t, J=7 Hz, 3H).

### Step 4. 2-Methyl-6-(4,4,5,5-tetramethyl-1,3,2-dioxaborolan-2-yl)-3-(trifluoromethyl)phenol

A mixture of 2-[2-(ethoxymethoxy)-3-methyl-4-(trifluoromethyl) phenyl]-4,4,5,5-tetramethyl-1,3,2-dioxaborolane (300 mg, 833 mol) and 4 M HCl in EtOAc (3 mL) was stirred at 25° C. for 2 h. The reaction mixture was concentrated under reduced pressure to give the title compound as a white solid, Y=99%.

### Intermediate C1. 2-(6-(Chloromethyl)pyridazin-3-yl)-3-methyl-5-(trifluoromethyl)phenol

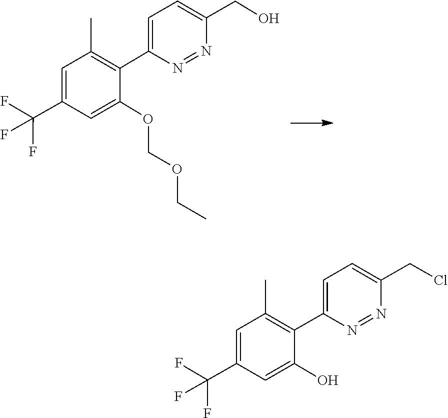 [SMILES: CCOCOc1cc(C(F)(F)F)cc(C)c1-c1ccc(CO)nn1 ; Cc1cc(C(F)(F)F)cc(O)c1-c1ccc(CCl)nn1]

The following reaction was performed three times in parallel and the crude reaction mixtures were combined before workup. To a solution of [6-[2-(ethoxymethoxy)-6-methyl-4-(trifluoromethyl)phenyl]pyridazin-3-yl]methanol (Intermediate C3, 4.33 g, 12.7 mmol) in DCM (220 mL) was added SOCl₂ (21.7 mL, 298 mmol) at 25° C. The reaction mixture was stirred at 25° C. for 1 h then TFA (21.7 mL, 291 mmol) was added and the mixture stirred for a further 1 h.

The combined reaction mixtures were concentrated under reduced pressure then diluted with H₂O (100 mL) and extracted with EtOAc (3×100 mL). The combined organic layers were washed with H₂O (3×100 mL) and concentrated under reduced pressure. The crude material was triturated with MTBE (100 mL), filtered and the filter cake dried under high vacuum to give the title compound as a yellow oil, Y=78%. ¹H NMR (400 MHz, DMSO-d₆) δ 10.36 (s, 1H), 7.93 (d, J=9 Hz, 1H), 7.79 (d, J=9 Hz, 1H), 7.18 (s, 1H), 7.11 (s, 1H), 5.07 (s, 2H), 2.09 (s, 3H).

### Intermediate C2. 5-Chloro-2-(6-(chloromethyl)pyridazin-3-yl)-3-methylphenol

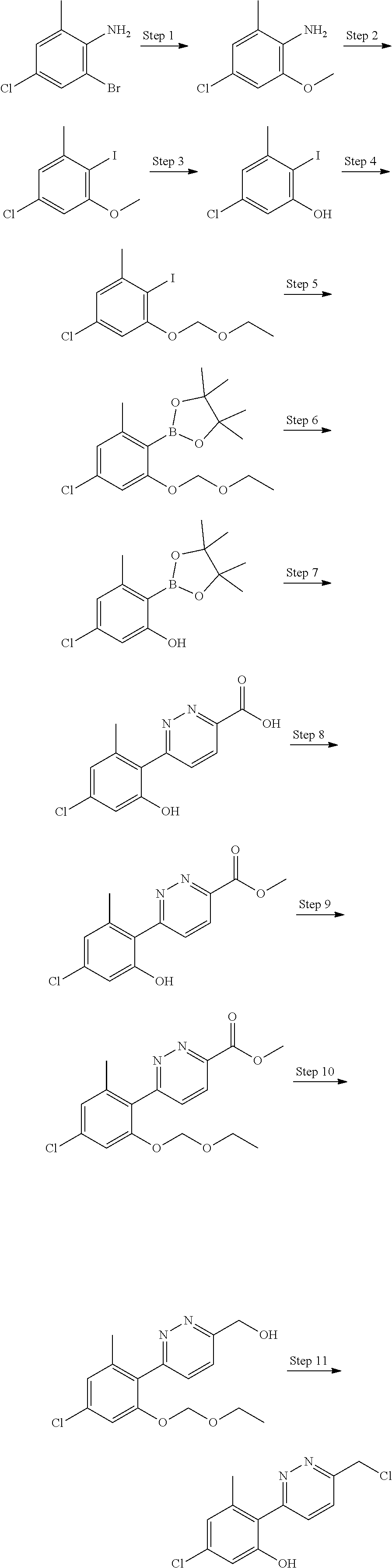

### Step 1. 4-Chloro-2-methoxy-6-methylaniline

To a solution of 2-bromo-4-chloro-6-methyl-aniline (125 g, 567 mmol) in MeOH (125 mL) at 25° C. were added NaOMe (30% wt, 666 mL, 567 mmol) and CuI (124 g, 652 mmol). The mixture was stirred at reflux temperature for 2 h then allowed to cool to room temperature. The reaction mixture was filtered and the filtrate was diluted with saturated aqueous NH₄Cl solution (400 mL) and extracted with EtOAc (3×400 mL). The combined organic layers were washed with brine (400 mL), dried over Na₂SO₄, filtered and concentrated under reduced pressure to give a residue. The residue was purified by column chromatography (SiO₂, petroleum ether:ethyl acetate=1:0 to 10:1) to give a set of pure fractions and a set of impure fractions. Impure fractions were further purified by prep-HPLC (column: Phenomenex Luna C18 250×150 mm, 15 μm; mobile phase: [(A) H₂O (10 mM NH₄HCO₃)—(B) ACN]; gradient: 35-65% B, 22 min), and the two product batches were combined and lyophilised to give the title compound as a yellow solid, Y=73%. ¹H NMR (400 MHz, DMSO-d₆) δ 6.72 (d, J=2 Hz, 1H), 6.65 (d, J=2 Hz, 1H), 4.54 (s, 2H), 3.76 (s, 3H), 2.05 (s, 3H).

### Step 2. 5-Chloro-2-iodo-1-methoxy-3-methylbenzene

The following reaction was performed two times in parallel. To a solution of 4-chloro-2-methoxy-6-methyl-aniline (37.0 g, 216 mmol) in ACN (370 mL) was added 12 (52.1 mL, 259 mmol) at 25° C. then tert-butyl nitrite (38.5 mL, 323 mmol) was added dropwise. The mixture was stirred at 25° C. for 12 h. The two reaction mixtures were combined and quenched with saturated aqueous Na₂S₂O₂ solution (740 mL), and extracted with EtOAc (3×740 mL). The combined organic phase was washed with brine (370 mL), dried with anhydrous Na₂SO₄, filtered and concentrated in vacuum. The residue was purified by column chromatography (SiO₂, petroleum ether:ethyl acetate=1:0 to 10:1) to give the title compound as a yellow solid, Y=48%. ¹H NMR (400 MHz, DMSO-d₆) δ 7.05 (d, J=2 Hz, 1H), 6.89 (d, J=2 Hz, 1H), 3.84 (s, 3H), 2.37 (s, 3H).

### Step 3. 5-Chloro-2-iodo-3-methylphenol

To a solution of 5-chloro-2-iodo-1-methoxy-3-methyl-benzene (58.0 g, 205 mmol) in DCM (770 mL) was added BBr₃ (39.6 mL, 411 mmol) at 0° C. The mixture was stirred at 25° C. for 8 h then poured into water (700 mL) and extracted with DCM (3×700 mL). The organic phases were combined, washed with brine (2×700 mL), dried with anhydrous Na₂SO₄, filtered and concentrated under reduced pressure to give the title compound as a yellow solid, Y=100%. ¹H NMR (400 MHz, DMSO-d₆) δ 10.73 (s, 1H), 6.88 (d, J=2 Hz, 1H), 6.72 (d, J=2 Hz, 1H), 2.34 (s, 3H).

### Step 4. 5-Chloro-1-(ethoxymethoxy)-2-iodo-3-methylbenzene

To a solution of 5-chloro-2-iodo-3-methyl-phenol (55.0 g, 205 mmol) in ACN (550 mL) was added Cs₂CO₃ (133 g, 410 mmol) and chloromethoxyethane (19.0 mL, 205 mmol). The mixture was stirred at 25° C. for 6 h then was filtered and concentrated under reduced pressure. The residue was purified by column chromatography (SiO₂, Petroleum ether:Ethyl acetate=1:0 to 10:1) to give the title compound as a colourless oil, Y=91%. ¹H NMR (400 MHz, DMSO-d₆) δ 7.10 (d, J=2 Hz, 1H), 6.99 (d, J=2 Hz, 1H), 5.34 (s, 2H), 3.69 (q, J=7 Hz, 2H), 2.39 (s, 3H), 1.13 (t, J=7 Hz, 3H).

### Step 5. 2-(4-Chloro-2-(ethoxymethoxy)-6-methylphenyl)-4,4,5,5-tetramethyl-1,3,2-dioxaborolane

The following reaction was performed two times in parallel. To a solution of 5-chloro-1-(ethoxymethoxy)-2-iodo-3-methyl-benzene (25.0 g, 76.6 mmol) in dioxane (500 mL) was added TEA (32.0 mL, 230 mmol) and 4,4,5,5-tetramethyl-1,3,2-dioxaborolane (22.2 mL, 153 mmol) at 25° C. under N₂. This was then treated with Pd(OAc)₂ (1.72 g, 7.66 mmol) and dicyclohexyl-(2-phenylphenyl)phosphane (5.37 g, 15.3 mmol) at 25° C. under N₂. The mixture was stirred at 80° C. for 5 h under N₂. Both crude reaction mixtures were combined and diluted with H₂O (500 mL) and extracted with EtOAc (3×500 mL). The combined organic layers were washed with brine (500 mL), dried over anhydrous Na₂SO₄, filtered and concentrated under reduced pressure. The residue was purified by prep-HPLC (column: Welch Xtimate C18 250×100 mm, 10 μm; mobile phase: [(A) H₂O (10 mM NH₄HCO₃)—(B) ACN]; gradient: 50-85% B, 20 min) and lyophilised to give the title compound as a white solid, Y=75%. ¹H NMR (400 MHz, CDCl₃) δ 6.88 (s, 1H), 6.81 (s, 1H), 5.17 (s, 2H), 3.72 (q, J=7 Hz, 2H), 2.33 (s, 3H), 1.38 (s, 12H), 1.23 (t, J=7 Hz, 3H).

### Step 6. 5-Chloro-3-methyl-2-(4,4,5,5-tetramethyl-1,3,2-dioxaborolan-2-yl)phenol

A mixture of 2-[4-chloro-2-(ethoxymethoxy)-6-methyl-phenyl]-4,4,5,5-tetramethyl-1,3,2-dioxaborolane (6.00 g, 18.4 mmol) and 4 M HCl in EtOAc (30 mL) was stirred at 25° C. for 2 h. The reaction mixture was concentrated under reduced pressure to give the title compound as a yellow oil, Y=99%. ¹H NMR (400 MHz, DMSO-d₆) δ 9.48 (s, 1H), 6.66 (s, 1H), 6.60 (s, 1H), 2.24 (s, 3H), 1.29 (s, 12H).

### Step 7. 6-(4-Chloro-2-hydroxy-6-methylphenyl)pyridazine-3-carboxylic acid

To a solution of 5-chloro-3-methyl-2-(4,4,5,5-tetramethyl-1,3,2-dioxaborolan-2-yl)phenol (4.67 g, 17.4 mmol) in dioxane (45 mL) and H₂O (15 mL) were added methyl 6-chloropyridazine-3-carboxylate (3.00 g, 17.4 mmol), Na₂CO₃ (5.53 g, 52.2 mmol) and Pd(PPh₃)₄ (1.00 g, 869 mol) at 25° C. under N₂. The mixture was stirred at 80° C. for 2 h then allowed to cool to room temperature, diluted with H₂O (50 mL) and extracted with EtOAc (40 mL). The aqueous phase was adjusted to pH=4 with aqueous 2 M HCl and extracted with EtOAc (3×50 mL). The combined organic layers were washed with brine (2×50 mL), dried over Na₂SO₄, filtered and concentrated under reduced pressure. The residue was triturated with MTBE at 25° C. for 10 min, filtered, and the filter cake dried under reduced pressure to give the title compound as a yellow solid, Y=72%. ¹H NMR (400 MHz, DMSO-d₆) δ 13.97 (br. s, 1H), 10.23 (s, 1H), 8.22 (d, J 9 Hz, 1H), 7.86 (d, J 9 Hz, 1H), 6.92 (s, 1H), 6.88 (s, 1H), 2.04 (s, 3H).

### Step 8. Methyl 6-(4-chloro-2-hydroxy-6-methylphenyl)pyridazine-3-carboxylate

To a solution of 6-(4-chloro-2-hydroxy-6-methyl-phenyl)pyridazine-3-carboxylic acid (3.30 g, 12.5 mmol) in MeOH (33 mL) was added SOCl₂ (1.81 mL, 24.9 mmol) at 25° C. The mixture was stirred at 80° C. for 2 h under N₂. The reaction mixture was quenched by addition of H₂O (5 mL), then filtered, and the filter cake was dried in vacuum to give a residue. The residue was triturated with MTBE at 25° C. for 10 min, then filtered, and the filter cake was dried under reduced pressure to give the title compound as a white solid, Y=95%.

### Step 9. Methyl 6-(4-chloro-2-(ethoxymethoxy)-6-methylphenyl)pyridazine-3-carboxylate

To a solution of methyl 6-(4-chloro-2-hydroxy-6-methyl-phenyl)pyridazine-3-carboxylate (3.0 g, 10.8 mmol) in DMF (30 mL) were added Cs₂CO₃ (7.01 g, 21.5 mmol) and chloromethoxyethane (1.0 mL, 10.8 mmol) at 25° C. The mixture was stirred at 25° C. for 2 h under N₂. The reaction mixture was diluted with H₂O (30 mL) and extracted with EtOAc (3×30 mL). The combined organic layers were washed with brine (30 mL), dried over Na₂SO₄, filtered and concentrated under reduced pressure. The residue was purified by column chromatography (SiO₂, petroleum ether:ethyl acetate=10:1 to 1:1) to give the title compound as a yellow solid, Y=48%. ¹H NMR (400 MHz, DMSO-d₆) δ 8.28 (d, J 9 Hz, 1H), 7.90 (d, J 9 Hz, 1H), 7.21 (s, 1H), 7.15 (s, 1H), 5.17 (s, 2H), 3.98 (s, 3H), 3.44 (q, J 7 Hz, 2H), 2.02 (s, 3H), 1.03 (t, J 7 Hz, 3H).

### Step 10. (6-(4-Chloro-2-(ethoxymethoxy)-6-methylphenyl)pyridazin-3-yl)methanol

To a solution of methyl 6-[4-chloro-2-(ethoxymethoxy)-6-methyl-phenyl]pyridazine-3-carboxylate (1.75 g, 5.20 mmol) in MeOH (18 mL) was added NaBH₄ (1.08 g, 28.6 mmol) at 0° C. under N₂. The mixture was stirred at 0° C. for 10 min, quenched by addition of aqueous 1 M HCl (10 mL), and extracted with EtOAc (3×20 mL). The combined organic layers were washed with brine (30 mL), dried over Na₂SO₄, filtered, and concentrated under reduced pressure to give the title compound as a white solid, Y=75%. ¹H NMR (400 MHz, DMSO-d₆) δ 7.78 (d, J 9 Hz, 1H), 7.66 (d, J 9 Hz, 1H), 7.19 (s, 1H), 7.12 (s, 1H), 5.69 (t, J 6 Hz, 1H), 5.17 (s, 2H), 4.82 (d, J 6 Hz, 2H), 3.46 (q, J 7 Hz, 2H), 2.00 (s, 3H), 1.04 (t, J 7 Hz, 3H).

### Step 11. 5-Chloro-2-(6-(chloromethyl)pyridazin-3-yl)-3-methylphenol

To a solution of [6-[4-chloro-2-(ethoxymethoxy)-6-methyl-phenyl]pyridazin-3-yl]methanol (1.20 g, 3.89 mmol) in DCM (13 mL) was added SOCl₂ (6.00 mL, 82.6 mmol) at 25° C. h under N₂. The mixture was stirred at 25° C. for 2 h then TFA (6.00 mL, 80.8 mmol) was added. The mixture was stirred at 25° C. for 2 h then concentrated under reduced pressure. The residue was triturated with EtOAc, then filtered, and the filter cake was dried under reduced pressure to give the title compound as a white solid, Y=76%. ¹H NMR (400 MHz, DMSO-d₆) δ 10.14 (s, 1H), 7.89 (d, J 9 Hz, 1H), 7.74 (d, J 9 Hz, 1H), 6.90 (s, 1H), 6.87 (s, 1H), 5.05 (s, 2H), 2.02 (s, 3H).

### Intermediate C3. (6-(2-(Ethoxymethoxy)-6-methyl-4-(trifluoromethyl)phenyl)pyridazin-3-yl)methanol

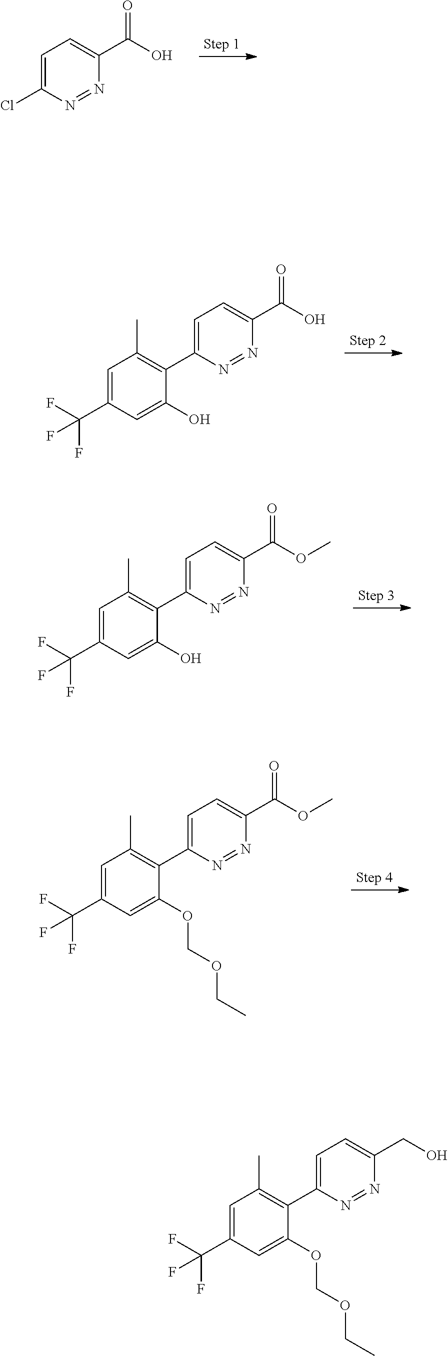 [SMILES: O=C(O)c1ccc(Cl)nn1 ; Cc1cc(C(F)(F)F)cc(O)c1-c1ccc(C(=O)O)nn1 ; COC(=O)c1ccc(-c2c(C)cc(C(F)(F)F)cc2O)nn1 ; CCOCOc1cc(C(F)(F)F)cc(C)c1-c1ccc(C(=O)OC)nn1 ; CCOCOc1cc(C(F)(F)F)cc(C)c1-c1ccc(CO)nn1]

### Step 1. 6-(2-Hydroxy-6-methyl-4-(trifluoromethyl)phenyl)pyridazine-3-carboxylic acid

To a solution of methyl 6-chloropyridazine-3-carboxylate (10.0 g, 58.0 mmol) in dioxane (500 mL) and H₂O (167 mL) were added 3-methyl-2-(4,4,5,5-tetramethyl-1,3,2-dioxaborolan-2-yl)-5-(trifluoromethyl)phenol (17.5 g, 58.0 mmol), Na₂CO₃ (18.4 g, 174 mmol) and Pd(PPh₃)₄ (3.35 g, 2.90 mmol) at 25° C. under N₂. The mixture was stirred at reflux for 8 h then allowed to cool to room temperature. The reaction mixture was diluted with H₂O (500 mL) and the extracted with EtOAc (500 mL). The aqueous phase was adjusted to pH 3 with 2 M HCl solution then extracted with EtOAc (2×500 mL). The combined organic layers were washed with brine (500 mL), dried over Na₂SO₄, filtered and concentrated under reduced pressure. The crude material was triturated with MTBE (2×100 mL) then filtered to give a solid. The solid was dissolved in MeOH (100 mL) and the solution was concentrated under reduced pressure to give the title compound as a white solid, Y=90%. ¹H NMR (400 MHz, DMSO-d₆) δ 13.92 (br. s, 1H), 10.41 (s, 1H), 8.26 (d, J=9 Hz, 1H), 7.92 (d, J=9 Hz, 1H), 7.20 (s, 1H), 7.12 (s, 1H), 2.11 (s, 3H).

### Step 2. Methyl 6-(2-hydroxy-6-methyl-4-(trifluoromethyl)phenyl)pyridazine-3-carboxylate

To a solution of 6-[2-hydroxy-6-methyl-4-(trifluoromethyl)phenyl]pyridazine-3-carboxylic acid (20.0 g, 67.1 mmol) in MeOH (200 mL) was added SOCl₂ (9.74 mL, 134 mmol) at 25° C. under N₂. The mixture was stirred at 80° C. for 2 h then allowed to cool to room temperature. The reaction mixture was poured into H₂O (200 mL) and the resulting precipitate filtered. The filter cake was dried under reduced pressure to give the title compound as a yellow solid, Y=87%. ¹H NMR (400 MHz, DMSO-d₆) δ 10.42 (s, 1H), 8.29 (d, J=9 Hz, 1H), 7.95 (d, J=9 Hz, 1H), 7.20 (s, 1H), 7.12 (s, 1H), 3.99 (s, 3H), 2.10 (s, 3H).

### Step 3. Methyl 6-(2-(ethoxymethoxy)-6-methyl-4-(trifluoromethyl)phenyl)pyridazine-3-carboxylate

To a solution of methyl 6-[2-hydroxy-6-methyl-4-(trifluoromethyl)phenyl]pyridazine-3-carboxylate (17.5 g, 56.1 mmol) in DMF (175 mL), under N₂, was added Cs₂CO₃ (36.5 g, 112 mmol) and chloromethoxyethane (15.6 mL, 168 mmol). The mixture was stirred at 25° C. for 2 h then filtered and the filtrate was concentrated under reduced pressure. The crude material was triturated with petroleum ether (150 mL) then filtered and the filter cake was dried under high vacuum to give the title compound as a yellow solid, Y=87%. ¹H NMR (400 MHz, DMSO-d₆) δ 8.33 (d, J=9 Hz, 1H), 7.97 (d, J=9 Hz, 1H), 7.44 (s, 2H), 5.24 (s, 2H), 4.00 (s, 3H), 3.46 (q, J=7 Hz, 2H), 2.10 (s, 3H), 1.03 (t, J=7 Hz, 3H).

### Step 4. (6-(2-(Ethoxymethoxy)-6-methyl-4-(trifluoromethyl)phenyl)pyridazin-3-yl)methanol

To a solution of methyl 6-[2-(ethoxymethoxy)-6-methyl-4-(trifluoromethyl)phenyl]pyridazine-3-carboxylate (17.4 g, 45.0 mmol) in MeOH (174 mL) at 0° C. was added NaBH₄ (9.78 g, 258 mmol). The mixture was stirred at 0° C. for 0.5 h then quenched by addition of H₂O (170 mL). The mixture was extracted with EtOAc (3×170 mL) and the combined organic layers washed with brine (170 mL), dried over Na₂SO₄, filtered and concentrated under reduced pressure. The crude material was purified by column chromatography (SiO₂, petroleum ether:ethyl acetate=1:0 to 1:1) to give the title compound as a yellow solid, Y=83%. ¹H NMR (400 MHz, DMSO-d₆) δ 7.81 (d, J=9 Hz, 1H), 7.72 (d, J=9 Hz, 1H), 7.41 (s, 2H), 5.69 (t, J=6 Hz, 1H), 5.23 (s, 2H), 4.83 (d, J=6 Hz, 2H), 3.47 (q, J=7 Hz, 2H), 2.08 (s, 3H), 1.04 (t, J=7 Hz, 3H).

### Intermediate C4. 6-(2-(Ethoxymethoxy)-6-methyl-4-(trifluoromethyl)phenyl)pyridazine-3-carbaldehyde

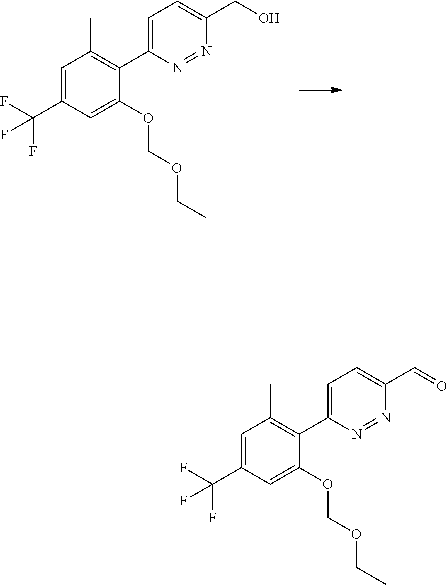 [SMILES: CCOCOc1cc(C(F)(F)F)cc(C)c1-c1ccc(CO)nn1 ; CCOCOc1cc(C(F)(F)F)cc(C)c1-c1ccc(C=O)nn1]

To a solution of [6-[2-(ethoxymethoxy)-6-methyl-4-(trifluoromethyl)phenyl]pyridazin-3-yl]methanol (Intermediate C3, 0.5 g, 1.46 mmol) in DCM (10 mL) was added Dess-Martin periodinane (679 μL, 2.19 mmol) at 0° C. The mixture was stirred at 25° C. for 2 h under N₂ atmosphere then diluted with H₂O (10 mL) and extracted with DCM (3×10 mL). The combined organic layers were washed with brine (10 mL), dried over Na₂SO₄, filtered and concentrated under reduced pressure. The residue was purified by column chromatography (SiO₂, Petroleum ether:Ethyl acetate=3:1 to 0:1) to give the title compound as a yellow oil, Y=91%. ¹H NMR (400 MHz, DMSO-d₆) δ 10.30 (s, 1H), 8.21 (d, J=9 Hz, 1H), 8.01 (d, J=9 Hz, 1H), 7.45 (s, 2H), 5.24 (s, 2H), 3.46 (q, J=7 Hz, 2H), 2.11 (s, 3H), 1.03 (t, J=7 Hz, 3H).

### Intermediate C5. 3-(Chloromethyl)-6-(2-(ethoxymethoxy)-6-methyl-4-(trifluoromethyl)phenyl)pyridazine

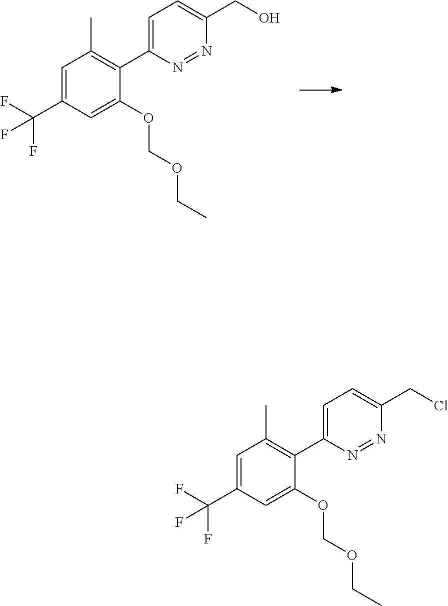 [SMILES: CCOCOc1cc(C(F)(F)F)cc(C)c1-c1ccc(CO)nn1 ; CCOCOc1cc(C(F)(F)F)cc(C)c1-c1ccc(CCl)nn1]

To a solution of [6-[2-(ethoxymethoxy)-6-methyl-4-(trifluoromethyl)phenyl]pyridazin-3-yl]methanol (Intermediate C3, 4.50 g, 13.2 mmol) in DCM (225 mL) was added SOCl₂ (955 L, 13.2 mmol) at 25° C. The mixture was stirred for 10 min then was poured into saturated aqueous Na₂CO₃ (100 mL). The mixture was extracted with DCM (3×100 mL) and the combined organic layers were washed with brine (100 mL), dried over Na₂SO₄, filtered and concentrated under reduced pressure. The residue was purified by column chromatography (SiO₂, petroleum ether:ethyl acetate=1:0 to 1:1) to give the title compound as a yellow solid, Y=70%. ¹H NMR (400 MHz, DMSO-d₆) δ 7.97 (d, J=9 Hz, 1H), 7.81 (d, J=9 Hz, 1H), 7.42 (s, 2H), 5.24 (s, 2H), 5.08 (s, 2H), 3.46 (q, J=7 Hz, 2H), 2.09 (s, 3H), 1.03 (t, J=7 Hz, 3H).

### Intermediate C6. 2-(6-(Aminomethyl)pyridazin-3-yl)-3-methyl-5-(trifluoromethyl)phenol hydrochloride

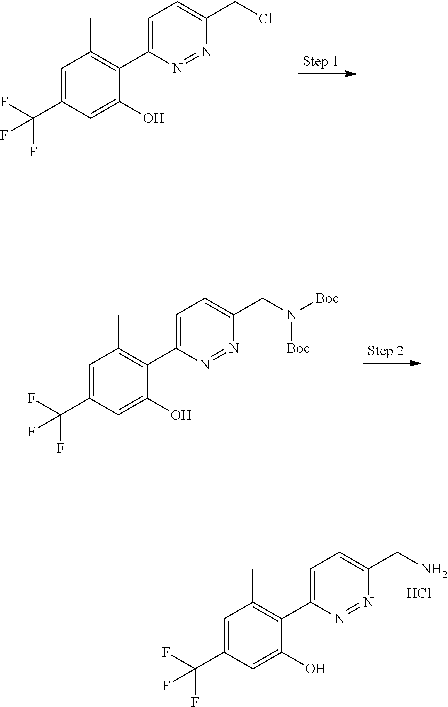 [SMILES: Cc1cc(C(F)(F)F)cc(O)c1-c1ccc(CCl)nn1 ; Cc1cc(C(F)(F)F)cc(O)c1-c1ccc(CN(C(=O)OC(C)(C)C)C(=O)OC(C)(C)C)nn1 ; Cc1cc(C(F)(F)F)cc(O)c1-c1ccc(CN)nn1 ; Cl]

### Step 1. Tert-butyl (tert-butoxycarbonyl)((6-(2-hydroxy-6-methyl-4-(trifluoromethyl)phenyl)pyridazin-3-yl)methyl)carbamate

To a solution of 3-(chloromethyl)-6-[2-(ethoxymethoxy)-6-methyl-4-(trifluoromethyl)phenyl]pyridazine (Intermediate C5, 1.50 g, 4.16 mmol) in DMF (15 mL) was added K₂CO₃ (1.15 g, 8.32 mmol) and tert-butyl N-tert-butoxycarbonylcarbamate (1.08 g, 4.99 mmol) at 25° C. under N₂. The mixture was stirred at 80° C. for 12 h then allowed to cool to room temperature. The reaction mixture was diluted with H₂O (15 mL) and extracted with EtOAc (3×15 mL). The combined organic layers were washed with brine (3×15 mL), dried over Na₂SO₄, filtered and concentrated under reduced pressure. The residue was purified by column chromatography (SiO₂, petroleum ether:ethyl acetate=1:0 to 2:1) to give the title compound as a white solid, Y=76%. ¹H NMR (400 MHz, DMSO-d₆) δ 7.71 (d, J=8 Hz, 1H), 7.62 (d, J=8 Hz, 1H), 7.41 (s, 2H), 5.22 (s, 2H), 5.06 (s, 2H), 3.45 (q, J=7 Hz, 2H), 2.08 (s, 3H), 1.38 (s, 18H), 1.03 (t, J=7 Hz, 3H).

### Step 2. 2-(6-(Aminomethyl)pyridazin-3-yl)-3-methyl-5-(trifluoromethyl)phenol hydrochloride

A mixture of tert-butyl N-tert-butoxycarbonyl-N-[[6-[2-(ethoxymethoxy)-6-methyl-4-(trifluoromethyl)phenyl]pyridazin-3-yl]methyl]carbamate (300 mg, 554 mol) and 4 M HCl in MeOH (10 mL) was stirred at 25° C. for 2 h. The reaction mixture was concentrated under reduced pressure to give the title compound as a white solid, Y=83%. ¹H NMR (400 MHz, DMSO-d₆) δ 10.52 (s, 1H), 8.69 (s, 2H), 8.01-7.67 (m, 2H), 7.44-6.98 (m, 3H), 4.48-4.44 (m, 2H), 2.10 (s, 3H).

Compound 1. 3-Methyl-2-(6-(((tetrahydro-2H-pyran-4-yl)amino)methyl)pyridazin-3-yl)-5-(trifluoromethyl)phenol

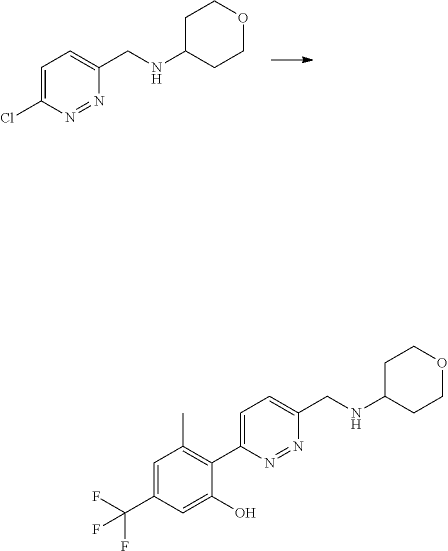 [SMILES: Clc1ccc(CNC2CCOCC2)nn1 ; Cc1cc(C(F)(F)F)cc(O)c1-c1ccc(CNC2CCOCC2)nn1]

To a solution of N-[(6-chloropyridazin-3-yl)methyl]tetrahydropyran-4-amine (Intermediate A1, 300 mg, 1.32 mmol) in dioxane (15 mL) and H₂O (5 mL) were added 3-methyl-2-(4,4,5,5-tetramethyl-1,3,2-dioxaborolan-2-yl)-5-(trifluoromethyl)phenol (478 mg, 1.58 mmol), Pd(PPh₃)₄ (30.5 mg, 26.4 mol) and Na₂CO₃ (419 mg, 3.95 mmol) at 25° C. The mixture was stirred at reflux for 8 h under N₂. The reaction mixture was diluted with H₂O (2 mL) and the resulting mixture was extracted with EtOAc (2×2 mL). The combined organic layers were washed with brine (2×2 mL), dried over Na₂SO₄, filtered and concentrated under reduced pressure. The crude material was purified by prep-HPLC (column: Waters Xbridge Prep OBD C18 150×40 mm, 10 μm; mobile phase: [(A) water (NH₄HCO₃)—(B) ACN]; Gradient: 25-45% B, 8 min) to give the title compound as a white solid, Y=54%. ¹H NMR (400 MHz, DMSO-d₆) δ 10.27 (br. s, 1H), 7.82 (d, J=9 Hz, 1H), 7.67 (d, J=9 Hz, 1H), 7.25-7.01 (m, 2H), 4.08 (s, 2H), 3.93-3.75 (m, 2H), 3.29-3.26 (m, 2H), 2.69-2.64 (m, 1H), 2.08 (s, 3H), 1.91-1.72 (m, 2H), 1.37-1.26 (m, 2H). LCMS (ESI): m/z [M+H]⁺=368.0.

### Compound 2. 2-(6-(((2-Oxaspiro[3.3]heptan-6-yl)amino)methyl)pyridazin-3-yl)-3-methyl-5-(trifluoromethyl)phenol

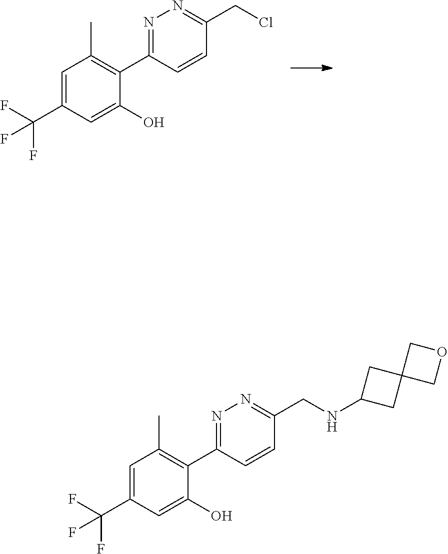 [SMILES: Cc1cc(C(F)(F)F)cc(O)c1-c1ccc(CCl)nn1 ; Cc1cc(C(F)(F)F)cc(O)c1-c1ccc(CNC2CC3(COC3)C2)nn1]

To a mixture of 2-[6-(chloromethyl)pyridazin-3-yl]-3-methyl-5-(trifluoromethyl)phenol (Intermediate C1, 60.0 mg, 198 mol) and 2-oxaspiro[3.3]heptan-6-amine hydrochloride (33.7 mg, 225 mol in DMSO (1 mL) was added DIPEA (104 μL, 595 mol,) at 25° C. The mixture was stirred at 120° C. for 1 h. The reaction mixture was purified by prep-HPLC (column: Waters Xbridge BEH C18 100×30 mm, 10 μm; mobile phase: [(A) H₂O (10 mM NH₄HCO₃)—(B) ACN]; gradient: 15-35% B, 8 min) and lyophilised to give the title compound as a white solid, Y=33%. ¹H NMR (400 MHz, DMSO-d₆) δ 9.09 (br. s, 1H), 7.75 (d, J=9 Hz, 1H), 7.64 (d, J=9 Hz, 1H), 7.14 (s, 1H), 7.08 (s, 1H), 7.10-7.07 (m, 1H), 4.54 (s, 2H), 4.46 (s, 2H), 3.92 (s, 2H), 3.15-3.00 (m, 1H), 2.70-2.50 (m, 1H), 2.42-2.35 (m, 2H), 2.07 (s, 3H), 1.92-1.84 (m, 2H). LCMS (ESI): m/z [M+H]⁺=380.2.

### Compound 3. 6-(((6-(2-Hydroxy-6-methyl-4-(trifluoromethyl)phenyl)pyridazin-3-yl)methyl)amino)-2-thiaspiro[3.3]heptane 2,2-dioxide

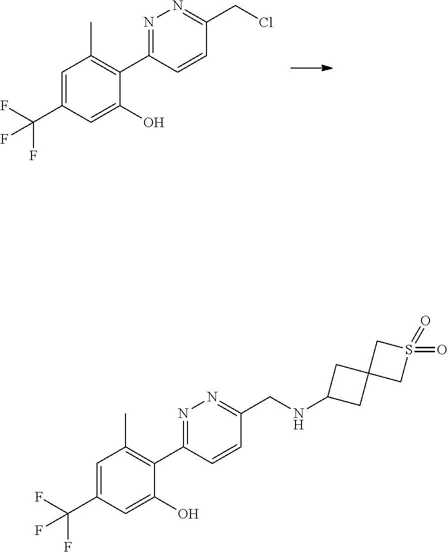 [SMILES: Cc1cc(C(F)(F)F)cc(O)c1-c1ccc(CCl)nn1 ; Cc1cc(C(F)(F)F)cc(O)c1-c1ccc(CNC2CC3(C2)CS(=O)(=O)C3)nn1]

To a mixture of 2-[6-(chloromethyl)pyridazin-3-yl]-3-methyl-5-(trifluoromethyl)phenol (Intermediate C1, 60.0 mg, 198 mol) and 2,2-dioxo-2thiaspiro[3.3]heptan-6-amine (48.0 mg, 297 mol) in DMSO (1 mL) was added DIPEA (69.1 μL, 396 mol) at 25° C. The mixture was stirred at 120° C. for 1 h then allowed to cool to room temperature. The reaction mixture was purified directly by prep-HPLC (column: Waters Xbridge BEH C18 100×30 mm, 10 μm; mobile phase: [(A) H₂O (10 mM NH₄HCO₃)—(B) ACN]; gradient: 10-40% B, 8 min) and lyophilised to give the title compound as a white solid, Y=29%. ¹H NMR (400 MHz, DMSO-d₆) δ 9.65 (br. s, 1H), 7.76 (d, J=9 Hz, 1H), 7.65 (d, J=9 Hz, 1H), 7.15 (s, 1H), 7.09 (s, 1H), 4.19 (s, 2H), 4.14 (s, 2H), 3.94 (s, 2H), 3.26-3.19 (m, 1H), 2.82-2.71 (m, 1H), 2.50-2.40 (m, 2H), 2.09-2.02 (m, 5H). LCMS (ESI): m/z [M+H]⁺ 428.1.

### Compound 4. 2-(6-(((2-Oxabicyclo[3.2.0]heptan-7-yl)amino)methyl)pyridazin-3-yl)-3-methyl-5-(trifluoromethyl)phenol

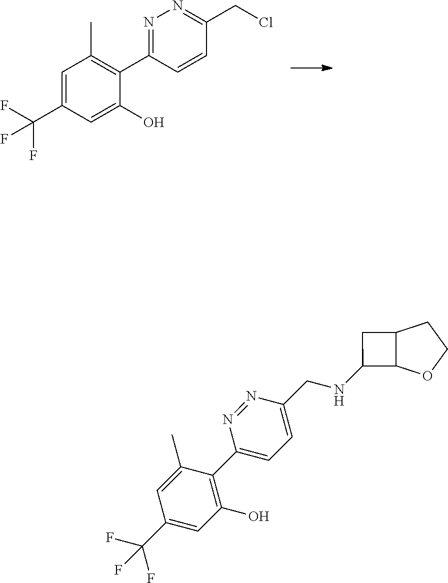 [SMILES: Cc1cc(C(F)(F)F)cc(O)c1-c1ccc(CCl)nn1 ; Cc1cc(C(F)(F)F)cc(O)c1-c1ccc(CNC2CC3CCOC32)nn1]

To a mixture of 2-[6-(chloromethyl)pyridazin-3-yl]-3-methyl-5-(trifluoromethyl)phenol (Intermediate C1, 60 mg, 198 mol) and 2-oxabicyclo[3.2.0]heptan-7-amine hydrochloride (44.5 mg, 297 mol) in DMSO (1 mL) was added DIPEA (104 μL, 595 mol) at 25° C. The mixture was stirred at 120° C. for 1 h then allowed to cool to room temperature. The reaction mixture was purified directly by prep-HPLC (column: Waters Xbridge BEH C18 100×30 mm, 10 μm; mobile phase: [(A) H₂O (10 mM NH₄HCO₃)—(B) ACN]; gradient: 25-45% B, 8 min) and lyophilised to give the title compound as a white solid, Y=32%. ¹H NMR (400 MHz, DMSO-d₆) δ 9.69 (br. s, 1H), 7.78 (d, J=9 Hz, 1H), 7.64 (d, J=9 Hz, 1H), 7.15 (s, 1H), 7.09 (s, 1H), 4.51-4.44 (m, 1H), 4.10-3.91 (m, 4H), 3.29-3.13 (m, 1H), 2.77-2.70 (m, 1H), 2.47-2.39 (m, 1H), 2.38-2.30 (m, 1H), 2.08 (s, 3H), 1.70-1.62 (m, 2H), 1.35-1.25 (m, 1H). LCMS (ESI): m/z [M+H]380.2.

### Compound 5. 3-Methyl-2-(6-(((4-methyltetrahydro-2H-pyran-4-yl)amino)methyl)pyridazin-3-yl)-5-(trifluoromethyl)phenol

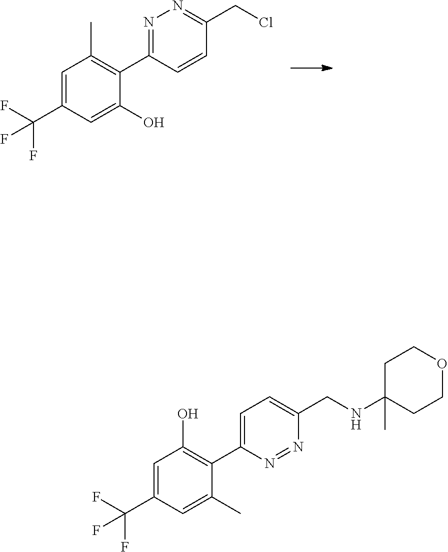 [SMILES: Cc1cc(C(F)(F)F)cc(O)c1-c1ccc(CCl)nn1 ; Cc1cc(C(F)(F)F)cc(O)c1-c1ccc(CNC2(C)CCOCC2)nn1]

To a solution of 2-[6-(chloromethyl)pyridazin-3-yl]-3-methyl-5-(trifluoromethyl)phenol (Intermediate C1, 60.0 mg, 198 mol) in DMSO (1 mL) were added DIPEA (69.1 μL, 396 mol) and 4-methyltetrahydropyran-4-amine (45.7 mg, 396 mol) at 25° C. The mixture was stirred at 120° C. for 1 h then allowed to cool to room temperature. The reaction mixture was purified directly by prep-HPLC (column: Waters Xbridge BEH C18 100×30 mm, 10 μm; mobile phase: [(A) H₂O (10 mM NH₄HCO₃)—(B) ACN]; gradient: 20-50% B, 8 min) and lyophilised to give the title compound as a white solid, Y=20%. ¹H NMR (400 MHz, DMSO-d₆): δ 10.21 (br. s, 1H), 7.85 (d, J=9 Hz, 1H), 7.66 (d, J=9 Hz, 1H), 7.22-7.01 (m, 2H), 4.08-3.93 (m, 2H), 3.80-3.66 (m, 2H), 3.56-3.44 (m, 2H), 2.35-2.22 (m, 1H), 2.08 (s, 3H), 1.63-1.54 (m, 2H), 1.52-1.43 (m, 2H), 1.16 (s, 3H). LCMS (ESI): m/z [M+H]⁺=382.1.

### Compound 6. 3-Methyl-2-(6-(((3-methyltetrahydrofuran-3-yl)amino)methyl)pyridazin-3-yl)-5-(trifluoromethyl)phenol hydrochloride

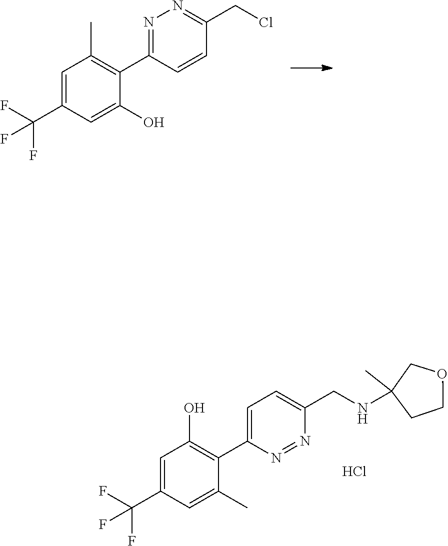 [SMILES: Cc1cc(C(F)(F)F)cc(O)c1-c1ccc(CCl)nn1 ; Cc1cc(C(F)(F)F)cc(O)c1-c1ccc(CNC2(C)CCOC2)nn1 ; Cl]

To a solution of 2-[6-(chloromethyl)pyridazin-3-yl]-3-methyl-5-(trifluoromethyl)phenol (Intermediate C1, 60 mg, 198 mol) in DMSO (1 mL) were added 3-methyltetrahydrofuran-3-amine (54.6 mg, 396 mol) and DIPEA (69.1 μL, 396 mol) at 25° C. The mixture was stirred at 120° C. for 1 h and then allowed to cool to room temperature. The reaction mixture was purified directly by prep-HPLC (column: Waters Xbridge BEH C18 100×30 mm, 10 μm; mobile phase: [(A) H₂O (10 mM NH₄HCO₃)—(B) ACN]; gradient: 20-50% B, 8 min) and lyophilised to give the title compound as a white solid, Y=100%. ¹H NMR (400 MHz, DMSO-d₆) δ 10.59-10.41 (m, 1H), 9.88-9.65 (m, 2H), 7.96 (d, J=9 Hz, 1H), 7.85 (d, J=9 Hz, 1H), 7.22-7.11 (m, 2H), 4.73-4.55 (m, 2H), 4.10 (d, J=10 Hz, 1H), 4.08-3.92 (m, 1H), 3.90-3.80 (m, 1H), 3.61-3.52 (m, 1H), 2.45-2.36 (m, 1H), 2.15-1.99 (m, 4H), 1.57 (s, 3H). LCMS (ESI): m/z [M+H]⁺=368.0.

### Compound 7. N-((6-(2-Hydroxy-6-methyl-4-(trifluoromethyl)phenyl)pyridazin-3-yl)methyl)tetrahydro-2H-pyran-4-carboxamide

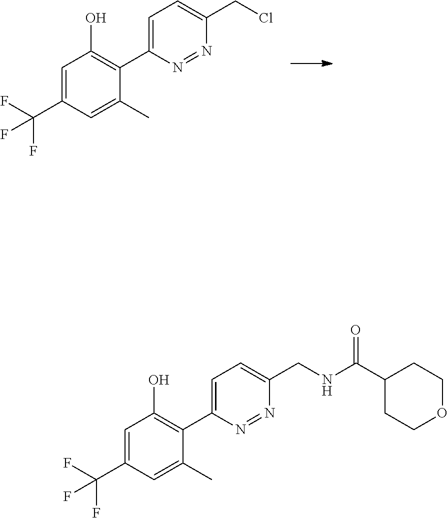 [SMILES: Cc1cc(C(F)(F)F)cc(O)c1-c1ccc(CCl)nn1 ; Cc1cc(C(F)(F)F)cc(O)c1-c1ccc(CNC(=O)C2CCOCC2)nn1]

To a solution of tetrahydropyran-4-carboxamide (171 mg, 1.32 mmol) in DMF (1 mL) was added LiHMDS solution (1 M in THF, 1.32 mL, 1.32 mmol) at 0° C. under N₂ and the reaction mixture was stirred at 0° C. for 30 min. The mixture was then treated with a solution of 2-[6-(chloromethyl)pyridazin-3-yl]-3-methyl-5-(trifluoromethyl)phenol (Intermediate C1, 80 mg, 264 mol) in DMF (1 mL) at 0° C. The mixture was stirred at 25° C. for 3 h then diluted with H₂O (2 mL) and extracted with EtOAc (3×2 mL). The combined organic layers were washed with brine (2 mL), dried over anhydrous Na₂SO₄, filtered and concentrated under reduced pressure. The crude material was purified by prep-HPLC (column: Waters Xbridge C18 150×50 mm, 10 μm; mobile phase: [(A) H₂O (10 mM NH₄HCO₃)—(B) ACN]; gradient: 25-45% B, 8 min) and lyophilised to give the title compound as a white solid, Y=28%. ¹H NMR (400 MHz, DMSO-d₆) δ 10.29 (s, 1H), 8.58 (t, J=6 Hz, 1H), 7.69 (d, J=9 Hz 1H), 7.58 (d, J=9 Hz, 1H), 7.16 (s, 1H), 7.10 (s, 1H), 4.62 (d, J=6 Hz, 2H), 3.89-3.86 (m, 2H), 3.36-3.29 (m, 2H), 2.53-2.42 (m, 1H), 2.08 (s, 3H), 1.67-1.61 (m, 4H). LCMS (ESI): m/z [M+H]⁺=396.1.

### Compound 8. N-((6-(2-Hydroxy-6-methyl-4-(trifluoromethyl)phenyl)pyridazin-3-yl)methyl)-2-methoxyacetamide

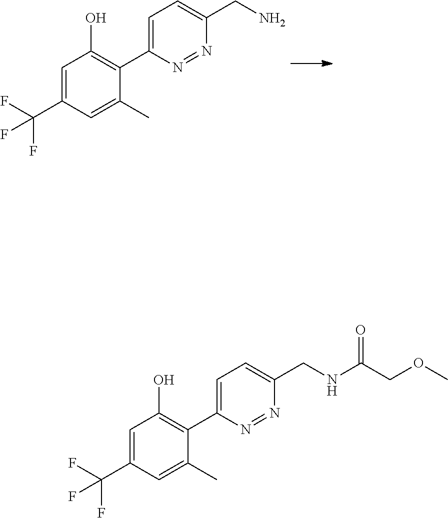 [SMILES: Cc1cc(C(F)(F)F)cc(O)c1-c1ccc(CN)nn1 ; COCC(=O)NCc1ccc(-c2c(C)cc(C(F)(F)F)cc2O)nn1]

To a solution of 2-methoxyacetic acid (10.3 μL, 134 mol) in DMF (1 mL) was added HATU (80.5 mg, 212 mol) at 0° C. under N₂. The reaction mixture was stirred at 0° C. for 0.5 h then to the above mixture were added 2-[6-(aminomethyl) pyridazin-3-yl]-3-methyl-5-(trifluoromethyl)phenol (Compound 20, 40 mg, 141 mol) and DIPEA (73.8 μL, 424 mol). The mixture was stirred at 0° C. for 6 h. The reaction mixture was quenched by addition of H₂O (1 mL) and extracted with EtOAc (3×1 mL). The combined organic layers were washed with brine (1 mL), dried over Na₂SO₄, filtered and concentrated under reduced pressure. The crude material was purified by prep-HPLC (column: Phenomenex Luna C18 100×40 mm, 5 μm; mobile phase: [(A) H₂O (0.04% HCl)—(B) ACN]; gradient: 20-45% B, 8 min) and lyophilised to give the title compound as a white solid, Y=70%. ¹H NMR (400 MHz, DMSO-d₆) δ 10.40 (br. s, 1H), 8.62 (t, J=6 Hz, 1H), 7.76 (d, J=9 Hz 1H), 7.70 (d, J=9 Hz 1H), 7.17 (s, 1H), 7.14 (s, 1H), 4.68 (d, J=6 Hz, 2H), 3.94 (s, 2H), 3.36 (s, 3H), 2.09 (s, 3H). LCMS (ESI): m/z: [M+H]⁺=356.0.

### Compound 9. 2-Methyl-6-(6-(((tetrahydro-2H-pyran-4-yl)amino)methyl)pyridazin-3-yl)-3-(trifluoromethyl)phenol

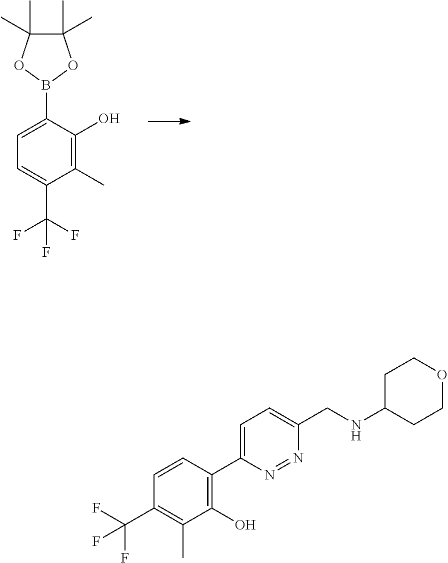 [SMILES: Cc1c(C(F)(F)F)ccc(B2OC(C)(C)C(C)(C)O2)c1O ; Cc1c(C(F)(F)F)ccc(-c2ccc(CNC3CCOCC3)nn2)c1O]

To a mixture of 2-methyl-6-(4,4,5,5-tetramethyl-1,3,2-dioxaborolan-2-yl)-3-(trifluoro methyl)phenol (Intermediate B2, 250 mg, 828 mol) and N-[(6-chloropyridazin-3-yl)methyl]tetrahydropyran-4-amine (Intermediate A1, 226 mg, 993 mol) in dioxane (2.5 mL) and H₂O (0.5 mL) was added Pd(dppf)Cl₂ (30.3 mg, 41.4 mol) and Na₂CO₃ (263 mg, 2.48 mmol) at 25° C. under N₂. The mixture was stirred at reflux for 8 h then allowed to cool to room temperature. The reaction mixture was diluted with H₂O (3 mL) and extracted with EtOAc (3×3 mL). The combined organic layers were washed with brine (3 mL), dried over Na₂SO₄, filtered and concentrated under reduced pressure. The crude material was purified by prep-HPLC (column: Waters Xbridge Prep OBD C18 150×40 mm, 10 μm; mobile phase: [(A) water (NH₄HCO₃)—(B) ACN]; gradient: 40-60% B, 8 min) to give the title compound as a white solid, Y=13%. ¹H NMR (400 MHz, DMSO-d₆) δ 8.66 (d, J=9 Hz, 1H), 8.13-8.05 (m, 2H), 7.28 (d, J=9 Hz, 1H), 4.10 (s, 2H), 3.85-3.80 (m, 2H), 3.29-3.21 (m, 2H), 2.66-2.60 (m, 1H), 2.37 (s, 3H), 1.81-1.77 (m, 2H), 1.34-1.23 (m, 2H). LCMS (ESI): m/z [M+H]⁺=368.0.

### Compound 10. 3-Methyl-2-(4-methyl-6-(((tetrahydro-2H-pyran-4-yl)amino)methyl)pyridazin-3-yl)-5-(trifluoromethyl)phenol

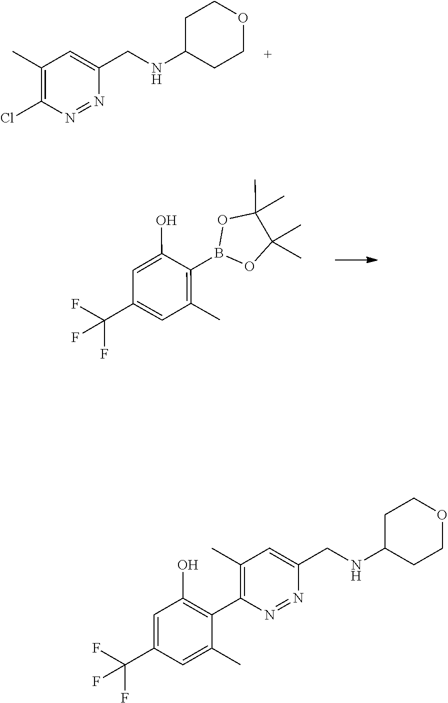 [SMILES: Cc1cc(CNC2CCOCC2)nnc1Cl ; Cc1cc(C(F)(F)F)cc(O)c1B1OC(C)(C)C(C)(C)O1 ; Cc1cc(CNC2CCOCC2)nnc1-c1c(C)cc(C(F)(F)F)cc1O]

To a solution of N-[(6-chloro-5-methyl-pyridazin-3-yl)methyl]tetrahydropyran-4-amine (Intermediate A2, 30 mg, 124 mol) in dioxane (1.2 mL) and H₂O (0.12 mL), under N₂, were added 3-methyl-2-(4,4,5,5-tetramethyl-1,3,2-dioxaborolan-2-yl)-5-(trifluoromethyl)phenol (Intermediate B1, 45.0 mg, 149 mol), Na₂CO₃ (39.5 mg, 372 mol) and Pd(dppf)Cl₂ (4.54 mg, 6.21 mol) at 25° C. The mixture was stirred at reflux for 2 h then diluted with H₂O (1 mL) and extracted with EtOAc (3×1 mL). The combined organic layers were washed with brine (3 mL), dried over Na₂SO₄, filtered and concentrated under reduced pressure. The crude material was purified by prep-HPLC (column: Waters Xbridge C18 150×50 mm, 10 μm; mobile phase: [(A) H₂O (10 mM NH₄HCO₃)—(B) ACN]; gradient: 25-45% B, 8 min) and lyophilised to give the title compound as a white solid, Y=36%. ¹H NMR (400 MHz, DMSO-d₆) δ 10.22 (br. s, 1H), 7.68 (s, 1H), 7.18 (s, 1H), 7.09 (s, 1H), 4.03 (s, 2H), 3.84 (d, J 12 Hz, 2H), 3.30-3.24 (m, 2H), 2.71-2.64 (m, 1H), 2.06 (s, 3H), 1.94 (s, 3H), 1.83 (d, J 12 Hz, 2H), 1.39-1.23 (m, 2H). LC-MS (ESI): m/z [M+H]⁺=382.1.

### Compound 11. 2-(4-Methyl-6-(((tetrahydro-2H-pyran-4-yl)amino)methyl)pyridazin-3-yl)-5-(trifluoromethyl)phenol

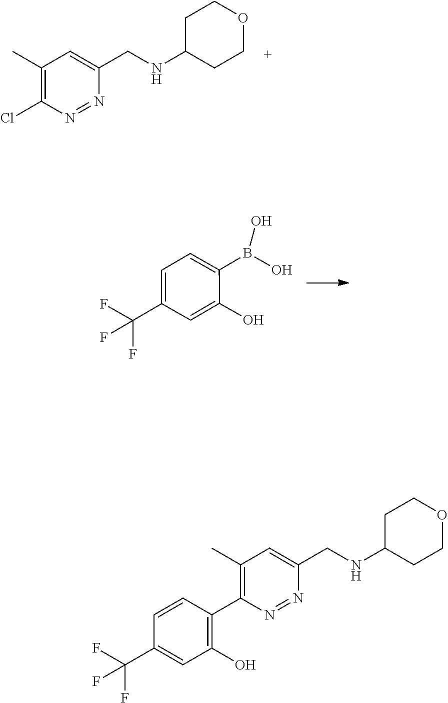 [SMILES: Cc1cc(CNC2CCOCC2)nnc1Cl ; OB(O)c1ccc(C(F)(F)F)cc1O ; Cc1cc(CNC2CCOCC2)nnc1-c1ccc(C(F)(F)F)cc1O]

To a solution of [2-hydroxy-4-(trifluoromethyl)phenyl]boronic acid (Intermediate A2, 30.7 mg, 149 mol) in dioxane (0.5 mL) and H₂O (0.05 mL), under N₂, were added N-[(6-chloro-5-methyl-pyridazin-3-yl)methyl]tetrahydropyran-4-amine (Intermediate B1, 30.0 mg, 124 mol), Na₂CO₃ (39.5 mg, 372 mol) and Pd(dppf)Cl₂ (4.54 mg, 6.21 mol) at 25° C. The mixture was stirred at reflux for 2 h then allowed to cool to room temperature. The reaction mixture was diluted with H₂O (1 mL) and extracted with EtOAc (3×1 mL). The combined organic layers were washed with brine (3 mL), dried over Na₂SO₄, filtered and concentrated under reduced pressure. The crude material was purified by prep-HPLC (column: Waters Xbridge C₁₈ 150×50 mm, 10 μm; mobile phase: [(A) H₂O (10 mM NH₄HCO₃)—(B) ACN]; gradient: 25-45% B, 8 min) and lyophilised to give the title compound as a white solid, Y=43%. ¹H NMR (400 MHz, DMSO-d₆) δ 10.57 (br. s, 1H), 7.65 (s, 1H), 7.44 (d, J 8 Hz, 1H), 7.28 (d, J 8 Hz, 1H), 7.25 (s, 1H), 4.02 (s, 2H), 3.87-3.81 (m, 2H), 3.30-3.24 (m, 2H), 2.67-2.63 (m, 1H), 2.18 (s, 3H), 1.89-1.76 (m, 2H), 1.40-1.22 (m, 2H). LC-MS (ESI): m/z [M+H]+=368.1.

### Compound 12. 5-Chloro-3-methyl-2-(6-(((tetrahydro-2H-pyran-4-yl)amino)methyl)pyridazin-3-yl)phenol

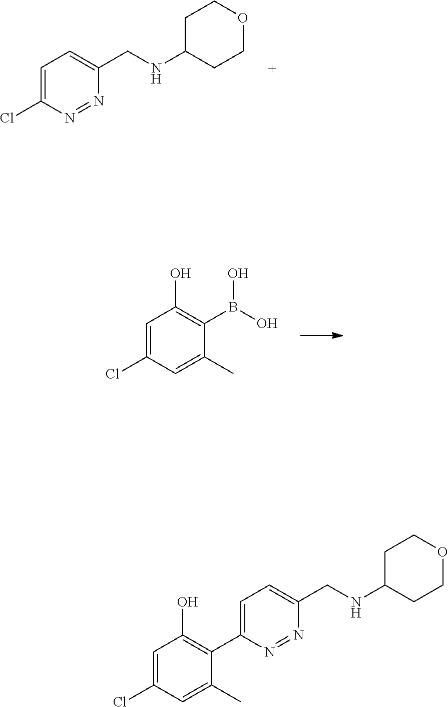 [SMILES: Clc1ccc(CNC2CCOCC2)nn1 ; Cc1cc(Cl)cc(O)c1B(O)O ; Cc1cc(Cl)cc(O)c1-c1ccc(CNC2CCOCC2)nn1]

To a solution of N-[(6-chloropyridazin-3-yl)methyl]tetrahydropyran-4-amine (Intermediate A1, 150 mg, 659 mol) in dioxane (7.5 mL) and H₂O (2.5 mL), under N₂, were added (4-chloro-2-hydroxy-6-methyl-phenyl)boronic acid (246 mg, 1.32 mmol), Pd(PPh₃)₄ (38.1 mg, 32.9 mol) and Na₂CO₃ (209 mg, 1.98 mmol) at 25° C. The mixture was stirred at reflux for 2 h then allowed to cool to room temperature. The mixture was diluted with H₂O (5 mL) and extracted with EtOAc (3×10 mL). The combined organic layers were washed with brine (10 mL), dried over Na₂SO₄, filtered and concentrated under reduced pressure. The residue was purified by prep-HPLC (column: Waters Xbridge C18 150×50 mm, 10 μm; mobile phase: [(A) H₂O (10 mM NH₄HCO₃)—(B) ACN]; gradient: 20-40% B, 8 min) and lyophilised to give the title compound as a white solid, Y=23%. ¹H NMR (400 MHz, DMSO-d₆) δ 9.38 (br. s, 1H), 7.77 (d, J=9 Hz, 1H), 7.60 (d, J=9 Hz, 1H), 6.91-6.80 (m, 2H), 4.06 (s, 2H), 3.86-3.81 (m, 2H), 3.29-3.25 (m, 2H), 2.70-2.61 (m, 1H), 2.01 (s, 3H), 1.84-1.80 (m, 2H), 1.36-1.22 (m, 2H). LCMS (ESI): m/z [M+H]⁺=334.0.

### Compound 13. 2-(6-(((Tetrahydro-2H-pyran-4-yl)amino)methyl)pyridazin-3-yl)-5-(trifluoromethyl)phenol

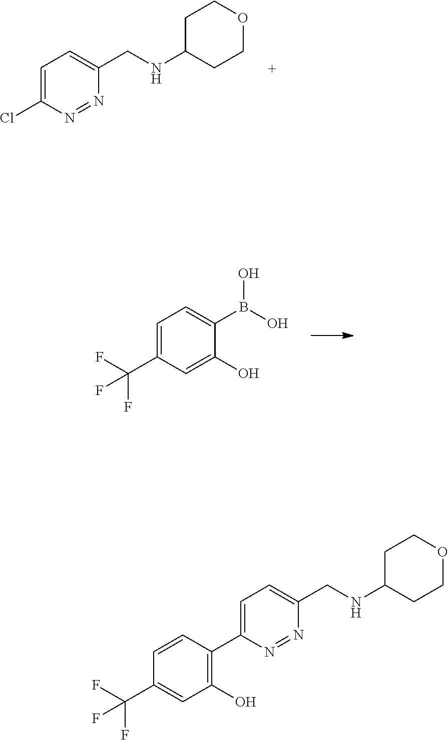 [SMILES: Clc1ccc(CNC2CCOCC2)nn1 ; OB(O)c1ccc(C(F)(F)F)cc1O ; Oc1cc(C(F)(F)F)ccc1-c1ccc(CNC2CCOCC2)nn1]

To a solution of N-[(6-chloropyridazin-3-yl)methyl]tetrahydropyran-4-amine (Intermediate A1, 50.0 mg, 220 mol) in dioxane (2.5 mL) and H₂O (0.25 mL), under N₂, were added [2-hydroxy-4-(trifluoromethyl)phenyl]boronic acid (54.3 mg, 264 mol), Na₂CO₃ (69.8 mg, 659 mol) and Pd(dppf)Cl₂ (8.03 mg, 11.0 mol) at 25° C. The mixture was stirred at reflux for 2 h then allowed to cool to room temperature. The mixture was diluted with H₂O (2 mL) and extracted with EtOAc (3×2 mL). The combined organic layers were washed with brine (6 mL), dried over Na₂SO₄, filtered and concentrated under reduced pressure. The crude material was purified by prep-HPLC (column: Waters Xbridge Prep OBD C18 150×40 mm, 10 μm; mobile phase: [(A) water (NH₄HCO₃)—(B) ACN]; gradient: 40-70% B, 8 min) to give a white solid. The material was further purified by prep-TLC (SiO₂, DCM:MeOH=10:1) to give the title compound as a white solid, Y=19%. ¹H NMR (400 MHz, DMSO-d₆) δ 13.03 (br. s, 1H), 8.53 (d, J 9 Hz, 1H), 8.24 (d, J 9 Hz, 1H), 8.03 (d, J 9 Hz, 1H), 7.40-7.34 (m, 2H), 4.15 (s, 2H), 3.92-3.84 (m, 2H), 3.35-3.28 (m, 2H), 2.75-2.65 (m, 1H), 1.90-1.80 (m, 2H), 1.40-1.30 (m, 2H). LC-MS (ESI): m/z [M+H]⁺=354.1.

### Compound 14. 3-Chloro-2-(6-(((tetrahydro-2H-pyran-4-yl)amino)methyl)pyridazin-3-yl)-5-(trifluoromethyl)phenol

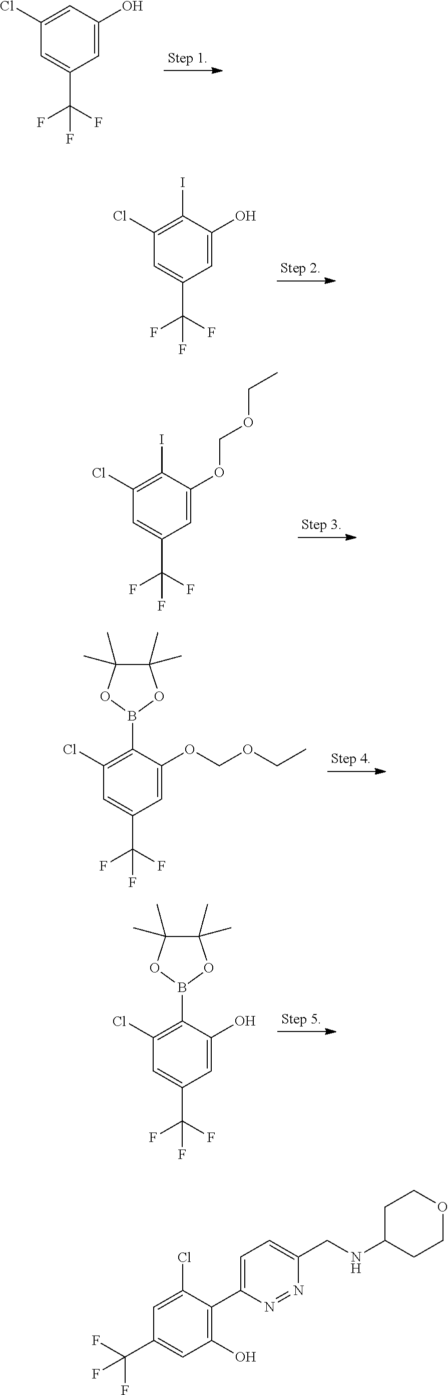

### Step 1. 3-Chloro-2-iodo-5-(trifluoromethyl)phenol

To a solution of 3-chloro-5-(trifluoromethyl)phenol (2.5 g, 12.7 mmol) in toluene (50 mL) was added NaH (1.02 g, 25.4 mmol, 60% wt in mineral oil) at 0° C. under N₂. The mixture was stirred at 0° C. for 30 min, then was treated with a solution of I₂ (2.56 mL, 12.7 mmol) in toluene (150 mL). The mixture was stirred at 25° C. for 4 h then quenched with sat. NH₄Cl solution (100 ml) at 0° C. the resulting mixture was extracted with EtOAc (3×100 mL). The combined organic layers were washed with brine (100 mL), dried over Na₂SO₄, filtered and concentrated under reduced pressure. The crude material was purified by column chromatography (SiO₂, petroleum ether:ethyl acetate=1:0 to 10:1) to give the title compound as a colorless oil, Y=90%. ¹H NMR (400 MHz, DMSO-d₆) δ 11.52 (s, 1H), 7.36 (d, J=2 Hz, 1H), 7.13 (d, J=2 Hz, 1H).

### Step 2. 1-Chloro-3-(ethoxymethoxy)-2-iodo-5-(trifluoromethyl)benzene

The following reaction was performed twice in parallel and the crude mixtures were combined prior to workup. To a solution of 3-chloro-2-iodo-5-(trifluoromethyl)phenol (3.70 g, 11.5 mmol) in DMF (37 mL) was added Cs₂CO₃ (7.48 g, 23.0 mmol) and chloromethoxyethane (1.06 mL, 11.5 mmol). The mixture was stirred at 25° C. for 4 h under N₂. Combined crude mixtures were diluted with H₂O (37 mL) and extracted with EtOAc (3×37 mL). The combined organic layers were washed with brine (37 mL), dried over Na₂SO₄, filtered and concentrated under reduced pressure. The crude material was purified by column chromatography (SiO₂, petroleum ether:ethyl acetate=1:0 to 10:1) to give the title compound as a colourless oil, Y=33%. ¹H NMR (400 MHz, DMSO-d₆) δ 7.59 (d, J=2 Hz, 1H), 7.31 (d, J=2 Hz, 1H), 5.47 (s, 2H), 3.73-3.68 (m, 2H), 1.15-1.12 (m, 3H).

### Step 3. 2-(2-Chloro-6-(ethoxymethoxy)-4-(trifluoromethyl)phenyl)-4,4,5,5-tetramethyl-1,3,2-dioxaborolane

To a solution of 1-chloro-3-(ethoxymethoxy)-2-iodo-5-(trifluoromethyl)benzene (2.90 g, 7.62 mmol) in dioxane (29 mL), under N₂, were added TEA (7.85 mL, 56.4 mmol), 4,4,5,5-tetramethyl-1,3,2-dioxaborolane (3.32 mL, 22.9 mmol), Pd(OAc)₂ (222 mg, 991 mol) and biphenyl-2-yl-dicyclohexylphosphane (641 mg, 1.83 mmol) at 25° C. The mixture was stirred at 80° C. for 18 h then allowed to cool to room temperature. The reaction mixture was diluted with H₂O (30 mL) and extracted with EtOAc (3×30 mL). The combined organic layers were washed with brine (30 mL), dried over Na₂SO₄, filtered and concentrated under reduced pressure. The residue was purified by column chromatography (SiO₂, petroleum ether:ethyl acetate=1:0 to 10:1) to give the title as a yellow oil, Y=48%.

### Step 4. 3-Chloro-2-(4,4,5,5-tetramethyl-1,3,2-dioxaborolan-2-yl)-5-(trifluoromethyl)phenol

A solution of 2-[2-chloro-6-(ethoxymethoxy)-4-(trifluoromethyl)phenyl]-4,4,5,5-tetramethyl-1,3,2-dioxaborolane (1.05 g, 2.76 mmol,) and 4 M HCl in EtOAc (21 mL) was stirred at 25° C. for 5 h. The reaction mixture was concentrated under reduced pressure to give the title compound as a yellow solid, Y=99%. ¹H NMR (400 MHz, DMSO-d₆) δ 10.61 (s, 1H), 7.17 (s, 1H), 6.99 (s, 1H), 1.31 (s, 12H).

### Step 5. 3-Chloro-2-(6-(((tetrahydro-2H-pyran-4-yl)amino)methyl)pyridazin-3-yl)-5-(trifluoromethyl)phenol

To a solution of N-[(6-chloropyridazin-3-yl)methyl]tetrahydropyran-4-amine (Intermediate A1, 310 mg, 1.36 mmol) in dioxane (15.5 mL) and H₂O (5.2 mL) under N₂, were added Na₂CO₃ (433 mg, 4.08 mmol), Pd(PPh₃)₄ (78.7 mg, 68.1 mol) and 3-chloro-2-(4,4,5,5-tetramethyl-1,3,2-dioxaborolan-2-yl)-5-(trifluoromethyl)phenol (878 mg, 2.72 mmol) at 25° C. The mixture was stirred at reflux for 8 h then allowed to cool to room temperature. The reaction mixture was diluted with H₂O (15 mL) and extracted with EtOAc (3×15 mL). The combined organic layers were washed with brine (15 mL), dried over Na₂SO₄, filtered and concentrated under reduced pressure. The residue was purified by prep-HPLC (column: Waters Xbridge C18 150×50 mm, 10 μm; mobile phase: [(A) H₂O (10 mM NH₄HCO₃)—(B) ACN]; gradient: 20-40% B, 8 min) and lyophilised to give a white solid. The afforded material which was further purified by prep-HPLC (column: Phenomenex Gemini-NX 150×30 mm, 5 μm; mobile phase: [(A) H₂O (10 mM NH₄HCO₃)—(B) ACN]; gradient: 10-40% B, 20 min) and lyophilised to give the title compound as a white solid, Y=3%. ¹H NMR (400 MHz, DMSO-d₆) δ 7.86 (d, J=8 Hz, 1H), 7.72 (d, J=8 Hz, 1H), 7.41 (s, 1H), 7.22 (s, 1H), 4.10 (s, 2H), 3.87-3.82 (m, 2H), 3.29-3.26 (m, 2H), 2.69-2.65 (m, 1H), 1.84-1.81 (m, 2H), 1.35-1.30 (m, 2H). LCMS (ESI): m/z [M+H]⁺=388.0.

### Compound 15. 4-Methyl-2-(6-(((tetrahydro-2H-pyran-4-yl)amino)methyl)pyridazin-3-yl)-5-(trifluoromethyl)phenol

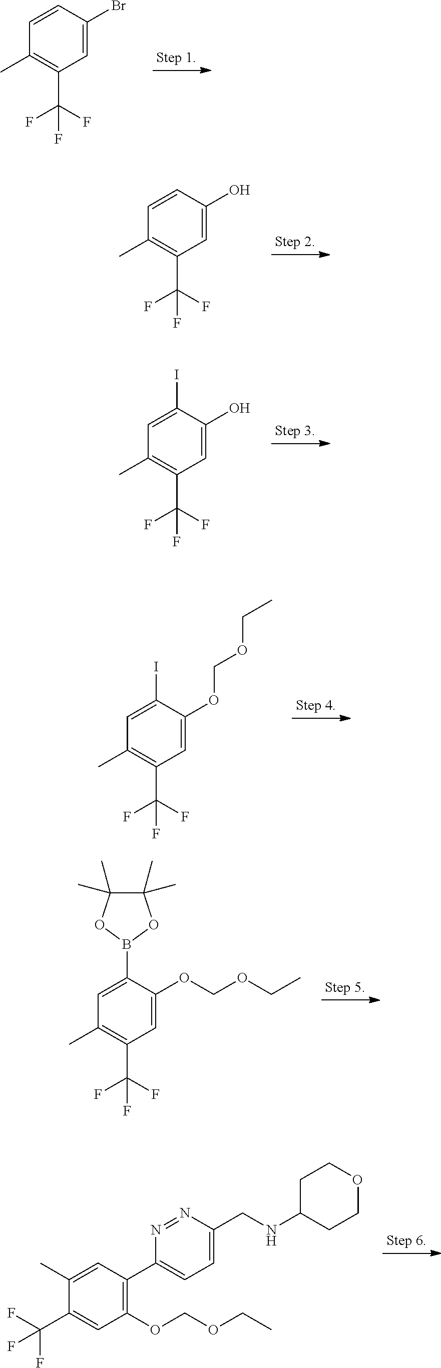

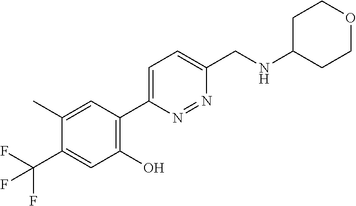 [SMILES: Cc1cc(-c2ccc(CNC3CCOCC3)nn2)c(O)cc1C(F)(F)F]

### Step 1. 4-Methyl-3-(trifluoromethyl)phenol

To a solution of 4-bromo-1-methyl-2-(trifluoromethyl)benzene (5.00 g, 20.9 mmol) in dioxane (25 mL) and H₂O (25 mL), at 25° C. under N₂, were added ditert-butyl-[2-(2,4,6-triisopropylphenyl) phenyl]phosphane (711 mg, 1.67 mmol), KOH (3.52 g, 62.8 mmol), Pd₂(dba)₃ (3.83 g, 4.18 mmol). The mixture was stirred at reflux for 2.5 h then allowed to cool to room temperature. The mixture was diluted with H₂O (50 mL) and extracted with ethyl acetate (3×50 mL). The combined organic layers were washed with brine (50 mL), dried over Na₂SO₄, filtered and concentrated under reduced pressure to give the title compound as a white solid.

### Step 2. 2-Iodo-4-methyl-5-(trifluoromethyl)phenol

To a solution of 4-methyl-3-(trifluoromethyl)phenol (2.50 g, 14.2 mmol) in toluene (25 mL), at 0° C. under N₂, was added NaH (341 mg, 8.52 mmol, 60% wt in mineral oil). The mixture was stirred at 0° C. for 1 h then I₂ (2.86 mL, 14.2 mmol) was added. The mixture was stirred at 0° C. for 2 h then diluted with H₂O (25 mL) and extracted with ethyl acetate (3×25 mL). The combined organic layers were washed with brine (25 mL), dried over Na₂SO₄, filtered and concentrated under reduced pressure to give the title compound as a white solid.

### Step 3. 1-(Ethoxymethoxy)-2-iodo-4-methyl-5-(trifluoromethyl)benzene

To a mixture of 2-iodo-4-methyl-5-(trifluoromethyl)phenol (4.00 g, 13.2 mmol) in DMF (40 mL) were added Cs₂CO₃ (8.63 g, 26.5 mmol) and chloromethoxyethane (1.23 mL, 13.2 mmol) at 25° C. The mixture was stirred for 2 h then diluted with H₂O (2 ml) and extracted with ethyl acetate (3×2 mL). The combined organic layers were washed with brine (2 mL), dried over Na₂SO₄, filtered and concentrated under reduced pressure. The residue was purified by prep-TLC (SiO₂, petroleum ether:ethyl acetate=10:1) to give the title compound as a white solid, Y=52%. ¹H NMR (400 MHz, DMSO-d₆) δ 7.90 (s, 1H), 7.30 (s, 1H), 5.36 (s, 2H), 3.69 (q, J=7 Hz, 2H), 2.33 (d, J=2 Hz, 3H), 1.12 (t, J=7 Hz, 3H).

### Step 4. 2-(2-(Ethoxymethoxy)-5-methyl-4-(trifluoromethyl)phenyl)-4,4,5,5-tetramethyl-1,3,2-dioxaborolane

To a solution of 1-(ethoxymethoxy)-2-iodo-4-methyl-5-(trifluoromethyl)benzene (1.00 g, 2.78 mmol) in dioxane (10 mL), under N₂, were added TEA (2.86 mL, 20.6 mmol), 4,4,5,5-tetramethyl-1,3,2-dioxaborolane (2.01 mL, 13.9 mmol), Pd(OAc)₂ (81.1 mg, 361 mol), dicyclohexyl-(2-phenylphenyl)phosphane (234 mg, 666 mol) at 25° C. The mixture was stirred at 80° C. for 12 h then allowed to cool to room temperature. The mixture was diluted with H₂O (10 mL) and extracted with ethyl acetate (3×10 mL). The combined organic layers were washed with brine (10 mL), dried over Na₂SO₄, filtered and concentrated under reduced pressure. The residue was purified by column chromatography (SiO₂, petroleum ether:ethyl acetate=1:1) to give the title compound as a white solid, Y=50%. ¹H NMR (400 MHz, DMSO-d₆) δ 7.53 (s, 1H), 7.31 (s, 1H), 5.25 (s, 2H), 3.69 (q, J=7 Hz, 2H), 2.36 (s, 3H), 1.29 (s, 12H), 1.11 (t, J=7 Hz, 3H).

### Step 5. N-((6-(2-(Ethoxymethoxy)-5-methyl-4-(trifluoromethyl)phenyl)pyridazin-3-yl)methyl)tetrahydro-2H-pyran-4-amine

To a mixture of 2-[2-(ethoxymethoxy)-5-methyl-4-(trifluoromethyl)phenyl]-4,4,5,5-tetramethyl-1,3,2-dioxaborolane (300 mg, 833 mol) and N-[(6-chloropyridazin-3-yl) methyl]tetrahydropyran-4-amine (Intermediate A1, 228 mg, 1.00 mmol) in dioxane (3 mL) and H₂O (0.6 mL) was added Pd(dppf)Cl₂ (30.5 mg, 41.7 mol), Na₂CO₃ (265 mg, 2.50 mmol) at 25° C. under N₂. The mixture was stirred at reflux for 8 h then allowed to cool to room temperature. The reaction mixture was diluted with H₂O (4 mL) and extracted with EtOAc (3×4 mL). The combined organic layers were washed with brine (4 mL), dried over Na₂SO₄, filtered and concentrated under reduced pressure. The crude material was purified by prep-HPLC (column: Waters Xbridge C18 150×50 mm, 10 μm; mobile phase: [(A) H₂O (10 mM NH₄HCO₃)—(B) ACN]; gradient: 35-55% B, 8 min) and lyophilised to give the title compound as white solid, Y=28%.

### Step 6. 4-Methyl-2-(6-(((tetrahydro-2H-pyran-4-yl)amino)methyl)pyridazin-3-yl)-5-(trifluoromethyl)phenol

A mixture of N-[[6-[2-(ethoxymethoxy)-5-methyl-4-(trifluoromethyl)phenyl]pyridazin-3-yl]methyl]tetrahydropyran-4-amine (100 mg, 235 mol) and a solution of 4 M HCl in EtOAc (2 mL) was stirred at 25° C. for 2 h. The reaction mixture was then concentrated under reduced pressure. The crude material was purified by prep-HPLC (column: Waters Xbridge Prep OBD C18 150×40 mm, 10 μm; mobile phase: [(A) H₂O (NH₄HCO₃)—(B) ACN]; gradient: 30-50% B, 8 min) and lyophilised to give the title compound as a white solid, Y=40%. ¹H NMR (400 MHz, DMSO-d₆) δ 8.49 (d, J=9 Hz, 1H), 8.04-7.95 (m, 2H), 7.27 (s, 1H), 4.08 (s, 2H), 3.85-3.80 (m, 2H), 3.30-3.26 (m, 2H), 2.63-2.61 (m, 1H), 2.42 (s, 3H), 1.85-1.75 (m, 2H), 1.31-1.27 (m, 2H). LCMS (ESI): m/z: [M+H]⁺=368.0.

### Compound 16. 5-Chloro-2-(6-(((tetrahydro-2H-pyran-4-yl)amino)methyl)pyridazin-3-yl)phenol

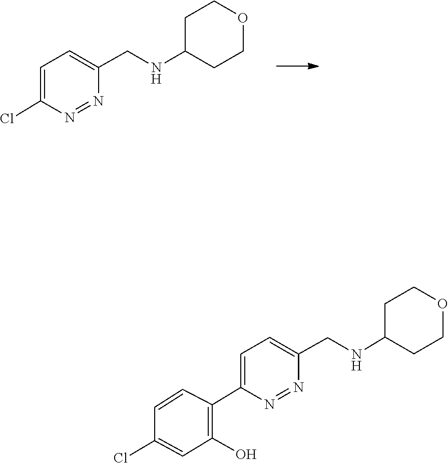 [SMILES: Clc1ccc(CNC2CCOCC2)nn1 ; Oc1cc(Cl)ccc1-c1ccc(CNC2CCOCC2)nn1]

To a solution of N-[(6-chloropyridazin-3-yl)methyl]tetrahydropyran-4-amine (Intermediate A1, 70.0 mg, 307 mol) in dioxane (0.6 mL) and H₂O (0.2 mL), under N₂, were added Na₂CO₃ (97.8 mg, 922 mol), (4-chloro-2-hydroxy-phenyl)boronic acid (63.6 mg, 369 mol) and Pd(dppf)Cl₂ (11.3 mg, 15.4 mol) at 25° C. The mixture was stirred at 120° C. for 8 h then allowed to cool to room temperature. The mixture was diluted with H₂O (1 mL) and extracted with EtOAc (3×1 mL). The combined organic layers were washed with brine (1 mL), dried over Na₂SO₄, filtered and concentrated under reduced pressure. The crude material was purified by prep-HPLC (column: Waters Xbridge Prep OBD C18 150×40 mm, 10 μm; mobile phase: [(A) H₂O (10 mM NH₄HCO₃)—(B) ACN]; gradient: 25-55% B, 8 min) and lyophilised to give the title compound as white solid, Y=16%. ¹H NMR (400 MHz, DMSO-d₆) δ 11.39 (br. s, 1H), 8.45 (d, J=9 Hz, 1H), 8.03 (d, J=9 Hz, 1H), 7.95 (d, J=9 Hz, 1H), 7.08 (d, J=2 Hz, 1H), 7.05-7.01 (m, 1H), 4.06 (s, 2H), 3.86-3.78 (m, 2H), 3.26 (d, J=2 Hz, 2H), 2.65-2.59 (m, 1H), 1.79 (d, J=12 Hz, 2H), 1.35-1.21 (m, 2H). LC-MS (ESI): m/z [M+H]⁺=320.1.

### Compound 17. 3-methyl-2-(6-(((tetrahydro-2H-pyran-4-yl)oxy)methyl)pyridazin-3-yl)-5-(trifluoromethyl)phenol

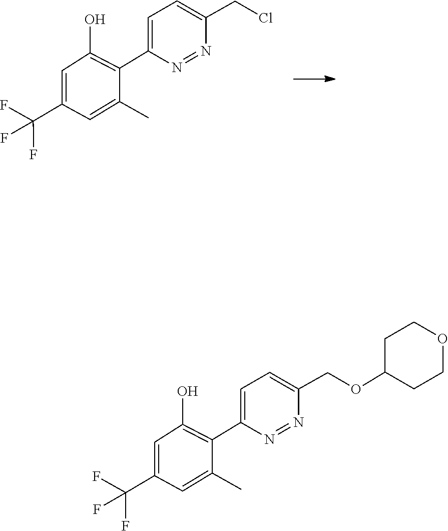 [SMILES: Cc1cc(C(F)(F)F)cc(O)c1-c1ccc(CCl)nn1 ; Cc1cc(C(F)(F)F)cc(O)c1-c1ccc(COC2CCOCC2)nn1]

To a solution of 2-[6-(chloromethyl)pyridazin-3-yl]-3-methyl-5-(trifluoromethyl)phenol (Intermediate C1, 80 mg, 264 mol) in THE (1 mL) were added tetrahydropyran-4-ol (264 μL, 2.64 mmol) and sodium tert-butoxide (127 mg, 1.32 mmol). The solution was stirred at 25° C. for 12 h under N₂. The reaction mixture was purified by prep-HPLC (column: Waters Xbridge BEH C18 100×30 mm, 10 μm; mobile phase: [(A) H₂O (10 mM NH₄HCO₃)—(B) ACN]; gradient: 20-55% B, 8 min) and lyophilised to give the title compound as a white solid, Y=25%. ¹H NMR (400 MHz, DMSO-d₆) δ 10.29 (s, 1H), 7.81 (d, J=9 Hz, 1H), 7.73 (d, J=9 Hz, 1H), 7.17 (s, 1H), 7.10 (s, 1H), 4.87 (s, 2H), 3.92-3.85 (m, 2H), 3.82-3.73 (m, 1H), 3.41-3.35 (m, 2H), 2.08 (s, 3H), 1.98-1.94 (m, 2H), 1.54-1.50 (m, 2H). LCMS (ESI): m/z: [M+H]⁺=369.0.

### Compound 18. 3-Methyl-2-(6-((piperidin-3-yloxy)methyl)pyridazin-3-yl)-5-(trifluoromethyl)phenol

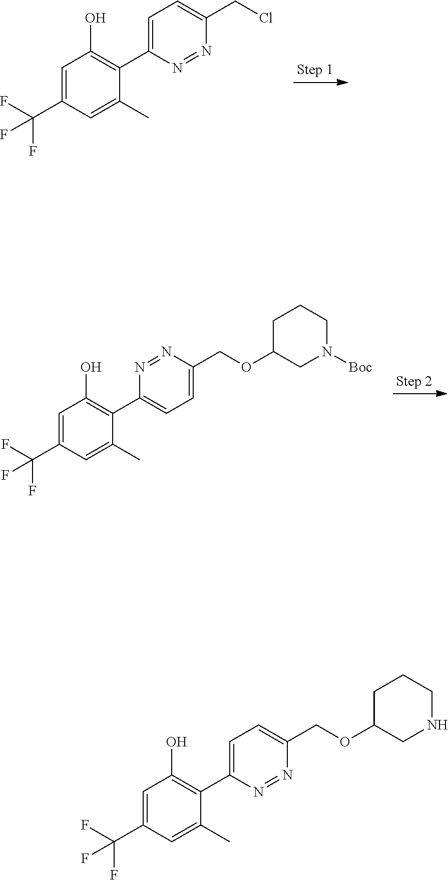 [SMILES: Cc1cc(C(F)(F)F)cc(O)c1-c1ccc(CCl)nn1 ; Cc1cc(C(F)(F)F)cc(O)c1-c1ccc(COC2CCCN(C(=O)OC(C)(C)C)C2)nn1 ; Cc1cc(C(F)(F)F)cc(O)c1-c1ccc(COC2CCCNC2)nn1]

### Step 1. Tert-butyl 3-((6-(2-hydroxy-6-methyl-4-(trifluoromethyl)phenyl)pyridazin-3-yl)methoxy)piperidine-1-carboxylate

To a solution of 2-[6-(chloromethyl)pyridazin-3-yl]-3-methyl-5-(trifluoromethyl)phenol (Intermediate C1, 400 mg, 1.32 mmol) in THE (7 mL) was added tert-butyl 3-hydroxypiperidine-1-carboxylate (1.06 g, 5.29 mmol) and sodium tert-butoxide (508 mg, 5.29 mmol) at 25° C. under N₂. The mixture was stirred at 80° C. for 12 h then allowed to cool to room temperature. The reaction mixture was concentrated under reduced pressure. The crude material was purified by prep-HPLC (column: Phenomenex Luna C18 100×40 mm, 3 μm; mobile phase: [(A) H₂O (10 mM NH₄HCO₃)—(B) ACN]; gradient: 30-60% B, 8 min) and lyophilised to give the title compound as a white solid, Y=29%. ¹H NMR (400 MHz, DMSO-d₆) δ 10.26 (br. s, 1H), 7.80-7.70 (m, 2H), 7.16 (s, 1H), 7.10 (s, 1H), 4.97-4.76 (m, 2H), 3.58 (s, 2H), 3.47-3.35 (m, 2H), 3.25-3.09 (m, 1H), 2.08 (s, 3H), 1.98-1.81 (m, 1H), 1.77-1.59 (m, 2H), 1.50-1.30 (m, 10H).

### Step 2. 3-Methyl-2-(6-((piperidin-3-yloxy)methyl)pyridazin-3-yl)-5-(trifluoromethyl)phenol

A mixture solution of tert-butyl 3-[[6-[2-hydroxy-6-methyl-4-(trifluoromethyl)phenyl]pyridazin-3-yl]methoxy]piperidine-1-carboxylate (20 mg, 42.8 mol) and 4 M HCl in EtOAc (0.2 mL) was stirred at 25° C. for 1 h under N₂. The reaction mixture was concentrated under reduced pressure. The crude material was purified by prep-HPLC (column: Waters Xbridge Prep OBD C18 150×40 mm, 10 μm; mobile phase: [(A) H₂O (10 mM NH₄HCO₃)—(B) ACN]; gradient: 20-50% B, 8 min) and lyophilised to give the title compound as a white solid, Y=88%. ¹H NMR (400 MHz, DMSO-d₆) δ 7.81 (d, J=9 Hz, 1H), 7.70 (d, J=9 Hz, 1H), 7.15 (s, 1H), 7.09 (s, 1H), 4.85 (s, 2H), 3.45-3.41 (m, 1H), 3.10-3.05 (m, 1H), 2.77-2.67 (m, 1H), 2.47-2.38 (m, 2H), 2.08 (s, 3H), 2.06-1.98 (m, 1H), 1.72-1.54 (m, 1H), 1.46-1.26 (m, 2H). LCMS (ESI): m/z: [M+H]⁺=368.1.

### Compound 19. 3-Methyl-2-(6-((piperidin-4-yloxy)methyl)pyridazin-3-yl)-5-(trifluoromethyl)phenol

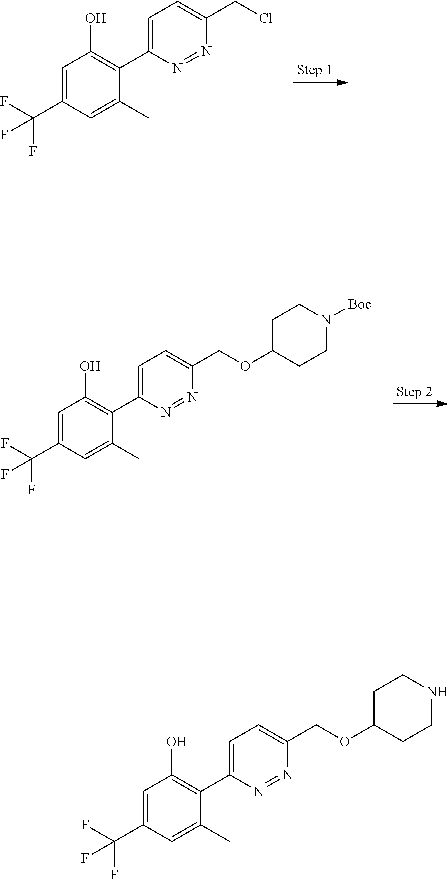 [SMILES: Cc1cc(C(F)(F)F)cc(O)c1-c1ccc(CCl)nn1 ; Cc1cc(C(F)(F)F)cc(O)c1-c1ccc(COC2CCN(C(=O)OC(C)(C)C)CC2)nn1 ; Cc1cc(C(F)(F)F)cc(O)c1-c1ccc(COC2CCNCC2)nn1]

### Step 1. Tert-butyl 4-((6-(2-hydroxy-6-methyl-4-(trifluoromethyl)phenyl)pyridazin-3-yl)methoxy)piperidine-1-carboxylate

To a solution of 2-[6-(chloromethyl)pyridazin-3-yl]-3-methyl-5-(trifluoromethyl)phenol (Intermediate C1, 200 mg, 661 mol) in THE (2 mL) were added tert-butyl 4-hydroxypiperidine-1-carboxylate (532 mg, 2.64 mmol) and sodium tert-butoxide (254 mg, 2.64 mmol) at 25° C. The solution was stirred for 12 h under N₂ then was cooled to 0° C., diluted with H₂O (5 mL) and extracted with EtOAc (3×3 mL). The combined organic phases were concentrated under reduced pressure to give the title compound as a white solid.

### Step 2. 3-Methyl-2-(6-((piperidin-4-yloxy)methyl)pyridazin-3-yl)-5-(trifluoromethyl)phenol

A mixture of tert-butyl 4-[[6-[2-hydroxy-6-methyl-4-(trifluoromethyl)phenyl]pyridazin-3-yl]methoxy]piperidine-1-carboxylate (300 mg, 642 mol) and 4 M HCl in EtOAc (3 mL) was stirred at 25° C. for 1 h under N₂. The reaction mixture was concentrated under reduced pressure to remove solvent. The residue was purified by prep-HPLC (column: Waters Xbridge C18 150×50 mm, 10 μm; mobile phase: [(A) H₂O (10 mM NH₄HCO₃)—(B) ACN]; gradient: 15-35% B, 8 min) and lyophilised to give the title compound as a white solid, Y=42%. ¹H NMR (400 MHz, DMSO-d₆) δ 7.79-7.69 (m, 2H), 7.14-7.08 (m, 2H), 4.85 (s, 2H), 3.58-3.54 (m, 1H), 2.97-2.93 (m, 2H), 2.50-2.41 (m, 2H), 2.09 (s, 3H), 1.94-1.92 (m, 2H), 1.40-1.37 (m, 2H).

LCMS (ESI): m/z: [M+H]⁺=368.1.

### Compound 20. 2-(6-(Aminomethyl)pyridazin-3-yl)-3-methyl-5-(trifluoromethyl)phenol

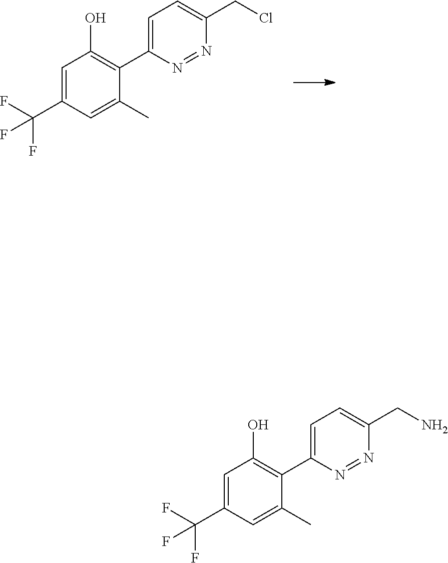 [SMILES: Cc1cc(C(F)(F)F)cc(O)c1-c1ccc(CCl)nn1 ; Cc1cc(C(F)(F)F)cc(O)c1-c1ccc(CN)nn1]

A mixture of 2-[6-(chloromethyl)pyridazin-3-yl]-3-methyl-5-(trifluoromethyl)phenol (Intermediate C1, 50 mg, 165 mol) and dioxane (3 mL) was stirred at 25° C. under NH₃ (gas, 15 psi). The mixture was stirred at 120° C. for 12 h then allowed to cool to room temperature and concentrated under reduced pressure. The crude material was purified by prep-HPLC (column: Waters Xbridge C18 150×50 mm, 10 μm; mobile phase: [(A) H₂O (10 mM NH₄HCO₃)—(B) ACN]; gradient: 10-40% B, 8.0 min) and lyophilised to give the title compound as a white solid, Y=36%. ¹H NMR (400 MHz, DMSO-d₆) δ 7.81 (d, J=9 Hz, 1H), 7.66 (d, J=9 Hz, 1H), 7.41 (br. s, 0.2H), 7.15 (s, 1H), 7.09 (s, 1H), 4.64-3.90 (m, 2H), 2.08 (s, 3H). LCMS (ESI): m/z: [M+H]⁺=284.0.

### Compound 29. 5-Bromo-3-methyl-2-(6-(((tetrahydro-2H-pyran-4-yl)amino)methyl)pyridazin-3-yl)phenol

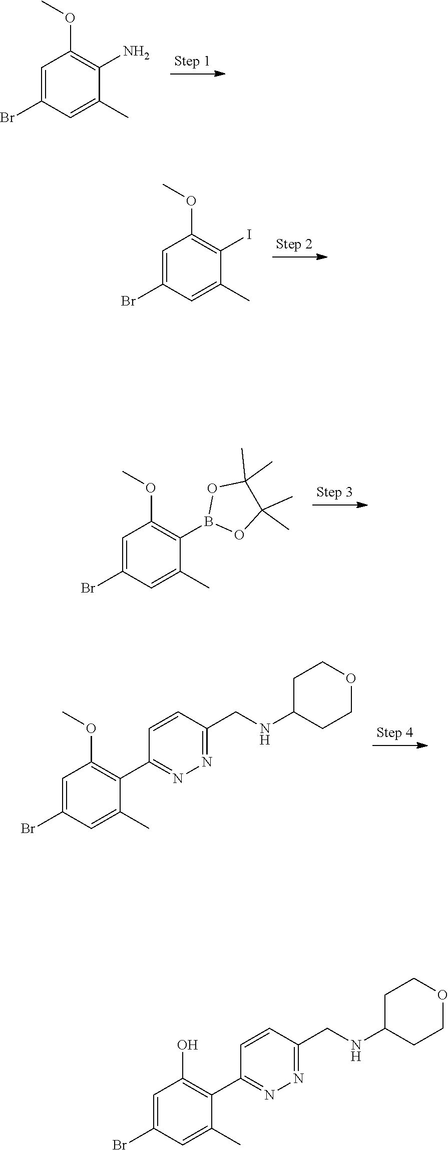 [SMILES: COc1cc(Br)cc(C)c1N ; COc1cc(Br)cc(C)c1I ; COc1cc(Br)cc(C)c1B1OC(C)(C)C(C)(C)O1 ; COc1cc(Br)cc(C)c1-c1ccc(CNC2CCOCC2)nn1 ; Cc1cc(Br)cc(O)c1-c1ccc(CNC2CCOCC2)nn1]

### Step 1. 5-Bromo-2-iodo-1-methoxy-3-methylbenzene

To a solution of 4-bromo-2-methoxy-6-methyl-aniline (3.00 g, 13.9 mmol) in conc. HCl (6 mL) and H₂O (6 mL) was added NaNO₂ (1.92 g, 27.8 mmol) at 0° C. The mixture was stirred at 0° C. for 30 min then a solution of KI (9.22 g, 55.5 mmol) in H₂O (6 mL) was added. The mixture was stirred at 25° C. for 12 h then was quenched by addition of saturated Na₂S₂O₃ solution (40 mL) at 0° C. and extracted with EtOAc (3×40 mL). The combined organic layers were washed with brine (2×30 mL), dried over Na₂SO₄, filtered and concentrated under reduced pressure. The residue was purified by column chromatography (SiO₂, petroleum ether:ethyl acetate=10:1 to 5:1) to give the title compound as a yellow solid, Y=88%. ¹H NMR (400 MHz, CDCl₃) δ 7.05 (s, 1H), 6.77 (s, 1H), 3.88 (s, 3H), 2.45 (s, 3H).

### Step 2. 2-(4-Bromo-2-methoxy-6-methylphenyl)-4,4,5,5-tetramethyl-1,3,2-dioxaborolane

To a solution of 5-bromo-2-iodo-1-methoxy-3-methyl-benzene (2.00 g, 6.12 mmol) in dioxane (30 mL) were added 4,4,5,5-tetramethyl-1,3,2-dioxaborolane (976 μL, 6.73 mmol), TEA (5.96 mL, 42.8 mmol), dicyclohexyl-(2-phenylphenyl)phosphane (534 mg, 1.53 mmol) and Pd(OAc)₂ (68.7 mg, 306 mol) at 25° C. under N₂. The mixture was stirred at 80° C. for 12 h under N₂. The reaction mixture was concentrated under reduced pressure, diluted with H₂O (20 mL) and extracted with EtOAc (3×15 mL). The combined organic layers were washed with brine (15 mL), dried over Na₂SO₄, filtered and concentrated under reduced pressure. The residue was purified by column chromatography (SiO₂, petroleum ether:ethyl acetate=10:1 to 5:1) to give the title compound as a yellow solid, Y=50%. ¹H NMR (400 MHz, CDCl₃) δ 6.92 (s, 1H), 6.79 (s, 1H), 3.76 (s, 3H), 2.32 (s, 3H), 1.38 (s, 12H).

### Step 3. N-((6-(4-Bromo-2-methoxy-6-methylphenyl)pyridazin-3-yl)methyl)tetrahydro-2H-pyran-4-amine

To a solution of 2-(4-bromo-2-methoxy-6-methyl-phenyl)-4,4,5,5-tetramethyl-1,3,2-dioxaborolane (500 mg, 1.53 mmol) in dioxane (25 mL) and H₂O (5 mL) were added N-[(6-chloropyridazin-3-yl)methyl]tetrahydropyran-4-amine (418 mg, 1.83 mmol), Na₂CO₃ (486 mg, 4.59 mmol) and Pd(PPh₃)₄ (35.3 mg, 30.6 mol) at 25° C. under N₂. The mixture was stirred at reflux for 12 h then allowed to cool to room temperature and concentrated under reduced pressure. The residue was diluted with H₂O (5 mL) and extracted with EtOAc (3×6 mL). The combined organic layers were washed with brine (2×5 mL), dried over Na₂SO₄, filtered and concentrated under reduced pressure to give a residue. The residue was purified by column chromatography (SiO2, petroleum ether/ethyl acetate=1/1 to 0/1) to give the title compound as a yellow solid, Y=13%.

### Step 4. 5-Bromo-3-methyl-2-(6-(((tetrahydro-2H-pyran-4-yl)amino)methyl)pyridazin-3-yl)phenol

To a solution of N-[[6-(4-bromo-2-methoxy-6-methyl-phenyl)pyridazin-3-yl]methyl]tetrahydropyran-4-amine (200 mg, 510 mol) in DCM (2 mL) was added BBr₃ (98.3 L, 1.02 mmol) at 0° C. under N₂. The mixture was stirred at 0° C. for 1 h then was quenched by addition of H₂O (4 mL) at 0° C. and extracted with DCM (3×2 mL). The combined organic layers were washed with brine (2×2 mL), dried over Na₂SO₄, filtered and concentrated under reduced pressure. The residue was purified by prep-HPLC (column: Waters Xbridge C18 150×50 mm, 10 μm; mobile phase: [(A) H₂O (10 mM NH₄HCO₃)—(B) ACN]; gradient: 20-40% B, 8.0 min) and lyophilised to give the title compound as a white solid, Y=4%. ¹H NMR (400 MHz, DMSO-d₆) δ 9.31 (br. s, 1H), 7.77 (d, J=9 Hz, 1H), 7.60 (d, J=9 Hz, 1H), 7.08-6.91 (m, 2H), 4.06 (s, 2H), 3.91-3.73 (m, 2H), 3.30-3.25 (m, 2H), 2.75-2.61 (m, 1H), 2.00 (s, 3H), 1.89-1.78 (m, 2H), 1.42-1.21 (m, 2H). LCMS (ESI): m/z: [M+H]⁺=378.0.

### Compound 30. 3-Methyl-2-(6-(((tetrahydro-2H-pyran-4-yl)amino)methyl)pyridazin-3-yl)-5-(trifluoromethoxy)phenol

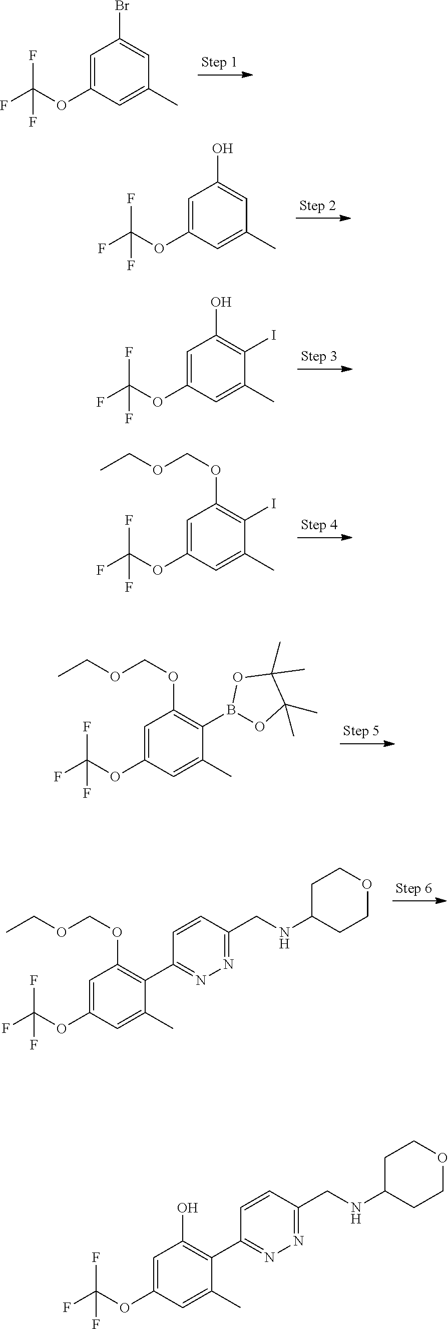

### Step 1. 3-Methyl-5-(trifluoromethoxy)phenol

To a solution of 1-bromo-3-methyl-5-(trifluoromethoxy)benzene (200 mg, 784 mol) in dioxane (1 mL) and H₂O (1 mL) were added (1E,4E)-1,5-diphenylpenta-1,4-dien-3-one;palladium (143 mg, 157 mol), ditert-butyl-[2-(2,4,6-triisopropylphenyl) phenyl]phosphane (26.6 mg, 62.7 mol) and KOH (132 mg, 2.35 mmol) at 25° C. under N₂. The mixture was stirred at reflux for 2 h then was allowed to cool to room temperature and H₂O (2 mL) was added. The mixture was extracted with EtOAc (3×2 mL). The combined organic layers were washed with brine (2 mL), dried over Na₂SO₄, filtered and concentrated under reduced pressure to give the title compound as a white solid, Y=66%.

### Step 2. 2-Iodo-3-methyl-5-(trifluoromethoxy)phenol

To a solution of 3-methyl-5-(trifluoromethoxy)phenol (120 mg, 625 mol) in toluene (5 mL) was added NaH (50.0 mg, 1.25 mmol, 60% wt. in mineral oil) at 0° C. under N₂. The mixture was stirred at 0° C. for 30 min under N₂. Then to above mixture was added I₂ (62.9 μL, 312 mol) at 0° C. under N₂. The mixture was stirred at 0° C. for 1 h then quenched by saturated aqueous NH₄Cl (5 mL) and extracted with EtOAc (3×5 mL). The combined organic layers were washed with brine (5 mL), dried over Na₂SO₄, filtered and concentrated under reduced pressure to give the title compound as a white solid, Y=50%.

### Step 3. 1-(Ethoxymethoxy)-2-iodo-3-methyl-5-(trifluoromethoxy)benzene

To a solution of 2-iodo-3-methyl-5-(trifluoromethoxy)phenol (300 mg, 943 mol) in DMF (5 mL) was added Cs₂CO₃ (615 mg, 1.89 mmol) and chloromethoxyethane (87.5 μL, 943 mol) at 25° C. The mixture was stirred at 25° C. for 2 h. To the mixture was added H₂O (7 mL), and extracted with EtOAc (3×2 mL). The combined organic layers were washed with brine (2 mL), dried over Na₂SO₄, filtered and concentrated under reduced pressure to give a residue. The residue was purified by column chromatography (SiO₂, petroleum ether:ethyl acetate=10:1) to give the title compound as a white solid, Y=56%. ¹H NMR (400 MHz, DMSO-d₆) δ 7.06 (s, 1H), 6.94 (s, 1H), 5.35 (s, 2H), 3.69 (q, J=7 Hz, 2H), 2.44 (s, 3H), 1.12 (t, J=7 Hz, 3H).

### Step 4. 2-(2-(Ethoxymethoxy)-6-methyl-4-(trifluoromethoxy)phenyl)-4,4,5,5-tetramethyl-1,3,2-dioxaborolane

To a solution of 1-(ethoxymethoxy)-2-iodo-3-methyl-5-(trifluoromethoxy)benzene (300 mg, 798 mol) in dioxane (5 mL) were added 4,4,5,5-tetramethyl-1,3,2-dioxaborolane (579 μL, 3.99 mmol), Pd(OAc)₂ (23.3 mg, 104 mol), TEA (822 μL, 5.90 mmol) and dicyclohexyl-(2-phenylphenyl)phosphane (67.1 mg, 191 mol) at 25° C. under N₂. The mixture was stirred at 80° C. for 12 h then was allowed to cool to room temperature and was diluted with H₂O (5 mL) and extracted with EtOAc (3×5 mL). The combined organic layers were washed with brine (5 mL), dried over Na₂SO₄, filtered and concentrated under reduced pressure. The residue was purified by column chromatography (SiO₂, petroleum ether:ethyl acetate=5:1) to give the title compound as a white solid, Y=67%. ¹H NMR (400 MHz, DMSO-d₆) δ 6.85-6.75 (m, 2H), 5.20 (s, 2H), 3.65 (q, J=7 Hz, 2H), 2.28 (s, 3H), 1.30 (s, 12H), 1.11 (t, J=7 Hz, 3H).

### Step 5. N-((6-(2-(Ethoxymethoxy)-6-methyl-4-(trifluoromethoxy)phenyl)pyridazin-3-yl)methyl)tetrahydro-2H-pyran-4-amine

To a solution of 2-[2-(ethoxymethoxy)-6-methyl-4-(trifluoromethoxy)phenyl]-4,4,5,5-tetramethyl-1,3,2-dioxaborolane (200 mg, 532 mol) in dioxane (8 mL) and H₂O (2.67 mL) were added N-[(6-chloropyridazin-3-yl)methyl]tetrahydropyran-4-amine (Intermediate A1, 121 mg, 532 mol), Na₂CO₃ (169 mg, 1.60 mmol) and Pd(PPh₃)₄ (30.7 mg, 26.6 mol) at 25° C. under N₂. The mixture was stirred at reflux for 2 h then allowed to cool to room temperature. The mixture was diluted with H₂O (3 mL) and extracted with ethyl acetate (3×3 mL). The combined organic layers were washed with brine (3 mL), dried over Na₂SO₄, filtered and concentrated under reduced pressure. The residue was purified by prep-HPLC (column: Waters Xbridge C18 150×50 mm, 10 μm; mobile phase: [(A) H₂O (10 mM NH₄HCO₃)—(B) ACN]; gradient: 35-55% B, 8 min) and lyophilised to give the title compound as a white solid, Y=38%. ¹H NMR (400 MHz, DMSO-d₆) δ 7.81 (d, J=9 Hz, 1H), 7.65 (d, J=9 Hz, 1H), 7.17-6.99 (m, 2H), 5.17 (s, 2H), 4.08 (s, 2H), 3.84 (m, 2H), 3.45 (q, J=7 Hz, 2H) 3.31-3.21 (m, 2H), 2.79-2.64 (m, 1H), 1.89-1.79 (m, 2H), 1.40-1.22 (m, 2H), 1.03 (t, J=7 Hz, 3H).

### Step 6. 3-Methyl-2-(6-(((tetrahydro-2H-pyran-4-yl)amino)methyl)pyridazin-3-yl)-5-(trifluoromethoxy)phenol

To a solution of N-[[6-[2-(ethoxymethoxy)-6-methyl-4-(trifluoromethoxy)phenyl]pyridazin-3-yl]methyl]tetrahydropyran-4-amine (100 mg, 227 mol) in DCM (1 mL) was added TFA (2.00 mL, 26.9 mmol) at 0° C. The mixture was stirred at 0° C. for 2 h and concentrated under reduced pressure. The residue was purified by prep-HPLC (column: Waters Xbridge Prep OBD C18 150×40 mm, 10 um; mobile phase: [(A) H₂O (10 mM NH₄HCO₃)—(B) ACN]; gradient: 20-60% B, 8 min) and lyophilised to give the title compound as a white solid, Y=34%. ¹H NMR (400 MHz, DMSO-d₆) δ 10.34 (br. s, 1H), 7.77 (d, J=9 Hz, 1H), 7.63 (d, J=9 Hz, 1H), 6.79-6.71 (m, 2H), 4.06 (s, 2H), 3.83 (m, 2H), 3.31-3.24 (m, 2H), 2.71-2.59 (m, 1H), 2.03 (s, 3H), 1.89-1.77 (m, 2H), 1.36-1.22 (m, 2H). LCMS (ESI): m/z: [M+H]⁺=384.1.

### Compound 31. 3-Methyl-2-(6-(((5-methylisoxazol-3-yl)amino)methyl)pyridazin-3-yl)-5-(trifluoromethyl)phenol

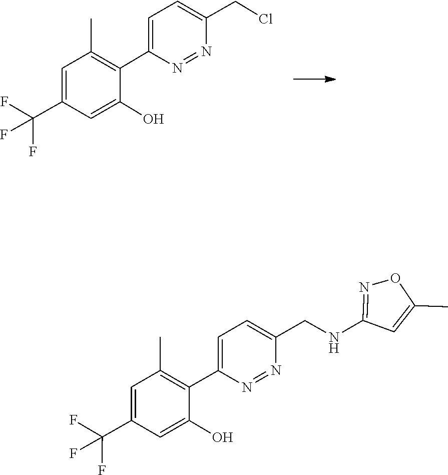 [SMILES: Cc1cc(C(F)(F)F)cc(O)c1-c1ccc(CCl)nn1 ; Cc1cc(NCc2ccc(-c3c(C)cc(C(F)(F)F)cc3O)nn2)no1]

To a solution of 2-[6-(chloromethyl)pyridazin-3-yl]-3-methyl-5-(trifluoromethyl)phenol (Intermediate C1, 150 mg, 496 mol) and 5-methylisoxazol-3-amine (292 mg, 2.97 mmol) in THE (3 mL) was added LiHMDS (1 M in THF, 842 μL, 842 mol) at 0° C. under N₂. The mixture was stirred at 0° C. for 1 h then was quenched by addition of saturated NH₄Cl aqueous solution (0.3 mL) and extracted with EtOAc (3×1 mL). The combined organic layers were washed with brine (1 mL), dried over Na₂SO₄, filtered and concentrated under reduced pressure. The residue was purified by prep-HPLC (column: Phenomenex Gemini-NX 150×30 mm, 5 μm; mobile phase: [(A) H₂O (10 mM NH₄HCO₃)—(B) ACN]; gradient: 25-55% B, 20 min) and lyophilised to give the title compound as a white solid, Y=8%. ¹H NMR (400 MHz, DMSO-d₆) δ 10.27 (br. s, 1H), 7.71-7.63 (m, 2H), 7.16 (s, 1H), 7.10 (s, 1H), 6.86 (t, J=6 Hz, 1H), 5.76 (s, 1H), 4.59 (d, J=6 Hz, 2H), 2.23 (s, 3H), 2.08 (s, 3H). LC-MS (ESI): m/z: [M+H]⁺=365.0.

### Compound 32. (R)-3-Methyl-2-(6-(((tetrahydrofuran-3-yl)amino)methyl)pyridazin-3-yl)-5-(trifluoromethyl)phenol

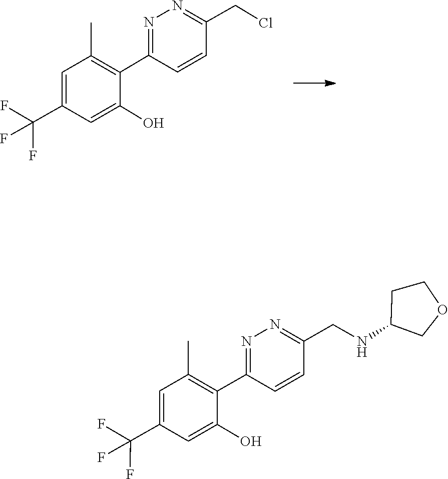 [SMILES: Cc1cc(C(F)(F)F)cc(O)c1-c1ccc(CCl)nn1 ; Cc1cc(C(F)(F)F)cc(O)c1-c1ccc(CN[C@@H]2CCOC2)nn1]

To a solution of 2-[6-(chloromethyl)pyridazin-3-yl]-3-methyl-5-(trifluoromethyl)phenol (Intermediate C1, 60 mg, 198 mol) in DMSO (0.3 mL) was added (3R)-tetrahydrofuran-3-amine (51.8 mg, 595 mol) and DIPEA (69.1 μL, 396 mol) at 25° C. under N₂. The mixture was stirred at 120° C. for 2 h then allowed to cool to room temperature. The reaction mixture was purified by prep-HPLC (column: Waters Xbridge C18 150×50 mm, 10 μm; mobile phase: [(A) H₂O (10 mM NH₄HCO₃)—(B) ACN]; gradient: 20-50% B, 8 min) and lyophilised to give the title compound as a white solid, Y=50%. ¹H NMR (400 MHz, DMSO-d₆) δ 9.69 (br. s, 1H), 7.79 (d, J=9 Hz, 1H), 7.66 (d, J=9 Hz, 1H), 7.25-6.97 (m, 2H), 4.10-3.97 (m, 2H), 3.85-3.63 (m, 3H), 3.50-3.45 (m, 1H), 3.39-3.31 (m, 1H), 2.72-2.53 (m, 1H), 2.08 (s, 3H), 2.03-1.95 (m, 1H), 1.79-1.68 (m, 1H). LCMS (ESI): m/z: [M+H]⁺=354.1.

### Compound 33. (S)-3-methyl-2-(6-(((tetrahydrofuran-3-yl)amino)methyl)pyridazin-3-yl)-5-(trifluoromethyl)phenol

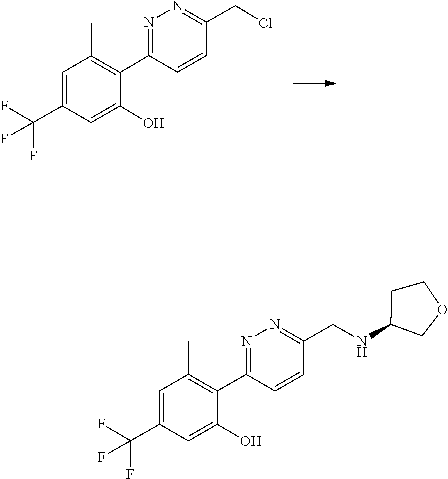 [SMILES: Cc1cc(C(F)(F)F)cc(O)c1-c1ccc(CCl)nn1 ; Cc1cc(C(F)(F)F)cc(O)c1-c1ccc(CN[C@H]2CCOC2)nn1]

To a solution of 2-[6-(chloromethyl)pyridazin-3-yl]-3-methyl-5-(trifluoromethyl)phenol (Intermediate C1, 200 mg, 661 mol) in DMSO (2 mL) was added (3S)-tetrahydrofuran-3-amine (69.1 mg, 793 mol) and DIPEA (231 μL, 1.32 mmol) at 25° C., the solution was stirred at 120° C. for 3 h under N₂. The reaction mixture was purified by prep-HPLC (column: Phenomenex Gemini-NX 150×30 mm, 5 μm; mobile phase: [(A) H₂O (10 mM NH₄HCO₃)—(B) ACN]; gradient: 20-50% B, 20 min) and lyophilised to give the title compound as a white solid, Y=39%. ¹H NMR (400 MHz, DMSO-d₆) δ 10.11 (br. s, 1H), 7.80 (d, J=9 Hz, 1H), 7.67 (d, J=9 Hz, 1H), 7.16 (s, 1H), 7.10 (s, 1H), 4.10-3.98 (m, 2H), 3.83-3.62 (m, 3H), 3.48-3.46 (m, 1H), 3.41-3.28 (m, 1H), 2.75-2.59 (m, 1H), 2.08 (s, 3H), 2.00-1.95 (m, 1H), 1.74-1.72 (m, 1H). LCMS (ESI): m/z: [M+H]⁺=354.2.

### Compound 34. (R)-2-(6-((3-Fluoropyrrolidin-1-yl)methyl)pyridazin-3-yl)-3-methyl-5-(trifluoromethyl)phenol

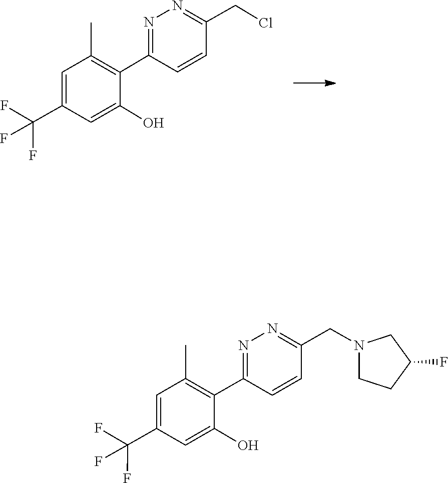 [SMILES: Cc1cc(C(F)(F)F)cc(O)c1-c1ccc(CCl)nn1 ; Cc1cc(C(F)(F)F)cc(O)c1-c1ccc(CN2CC[C@@H](F)C2)nn1]

To a solution of 2-[6-(chloromethyl)pyridazin-3-yl]-3-methyl-5-(trifluoromethyl)phenol (70.0 mg, 231 mol) in DMSO (1 mL) were added (3R)-3-fluoropyrrolidine (30.9 mg, 347 mol) and DIPEA (121 μL, 694 mol) at 25° C. The mixture was stirred at 80° C. for 2 h under N₂. The reaction mixture was purified by prep-HPLC (column: Waters Xbridge C18 150×50 mm, 10 μm; mobile phase: [(A) H₂O (10 mM NH₄HCO₃)—ACN]; gradient: 20-60% B, 8 min) and lyophilised to give the title compound as a white solid, Y=36%. ¹H NMR (400 MHz, DMSO-d₆) δ 10.32 (s, 1H), 7.79-7.65 (m, 2H), 7.16 (s, 1H), 7.10 (s, 1H), 5.34-5.13 (m, 1H), 3.99 (s, 2H), 2.95-2.83 (m, 2H), 2.79-2.66 (m, 1H), 2.47-2.42 (m, 1H), 2.27-2.11 (m, 1H), 2.08 (s, 3H), 2.00-1.82 (m, 1H). LC-MS (ESI): m/z: [M+H]⁺=356.1.

### Compound 35. (S)-2-(6-((3-Fluoropyrrolidin-1-yl)methyl)pyridazin-3-yl)-3-methyl-5-(trifluoromethyl)phenol

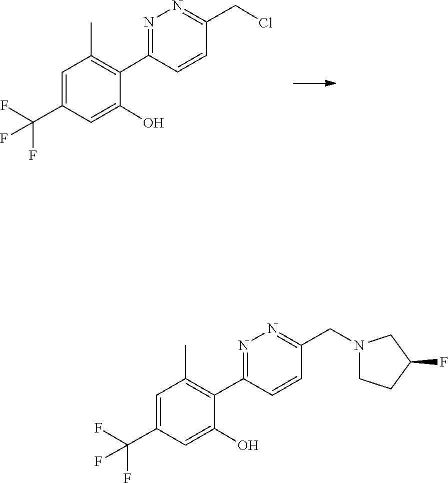 [SMILES: Cc1cc(C(F)(F)F)cc(O)c1-c1ccc(CCl)nn1 ; Cc1cc(C(F)(F)F)cc(O)c1-c1ccc(CN2CC[C@H](F)C2)nn1]

To a solution of 2-[6-(chloromethyl)pyridazin-3-yl]-3-methyl-5-(trifluoromethyl)phenol (Intermediate C1, 70 mg, 231 mol) in DMSO (1.5 mL) were added DIPEA (161 μL, 925 mol) and (3S)-3-fluoropyrrolidine (29.0 mg, 231 mol) at 25° C. The mixture was stirred at 80° C. for 1 h then allowed to cool to room temperature. The reaction mixture was purified by prep-HPLC (column: Waters Xbridge Prep OBD C18 150×40 mm, 10 μm; mobile phase: [(A) H₂O (10 mM NH₄HCO₃)—(B) ACN]; gradient: 30-60% B, 8 min) and lyophilised to give the title compound as a white solid, Y=36%. ¹H NMR (400 MHz, DMSO-d₆) δ 10.24 (br. s, 1H), 7.75 (d, J=9 Hz, 1H), 7.67 (d, J=9 Hz, 1H), 7.16 (s, 1H), 7.10 (s, 1H), 5.47-5.07 (m, 1H), 3.99 (s, 2H), 2.97-2.83 (m, 2H), 2.82-2.66 (m, 1H), 2.47-2.41 (m, 1H), 2.25-2.11 (m, 1H), 2.08 (s, 3H), 2.01-1.81 (m, 1H). LCMS (ESI): m/z: [M+H]⁺=356.0.

### Compound 36. 3,5-Dichloro-2-(6-(((tetrahydro-2H-pyran-4-yl)amino)methyl)pyridazin-3-yl)phenol

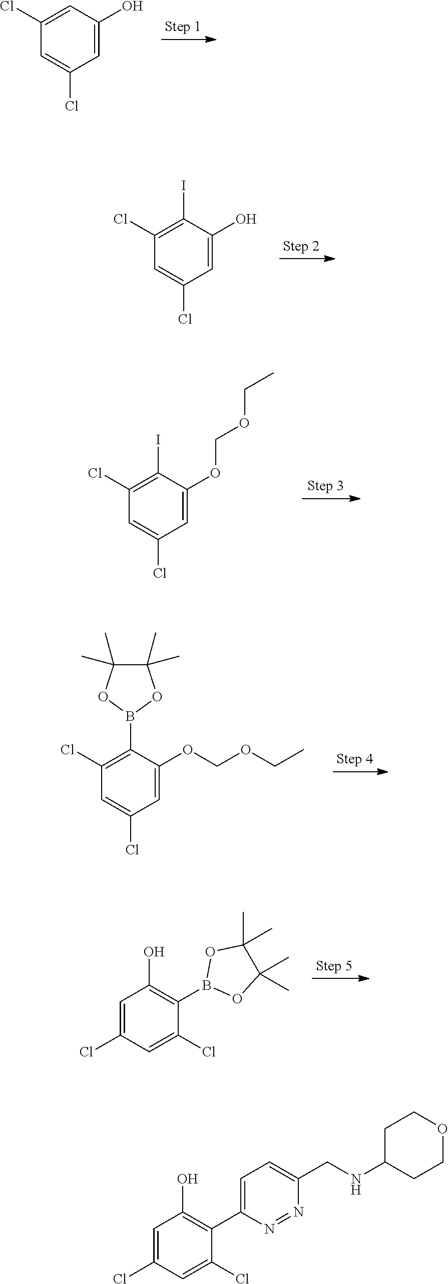 [SMILES: Oc1cc(Cl)cc(Cl)c1 ; Oc1cc(Cl)cc(Cl)c1I ; CCOCOc1cc(Cl)cc(Cl)c1I ; CCOCOc1cc(Cl)cc(Cl)c1B1OC(C)(C)C(C)(C)O1 ; CC1(C)OB(c2c(O)cc(Cl)cc2Cl)OC1(C)C ; Oc1cc(Cl)cc(Cl)c1-c1ccc(CNC2CCOCC2)nn1]

### Step 1. 3,5-Dichloro-2-iodophenol

To a solution of 3,5-dichlorophenol (10.0 g, 61.4 mmol) in toluene (100 mL) was added NaH (4.91 g, 123 mmol, 60% wt in mineral oil) at 0° C. under N₂. The mixture was stirred at 0° C. for 30 min then I₂ (12.4 mL, 61.4 mmol) was added. The mixture was stirred at 0° C. for 1 h then poured into saturated aqueous NH₄Cl (100 mL), and extracted with EtOAc (3×100 mL). The combined organic phase was washed with brine (3×100 mL), dried with anhydrous Na₂SO₄, filtered and concentrated under reduced pressure. The residue was purified by column chromatography (SiO₂, petroleum ether:ethyl acetate=5:1) to give the title compound as a colourless oil.

### Step 2. 1,5-Dichloro-3-(ethoxymethoxy)-2-iodobenzene

To a solution of 3,5-dichloro-2-iodo-phenol (21.0 g, 72.7 mmol) in ACN (210 mL) was added Cs₂CO₃ (47.4 g, 145 mmol) and chloromethoxyethane (10.1 mL, 109 mmol) at 25° C. under N₂. The mixture was stirred at 25° C. for 1 h. The reaction mixture was concentrated under reduced pressure to give a residue. The residue was diluted with H₂O (200 mL) and extracted with EtOAc (3×200 mL). The combined organic phase was washed with brine (3×200 mL), dried with anhydrous Na₂SO₄, filtered and concentrated under reduced pressure. The residue was purified by column chromatography (SiO₂, petroleum ether:ethyl acetate=5:1) to give the title compound as a white solid, Y=95%.

### Step 3. 2-(2,4-Dichloro-6-(ethoxymethoxy)phenyl)-4,4,5,5-tetramethyl-1,3,2-dioxaborolane

To a solution of 1,5-dichloro-3-(ethoxymethoxy)-2-iodo-benzene (8.00 g, 23.1 mmol) and 4,4,5,5-tetramethyl-1,3,2-dioxaborolane (16.7 mL, 115 mmol) in dioxane (80 mL) were added TEA (23.8 mL, 171 mmol), Pd(OAc)₂ (673 mg, 3.00 mmol) and dicyclohexyl-(2-phenylphenyl)phosphane (1.94 g, 5.53 mmol) at 25° C. under N₂. The mixture was stirred at 80° C. for 12 h then allowed to cool to room temperature. The reaction mixture was diluted with H₂O (80 mL) and extracted with EtOAc (3×80 mL). The combined organic phase was washed with brine (80 mL), dried with anhydrous Na₂SO₄, filtered and concentrated under reduced pressure. The residue was purified by prep-HPLC (column: Welch Xtimate C18 250×100 mm, m; mobile phase: [(A) H₂O (10 mM NH₄HCO₃)—(B) ACN]; gradient: 60-85% B, 27 min) to give the title compound as a white solid, Y=97%. ¹H NMR (400 MHz, DMSO-d₆) δ 7.17 (d, J=2 Hz, 1H), 7.11 (d, J=2 Hz, 1H), 5.28 (s, 2H), 3.65 (q, J=7 Hz, 2H), 1.30 (s, 12H), 1.12 (t, J=7 Hz, 3H).

### Step 4. 3,5-Dichloro-2-(4,4,5,5-tetramethyl-1,3,2-dioxaborolan-2-yl)phenol

A mixture of 2-[2,4-dichloro-6-(ethoxymethoxy)phenyl]-4,4,5,5-tetramethyl-1,3,2-dioxaborolane (700 mg, 2.02 mmol) and 4 M HCl in EtOAc (7 mL) was stirred at 25° C. for 2 h. The reaction mixture was concentrated under reduced pressure to give the title compound as a white solid, Y=94%. ¹H NMR (400 MHz, DMSO-d₆) δ 10.31 (s, 1H), 6.93 (d, J=2 Hz, 1H), 6.75 (d, J=2 Hz, 1H), 1.29 (s, 12H).

### Step 5. 3,5-Dichloro-2-(6-(((tetrahydro-2H-pyran-4-yl)amino)methyl)pyridazin-3-yl)phenol

To a solution of 3,5-dichloro-2-(4,4,5,5-tetramethyl-1,3,2-dioxaborolan-2-yl)phenol (305 mg, 1.05 mmol) and N-[(6-chloropyridazin-3-yl)methyl]tetrahydropyran-4-amine (200 mg, 878 mol) in dioxane (5 mL) and H₂O (1.7 mL) were added Na₂CO₃ (279 mg, 2.64 mmol) and Pd(PPh₃)₄ (20.3 mg, 17.6 mol) at 25° C. under N₂. The mixture was stirred at reflux temperature for 15 h. The reaction mixture was diluted with H₂O (2 mL) and extracted with EtOAc (3×2 mL). The combined organic phase was washed with brine (3×2 mL), dried with anhydrous Na₂SO₄, filtered and concentrated under vacuum. The residue was purified by prep-HPLC (column: Waters Xbridge Prep OBD C18 150×40 mm, 10 μm; mobile phase: [(A) H₂O (10 mM NH₄HCO₃)—(B) ACN]; gradient: 15-55% B, 8 min) and lyophilised to give the title compound as a white solid, Y=64%. 1H NMR (400 MHz, DMSO-d₆) δ 7.81 (d, J=9 Hz, 1H), 7.65 (d, J=9 Hz, 1H), 7.17 (d, J=2 Hz, 1H), 6.98 (d, J=2 Hz, 1H), 4.08 (s, 2H), 3.95-3.80 (m, 2H), 3.40-3.20 (m, 2H), 2.72-2.62 (m, 1H), 1.86-1.78 (m, 2H), 1.37-1.25 (m, 2H). LCMS (ESI): m/z: [M+H]⁺=354.0.

### Compound 37. (R)-4-(((6-(2-Hydroxy-6-methyl-4-(trifluoromethyl)phenyl)pyridazin-3-yl)methyl)amino)-1-methylpiperidin-2-one

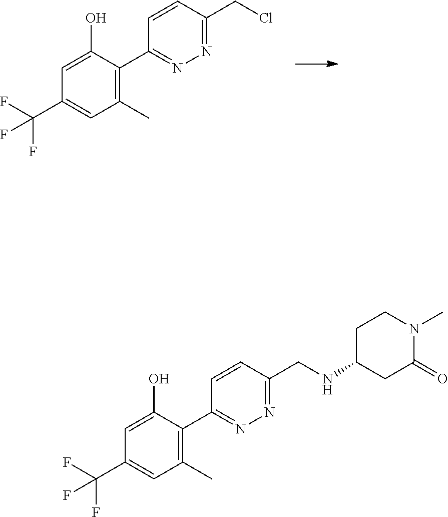 [SMILES: Cc1cc(C(F)(F)F)cc(O)c1-c1ccc(CCl)nn1 ; Cc1cc(C(F)(F)F)cc(O)c1-c1ccc(CN[C@@H]2CCN(C)C(=O)C2)nn1]

To a solution of 2-[6-(chloromethyl)pyridazin-3-yl]-3-methyl-5-(trifluoromethyl)phenol (Intermediate C1, 60.0 mg, 198 mol) in DMSO (1 mL) were added (4R)-4-amino-1-methyl-piperidin-2-one (25.4 mg, 198 mol) and DIPEA (104 μL, 595 mol) at 25° C. The mixture was stirred at 80° C. for 2 h then allowed to cool to room temperature. The reaction mixture was purified by prep-HPLC (column: Waters Xbridge Prep OBD C18 150×40 mm, 10 μm; mobile phase: [(A) H₂O (10 mM NH₄HCO₃)—(B) ACN]; gradient: 20-50% B, 8 min) and lyophilised to give the title compound as a yellow solid, Y=51%. ¹H NMR (400 MHz, DMSO-d₆) δ 9.90 (br. s, 1H), 7.79 (d, J=9 Hz, 1H), 7.66 (d, J=9 Hz, 1H), 7.15 (s, 1H), 7.09 (s, 1H), 4.12-4.01 (m, 2H), 3.37-3.35 (m, 1H), 3.22-3.14 (m, 1H), 3.01-2.92 (m, 1H), 2.79 (s, 3H), 2.72-2.61 (m, 1H), 2.49-2.43 (m, 1H), 2.18-2.05 (m, 4H), 2.04-1.95 (m, 1H), 1.75-1.62 (m, 1H). LCMS (ESI): m/z: [M+H]⁺=395.1.

### Compound 38. 2-(6-((3,3-Difluoropyrrolidin-1-yl)methyl)pyridazin-3-yl)-3-methyl-5-(trifluoromethyl)phenol

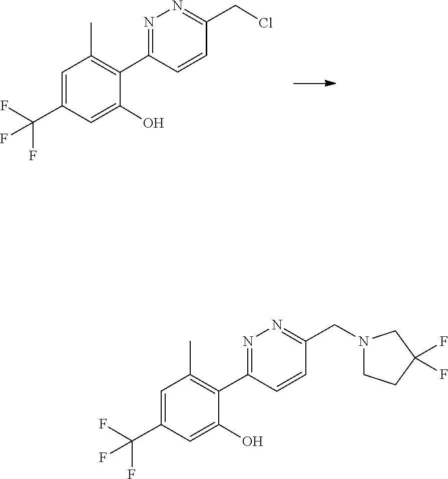 [SMILES: Cc1cc(C(F)(F)F)cc(O)c1-c1ccc(CCl)nn1 ; Cc1cc(C(F)(F)F)cc(O)c1-c1ccc(CN2CCC(F)(F)C2)nn1]

To a solution of 2-[6-(chloromethyl)pyridazin-3-yl]-3-methyl-5-(trifluoromethyl)phenol (Intermediate C1, 70.0 mg, 231 mol) in DMSO (1 mL) was added 3,3-difluoropyrrolidine (37.2 mg, 347 mol) and DIPEA (80.6 μL 462 mol) at 25° C. The mixture was stirred at 80° C. for 2 h then allowed to cool to room temperature. The reaction mixture was purified by prep-HPLC (column: Waters Xbridge C18 150×50 mm, 10 um; mobile phase: [(A) H₂O (10 mM NH₄HCO₃)—(B) ACN]; gradient: 25-65% B, 8 min) and lyophilised to give the title compound as a white solid, Y=35%. ¹H NMR (400 MHz, DMSO-d₆) δ 10.29 (s, 1H), 7.76 (d, J=9 Hz, 1H), 7.69 (d, J=9 Hz, 1H), 7.16 (s, 1H), 7.10 (s, 1H), 4.01 (s, 2H), 3.01 (t, J=13 Hz, 2H), 2.82 (t, J=7 Hz, 2H), 2.38-2.23 (m, 2H), 2.08 (s, 3H). LCMS (ESI): m/z: [M+H]⁺=374.1.

### Compound 39. (R)-2-(6-((3-Methoxypyrrolidin-1-yl)methyl)pyridazin-3-yl)-3-methyl-5-(trifluoromethyl)phenol

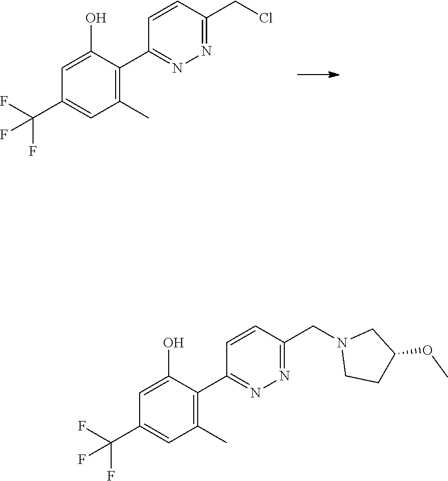 [SMILES: Cc1cc(C(F)(F)F)cc(O)c1-c1ccc(CCl)nn1 ; CO[C@@H]1CCN(Cc2ccc(-c3c(C)cc(C(F)(F)F)cc3O)nn2)C1]

To a solution of 2-[6-(chloromethyl)pyridazin-3-yl]-3-methyl-5-(trifluoromethyl)phenol (Intermediate C1, 70.0 mg, 231 mol) in DMSO (1 mL) were added DIPEA (80.6 μL, 463 mol) and (3R)-3-methoxypyrrolidine (23.4 mg, 231 mol) at 25° C. The mixture was stirred at 80° C. for 2 h. The reaction mixture was purified by prep-HPLC (column: Waters Xbridge C18 150×50 mm, 10 μm; mobile phase: [(A) H₂O (10 mM NH₄HCO₃)—(B) ACN]; gradient: 25-55% B, 8 min) and lyophilised to give the title compound as a white solid, Y=37%. ¹H NMR (400 MHz, DMSO-d₆) δ 10.18 (br. s, 1H), 7.76-7.71 (d, J=9 Hz, 1H), 7.69-7.64 (d, J=9 Hz, 1H), 7.15 (s, 1H), 7.10 (s, 1H), 3.94 (s, 2H), 3.93-3.89 (m, 1H), 3.17 (s, 3H), 2.80-2.72 (m, 1H), 2.70-2.61 (m, 1H), 2.60-2.50 (m, 1H), 2.47-2.42 (m, 1H), 2.08 (s, 3H), 2.09-1.98 (m, 1H), 1.76-1.62 (m, 1H). LCMS (ESI): m/z: [M+H]⁺=368.0.

### Compound 40. (S)-2-(6-((3-Methoxypyrrolidin-1-yl)methyl)pyridazin-3-yl)-3-methyl-5-(trifluoromethyl)phenol

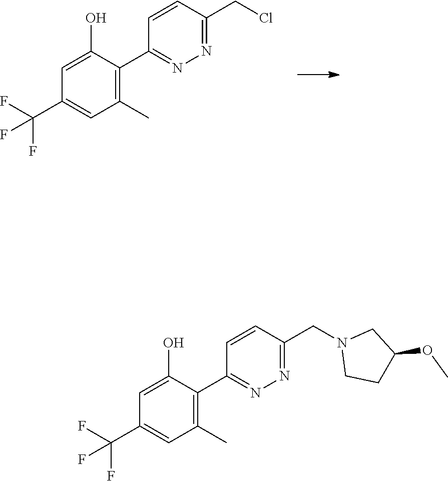 [SMILES: Cc1cc(C(F)(F)F)cc(O)c1-c1ccc(CCl)nn1 ; CO[C@H]1CCN(Cc2ccc(-c3c(C)cc(C(F)(F)F)cc3O)nn2)C1]

To a solution of 2-[6-(chloromethyl)pyridazin-3-yl]-3-methyl-5-(trifluoromethyl)phenol (Intermediate C1, 60.0 mg, 198 mol) in DMSO (0.6 mL) were added DIPEA (69.1 μL, 396 mol) and (3S)-3-methoxypyrrolidine (20.1 mg, 198 mol) at 25° C. under N₂. The mixture was stirred at 120° C. for 2 h. The reaction mixture was purified by prep-HPLC (column: Waters Xbridge C18 150×50 mm, 10 μm; mobile phase: [(A) H₂O (10 mM NH₄HCO₃)—(B) ACN]; gradient: 20-60% B, 8 min) and lyophilised to give the title compound as a yellow solid, Y=48%. ¹H NMR (400 MHz, DMSO-d₆) δ 10.24 (br. s, 1H), 7.76 (d, J=9 Hz, 1H), 7.67 (d, J=9 Hz, 1H), 7.20-7.07 (m, 2H), 3.98-3.87 (m, 3H), 3.17 (s, 3H), 2.80-2.73 (m, 1H), 2.71-2.63 (m, 1H), 2.59-2.51 (m, 2H), 2.08 (s, 3H), 2.06-1.97 (m, 1H), 1.76-1.61 (m, 1H). LCMS (ESI): m/z: [M+H]⁺=368.1.

### Compound 41. 3-Methyl-2-(6-(((1-methylpiperidin-3-yl)oxy)methyl)pyridazin-3-yl)-5-(trifluoromethyl)phenol hydrochloride

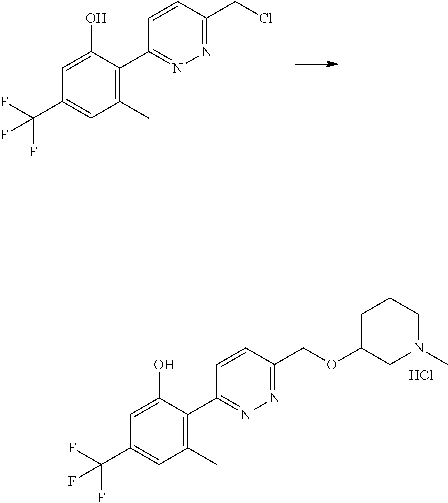 [SMILES: Cc1cc(C(F)(F)F)cc(O)c1-c1ccc(CCl)nn1 ; Cc1cc(C(F)(F)F)cc(O)c1-c1ccc(COC2CCCN(C)C2)nn1 ; Cl]

To a solution of 1-methylpiperidin-3-ol (61.4 μL, 521 mol) in DMF (1.4 mL) was added NaH (41.7 mg, 1.04 mmol, 60% purity in mineral oil) at 0° C. under N₂. The mixture was stirred at 0° C. for 0.5 h then 2-[6-(chloromethyl)pyridazin-3-yl]-3-methyl-5-(trifluoromethyl)phenol (Intermediate C1, 173 mg, 573 mol) was added at 0° C. The mixture was stirred at 25° C. for 12 h then quenched with saturated aqueous NH₄Cl (1 mL) and the resulting mixture was extracted with EtOAc (3×1 mL). The combined organic layers were washed with brine (1 mL), dried over Na₂SO₄, filtered and concentrated under reduced pressure. The residue was purified by prep-HPLC (column: Phenomenex Luna C18 100×30 mm, 3 um; mobile phase: [(A) H₂O (0.04% HCl)—(B) ACN]; gradient: 5-40% B, 8 min) and lyophilised to give the title compound as a white solid, Y=13%. ¹H NMR (400 MHz, DMSO-d₆) δ 10.70-9.25 (m, 2H), 8.28-7.66 (m, 2H), 7.18 (s, 2H), 5.04-4.81 (m, 2H), 4.08-3.83 (m, 1H), 3.73-3.68 (m, 1H), 3.38-3.27 (m, 1H), 3.23-3.11 (m, 1H), 3.02-2.75 (m, 4H), 2.26-1.85 (m, 5H), 1.75-1.32 (m, 2H). LCMS (ESI): m/z: [M+H]⁺=382.1.

### Compound 42. 3-Fluoro-2-(6-(((tetrahydro-2H-pyran-4-yl)amino)methyl)pyridazin-3-yl)-5-(trifluoromethyl)phenol

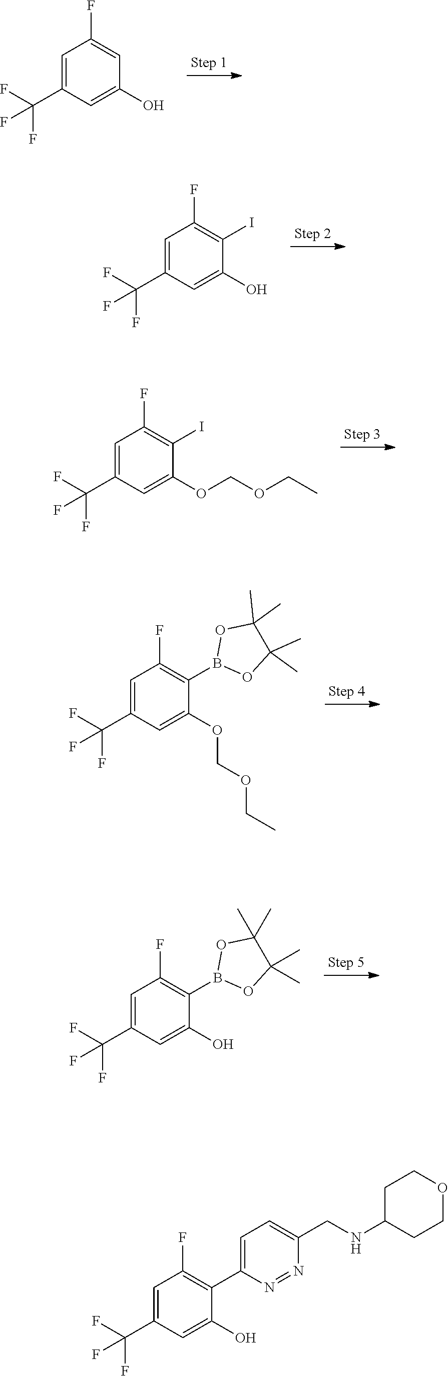 [SMILES: Oc1cc(F)cc(C(F)(F)F)c1 ; Oc1cc(C(F)(F)F)cc(F)c1I ; CCOCOc1cc(C(F)(F)F)cc(F)c1I ; CCOCOc1cc(C(F)(F)F)cc(F)c1B1OC(C)(C)C(C)(C)O1 ; CC1(C)OB(c2c(O)cc(C(F)(F)F)cc2F)OC1(C)C ; Oc1cc(C(F)(F)F)cc(F)c1-c1ccc(CNC2CCOCC2)nn1]

### Step 1. 3-Fluoro-2-iodo-5-(trifluoromethyl)phenol

To a solution of 3-fluoro-5-(trifluoromethyl)phenol (3.00 g, 16.7 mmol) in toluene (90 mL) was added NaH (1.33 g, 33.3 mmol, 60% wt. in mineral oil) at 0° C. under N₂. The mixture was stirred at 0° C. for 40 min then I₂ (3.36 mL, 16.7 mmol) was added. The mixture was stirred at 0° C. for 1 h then was quenched by addition of saturated aqueous NH₄Cl (30 mL) and extracted with EtOAc (3×30 mL). The combined organic layers were washed with brine (30 mL), dried over Na₂SO₄, filtered and concentrated under reduced pressure to give the title compound as a white solid, Y=98%.

### Step 2. 1-(Ethoxymethoxy)-3-fluoro-2-iodo-5-(trifluoromethyl)benzene

To a solution of 3-fluoro-2-iodo-5-(trifluoromethyl)phenol (5.00 g, 16.3 mmol) in DMF (50 mL) was added Cs₂CO₃ (5.32 g, 16.3 mmol) and chloromethoxyethane (1.52 mL, 16.3 mmol) at 25° C. The mixture was stirred at 25° C. for 2 h. The mixture diluted with H₂O (50 mL), and extracted with EtOAc (3×50 mL). The combined organic layers were washed with brine (50 mL), dried over Na₂SO₄, filtered and concentrated under reduced pressure. The residue was purified by column chromatography (SiO₂, petroleum ether:ethyl acetate=20:1 to 10:1) to give the title compound as a white solid, Y=76%.

### Step 3. 2-(2-(Ethoxymethoxy)-6-fluoro-4-(trifluoromethyl)phenyl)-4,4,5,5-tetramethyl-1,3,2-dioxaborolane

To a solution of 1-(ethoxymethoxy)-3-fluoro-2-iodo-5-(trifluoromethyl)benzene (3.50 g, 9.61 mmol) in dioxane (35 mL) were added TEA (9.90 mL, 71.14 mmol) and 4,4,5,5-tetramethyl-1,3,2-dioxaborolane (6.97 mL, 48.1 mmol), Pd(OAc)₂ (281 mg, 1.25 mmol) and biphenyl-2-yl-dicyclohexylphosphane (809 mg, 2.31 mmol) at 25° C. under N₂. The mixture was stirred at 80° C. for 18 h then was allowed to cool to room temperature. The reaction mixture was diluted with H₂O (35 mL) and extracted with EtOAc (3×35 mL). The combined organic layers were washed with brine (35 mL), dried over Na₂SO₄, filtered and concentrated under reduced pressure. The residue was purified by column chromatography (SiO₂, petroleum ether:ethyl acetate=1:0 to 10:1) to give the title compound as a yellow oil, Y=43%. ¹H NMR (400 MHz, DMSO-d₆) δ 7.25-7.19 (m, 2H), 5.34 (s, 2H), 3.67 (q, J=7 Hz, 2H), 1.31 (s, 12H), 1.12 (t, J=7 Hz, 3H).

### Step 4. 3-Fluoro-2-(4,4,5,5-tetramethyl-1,3,2-dioxaborolan-2-yl)-5-(trifluoromethyl)phenol

A mixture of 2-[2-(ethoxymethoxy)-6-fluoro-4-(trifluoromethyl)phenyl]-4,4,5,5-tetramethyl-1,3,2-dioxaborolane (140 mg, 384 mol) and 4 M HCl in EtOAc (5 mL) was stirred at 25° C. for 2 h. The reaction mixture was concentrated under reduced pressure to give the title compound as a yellow oil, Y=99%.

### Step 5. 3-Fluoro-2-(6-(((tetrahydro-2H-pyran-4-yl)amino)methyl)pyridazin-3-yl)-5-(trifluoromethyl)phenol

To a solution of N-[(6-chloropyridazin-3-yl)methyl]tetrahydropyran-4-amine (Intermediate A1, 85 mg, 373 mol) in dioxane (5 mL) and H₂O (1.7 mL) was added Na₂CO₃ (119 mg, 1.12 mmol), Pd(PPh₃)₄ (21.6 mg, 18.7 mol) and 3-fluoro-2-(4,4,5,5-tetramethyl-1,3,2-dioxaborolan-2-yl)-5-(trifluoromethyl)phenol (114 mg, 373 mol) at 25° C. under N₂. The mixture was stirred at reflux temperature for 8 h under N₂ then was allowed to cool to room temperature. The reaction mixture was diluted with H₂O (5 mL) and extracted with EtOAc (3×5 mL). The combined organic layers were washed with brine (5 mL), dried over anhydrous Na₂SO₄, filtered and concentrated under reduced pressure. The residue was purified by prep-HPLC (column: Phenomenex Luna C18 100×40 mm, 5 μm; mobile phase: [(A) H₂O (0.04% HCl)—(B) ACN]; gradient: 10-40% B, 8 min) and lyophilised to give the title compound as an off-white solid, Y=10%. ¹H NMR (400 MHz, DMSO-d₆) δ 11.36 (s, 1H), 9.60 (s, 2H), 8.02 (d, J=9 Hz, 1H), 7.97 (d, J=9 Hz, 1H), 7.29 (d, J=10 Hz, 1H), 7.22 (s, 1H), 4.65 (t, J=6 Hz, 2H), 3.98-3.94 (m, 2H), 3.53-3.42 (m, 1H), 3.35 (t, J=12 Hz, 2H), 2.10-2.00 (m, 2H), 1.80-1.60 (m, 2H). LCMS (ESI): m/z: [M+H]⁺=372.0.

### Compound 43. (R)-5-(((6-(2-Hydroxy-6-methyl-4-(trifluoromethyl)phenyl)pyridazin-3-yl)methyl)amino)-1-methylpiperidin-2-one

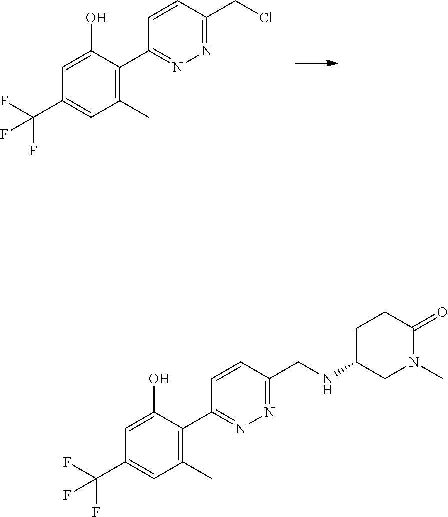 [SMILES: Cc1cc(C(F)(F)F)cc(O)c1-c1ccc(CCl)nn1 ; Cc1cc(C(F)(F)F)cc(O)c1-c1ccc(CN[C@@H]2CCC(=O)N(C)C2)nn1]

To a solution of 2-[6-(chloromethyl)pyridazin-3-yl]-3-methyl-5-(trifluoromethyl)phenol (Intermediate C1, 70 mg, 231 mol) in DMSO (2 mL) were added DIPEA (80.6 μL, 463 mol) and (5R)-5-amino-1-methyl-piperidin2-one (38.1 mg, 231 mol) at 25° C. The mixture was stirred at 80° C. for 1 h. The reaction mixture was purified by prep-HPLC (column: Waters Xbridge C18 150×50 mm, 10 μm; mobile phase: [(A) H₂O (10 mM NH₄HCO₃)—(B) ACN]; gradient: 15-45% B, 8 min) and lyophilised to give the title compound as a white solid, Y=33%. ¹H NMR (400 MHz, DMSO-d₆) δ 10.05 (br. s, 1H), 7.80 (d, J=9 Hz, 1H), 7.67 (d, J=9 Hz, 1H), 7.16 (s, 1H), 7.10 (s, 1H), 4.21-3.98 (m, 2H), 3.50-3.40 (m, 1H), 3.15-3.03 (m, 1H), 3.05-2.90 (m, 1H), 2.77 (s, 3H), 2.75-2.65 (m, 1H), 2.39-2.29 (m, 1H), 2.23-2.13 (m, 1H), 2.08 (s, 3H), 2.01-1.88 (m, 1H), 1.75-1.61 (m, 1H). LCMS (ESI): m/z: [M+H]⁺=395.0.

### Compound 44. 3-((6-(2-Hydroxy-6-methyl-4-(trifluoromethyl)phenyl)pyridazin-3-yl)methyl)oxazolidin-2-one

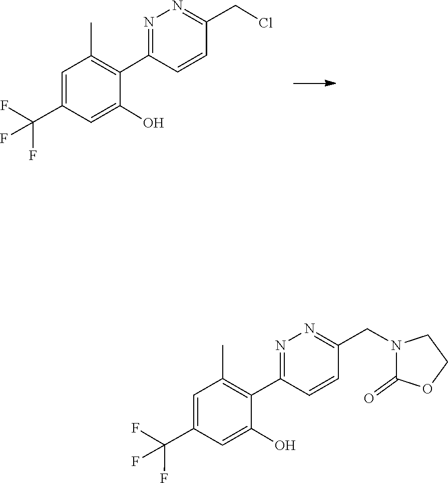 [SMILES: Cc1cc(C(F)(F)F)cc(O)c1-c1ccc(CCl)nn1 ; Cc1cc(C(F)(F)F)cc(O)c1-c1ccc(CN2CCOC2=O)nn1]

To a solution of oxazolidin-2-one (86.3 mg, 991 mol) in DMF (3 mL) was added NaH (39.6 mg, 991 mol, 60% wt in mineral oil) at 0° C. under N₂. The mixture was stirred at 0° C. for 30 min then a solution of 2-[6-(chloromethyl)pyridazin-3-yl]-3-methyl-5-(trifluoromethyl)phenol (Intermediate C1, 150 mg, 496 mol) in DMF (1.5 mL) was added. The mixture was stirred at 25° C. for 1 h under N₂ then was quenched by addition of saturated NH₄Cl aqueous solution (1 mL) at 0° C. and extracted with EtOAc (3×1 mL). The combined organic layers were washed with brine (1 mL), dried over Na₂SO₄, filtered and concentrated under reduced pressure. The residue was purified by prep-HPLC (column: Waters Xbridge Prep OBD C18 150×40 mm, 10 μm; mobile phase: [(A) H₂O (10 mM NH₄HCO₃)—(B) ACN]; gradient: 20 60% B, 8 min) and lyophilised to give the title compound as a white solid, Y=99%. ¹H NMR (400 MHz, DMSO-d₆) δ 10.28 (s, 1H), 7.72 (s, 2H), 7.17 (s, 1H), 7.11 (s, 1H), 4.73 (s, 2H), 4.36 (dd, J=9, 7 Hz, 2H), 3.67 (dd, J=9, 7 Hz, 2H), 2.09 (s, 3H). LCMS (ESI): m/z: [M+H]⁺=354.1.

### Compound 45. 3-Methyl-2-(6-(((1-methyl piperidin-4-yl)oxy)methyl)pyridazin-3-yl)-5-(trifluoromethyl)phenol hydrochloride

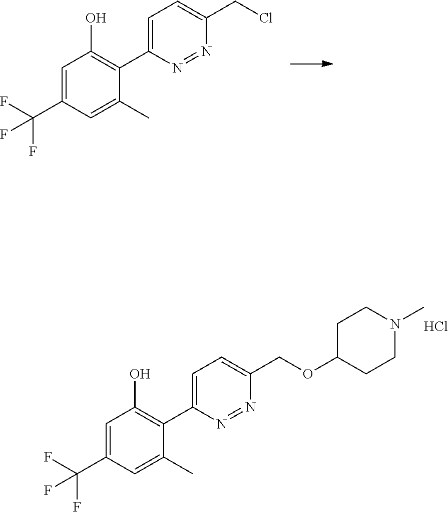 [SMILES: Cc1cc(C(F)(F)F)cc(O)c1-c1ccc(CCl)nn1 ; Cc1cc(C(F)(F)F)cc(O)c1-c1ccc(COC2CCN(C)CC2)nn1 ; Cl]

To a solution of 1-methylpiperidin-4-ol (60.9 μL, 521 mol) in DMF (2 mL) was added NaH (5.51 μL, 1.04 mmol, 60% wt in mineral oil) at 0° C. under N₂. The mixture was stirred at 0° C. for 1 h then 2-[6-(chloromethyl)pyridazin-3-yl]-3-methyl-5-(trifluoromethyl)phenol (Intermediate C1, 173 mg, 573 mol) at 0° C. under N₂ was added. The mixture was stirred at 25° C. for 12 h under N₂ then was quenched by addition of aqueous NH₄Cl (1 mL) at 0° C., and extracted with EtOAc (3×2 mL). The combined organic layers were washed with brine (2×2 mL), dried over Na₂SO₄, filtered and concentrated under reduced pressure. The residue was purified by prep-HPLC (column: Phenomenex Luna C18 75×30 mm, 3 μm; mobile phase: [(A) water (0.04% HCl)—(B) ACN]; gradient: 20-50% B, 8 min) and lyophilised to give the title compound as a white solid, Y=96%. ¹H NMR (400 MHz, DMSO-d₆) δ 11.04-10.41 (m, 2H), 8.13-7.81 (m, 2H), 7.26 (s, 1H), 7.17 (s, 1H), 4.91 (d, J=7 Hz, 2H), 3.99-3.67 (m, 1H), 3.48-3.41 (m, 1H), 3.29-3.09 (m, 2H), 3.05-2.92 (m, 1H), 2.91-2.62 (m, 3H), 2.27-2.19 (m, 1H), 2.16-1.98 (m, 4H), 2.08-1.97 (m, 1H), 1.92-1.74 (m, 1H). LCMS (ESI): m/z: [M+H]⁺=382.2.

### Compound 46. 1-((6-(2-Hydroxy-6-methyl-4-(trifluoromethyl)phenyl)pyridazin-3-yl)methyl)-4-methylpyrrolidin-2-one

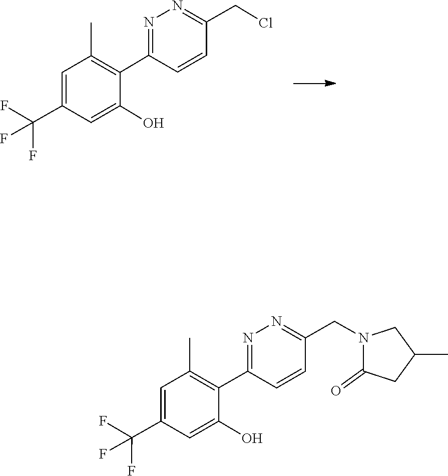 [SMILES: Cc1cc(C(F)(F)F)cc(O)c1-c1ccc(CCl)nn1 ; Cc1cc(C(F)(F)F)cc(O)c1-c1ccc(CN2CC(C)CC2=O)nn1]

To a solution of 4-methylpyrrolidin-2-one (131 mg, 1.32 mmol) in DMF (1 mL) was added LiHMDS (1 M in THF, 1.32 mL, 1.32 mmol) and 2-[6-(chloromethyl)pyridazin-3-yl]-3-methyl-5-(trifluoromethyl)phenol (Intermediate C1, 80 mg, 264 mol) at 0° C. under N₂. The mixture was stirred at 25° C. for 3 h then was quenched by addition of saturated aqueous NH₄Cl (1 mL) at 0° C. and extracted with EtOAc (3×1 mL). The combined organic layers were washed with brine (1 mL), dried over Na₂SO₄, filtered and concentrated under reduced pressure. The residue was purified by prep-HPLC (column: Waters Xbridge C18 150×50 mm, 10 μm; mobile phase: [(A) H₂O (10 mM NH₄HCO₃)—(B) ACN]; gradient: 30-50% B, 8 min) and lyophilised to give the title compound as a white solid, Y=98. ¹H NMR (400 MHz, DMSO-d₆) δ 10.26 (br. s, 1H), 7.70 (d, J=9 Hz, 1H), 7.61 (d, J=9 Hz, 1H), 7.16 (s, 1H), 7.10 (s, 1H), 4.72 (s, 2H), 3.60-3.50 (m, 1H), 3.05-2.95 (m, 1H), 2.58-2.52 (m, 1H), 2.47-2.38 (m, 1H), 2.13-1.96 (m, 4H), 1.06 (d, J=7 Hz, 3H). LCMS (ESI): m/z: [M+H]⁺=366.1.

### Compound 47. 1-((6-(2-Hydroxy-6-methyl-4-(trifluoromethyl)phenyl)pyridazin-3-yl)methyl)-5,5-dimethylpyrrolidin-2-one

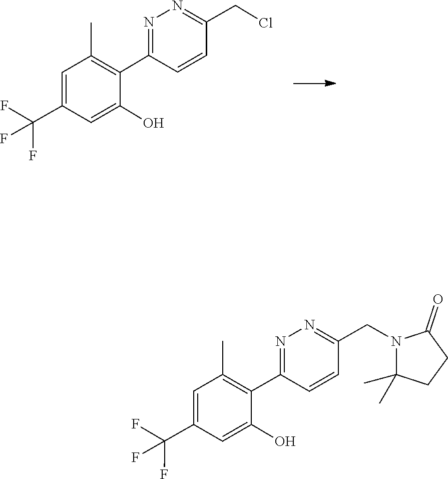 [SMILES: Cc1cc(C(F)(F)F)cc(O)c1-c1ccc(CCl)nn1 ; Cc1cc(C(F)(F)F)cc(O)c1-c1ccc(CN2C(=O)CCC2(C)C)nn1]

To a solution of 5,5-dimethylpyrrolidin-2-one (280 mg, 2.48 mmol) in DMF (2.5 mL) was added LiHMDS (1 M in THF, 2.48 mL, 2.48 mmol) at 0° C. under N₂. The mixture was stirred at 0° C. for 30 min under N₂. The mixture was then treated with 2-[6-(chloromethyl)pyridazin-3-yl]-3-methyl-5-(trifluoromethyl)phenol (Intermediate C1, 150 mg, 496 mol) in DMF (2.5 mL) under N₂. The mixture was stirred at 25° C. for 8 h then was quenched by addition saturated NH₄Cl aqueous solution (3 mL) at 0° C., and extracted with EtOAc (3×3 mL). The combined organic layers were washed with brine (3 mL), dried over anhydrous Na₂SO₄, filtered and concentrated under reduced pressure. The residue was purified by prep-HPLC (column: Phenomenex Luna C18 100×40 mm, 5 μm; mobile phase: [(A) H₂O (0.04% HCl)—(B) ACN]; gradient: 10-50% B, 8 min) and lyophilised to give the title compound as a yellow solid, Y=4%. ¹H NMR (400 MHz, DMSO-d₆) δ 10.28 (s, 1H), 7.67 (d, J=9 Hz, 1H), 7.61 (d, J=9 Hz, 1H), 7.16 (s, 1H), 7.10 (s, 1H), 4.67 (s, 2H), 2.40 (t, J=8 Hz, 2H), 2.07 (s, 3H), 1.91 (t, J=8 Hz, 2H), 1.17 (s, 6H). ¹H NMR (400 MHz, DMSO-d₆+D₂O) δ 7.64 (d, J=9 Hz, 1H), 7.59 (d, J=9 Hz, 1H), 7.15 (s, 1H), 7.09 (s, 1H), 4.65 (s, 2H), 2.39 (t, J=8 Hz, 2H), 2.04 (s, 3H), 1.90 (t, J=8 Hz, 2H), 1.15 (s, 6H). LCMS (ESI): m/z: [M+H]⁺=380.1.

### Compound 48. 5-Cyclopropyl-3-methyl-2-(6-(((tetrahydro-2H-pyran-4-yl)amino)methyl)pyridazin-3-yl)phenol

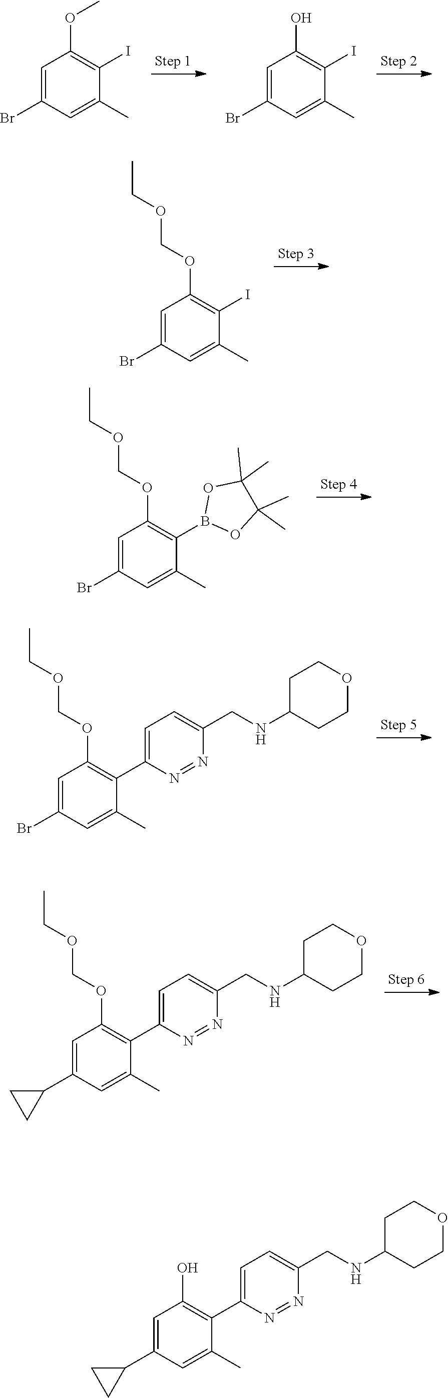

### Step 1. 5-Bromo-2-iodo-3-methylphenol

To a solution of 5-bromo-2-iodo-1-methoxy-3-methyl-benzene (23.1 g, 70.7 mmol) in DCM (300 mL) was added BBr₃ (13.6 mL, 141 mmol) at 0° C. under N₂. The mixture was stirred at 0° C. for 1 h then was quenched by addition of H₂O (500 mL), and extracted with DCM (3×400 mL). The combined organic layers were washed with brine (200 mL), dried over Na₂SO₄, filtered and concentrated under reduced pressure to give as a yellow oil, Y=99%. ¹H NMR (400 MHz, CDCl₃) δ 7.06-6.93 (m, 2H), 2.42 (s, 3H).

### Step 2. 5-Bromo-1-(ethoxymethoxy)-2-iodo-3-methylbenzene

To a solution of 5-bromo-2-iodo-3-methyl-phenol (22.8 g, 72.9 mmol) in ACN (300 mL) were added Cs₂CO₃ (23.7 g, 72.9 mmol) and chloromethoxyethane (6.76 mL, 72.9 mmol) at 25° C. The mixture was stirred at 25° C. for 2 h under N₂. The reaction mixture was filtered and concentrated under reduced pressure. The residue was purified by column chromatography (SiO₂, Petroleum ether:Ethyl acetate=10:1 to 5:1) to give the title compound as a white solid, Y=100%.

### Step 3. 2-(4-Bromo-2-(ethoxymethoxy)-6-methylphenyl)-4,4,5,5-tetramethyl-1,3,2-dioxaborolane

To a solution of 5-bromo-1-(ethoxymethoxy)-2-iodo-3-methyl-benzene (22.0 g, 59.3 mmol) in dioxane (360 mL) were added 4,4,5,5-tetramethyl-1,3,2-dioxaborolane (9.46 mL, 65.2 mmol), TEA (57.8 mL, 415 mmol), dicyclohexyl-(2-phenylphenyl)phosphane (5.20 g, 14.8 mmol) and Pd(OAc)₂ (666 mg, 2.96 mmol) at 25° C. under N₂. The mixture was stirred at 80° C. for 12 h then was allowed to cool to room temperature and was concentrated under reduced pressure. The residue was diluted with H₂O (200 mL) and extracted with EtOAc (3×200 mL). The combined organic layers were washed with brine (200 mL), dried over Na₂SO₄, filtered and concentrated under reduced pressure to give a residue. The residue was purified by prep-TLC (SiO₂, Petroleum ether:Ethyl acetate=10:1) to give the title compound as a white solid, Y=59%. ¹H NMR (400 MHz, CDCl₃) δ 7.02 (s, 1H), 6.97 (s, 1H), 5.16 (s, 2H), 3.72 (q, J=7 Hz, 2H), 2.32 (s, 3H), 1.38 (s, 12H), 1.22 (t, J=7 Hz, 3H).

### Step 4. N-((6-(4-Bromo-2-(ethoxymethoxy)-6-methylphenyl)pyridazin-3-yl)methyl)tetrahydro-2H-pyran-4-amine

To a solution of 2-[4-bromo-2-(ethoxymethoxy)-6-methyl-phenyl]-4,4,5,5-tetramethyl-1,3,2-dioxaborolane (3.30 g, 8.89 mmol) in H₂O (33 mL) and dioxane (165 mL) was added N-[(6-chloropyridazin-3-yl)methyl]tetrahydropyran-4-amine (Intermediate A1, 2.43 g, 10.7 mmol), Na₂CO₃ (2.83 g, 26.7 mmol) and Pd(PPh₃)₄ (206 mg, 178 mol) at 25° C. under N₂. The mixture was stirred at reflux temperature for 2 h then was allowed to cool to room temperature and concentrated under reduced pressure. The residue was diluted with H₂O (40 mL) and extracted with EtOAc (3×30 mL). The combined organic layers were washed with brine (2×20 mL), dried over Na₂SO₄, filtered and concentrated under reduced pressure. The residue was purified by prep-HPLC (column: Agela DuraShell C18 250×70 mm, 10 μm; mobile phase: [(A) H₂O (10 mM NH₄HCO₃)—(B) ACN]; gradient: 30-53% B, 17 min) and lyophilised to give the title compound as a yellow solid, Y=4%. ¹H NMR (400 MHz, DMSO-d₆) δ 7.80 (d, J=9 Hz, 1H), 7.66-7.61 (m, 1H), 7.30 (s, 1H), 7.25 (s, 1H), 5.16 (s, 2H), 4.07 (s, 2H), 3.87-3.76 (m, 2H), 3.44 (q, J=7 Hz, 2H), 3.31-3.16 (m, 2H), 2.69-2.59 (m, 1H), 2.00 (s, 3H), 1.87-1.74 (m, 2H), 1.36-1.25 (m, 2H), 1.03 (t, J=7 Hz, 3H).

### Step 5. N-((6-(4-Cyclopropyl-2-(ethoxymethoxy)-6-methylphenyl)pyridazin-3-yl)methyl)tetrahydro-2H-pyran-4-amine

To a solution of N-[[6-[4-bromo-2-(ethoxymethoxy)-6-methyl-phenyl]pyridazin-3-yl]methyl]tetrahydropyran-4-amine (150 mg, 344 mol) in toluene (3 mL) and H₂O (0.6 mL) were added cyclopropylboronic acid (88.6 mg, 1.03 mmol), K₃PO₄ (146 mg, 688 mol), tricyclohexylphosphane (16.7 μL, 51.6 mol) and Pd(OAc)₂ (772 g, 3.44 mol) at 25° C. under N₂. The mixture was stirred at reflux temperature for 12 h under N₂ then was allowed to cool to room temperature and concentrated under reduced pressure. The residue was purified by prep-TLC (SiO₂, petroleum ether:ethyl acetate=10:1) to give the title compound as a white solid, Y=48%.

### Step 6. 5-Cyclopropyl-3-methyl-2-(6-(((tetrahydro-2H-pyran-4-yl)amino)methyl)pyridazin-3-yl)phenol

A mixture of N-[[6-[4-cyclopropyl-2-(ethoxymethoxy)-6-methyl-phenyl]pyridazin-3-yl]methyl]tetrahydropyran-4-amine (40 mg, 101 mol) and 4 M HCl in EtOAc (0.3 mL) was stirred at 25° C. for 2 h. The reaction mixture was concentrated under reduced pressure and the residue was purified by prep-HPLC (column: Waters Xbridge BEH C18 100×30 mm, 10 μm; mobile phase: [(A) H₂O (10 mM NH₄HCO₃)—(B) ACN]; gradient: 15-55% B, 8 min) and lyophilised to give the title compound as a white solid, Y=54%. ¹H NMR (400 MHz, DMSO-d₆) δ 9.42 (s, 1H), 7.73 (d, J=9 Hz, 1H), 7.56 (d, J=9 Hz, 1H), 6.57-6.43 (m, 2H), 4.06 (s, 2H), 3.87-3.83 (m, 2H), 3.30-3.26 (m, 2H), 2.71-2.66 (m, 1H), 2.00 (s, 3H), 1.85-1.82 (m, 3H), 1.38-1.26 (m, 2H), 0.96-0.93 (m, 2H), 0.70-0.55 (m, 2H). LCMS (ESI): m/z: [M+H]⁺=340.3.

### Compound 49. (S)-4-(((6-(2-Hydroxy-6-methyl-4-(trifluoromethyl)phenyl)pyridazin-3-yl)methyl)amino)-1-methylpyrrolidin-2-one

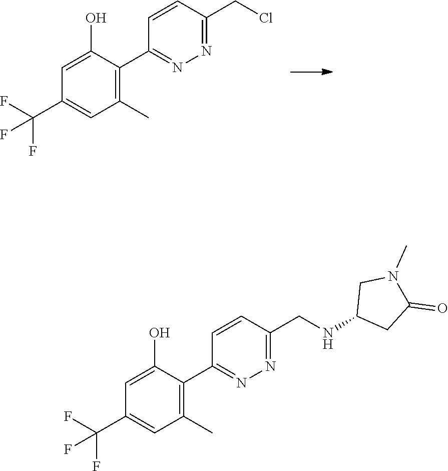 [SMILES: Cc1cc(C(F)(F)F)cc(O)c1-c1ccc(CCl)nn1 ; Cc1cc(C(F)(F)F)cc(O)c1-c1ccc(CN[C@H]2CC(=O)N(C)C2)nn1]

To a solution of 2-[6-(chloromethyl)pyridazin-3-yl]-3-methyl-5-(trifluoromethyl)phenol (Intermediate C1, 70 mg, 231 mol) in DMSO (1 mL) was added DIPEA (80.6 μL, 463 mol) and (4S)-4-amino-1-methyl-pyrrolidin2-one (26.4 mg, 231 mol) at 25° C. The mixture was stirred at 80° C. for 1 h then was allowed to cool to room temperature. The reaction mixture was purified directly by prep-HPLC (column: Waters Xbridge Prep OBD C18 150×40 mm, 10 μm; mobile phase: [(A) H₂O (10 mM NH₄HCO₃)—(B) ACN]; gradient: 30-60% B, 8 min) and lyophilised to give the title compound as a white solid, Y=46%. ¹H NMR (400 MHz, DMSO-d₆) δ 10.16 (br. s, 1H), 7.78 (d, J=9 Hz, 1H), 7.67 (d, J=9 Hz, 1H), 7.16 (s, 1H), 7.09 (s, 1H), 4.03 (d, J=7 Hz, 2H), 3.60-3.51 (m, 1H), 3.45-3.35 (m, 1H), 3.20-3.10 (m, 1H), 2.88-2.83 (m, 1H), 2.71 (s, 3H), 2.51-2.43 (m, 1H), 2.16-2.10 (m, 1H), 2.08 (s, 3H). LCMS (ESI): m/z: [M+H]⁺=381.3.

### Compound 50. (R)-4-(((6-(2-Hydroxy-6-methyl-4-(trifluoromethyl)phenyl)pyridazin-3-yl)methyl)amino)-1-methylpyrrolidin-2-one

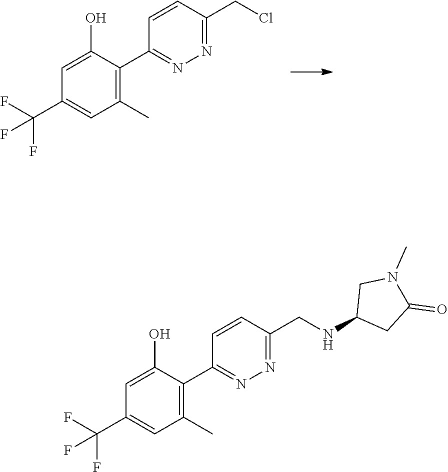 [SMILES: Cc1cc(C(F)(F)F)cc(O)c1-c1ccc(CCl)nn1 ; Cc1cc(C(F)(F)F)cc(O)c1-c1ccc(CN[C@@H]2CC(=O)N(C)C2)nn1]

To a solution of 2-[6-(chloromethyl)pyridazin-3-yl]-3-methyl-5-(trifluoromethyl)phenol (Intermediate C1, 60 mg, 198 mol) in DMSO (1 mL) were added (4R)-4-amino-1-methyl-pyrrolidin-2-one (33.9 mg, 297 mol) and DIPEA (69.1 μL, 396 mol) at 25° C. The mixture was stirred at 80° C. for 2 h then concentrated under room temperature. The reaction mixture was concentrated under reduced pressure to give a residue. The residue was purified by prep-HPLC (column: Waters Xbridge C18 150×50 mm, 10 μm; mobile phase: [(A) H₂O (10 mM NH₄HCO₃)—(B) ACN]; gradient: 10-45% B, 8 min) and lyophilised to give the title compound as a white solid, Y=40%. ¹H NMR (400 MHz, DMSO-d₆) δ 10.23 (br. s, 1H), 7.78 (d, J=9 Hz, 1H), 7.67 (d, J=9 Hz, 1H), 7.16 (s, 1H), 7.09 (s, 1H), 4.03 (s, 2H), 3.56-3.49 (m, 1H), 3.48-3.39 (m, 1H), 3.18-3.11 (m, 1H), 2.95-2.81 (m, 1H), 2.71 (s, 3H), 2.49-2.44 (m, 1H), 2.16-2.09 (m, 1H), 2.08 (s, 3H). LCMS (ESI): m/z: [M+H]⁺=381.3.

### Compound 51. 2-(6-(((3,5-Dimethylisoxazol-4-yl)amino)methyl)pyridazin-3-yl)-3-methyl-5-(trifluoromethyl)phenol

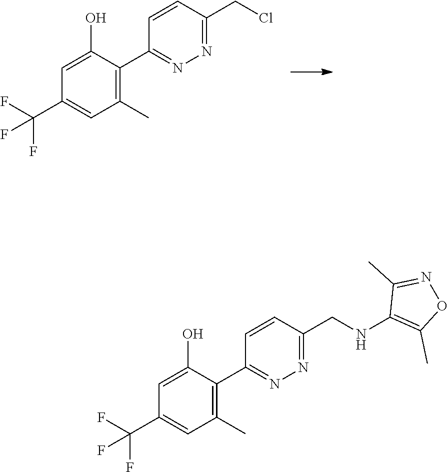 [SMILES: Cc1cc(C(F)(F)F)cc(O)c1-c1ccc(CCl)nn1 ; Cc1cc(C(F)(F)F)cc(O)c1-c1ccc(CNc2c(C)noc2C)nn1]

To a solution of 2-[6-(chloromethyl)pyridazin-3-yl]-3-methyl-5-(trifluoromethyl)phenol (Intermediate C1, 80 mg, 264 mol) in DMSO (1 mL) was added DIPEA (92.1 μL, 529 mol) and 3,5-dimethylisoxazol-4-amine (29.6 mg, 264 mol) at 25° C. The mixture was stirred at 120° C. for 2 h then allowed to cool to room temperature. The reaction mixture was purified by prep-HPLC (column: Waters Xbridge Prep OBD C18 150×40 mm, 10 μm; mobile phase: [(A) H₂O (10 mM NH₄HCO₃)—(B) ACN]; gradient: 30-60% B over 8.0 min) and lyophilised to give the title compound as a white solid, Y=28%. ¹H NMR (400 MHz, DMSO-d₆) δ 10.27 (br. s, 1H), 7.76 (d, J=9 Hz, 1H), 7.69 (d, J=9 Hz, 1H), 7.16 (s, 1H), 7.09 (s, 1H), 4.77 (t, J=7 Hz, 1H), 4.37 (d, J=7 Hz, 2H), 2.14 (s, 3H), 2.12 (s, 3H), 2.06 (s, 3H). LCMS (ESI): m/z: [M+H]⁺=379.2.

### Compound 52. 5-Fluoro-3-methyl-2-(6-(((tetrahydro-2H-pyran-4-yl)amino)methyl)pyridazin-3-yl)phenol

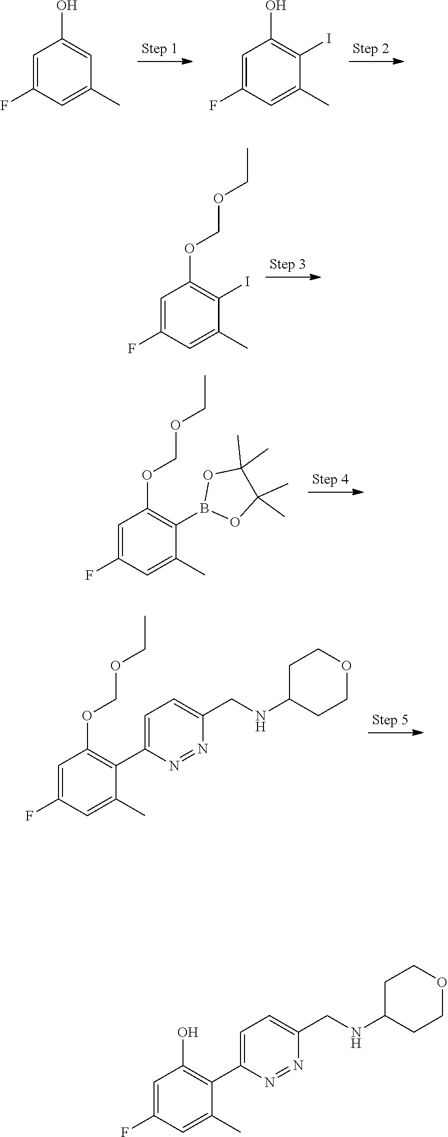

### Step 1. 5-Fluoro-2-iodo-3-methylphenol

To a solution of 3-fluoro-5-methyl-phenol (5.00 g, 39.6 mmol) in toluene (100 mL) was added NaH (2.38 g, 59.5 mmol, 60% wt in mineral oil) at 0° C. under N₂. The mixture was stirred at 0° C. for 0.5 h then I₂ (7.99 mL, 39.6 mmol) was added. The mixture was stirred at 0° C. for 2 h then was quenched by addition of saturated aqueous NH₄Cl solution (25 mL) at 0° C. and extracted with EtOAc (3×50 mL). The combined organic layers were washed with brine (5 mL), dried over Na₂SO₄, filtered and concentrated under reduced pressure. The residue was purified by column chromatography (SiO₂, petroleum ether:ethyl acetate=100:0 to 99:1) to give the title compound as a white solid, Y=10%. ¹H NMR (400 MHz, DMSO-d₆) δ 10.72 (s, 1H), 6.71 (dd, J=10, 3 Hz, 1H), 6.52 (dd, J=10, 3 Hz, 1H), 2.35 (s, 3H).

### Step 2. 1-(Ethoxymethoxy)-5-fluoro-2-iodo-3-methylbenzene

To a solution of 5-fluoro-2-iodo-3-methyl-phenol (2.50 g, 9.92 mmol) in ACN (25 mL) was added Cs₂CO₃ (6.46 g, 19.8 mmol) and chloromethoxyethane (920 μL, 9.92 mmol,) at 25° C. The mixture was stirred at 25° C. for 2 h then concentrated under reduced pressure. The residue was diluted with H₂O (25 mL) and extracted with EtOAc (3×25 mL). The combined organic layers were washed with brine (3×25 mL), dried over Na₂SO₄, filtered and concentrated under reduced pressure. The residue was purified by prep-TLC (SiO₂, petroleum ether:ethyl acetate=100:1 to 99:1) to give the title compound as a white oil, Y=81%. ¹H NMR (400 MHz, DMSO-d₆) δ 6.90 (dd, J=10, 3 Hz, 1H), 6.84 (dd, J=10, 3 Hz, 1H), 5.32 (s, 2H), 3.68 (q, J=7 Hz, 2H), 2.39 (s, 3H), 1.13 (t, J=7 Hz, 3H).

### Step 3. 2-(2-(Ethoxymethoxy)-4-fluoro-6-methylphenyl)-4,4,5,5-tetramethyl-1,3,2-dioxaborolane

To a solution of 1-(ethoxymethoxy)-5-fluoro-2-iodo-3-methyl-benzene (2.50 g, 8.06 mmol) in dioxane (28 mL) were added TEA (8.30 mL, 59.7 mmol), Pd(OAc)₂ (235 mg, 1.05 mmol), dicyclohexyl-(2-phenylphenyl)phosphane (678 mg, 1.93 mmol) and 4,4,5,5-tetramethyl-1,3,2-dioxaborolane (3.51 mL, 24.2 mmol) at 25° C. under N₂. The mixture was stirred at 80° C. for 18 h then allowed to cool to room temperature. The mixture was diluted with H₂O (30 mL) and extracted with EtOAc (3×30 mL). The residue was purified by prep-TLC (SiO₂, petroleum ether:ethyl acetate=1:1) to give the title compound as an oil, Y=28%. ¹H NMR (400 MHz, DMSO-d₆) δ 6.69 (dd, J=10, 3 Hz, 1H), 6.63 (dd, J=10, 3 Hz, 1H), 5.18 (s, 2H), 3.64 (q, J=7 Hz, 2H), 2.25 (s, 3H), 1.29 (s, 12H), 1.11 (t, J=7 Hz, 3H).

### Step 4. N-((6-(2-(Ethoxymethoxy)-4-fluoro-6-methylphenyl)pyridazin-3-yl)methyl)tetrahydro-2H-pyran-4-amine

To a solution of 2-[2-(ethoxymethoxy)-4-fluoro-6-methyl-phenyl]-4,4,5,5-tetramethyl-1,3,2-dioxaborolane (429 mg, 1.38 mmol) in dioxane (4 mL) and H₂O (1 mL) were added Pd(dppf)Cl₂ (33.7 mg, 46.1 mol), Na₂CO₃ (293 mg, 2.77 mmol) and N-[(6-chloropyridazin-3-yl)methyl]tetrahydropyran-4-amine (Intermediate A1, 210 mg, 922 mol) at 25° C. under N₂. The mixture was stirred at reflux temperature for 12 h then allowed to cool to room temperature. The reaction mixture was diluted with H₂O (5 mL) and extracted with EtOAc (3×5 mL). The combined organic layers were washed with brine (5 mL), dried over Na₂SO₄, filtered and concentrated under reduced pressure. The residue was purified by prep-HPLC (column: Phenomenex Luna C18 100×40 mm, 5 μm; mobile phase: [(A) H₂O (0.04% HCl)—(B) ACN]; gradient: 15-30% B, 8 min) and lyophilised to give the title compound as a white solid, Y=87%.

### Step 5. 5-Fluoro-3-methyl-2-(6-(((tetrahydro-2H-pyran-4-yl)amino)methyl)pyridazin-3-yl)phenol

A mixture of N-[[6-[2-(ethoxymethoxy)-4-fluoro-6-methyl-phenyl]pyridazin-3-yl]methyl]tetrahydropyran-4-amine (300 mg, 799 mol) and 4 M HCl in EtOAc (10 mL) was stirred at 25° C. for 1 h then was concentrated under reduced pressure. The residue was purified by prep-HPLC (column: Waters Xbridge BEH C18 100×30 mm, 10 μm; mobile phase: [(A) H₂O (10 mM NH₄HCO₃)—(B) ACN]; gradient: 5-45% B, 8 min) and lyophilised to give the title compound as a white solid, Y=18%. ¹H NMR (400 MHz, DMSO-d₆) δ 7.75 (d, J=9 Hz, 1H), 7.59 (d, J=9 Hz, 1H), 6.67-6.51 (m, 2H), 4.05 (s, 2H), 3.89-3.77 (m, 2H), 3.32-3.26 (m, 2H), 2.72-2.61 (m, 1H), 2.02 (s, 3H), 1.88-1.78 (m, 2H), 1.37-1.24 (m, 2H). LCMS (ESI): m/z: [M+H]⁺=318.2.

### Compound 53. (R)-5-Chloro-3-methyl-2-(6-(((tetrahydrofuran-3-yl)amino)methyl)pyridazin-3-yl)phenol

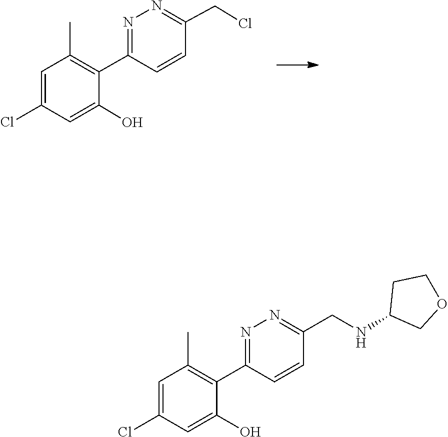 [SMILES: Cc1cc(Cl)cc(O)c1-c1ccc(CCl)nn1 ; Cc1cc(Cl)cc(O)c1-c1ccc(CN[C@@H]2CCOC2)nn1]

To a solution of 5-chloro-2-[6-(chloromethyl)pyridazin-3-yl]-3-methyl-phenol (Intermediate C2, 80 mg, 297 mol) in DMSO (1 mL) was added (3R)-tetrahydrofuran-3-amine (25.9 mg, 297 mol) and DIPEA (155 μL, 892 mol) at 25° C. under N₂. The mixture was stirred at 80° C. for 2 h then was allowed to cool to room temperature. The reaction mixture was purified directly by prep-HPLC (column: Waters Xbridge BEH C18 100×30 mm, 10 μm; mobile phase: [(A) H₂O (10 mM NH₄HCO₃)—(B) ACN]; gradient: 10-45% B, 8 min) and lyophilised to give the title compound as a white solid, Y=32%. ¹H NMR (400 MHz, DMSO-d₆) δ 10.05 (s, 1H), 7.76 (d, J 9 Hz, 1H), 7.61 (d, J 9 Hz, 1H), 6.88 (s, 1H), 6.85 (s, 1H), 4.07-3.93 (m, 2H), 3.83-3.65 (m, 3H), 3.51-3.45 (m, 1H), 3.38-3.34 (m, 1H), 2.71-2.60 (m, 1H), 2.01 (s, 3H), 1.99-1.92 (m, 1H), 1.77-1.68 (m, 1H). LC-MS (ESI): m/z: [M+H]⁺=320.2.

### Compound 54. 5-Chloro-3-methyl-2-(6-(pyrrolidin-1-ylmethyl)pyridazin-3-yl)phenol

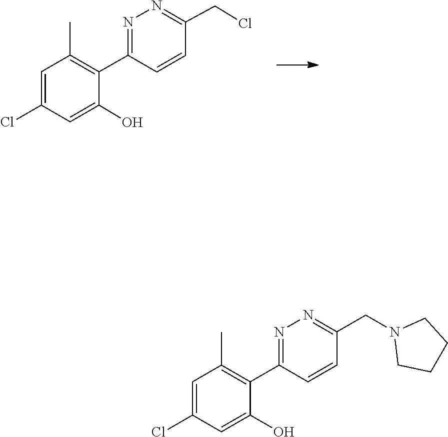 [SMILES: Cc1cc(Cl)cc(O)c1-c1ccc(CCl)nn1 ; Cc1cc(Cl)cc(O)c1-c1ccc(CN2CCCC2)nn1]

To a solution of 5-chloro-2-[6-(chloromethyl)pyridazin-3-yl]-3-methyl-phenol (Intermediate C2, 70 mg, 260 mol) in DMSO (2 mL) were added DIPEA (136 μL, 780 mol) and pyrrolidine (32.6 μL, 390 mol) at 25° C. under N₂. The mixture was stirred at 80° C. for 1 h. The reaction mixture was purified by prep-HPLC (column: Waters Xbridge BEH C18 100×30 mm, 10 μm; mobile phase: [(A) H₂O (10 mM NH₄HCO₃)—(B) ACN]; gradient: 25-55% B, 8 min) and lyophilised to give the title compound as a white solid, Y=39%. ¹H NMR (400 MHz, DMSO-d₆) δ 9.85 (br. s, 1H), 7.71 (d, J=9 Hz, 1H), 7.60 (d, J=9 Hz, 1H), 6.88 (s, 1H), 6.85 (s, 1H), 3.93 (s, 2H), 2.57-2.53 (m, 4H), 2.00 (s, 3H), 1.81-1.65 (m, 4H). LC-MS (ESI): m/z: [M+H]⁺=304.2.

### Compound 55. 3-Methyl-2-(6-(((3-methylisoxazol-4-yl)amino)methyl)pyridazin-3-yl)-5-(trifluoromethyl)phenol

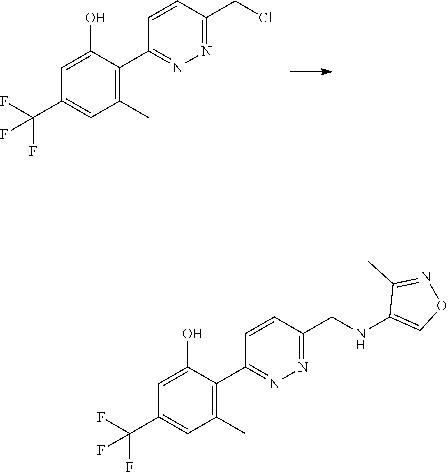 [SMILES: Cc1cc(C(F)(F)F)cc(O)c1-c1ccc(CCl)nn1 ; Cc1cc(C(F)(F)F)cc(O)c1-c1ccc(CNc2conc2C)nn1]

To a solution of 2-[6-(chloromethyl)pyridazin-3-yl]-3-methyl-5-(trifluoromethyl)phenol (Intermediate C1, 100 mg, 330 mol) in DMSO (6 mL) were added DIPEA (173 μL, 991 mol) and 3-methylisoxazol-4-amine (64.8 mg, 661 mol) at 25° C. under N₂. The mixture was stirred at 80° C. for 12 h then allowed to cool to room temperature. The reaction mixture was purified directly by prep-HPLC (column: Waters Xbridge BEH C18 100×30 mm, 10 μm; mobile phase: [(A) H₂O (10 mM NH₄HCO₃)—(B) ACN]; gradient: 15-50% B, 8 min) and lyophilised to give the title compound as a yellow solid, Y=19%. ¹H NMR (400 MHz, DMSO-d₆) δ 10.24 (br. s, 1H), 8.09 (s, 1H), 7.76 (d, J=9 Hz, 1H), 7.68 (d, J=9 Hz, 1H), 7.16 (s, 1H), 7.10 (s, 1H), 5.51 (t, J=6 Hz, 1H), 4.44 (d, J=6 Hz, 2H), 2.20 (s, 3H), 2.08 (s, 3H). LCMS (ESI): m/z: [M+H]⁺=365.1.

### Compound 56. 5-Chloro-2-(6-(((3,5-dimethylisoxazol-4-yl)amino)methyl)pyridazin-3-yl)-3-methylphenol

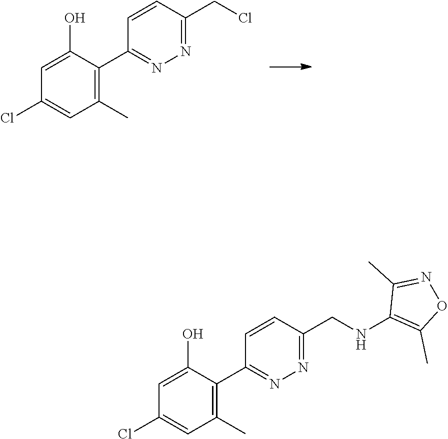 [SMILES: Cc1cc(Cl)cc(O)c1-c1ccc(CCl)nn1 ; Cc1cc(Cl)cc(O)c1-c1ccc(CNc2c(C)noc2C)nn1]

To a solution of 5-chloro-2-[6-(chloromethyl)pyridazin-3-yl]-3-methyl-phenol (Intermediate C2, 100 mg, 372 mol) in DMSO (1 mL) were added DIPEA (129 μL, 743 mol) and 3,5-dimethylisoxazol-4-amine (83.3 mg, 743 mol) at 25° C. The mixture was stirred at 120° C. for 2 h. The reaction mixture was purified by prep-HPLC (column: Waters Xbridge BEH C18 100×30 mm, 10 μm; mobile phase: [(A) H₂O (10 mM NH₄HCO₃)—(B) ACN]; gradient: 10-45% B, 8 min) and lyophilised to give the title compound as a yellow solid, Y=25%. ¹H NMR (400 MHz, DMSO-d₆) δ 7.72 (d, J=8 Hz, 1H), 7.62 (d, J=8 Hz, 1H), 6.92-6.76 (m, 2H), 4.76 (t, J=7 Hz, 1H), 4.35 (d, J=7 Hz, 2H), 2.14 (s, 3H), 2.11 (s, 3H), 1.99 (s, 3H). LCMS (ESI): m/z: [M+H]⁺=345.3.

### Compound 57. 3-Methyl-5-nitro-2-(6-(((tetrahydro-2H-pyran-4-yl)amino)methyl)pyridazin-3-yl)phenol hydrochloride

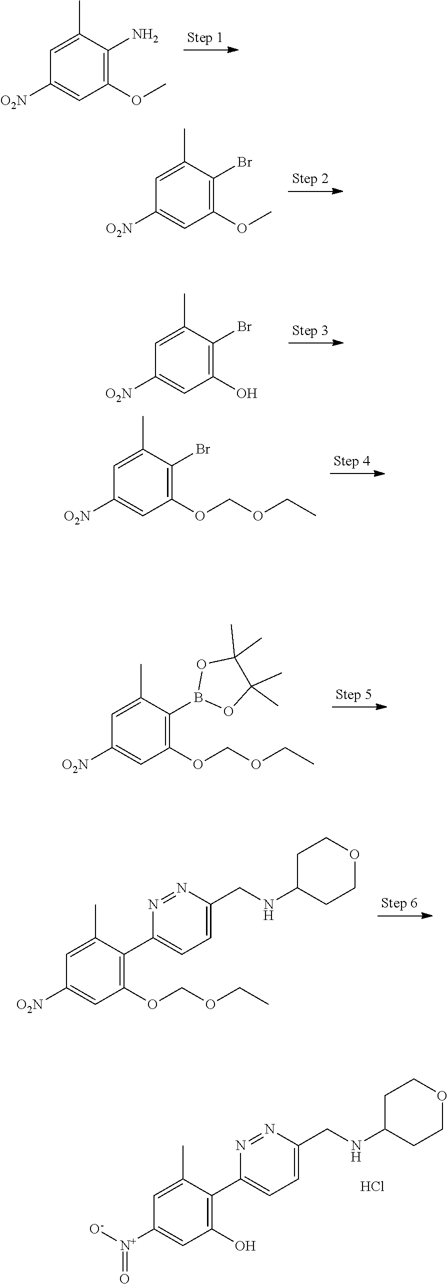

### Step 1. 2-Bromo-1-methoxy-3-methyl-5-nitrobenzene

To a solution of 2-methoxy-6-methyl-4-nitro-aniline (1.00 g, 5.49 mmol) in H₂SO₄ (4.88 mL, 27.5 mmol, 30% wt in H₂O) was added NaNO₂ (492 mg, 7.14 mmol) at 0° C., the mixture was stirred at 0° C. for 1 h. The RM was treated with CuBr (83.6 μL, 2.74 mmol) and aqueous HBr (1.19 mL, 6.59 mmol, 30% wt in H₂O) at 25° C., then the resulting solution stirred at 50° C. for 1 h. The reaction mixture was quenched by addition of H₂O (12 mL) at 0° C., and extracted with EtOAc (3×12 mL). The combined organic layers were washed with brine (3×10 mL), dried over Na₂SO₄, filtered and concentrated under reduced pressure. The residue was purified by column chromatography (SiO₂, petroleum ether:ethyl acetate=5:1 to 3:1) to give the title compound as a yellow solid, Y=81%. ¹H NMR (400 MHz, DMSO-d₆) δ 7.90 (d, J=2 Hz, 1H), 7.68 (d, J=2 Hz, 1H), 3.98 (s, 3H), 2.47 (s, 3H).

### Step 2. 2-Bromo-3-methyl-5-nitrophenol

A mixture of 2-bromo-1-methoxy-3-methyl-5-nitro-benzene (600 mg, 2.44 mmol) and conc. H₂SO₄ (1.30 mL, 24.4 mmol) was stirred at 100° C. for 1 h then allowed to cool to room temperature. The reaction mixture was poured into H₂O (20 mL) at 0° C., and extracted with EtOAc (3×10 mL). The combined organic layers were washed with brine (8 mL), dried over Na₂SO₄, filtered and concentrated under reduced pressure to give the title compound as a yellow solid, Y=95%. ¹H NMR (400 MHz, DMSO-d₆) δ 11.24 (s, 1H), 7.70 (d, J=2 Hz, 1H), 7.57 (d, J=2 Hz, 1H), 2.44 (s, 3H).

### Step 3. 2-Bromo-1-(ethoxymethoxy)-3-methyl-5-nitrobenzene

To a solution of 2-bromo-3-methyl-5-nitro-phenol (280 mg, 1.21 mmol) in DMF (4.5 mL) was added NaH (96.5 mg, 2.41 mmol, 60% wt. in mineral oil) at 0° C. under N₂. The mixture was stirred at 0° C. for 1 h then chloromethoxyethane (123 μL, 1.33 mmol) was added. The solution was stirred at 0° C. for 3 h then quenched by addition of saturated NH₄Cl solution (1 mL), diluted with H₂O (5 mL) and extracted with EtOAc (3×4 mL). The combined organic layers were washed with brine (4 mL), dried over Na₂SO₄, filtered and concentrated under reduced pressure. The residue was purified by column chromatography (SiO₂, petroleum ether:ethyl acetate=10:1 to 5:1) to give the title compound as a yellow oil, Y=91%. ¹H NMR (400 MHz, DMSO-d₆) δ 7.93 (d, J=2 Hz, 1H), 7.84 (d, J=2 Hz, 1H), 5.48 (s, 2H), 3.74 (q, J=7 Hz, 2H), 2.49 (s, 3H), 1.14 (t, J=7 Hz, 3H).

### Step 4. 2-(2-(Ethoxymethoxy)-6-methyl-4-nitrophenyl)-4,4,5,5-tetramethyl-1,3,2-dioxaborolane

To a solution of 2-bromo-1-(ethoxymethoxy)-3-methyl-5-nitro-benzene (100 mg, 345 mol) in dioxane (2.5 mL) were added 4,4,5,5-tetramethyl-2-(4,4,5,5-tetramethyl-1,3,2-dioxaborolan-2-yl)-1,3,2-dioxaborolane (131 mg, 517 mol), potassium acetate (101 mg, 1.03 mmol) and bis(triphenylphosphine)palladium(II) dichloride (12.1 mg, 17.2 mol) at 25° C. under N₂. The solution was stirred at reflux temperature for 12 h then allowed to cool to room temperature. The reaction mixture was diluted with H₂O (1 mL) and extracted with EtOAc (3×1 mL). The combined organic layers were washed with brine (1 mL), dried over Na₂SO₄, filtered and concentrated under reduced pressure. The residue was purified by prep-TLC (SiO₂, petroleum ether:ethyl acetate=10:1) to give the title compound as a white solid, Y=69%. ¹H NMR (400 MHz, DMSO-d₆) δ 7.69 (d, J=2 Hz, 1H), 7.63 (d, J=2 Hz, 1H), 5.32 (s, 2H), 3.68 (q, J=7 Hz, 2H), 2.37 (s, 3H), 1.33 (s, 12H), 1.12 (t, J=7 Hz, 3H).

### Step 5. N-((6-(2-(Ethoxymethoxy)-6-methyl-4-nitrophenyl)pyridazin-3-yl)methyl)tetrahydro-2H-pyran-4-amine

To a solution of 2-[2-(ethoxymethoxy)-6-methyl-4-nitro-phenyl]-4,4,5,5-tetramethyl-1,3,2-dioxaborolane (250 mg, 741 mol) in dioxane (10 mL) and H₂O (2.5 mL) were added N—[(6-chloropyridazin-3-yl)methyl]tetrahydropyran-4-amine (Intermediate A1, 338 mg, 1.48 mmol), (2-dicyclohexylphosphino-2′,6′-dimethoxybiphenyl)[2-(2′-amino-1,1′-biphenyl)]palladium(II) methanesulfonate (86.8 mg, 111 mol) and K₂CO₃ (410 mg, 2.97 mmol) at 25° C. under N₂. The reaction mixture was stirred at reflux temperature for 3 h under N₂ then allowed to cool to room temperature. The reaction mixture was diluted with H₂O (5 mL) and extracted with EtOAc (3×6 mL). The combined organic layers were washed with brine (2×5 mL), dried over Na₂SO₄, filtered and concentrated under reduced pressure. The residue was purified by prep-HPLC (column: Waters Xbridge BEH C18 250×50 mm, 10 μm; mobile phase: [(A) H₂O (10 mM NH₄HCO₃)—(B) ACN]; gradient: 20-50% B, 10 min) and lyophilised to give the title compound as a white solid, Y=25%. ¹H NMR (400 MHz, DMSO-d₆) δ 7.95-7.92 (m, 2H), 7.88 (d, J=9 Hz, 1H), 7.71 (d, J=9 Hz, 1H), 5.28 (s, 2H), 4.10 (s, 2H), 3.86-3.82 (m, 2H), 3.51 (t, J=7 Hz, 2H), 3.47-3.28 (m, 2H), 2.68-2.66 (m, 1H), 2.13 (s, 3H), 1.83-1.80 (m, 2H), 1.32-1.29 (m, 2H), 1.05 (t, J=7 Hz, 3H).

### Step 6. 3-Methyl-5-nitro-2-(6-(((tetrahydro-2H-pyran-4-yl)amino)methyl)pyridazin-3-yl)phenol hydrochloride

A mixture of N-[[6-[2-(ethoxymethoxy)-6-methyl-4-nitro-phenyl]pyridazin-3-yl]methyl]tetra hydropyran-4-amine (110 mg, 273 mol) and 4 M HCl in EtOAc (1.5 mL) was stirred at 25° C. for 1 h then concentrated under reduced pressure. The residue was purified by prep-HPLC (column: Phenomenex Luna C18 100×30 mm, 3 μm; mobile phase: [(A) H₂O (0.04% HCl)—(B) ACN]; gradient: 1-15% B, 8 min) and lyophilised to give the title compound as a white solid, Y=42%. ¹H NMR (400 MHz, DMSO-d₆) δ 11.05 (br. s, 1H), 9.92 (s, 2H), 8.08 (d, J=9 Hz, 1H), 7.88 (d, J=9 Hz, 1H), 7.80 (s, 1H), 7.72 (s, 1H), 4.62 (s, 2H), 3.97-3.93 (m, 2H), 3.45-3.40 (m, 1H), 3.36-3.31 (m, 2H), 2.14 (s, 3H), 2.11-2.07 (m, 2H), 1.82-1.77 (m, 2H). LCMS (ESI): m/z: [M+H]⁺=345.1.

### Compound 58. (R)-5-Chloro-3-methyl-2-(6-((methyl(tetrahydrofuran-3-yl)amino)methyl)pyridazin-3-yl)phenol

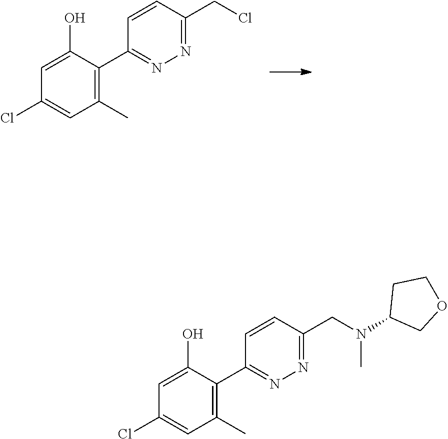 [SMILES: Cc1cc(Cl)cc(O)c1-c1ccc(CCl)nn1 ; Cc1cc(Cl)cc(O)c1-c1ccc(CN(C)[C@@H]2CCOC2)nn1]

To a solution of 5-chloro-2-[6-(chloromethyl)pyridazin-3-yl]-3-methyl-phenol (Intermediate C2, 70 mg, 260 mol) in DMSO (0.7 mL) was added (3R)—N-methyltetrahydrofuran-3-amine (43.0 mg, 312 mol) and DIPEA (227 μL, 1.30 mmol) at 25° C. under N₂. The mixture was stirred at 80° C. for 2 h then allowed to cool to room temperature. The reaction mixture was purified by prep-HPLC (column: Waters Xbridge Prep OBD C18 150×40 mm, 10 μm; mobile phase: [(A) H₂O (10 mM NH₄HCO₃)—(B) ACN]; gradient: 10-50% B, 8 min) and lyophilised to give the title compound as a white solid, Y=40%. ¹H NMR (400 MHz, DMSO-d₆) δ 10.06 (br. s, 1H), 7.72 (d, J 9 Hz, 1H), 7.62 (d, J 9 Hz, 1H), 6.88 (s, 1H), 6.85 (s, 1H), 3.91-3.74 (m, 4H), 3.70-3.59 (m, 2H), 3.29-3.26 (m, 1H), 2.13 (s, 3H), 2.10-2.04 (m, 1H), 2.03 (s, 3H), 1.91-1.81 (m, 1H). LC-MS (ESI): m/z: [M+H]⁺=334.2.

### Compound 59. (S)-5-Chloro-3-methyl-2-(6-((methyl(tetrahydrofuran-3-yl)amino)methyl)pyridazin-3-yl)phenol

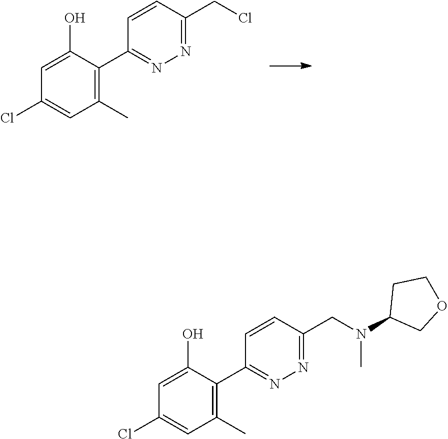 [SMILES: Cc1cc(Cl)cc(O)c1-c1ccc(CCl)nn1 ; Cc1cc(Cl)cc(O)c1-c1ccc(CN(C)[C@H]2CCOC2)nn1]

To a solution of 5-chloro-2-[6-(chloromethyl)pyridazin-3-yl]-3-methyl-phenol (Intermediate C2, 80 mg, 297 mol) in DMSO (1 mL) was added DIPEA (104 μL, 595 mol) and (3S)—N-methyltetrahydrofuran-3-amine (53.2 mg, 386 mol) at 25° C. under N₂. The mixture was stirred at 80° C. for 2 h then allowed to cool to room temperature. The reaction mixture was purified by directly prep-HPLC (column: Waters Xbridge Prep OBD C18 150×40 mm, 10 μm; mobile phase: [(A) H₂O (10 mM NH₄HCO₃)—(B) ACN]; gradient: 15-45% B, 8 min) and lyophilised to give the title compound as a white solid, Y=31%. ¹H NMR (400 MHz, DMSO-d₆) δ 10.08 (s, 1H), 7.72 (d, J=9 Hz, 1H), 7.62 (d, J=9 Hz, 1H), 6.88 (s, 1H), 6.85 (s, 1H), 3.91-3.83 (m, 2H), 3.81-3.74 (m, 2H), 3.70-3.57 (m, 2H), 3.29-3.22 (m, 1H), 2.13 (s, 3H), 2.09-2.03 (m, 1H), 2.01 (s, 3H), 1.92-1.79 (m, 1H). LCMS (ESI): m/z: [M+H]⁺=334.2.

### Compound 60. (R)-5-Chloro-2-(6-((3-methoxypyrrolidin-1-yl)methyl)pyridazin-3-yl)-3-methylphenol

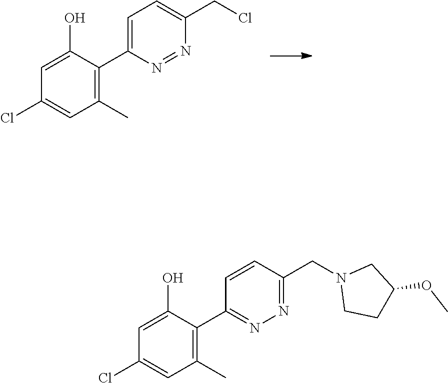 [SMILES: Cc1cc(Cl)cc(O)c1-c1ccc(CCl)nn1 ; CO[C@@H]1CCN(Cc2ccc(-c3c(C)cc(Cl)cc3O)nn2)C1]

To a solution of (3R)-3-methoxypyrrolidine (26.3 mg, 260 mol) in DMSO (0.2 mL) were added DIPEA (90.6 μL, 520 mol) and 5-chloro-2-[6-(chloromethyl)pyridazin-3-yl]-3-methyl-phenol (Intermediate C2, 70 mg, 260 mol) at 25° C. The mixture was stirred at 80° C. for 1 h then allowed to cool to room temperature. The reaction mixture was purified directly by prep-HPLC (column: Waters Xbridge Prep OBD C18 150×40 mm, 10 μm; mobile phase: [(A) H₂O (10 mM NH₄HCO₃)—(B) ACN]; gradient: 15-55% B, 8 min) and lyophilised to give the title as a white solid, Y=35%. ¹H NMR (400 MHz, DMSO-d₆) δ 7.70 (d, J=9 Hz, 1H), 7.61 (d, J=9 Hz, 1H), 6.92-6.80 (m, 2H), 3.97-3.88 (m, 3H), 3.17 (s, 3H), 2.76-2.74 (m, 1H), 2.66-2.60 (m, 1H), 2.57-2.50 (m, 1H), 2.50-2.47 (m, 1H), 2.05-1.97 (m, 4H), 1.73-1.64 (m, 1H). LCMS (ESI): m/z: [M+H]⁺=334.2.

### Compound 61. (S)-5-Chloro-2-(6-((3-methoxypyrrolidin-1-yl)methyl)pyridazin-3-yl)-3-methylphenol

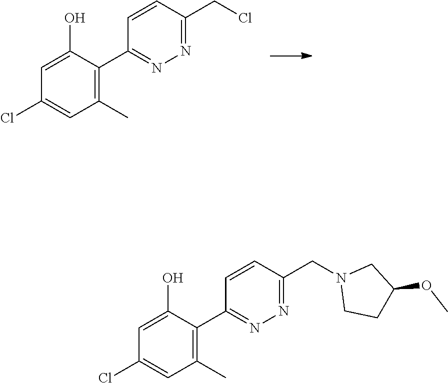 [SMILES: Cc1cc(Cl)cc(O)c1-c1ccc(CCl)nn1 ; CO[C@H]1CCN(Cc2ccc(-c3c(C)cc(Cl)cc3O)nn2)C1]

To a solution of 5-chloro-2-[6-(chloromethyl)pyridazin-3-yl]-3-methyl-phenol (Intermediate C2, 70.0 mg, 260 mol) in DMSO (1 mL) were added DIPEA (90.6 μL, 520 mol) and (3S)-3-methoxypyrrolidine (26.3 mg, 260 mol) at 25° C. The mixture was stirred at 60° C. for 2 h then allowed to cool to room temperature. The reaction mixture was purified directly by prep-HPLC (column: Waters Xbridge Prep OBD C18 150×40 mm, 10 μm; mobile phase: [(A) H₂O (10 mM NH₄HCO₃)—(B) ACN]; gradient: 15-55% B, 8 min) and lyophilised to give the title compound as a white solid, Y=34%.

### Compound 62. 1-((6-(4-Chloro-2-hydroxy-6-methylphenyl)pyridazin-3-yl)methyl)pyrrolidin-2-one

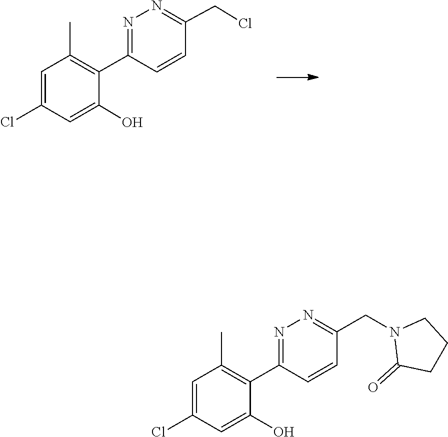 [SMILES: Cc1cc(Cl)cc(O)c1-c1ccc(CCl)nn1 ; Cc1cc(Cl)cc(O)c1-c1ccc(CN2CCCC2=O)nn1]

To a solution of pyrrolidin-2-one (57.0 μL, 743 mol) in DMF (1 mL) was added NaH (14.9 mg, 372 mol, 60% wt in mineral oil) at 0° C. under N₂. The mixture was stirred at 0° C. for 0.5 h then was treated with a solution of 5-chloro-2-[6-(chloromethyl)pyridazin-3-yl]-3-methylphenol (Intermediate C2, 100 mg, 372 mol) in DMF (1 mL). The mixture was stirred at 25° C. for 2 h then quenched by saturated NH₄Cl solution (2 ml) at 0° C. and extracted with EtOAc (3×2 mL). The combined organic layers were washed with brine (2 mL), dried over Na₂SO₄, filtered and concentrated under reduced pressure. The residue was purified by prep-HPLC (column: Waters Xbridge BEH C18 100×30 mm, 10 μm; mobile phase: [(A) H₂O (10 mM NH₄HCO₃)—(B) ACN]; gradient: 15-45% B, 8 min) and lyophilised to give the title compound as yellow solid, Y=25%. ¹H NMR (400 MHz, DMSO-d₆) δ 10.04 (br. s, 1H), 7.64 (d, J=8 Hz, 1H), 7.56 (d, J=8 Hz, 1H), 6.88 (s, 1H), 6.85 (s, 1H), 4.71 (s, 2H), 3.43 (t, J=8 Hz, 2H), 2.34 (t, J=8 Hz, 2H), 2.06-1.93 (m, 5H). LCMS (ESI): m/z: [M+H]⁺=318.2.

### Compound 63. (R)-5-Chloro-3-methyl-2-(6-(((tetrahydrofuran-3-yl)oxy)methyl)pyridazin-3-yl)phenol

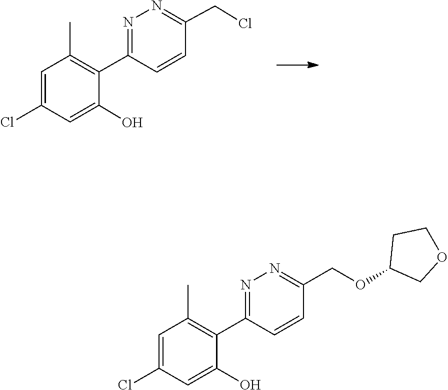 [SMILES: Cc1cc(Cl)cc(O)c1-c1ccc(CCl)nn1 ; Cc1cc(Cl)cc(O)c1-c1ccc(CO[C@@H]2CCOC2)nn1]

To a solution of (3R)-tetrahydrofuran-3-ol (59.7 μL, 743 mol) in DMF (2 mL) was added NaH (29.7 mg, 743 mol, 60% wt in mineral oil) at 0° C. under N₂. The mixture was stirred at 0° C. for 0.5 h then 5-chloro-2-[6-(chloromethyl)pyridazin-3-yl]-3-methyl-phenol (Intermediate C2, 50 mg, 186 mol) was added. The mixture was stirred at 25° C. for 1 h then was quenched with saturated NH₄Cl solution (2 mL) at 0° C. and concentrated under reduced pressure. The residue was purified by prep-HPLC (column: Waters Xbridge BEH C18 100×30 mm, 10 μm; mobile phase: [(A) H₂O (10 mM NH₄HCO₃)—(B) ACN]; gradient: 5-55% B, 8 min) and lyophilised to give the title compound as a white solid, Y=52%. ¹H NMR (400 MHz, DMSO-d₆) δ 10.10 (s, 1H), 7.73 (d, J=8 Hz, 1H), 7.67 (d, J=8 Hz, 1H), 6.88 (s, 1H), 6.85 (s, 1H), 4.92-4.69 (m, 2H), 4.46-4.27 (m, 1H), 3.86-3.76 (m, 2H), 3.75-3.65 (m, 2H), 2.13-1.90 (m, 5H). LC-MS (ESI): m/z: [M+H]⁺=321.2.

### Compound 64. (S)-5-Chloro-3-methyl-2-(6-(((tetrahydrofuran-3-yl)oxy)methyl)pyridazin-3-yl)phenol

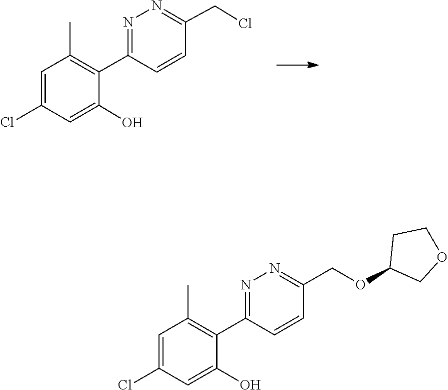 [SMILES: Cc1cc(Cl)cc(O)c1-c1ccc(CCl)nn1 ; Cc1cc(Cl)cc(O)c1-c1ccc(CO[C@H]2CCOC2)nn1]

To a solution of (3S)-tetrahydrofuran-3-ol (26.2 mg, 297 mol) in DMF (0.8 mL) was added NaH (11.9 mg, 297 mol, 60% wt in mineral oil) at 0° C. under N₂. The mixture was stirred at 0° C. for 0.5 h then was treated with a solution of 5-chloro-2-[6-(chloromethyl)pyridazin-3-yl]-3-methyl-phenol (Intermediate C2, 80 mg, 297 mol) in DMF (1.6 mL). The mixture was stirred at 25° C. for 2 h then quenched by saturated aqueous NH₄Cl (2 mL) at 0° C. and extracted with EtOAc (3×2 mL). The combined organic layers were washed with brine (2 mL), dried over Na₂SO₄, filtered and concentrated under reduced pressure. The residue was purified by prep-HPLC (column: Waters Xbridge BEH C18 100×30 mm, 10 μm; mobile phase: [(A) H₂O (10 mM NH₄HCO₃)—(B) ACN]; gradient: 5-55% B, 8 min) and lyophilised to give the title compound as a white solid, Y=31%. ¹H NMR (400 MHz, DMSO-d₆) δ 10.12 (br. s, 1H), 7.74 (d, J=8 Hz, 1H), 7.67 (d, J=8 Hz, 1H), 6.89 (s, 1H), 6.85 (s, 1H), 4.86-4.77 (m, 2H), 4.36-4.35 (m, 1H), 3.86-3.67 (m, 4H), 2.07-1.98 (m, 5H). LCMS (ESI): m/z: [M+H]⁺=321.2.

### Compound 65. 5-Chloro-3-methyl-2-(6-((oxetan-3-yloxy)methyl)pyridazin-3-yl)phenol

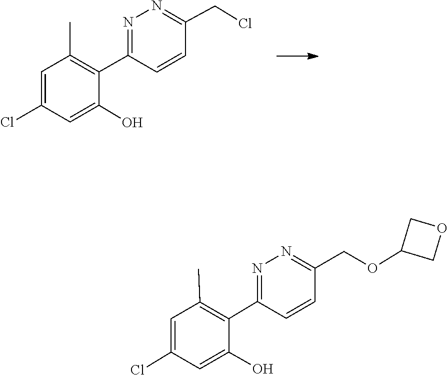 [SMILES: Cc1cc(Cl)cc(O)c1-c1ccc(CCl)nn1 ; Cc1cc(Cl)cc(O)c1-c1ccc(COC2COC2)nn1]

To a solution of 5-chloro-2-[6-(chloromethyl)pyridazin-3-yl]-3-methyl-phenol (Intermediate C2, 100 mg, 372 mol) in DMF (2 mL) was added NaH (44.6 mg, 1.11 mmol, 60% wt in mineral oil) and oxetan-3-ol (82.6 mg, 1.11 mmol) at 0° C. under N₂. The mixture was stirred at 25° C. for 2 h. then concentrated under reduced pressure. The residue was purified by prep-HPLC (column: Waters Xbridge BEH C18 100×30 mm, 10 μm; mobile phase: [(A) H₂O (10 mM NH₄HCO₃)—(B) ACN]; gradient: 5-55% B, 8 min) to give the title compound as a white solid, Y=33%. ¹H NMR (400 MHz, DMSO-d₆) δ 10.11 (s, 1H), 7.79 (d, J=9 Hz, 1H), 7.69 (m, 1H), 6.95-6.80 (m, 2H), 4.82-4.74 (m, 3H), 4.73-4.67 (m, 2H), 4.50-4.40 (m, 2H), 2.01 (s, 3H). LCMS (ESI): m/z: [M+H]⁺=307.1.

### Compound 66. (R)-5-(((6-(4-Chloro-2-hydroxy-6-methylphenyl)pyridazin-3-yl)methyl)amino)-1-methylpiperidin-2-one

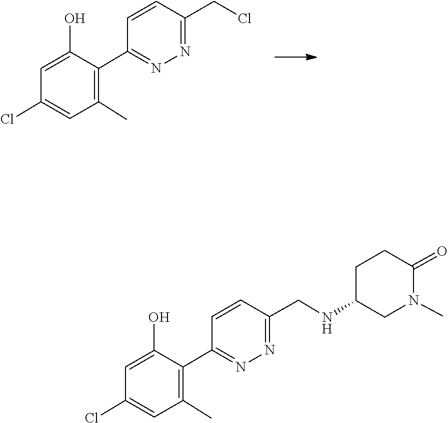 [SMILES: Cc1cc(Cl)cc(O)c1-c1ccc(CCl)nn1 ; Cc1cc(Cl)cc(O)c1-c1ccc(CN[C@@H]2CCC(=O)N(C)C2)nn1]

To a solution of 5-chloro-2-[6-(chloromethyl)pyridazin-3-yl]-3-methyl-phenol (Intermediate C2, 70.0 mg, 260 mol) in DMSO (1.5 mL) were added DIPEA (90.6 μL, 520 mol) and (5R)-5-amino-1-methyl-piperidin-2-one (33.3 mg, 260 mol) at 25° C. The mixture was stirred at 80° C. for 1 h then allowed to cool to room temperature and concentrated under reduced pressure. The residue was purified by prep-HPLC (column: Waters Xbridge Prep OBD C18 150×40 mm, 10 μm; mobile phase: [(A) H₂O (10 mM NH₄HCO₃)—(B) ACN]; gradient: 10-40% B, 8 min) and lyophilised to give the title compound as a white solid, Y=26%. ¹H NMR (400 MHz, DMSO-d₆) δ 10.03 (br. s, 1H), 7.76 (d, J=9 Hz, 1H), 7.62 (d, J=9 Hz, 1H), 6.88-6.85 (m, 2H), 4.14-4.02 (m, 2H), 3.44-3.40 (m, 1H), 3.11-3.07 (m, 1H), 2.97-2.95 (m, 1H), 2.76 (s, 3H), 2.40-2.35 (m, 0.5H), 2.30-2.25 (m, 0.5H), 2.20-2.10 (m, 1H), 2.01 (s, 3H), 1.98-1.86 (m, 1H), 1.77-1.61 (m, 1H). LCMS (ESI): m/z: [M+H]⁺=361.2.

### Compound 67. 3-((6-(4-Chloro-2-hydroxy-6-methylphenyl)pyridazin-3-yl)methyl)oxazolidin-2-one

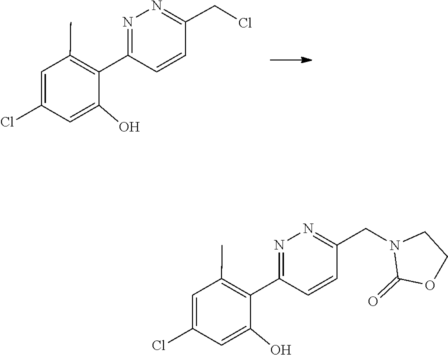 [SMILES: Cc1cc(Cl)cc(O)c1-c1ccc(CCl)nn1 ; Cc1cc(Cl)cc(O)c1-c1ccc(CN2CCOC2=O)nn1]

To a solution of oxazolidin-2-one (64.7 mg, 743 mol) in DMF (2 mL) was added NaH (29.7 mg, 743 mol, 60% wt in mineral oil) at 0° C. under N₂. The mixture was stirred at 0° C. for 0.5 h then 5-chloro-2-[6-(chloromethyl)pyridazin-3-yl]-3-methyl-phenol (Intermediate C2, 100 mg, 372 mol) was added. The mixture was stirred at 25° C. for 1 h then was quenched by addition of H₂O (1 mL) at 0° C. and concentrated under reduced pressure. The residue was purified by prep-HPLC (column: Waters Xbridge BEH C18 100×30 mm, 10 μm; mobile phase: [(A) H₂O (10 mM NH₄HCO₃)—(B) ACN]; gradient: 5-45% B, 8 min) and lyophilised to give the title compound as a white solid, Y=29%. 1H NMR (400 MHz, DMSO-d₆) δ 10.07 (s, 1H), 7.76-7.54 (m, 2H), 6.89 (s, 1H), 6.86 (s, 1H), 4.71 (s, 2H), 4.35 (t, J=8 Hz, 2H), 3.66 (t, J=8 Hz, 2H), 2.02 (s, 3H). LC-MS (ESI): m/z: [M+H]⁺=320.2.

### Compound 68. 1-(3-((6-(4-Chloro-2-hydroxy-6-methylphenyl)pyridazin-3-yl)methoxy)pyrrolidin-1-yl)ethan-1-one

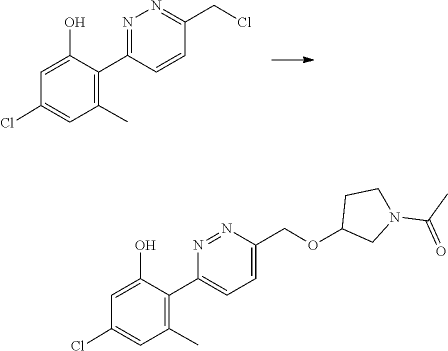 [SMILES: Cc1cc(Cl)cc(O)c1-c1ccc(CCl)nn1 ; CC(=O)N1CCC(OCc2ccc(-c3c(C)cc(Cl)cc3O)nn2)C1]

To a solution of 5-chloro-2-[6-(chloromethyl)pyridazin-3-yl]-3-methyl-phenol (Intermediate C2, 70 mg, 260 mol) and 1-(3-hydroxypyrrolidin-1-yl)ethanone (33.6 mg, 260 mol) in DMF (0.7 mL) was added NaH (20.8 mg, 520 mol, 60% wt in mineral oil) at 0° C. under N₂. The mixture was stirred at 25° C. for 2 h then was quenched by addition of saturated aqueous NH₄Cl (1 ml) at 0° C. and concentrated under reduced pressure. The residue was purified by prep-HPLC (column: Waters Xbridge BEH C18 100×30 mm, 10 μm; mobile phase: [(A) H₂O (10 mM NH₄HCO₃)—(B) ACN]; gradient: 5-50% B, 8 min) and lyophilised to give the title compound as a white solid, Y=26%. ¹H NMR (400 MHz, DMSO-d₆) δ 10.05 (br. s, 1H), 7.74 (d, J=8 Hz, 1H), 7.67 (d, J=8 Hz, 1H), 6.89 (d, J=2 Hz, 1H), 6.85 (d, J=2 Hz, 1H), 4.93-4.78 (m, 2H), 4.39-4.26 (m, 1H), 3.70-3.33 (m, 4H), 2.23-2.03 (m, 2H), 2.01 (s, 3H), 1.94 (d, J=8 Hz, 3H). LCMS (ESI): m/z: [M+H]⁺=362.2.

### Compound 69. 5-Chloro-3-fluoro-2-(6-(((tetrahydro-2H-pyran-4-yl)amino)methyl)pyridazin-3-yl)phenol hydrochloride

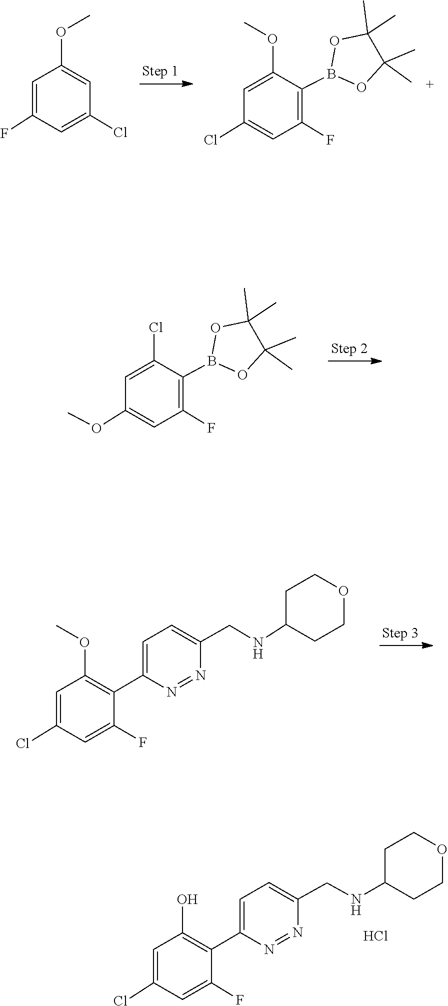

### Step 1. 2-(4-Chloro-2-fluoro-6-methoxyphenyl)-4,4,5,5-tetramethyl-1,3,2-dioxaborolane

To a solution of 1-chloro-3-fluoro-5-methoxy-benzene (3.80 g, 23.7 mmol) in THE (40 mL) was added LDA (2 M in THF, 11.8 mL, 23.6 mmol) at −78° C. under N₂ atmosphere. The mixture was stirred at −78° C. for 30 min then 2-isopropoxy-4,4,5,5-tetramethyl-1,3,2-dioxaborolane (4.83 mL, 23.7 mmol) was added. The mixture was stirred at −78° C. for 2 h then was quenched by addition of saturated NH₄Cl solution (40 mL) at 0° C. and extracted with EtOAc (3×40 mL). The combined organic layers were washed with brine (40 mL), dried over Na₂SO₄, filtered and concentrated under reduced pressure. The residue was purified by column chromatography (SiO₂, petroleum ether:ethyl acetate=1:1) to give a mixture of the title compound and side product 2-(2-chloro-6-fluoro-4-methoxyphenyl)-4,4,5,5-tetramethyl-1,3,2-dioxaborolane as a white solid.

### Step 2. N-((6-(4-Chloro-2-fluoro-6-methoxyphenyl)pyridazin-3-yl)methyl)tetrahydro-2H-pyran-4-amine

To a solution of an inseparable mixture product of 2-(4-chloro-2-fluoro-6-methoxy-phenyl)-4,4,5,5-tetramethyl-1,3,2-dioxaborolane and 2-(2-chloro-6-fluoro-4-methoxyphenyl)-4,4,5,5-tetramethyl-1,3,2-dioxaborolane (1.00 g, mixture) in dioxane (8 mL) and H₂O (2 mL) were added K₂CO₃ (1.93 g, 14.0 mmol), N-[(6-chloropyridazin-3-yl)methyl]tetrahydropyran-4-amine (Intermediate A1, 954 mg, 4.19 mmol) and (2-dicyclohexylphosphino-2′,6′-dimethoxybiphenyl)[2-(2′-amino-1,1′-biphenyl)]palladium(II) methanesulfonate (272 mg, 349 mol) at 25° C. under N₂ atmosphere. The mixture was stirred at reflux temperature for 2 h then allowed to cool to room temperature. The reaction mixture was diluted with H₂O (10 mL) and the resulting mixture was extracted with EtOAc (3×10 mL). The combined organic layers were washed with brine (2×10 mL), dried over anhydrous Na₂SO₄, filtered and concentrated under reduced pressure. The residue was purified by prep-HPLC (column: Waters Xbridge BEH C18 100×30 mm, 10 μm; mobile phase: [(A) H₂O (10 mM NH₄HCO₃)—(B) ACN]; gradient: 20-40% B, 8 min) and lyophilised to give the title compound as a yellow solid. ¹H NMR (400 MHz, DMSO-d₆) δ 7.82 (d, J=9 Hz, 1H), 7.72 (d, J=9 Hz, 1H), 7.22-7.20 (m, 1H), 7.17-7.15 (m, 1H), 4.11 (s, 2H), 3.95-3.75 (m, 5H), 3.31-3.24 (m, 2H), 2.72-2.62 (m, 1H), 1.83-1.80 (m, 2H), 1.43-1.18 (m, 2H). LCMS (ESI): m/z: [M+H]⁺=352.2.

### Step 3. 5-Chloro-3-fluoro-2-(6-(((tetrahydro-2H-pyran-4-yl)amino)methyl)pyridazin-3-yl)phenol hydrochloride

To a solution of N-[[6-(4-chloro-2-fluoro-6-methoxy-phenyl)pyridazin-3-yl]methyl]tetra hydropyran-4-amine (30.0 mg, 85.3 mol) in DCM (0.5 mL) was added BBr₃ (16.4 μL, 171 mol) at 0° C. under N₂. The mixture was stirred at 25° C. for 1 h then was quenched by addition of H₂O (0.1 mL) at 0° C. and concentrated under reduced pressure. The residue was purified by prep-HPLC (column: Phenomenex Luna C18 100×40 mm, 5 μm; mobile phase: [(A) H₂O (0.04% HCl)—(B) ACN]; gradient: 10-35% B, 8 min) and lyophilised to give the title compound as a white solid, Y=34%. ¹H NMR (400 MHz, DMSO-d₆) δ 11.24 (s, 1H), 9.61 (s, 2H), 8.03-7.86 (m, 2H), 7.07-7.04 (m, 1H), 7.01-6.98 (m, 1H), 4.63 (t, J=6 Hz, 2H), 3.98-3.94 (m, 2H), 3.52-3.39 (m, 1H), 3.37-3.32 (m, 2H), 2.13-2.01 (m, 2H), 1.74-1.69 (m, 2H). LCMS (ESI): m/z: [M+H]⁺=338.1.

### Compound 70. 5-(Difluoromethoxy)-3-methyl-2-(6-(((tetrahydro-2H-pyran-4-yl)amino)methyl)pyridazin-3-yl)phenol

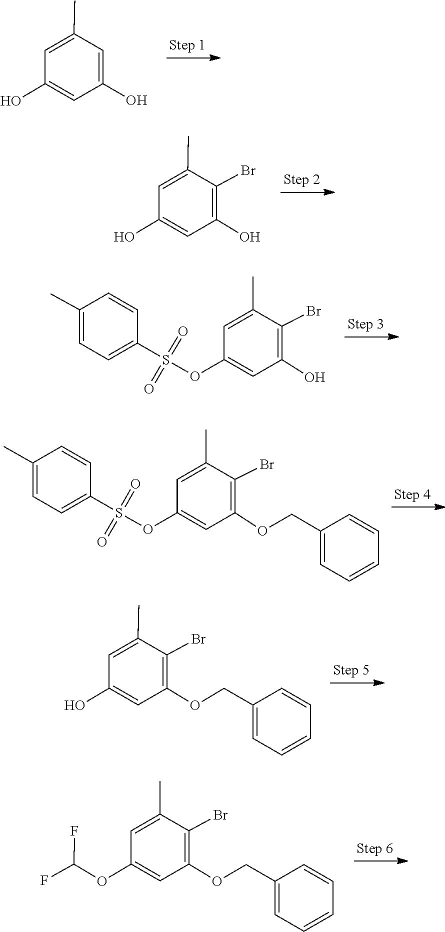

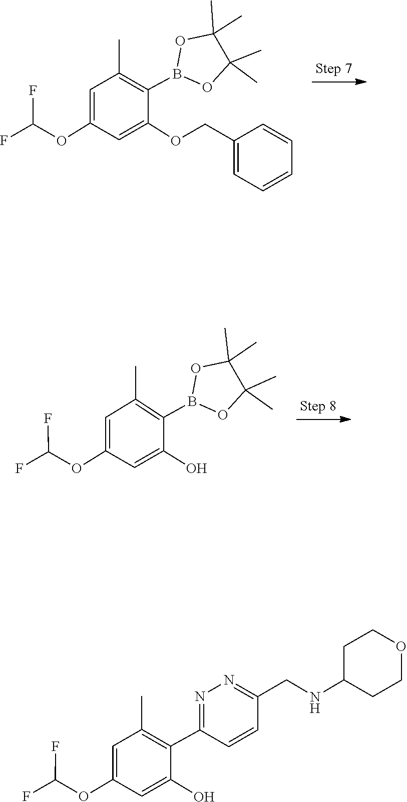 [SMILES: Cc1cc(OC(F)F)cc(OCc2ccccc2)c1B1OC(C)(C)C(C)(C)O1 ; Cc1cc(OC(F)F)cc(O)c1B1OC(C)(C)C(C)(C)O1 ; Cc1cc(OC(F)F)cc(O)c1-c1ccc(CNC2CCOCC2)nn1]

### Step 1. 4-Bromo-5-methylbenzene-1,3-diol

To a solution of 5-methylbenzene-1,3-diol (20.0 g, 161 mmol) in DCM (120 mL) and MeOH (80 mL) was slowly added tetrabutylammonium tribromide (77.7 g, 161 mmol) at 25° C. under N₂. The mixture was stirred at 25° C. for 4 h then concentrated under reduced pressure. The residue was diluted with H₂O (50 mL) and extracted with EtOAc (3×50 mL). The combined organic layers were washed with brine (150 mL), dried over Na₂SO₄, filtered and concentrated under reduced pressure. The residue was purified by prep-HPLC (column: Welch Xtimate C18 250×100 mm, 10 μm; mobile phase: [(A) H₂O (10 mM NH₄HCO₃)—(B) ACN]; gradient: 10-45% B, 20 min) and lyophilised to give the title compound as a white solid, Y=58%. ¹H NMR (400 MHz, DMSO-d₆) δ 9.62 (br. s, 2H), 6.27 (d, J 2 Hz, 1H), 6.21 (d, J 2 Hz, 1H), 2.20 (s, 3H).

### Step 2. 4-Bromo-3-hydroxy-5-methylphenyl 4-methylbenzenesulfonate

To a solution of 4-bromo-5-methyl-benzene-1,3-diol (10.0 g, 49.3 mmol) in acetone (200 mL) were added K₂CO₃ (20.4 g, 148 mmol) and TsCl (3.82 g, 54.2 mmol) at 25° C. under N₂. The mixture was stirred at 60° C. for 24 h then allowed to cool to room temperature. The reaction mixture was diluted with H₂O (100 mL) and extracted with EtOAc (3×100 mL). The combined organic layers were washed with brine (300 mL), dried over Na₂SO₄, filtered and concentrated under reduced pressure. The residue was purified by prep-HPLC (column: Phenomenex Luna C18 250×150 mm, 15 μm; mobile phase: [(A) H₂O (10 mM NH₄HCO₃)—(B) ACN]; gradient: 40-75% B, 22 min) and lyophilised to give the title compound as a white solid, Y=26%. ¹H NMR (400 MHz, DMSO-d₆) δ 10.63 (br. s, 1H), 7.76-7.74 (m, 2H), 7.53-7.44 (m, 2H), 6.51 (d, J 2 Hz, 1H), 6.44 (d, J 2 Hz, 1H), 2.43 (s, 3H), 2.24 (s, 3H).

### Step 3. 3-(Benzyloxy)-4-bromo-5-methylphenyl 4-methylbenzenesulfonate

To a solution of (4-bromo-3-hydroxy-5-methyl-phenyl) 4-methylbenzenesulfonate (6.50 g, 18.2 mmol) in acetone (130 mL) were added bromomethylbenzene (2.16 mL, 18.2 mmol) and K₂CO₃ (7.54 g, 54.6 mmol) at 25° C. under N₂. The mixture was stirred at 60° C. for 1 h then allowed to cool to room temperature. The reaction mixture was diluted with H₂O (130 mL) and extracted with EtOAc (3×130 mL). The combined organic layers were washed with brine (400 mL), dried over Na₂SO₄, filtered and concentrated under reduced pressure. The residue was purified by column chromatography (SiO₂, petroleum ether:ethyl acetate=10:1 to 1:1) to give the title compound as a white solid, Y=74%. ¹H NMR (400 MHz, DMSO-d₆) δ 7.77-7.70 (m, 2H), 7.48-0.46 (m, 2H), 7.43-7.38 (m, 4H), 7.37-7.32 (m, 1H), 6.72 (d, J 2 Hz, 1H), 6.67 (d, J 2 Hz, 1H), 5.03 (s, 2H), 2.42 (s, 3H), 2.29 (s, 3H).

### Step 4. 3-(Benzyloxy)-4-bromo-5-methylphenol

To a solution of (3-benzyloxy-4-bromo-5-methyl-phenyl) 4-methylbenzenesulfonate (6.00 g, 13.4 mmol) in MeOH (156 mL) was added NaOH (2 M in H₂O, 33.5 mL, 67 mmol) at 25° C. The mixture was stirred at 60° C. for 2 h under N₂ then allowed to cool to room temperature. The reaction mixture was concentrated under reduced pressure to remove MeOH, then the residue was diluted with H₂O (30 mL) and extracted with EtOAc (3×40 mL). The combined organic layers were washed with brine (90 mL), dried over Na₂SO₄, filtered and concentrated under reduced pressure to give the title compound as a white solid, Y=76%. ¹H NMR (400 MHz, DMSO-d₆) δ 9.58 (s, 1H), 7.48-7.44 (m, 2H), 7.43-7.38 (m, 2H), 7.37-7.28 (m, 1H), 6.42 (d, J 2 Hz, 1H), 6.39 (d, J 2 Hz, 1H), 5.11 (s, 2H), 2.25 (s, 3H).

### Step 5. 1-(Benzyloxy)-2-bromo-5-(difluoromethoxy)-3-methylbenzene

To a solution of 3-benzyloxy-4-bromo-5-methyl-phenol (1.00 g, 3.41 mmol) in ACN (6 mL) and H₂O (6 mL) was added KOH (3.83 g, 68.2 mmol) and 1-[[bromo(difluoro)methyl]-ethoxy-phosphoryl]oxyethane (1.82 g, 6.82 mmol, 2 eq) at 0° C. under N₂. The mixture was stirred at 25° C. for 2 h then concentrated under reduced pressure to remove ACN and extracted with EtOAc (3×6 mL). The combined organic layers were washed with brine (18 mL), dried over Na₂SO₄, filtered and concentrated under reduced pressure. The residue was purified by column chromatography (SiO₂, petroleum ether:ethyl acetate=10:1 to 5:1) to give the title compound as a white solid, Y=89%. ¹H NMR (400 MHz, DMSO-d₆) δ 7.51-7.45 (m, 2H), 7.43-7.41 (m, 2H), 7.37-7.31 (m, 1H), 7.27-7.02 (m, 1H), 6.92-6.87 (m, 1H), 6.86-6.82 (m, 1H), 5.21 (s, 2H), 2.36 (s, 3H).

### Step 6. 2-(2-(Benzyloxy)-4-(difluoromethoxy)-6-methylphenyl)-4,4,5,5-tetramethyl-1,3,2-dioxaborolane

To a solution of 1-benzyloxy-2-bromo-5-(difluoromethoxy)-3-methyl-benzene (1.00 g, 2.91 mmol) in dioxane (10 mL) was added TEA (1.22 mL, 8.74 mmol), then to above mixture was slowly added 4,4,5,5-tetramethyl-1,3,2-dioxaborolane (846 μL, 5.83 mmol) at 25° C. under N₂. Then to above mixture was added Pd(OAc)₂ (65.4 mg, 291 mol) and dicyclohexyl-(2-phenylphenyl)phosphane (204 mg, 583 mol) at 25° C. The mixture was stirred at 80° C. for 5 h then allowed to cool to room temperature. The reaction mixture was diluted with H₂O (10 mL) and extracted with EtOAc (3×10 mL). The combined organic layers were washed with brine (30 mL), dried over Na₂SO₄, filtered and concentrated under reduced pressure. The residue was purified by column chromatography (SiO₂, petroleum ether:ethyl acetate=10:1 to 5:1) to give the title compound as a yellow solid, Y=62%.

### Step 7. 5-(Difluoromethoxy)-3-methyl-2-(4,4,5,5-tetramethyl-1,3,2-dioxaborolan-2-yl)phenol

To a solution of 2-[2-benzyloxy-4-(difluoromethoxy)-6-methyl-phenyl]-4,4,5,5-tetramethyl-1,3,2-dioxaborolane (0.30 g, 769 mol) in MeOH (3 mL) was added Pd/C (818 mg) (10% wt on carbon, 50% in water, w/w) under N₂ atmosphere. The suspension was vacuum purged and backfilled with H₂ three times. The mixture was stirred under an atmosphere of H₂ (15 Psi) at 25° C. for 0.5 h then filtered through a pad of celite and the filtrate was concentrated under reduced pressure to give the title compound as a yellow oil, Y=56%.

### Step 8. 5-(Difluoromethoxy)-3-methyl-2-(6-(((tetrahydro-2H-pyran-4-yl)amino)methyl)pyridazin-3-yl)phenol

To a solution of N-[(6-chloropyridazin-3-yl)methyl]tetrahydropyran-4-amine (Intermediate A1, 100 mg, 439 mol) in dioxane (3 mL) and H₂O (1 mL) were added 5-(difluoromethoxy)-3-methyl-2-(4,4,5,5-tetramethyl-1,3,2-dioxaborolan-2-yl)phenol (132 mg, 439 mol), Na₂CO₃ (140 mg, 1.32 mmol) and Pd(dppf)Cl₂ (32.1 mg, 43.9 mol) at 25° C. under N₂. The mixture was stirred at 80° C. for 1 h then allowed to cool to room temperature, diluted with H₂O (3 mL) and extracted with EtOAc (3×3 mL). The combined organic layers were washed with brine (9 mL), dried over Na₂SO₄, filtered and concentrated under reduced pressure. The residue was purified by prep-HPLC (column: Waters Xbridge BEH C18 100×30 mm, 10 m; mobile phase: [(A) H₂O (10 mM NH₄HCO₃)—(B) ACN]; gradient: 5-45% B, 8 min) and lyophilised to give the title compound as a white solid, Y=23%. ¹H NMR (400 MHz, DMSO-d₆) δ 9.15 (br. s, 1H), 7.76 (d, J 9 Hz, 1H), 7.60 (d, J 9 Hz, 1H), 7.41-7.03 (m, 1H), 6.61-6.59 (m, 2H), 4.06 (s, 2H), 3.91-3.76 (m, 2H), 3.29-3.24 (m, 2H), 2.70-2.61 (m, 1H), 2.03 (s, 3H), 1.84-1.80 (m, 2H), 1.37-1.25 (m, 2H). LC-MS (ESI): m/z: [M+H]⁺=366.3.

### Compound 71. 3-Methyl-2-(6-(((1-methylpyrrolidin-3-yl)amino)methyl)pyridazin-3-yl)-5-(trifluoromethyl)phenol dihydrochloride

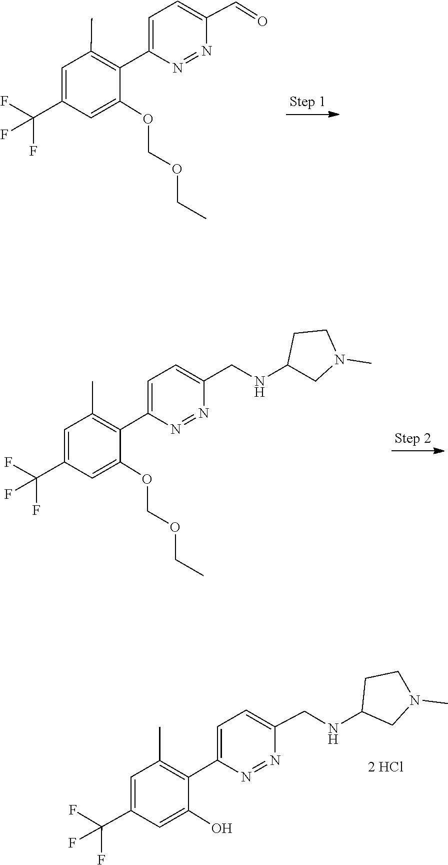 [SMILES: CCOCOc1cc(C(F)(F)F)cc(C)c1-c1ccc(C=O)nn1 ; CCOCOc1cc(C(F)(F)F)cc(C)c1-c1ccc(CNC2CCN(C)C2)nn1 ; Cc1cc(C(F)(F)F)cc(O)c1-c1ccc(CNC2CCN(C)C2)nn1]

### Step 1. N-((6-(2-(Ethoxymethoxy)-6-methyl-4-(trifluoromethyl)phenyl)pyridazin-3-yl)methyl)-1-methylpyrrolidin-3-amine

To a solution of 6-[2-(ethoxymethoxy)-6-methyl-4-(trifluoromethyl)phenyl]pyridazine-3-carbaldehyde (Intermediate C4, 204 mg, 599 mol) in DCE (1.5 mL) were added AcOH (44.6 μL, 779 mol) and 1-methylpyrrolidin-3-amine (60.0 mg, 599 mol) at 0° C. under N₂ atmosphere. The mixture was stirred at 0° C. for 1 h then NaBH(OAc)₃ (190 mg, 899 mol) was added at 0° C. The mixture was stirred at 25° C. for 3 h then diluted with H₂O (8 mL) and extracted with EtOAc (3×5 mL). The combined organic phases were concentrated under reduced pressure to give the title compound as a yellow solid. LCMS (ESI): m/z: [M+H]⁺=425.2.

### Step 2. 3-Methyl-2-(6-(((1-methylpyrrolidin-3-yl)amino)methyl)pyridazin-3-yl)-5-(trifluoromethyl)phenol dihydrochloride

A mixture of N-[[6-[2-(ethoxymethoxy)-6-methyl-4-(trifluoromethyl)phenyl]pyridazin-3-yl]methyl]-1-methyl-pyrrolidin-3-amine (200 mg, 471 mol) and 4 M HCl in EtOAc (2 mL) was stirred at 25° C. for 1 h then concentrated under reduced pressure. The residue was purified by prep-HPLC (column: Phenomenex Luna C18 100×40 mm, 5 μm; mobile phase: [(A) H₂O (0.04% HCl)—(B) ACN]; gradient: 5-30% B, 8 min) and lyophilised to give the title compound as a yellow solid, Y=34%. ¹H NMR (400 MHz, DMSO-d₆) δ 11.58-10.83 (m, 1H), 10.62-10.13 (m, 3H), 7.99-7.96 (m, 1H), 7.87-7.85 (m, 1H), 7.20-7.19 (m, 2H), 4.76-4.59 (m, 2H), 4.37-4.12 (m, 2H), 4.06-3.94 (m, 1H), 3.86-3.58 (m, 1H), 3.47-3.11 (m, 1H), 2.90 (d, J=6 Hz, 3H), 2.66-2.53 (m, 1H), 2.43-2.27 (m, 1H), 2.11 (s, 3H). LCMS (ESI): m/z: [M+H]⁺=367.3.

### Compound 72. (R)-1-(3-(((6-(4-Chloro-2-hydroxy-6-methylphenyl)pyridazin-3-yl)methyl)amino)pyrrolidin-1-yl)ethan-1-one

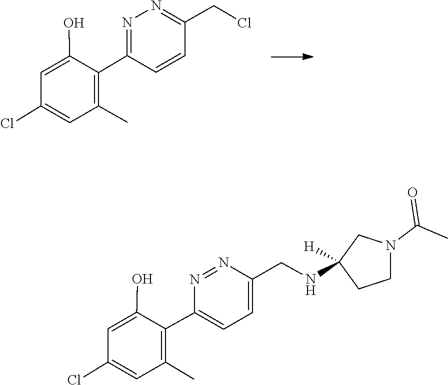 [SMILES: Cc1cc(Cl)cc(O)c1-c1ccc(CCl)nn1 ; CC(=O)N1CC[C@@H](NCc2ccc(-c3c(C)cc(Cl)cc3O)nn2)C1]

To a solution of 5-chloro-2-[6-(chloromethyl)pyridazin-3-yl]-3-methyl-phenol (Intermediate C2, 47.4 mg, 176 mol) in DMSO (0.5 mL) was added 1-[(3R)-3-aminopyrrolidin-1-yl]ethanone (29.0 mg, 176 mol) and DIPEA (92.0 μL, 528 mol) at 25° C. under N₂ atmosphere. The mixture was stirred at 80° C. for 2 h then allowed to cool to room temperature. The reaction mixture was purified directly by prep-HPLC (column: Waters Xbridge BEH C18 100×30 mm, 10 μm; mobile phase: [(A) H₂O (10 mM NH₄HCO₃)—(B) ACN]; gradient: 1-45% B, 8 min) and lyophilised to give the title compound as a white solid, Y=29%. ¹H NMR (400 MHz, DMSO-d₆) δ 10.04 (s, 1H), 7.75 (d, J=9 Hz, 1H), 7.61 (d, J=9 Hz, 1H), 6.87 (s, 1H), 6.85 (s, 1H), 4.09-3.99 (m, 2H), 3.61-3.50 (m, 1H), 3.46-3.37 (m, 2H), 3.28-3.13 (m, 2H), 2.80-2.65 (m, 1H), 2.06-1.97 (m, 4H), 1.91 (d, J=8 Hz, 3H), 1.87-1.67 (m, 1H). LC-MS (ESI): m/z: [M+H]⁺=361.2.

### Compound 73. (S)-1-(3-(((6-(4-Chloro-2-hydroxy-6-methylphenyl)pyridazin-3-yl)methyl)amino)pyrrolidin-1-yl)ethan-1-one

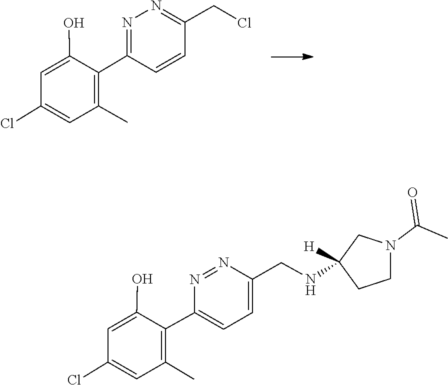 [SMILES: Cc1cc(Cl)cc(O)c1-c1ccc(CCl)nn1 ; CC(=O)N1CC[C@H](NCc2ccc(-c3c(C)cc(Cl)cc3O)nn2)C1]

To a solution of 5-chloro-2-[6-(chloromethyl)pyridazin-3-yl]-3-methyl-phenol (Intermediate C2, 80.0 mg, 297 mol) in DMSO (1 mL) was added 1-[(3S)-3-aminopyrrolidin-1-yl]ethanone (53.8 mg, 327 mol) and DIPEA (155 μL, 892 mol) at 25° C. under N₂ atmosphere. The mixture was stirred at 80° C. for 4 h then allowed to cool to room temperature. The reaction mixture was purified directly by prep-HPLC (column: Waters Xbridge Prep OBD C18 150×40 mm, 10 μm; mobile phase: [(A) H₂O (10 mM NH₄HCO₃)—(B) ACN]; gradient: 15-45% B, 8 min) and lyophilised to give the title compound as a white solid, Y=22%. ¹H NMR (400 MHz, DMSO-d₆) δ 7.75 (d, J=9 Hz, 1H), 7.61 (d, J=9 Hz, 1H), 6.89-6.81 (m, 2H), 4.10-4.00 (m, 2H), 3.59-3.50 (m, 1H), 3.44-3.37 (m, 2H), 3.25-3.13 (m, 2H), 2.81-2.69 (m, 1H), 2.08-1.94 (m, 4H), 1.91 (d, J=8 Hz, 3H), 1.87-1.68 (m, 1H). LCMS (ESI): m/z: [M+H]⁺=361.2.

### Compound 74. 3-Methyl-2-(6-((pyrimidin-2-ylamino)methyl)pyridazin-3-yl)-5-(trifluoromethyl)phenol

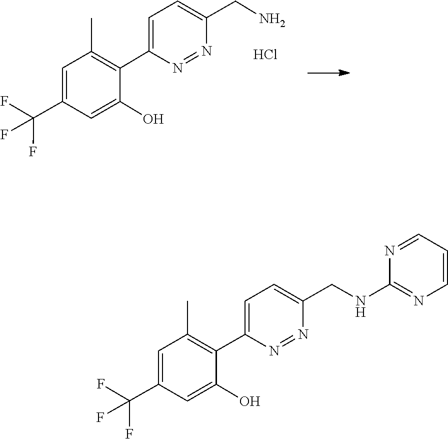 [SMILES: Cc1cc(C(F)(F)F)cc(O)c1-c1ccc(CN)nn1 ; Cl ; Cc1cc(C(F)(F)F)cc(O)c1-c1ccc(CNc2ncccn2)nn1]

To a solution of 2-fluoropyrimidine (16.6 mg, 169 mol) in DMSO (1 mL) were added 2-[6-(aminomethyl)pyridazin-3-yl]-3-methyl-5-(trifluoromethyl)phenol (Intermediate C6, 40 mg, 125 mol) and DIPEA (98.4 μL, 565 mol) at 25° C. The resulting mixture was stirred at 80° C. for 1 h then allowed to cool to room temperature. The reaction mixture was purified directly by prep-HPLC (column: Waters Xbridge Prep OBD C18 150×40 mm, 10 μm; mobile phase: [(A) H₂O (10 mM NH₄HCO₃)—(B) ACN]; gradient: 20-50% B, 8 min) and lyophilised to give the title compound as a white solid, Y=44%. ¹H NMR (400 MHz, DMSO-d₆) δ 9.14 (br. s, 1H), 8.32 (d, J=5 Hz, 2H), 7.86 (t, J=6 Hz, 1H), 7.68-7.56 (m, 2H), 7.14 (s, 1H), 7.08 (s, 1H), 6.65 (t, J=5 Hz, 1H), 4.85 (d, J=6 Hz, 2H), 2.07 (s, 3H). LCMS (ESI): m/z: [M+H]⁺=362.2.

### Compound 75. 3-Methyl-2-(6-((pyrimidin-4-ylamino)methyl)pyridazin-3-yl)-5-(trifluoromethyl)phenol hydrochloride

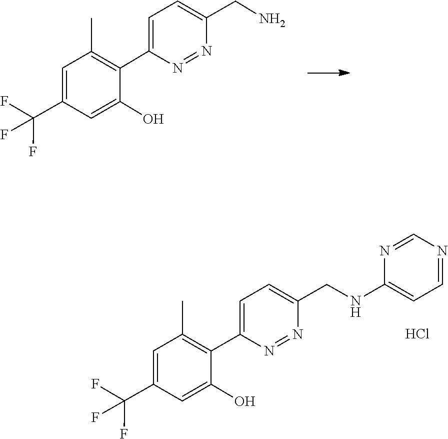 [SMILES: Cc1cc(C(F)(F)F)cc(O)c1-c1ccc(CN)nn1 ; Cc1cc(C(F)(F)F)cc(O)c1-c1ccc(CNc2ccncn2)nn1 ; Cl]

To a solution of 2-[6-(aminomethyl)pyridazin-3-yl]-3-methyl-5-(trifluoromethyl)phenol (Intermediate C6, 80 mg, 250 mol) in DMSO (0.8 mL) was added DIPEA (262 μL, 1.50 mmol) and 4-chloropyrimidine (43.0 mg, 375 mol) at 25° C. The mixture was stirred at 80° C. for 12 h then allowed to cool to room temperature and concentrated under reduced pressure. The residue was purified by prep-HPLC (column: Phenomenex Luna C18 100×40 mm, 5 μm; mobile phase: [(A) H₂O (0.04% HCl)—(B) ACN]; gradient: 10-35% B, 8 min) and lyophilised to give the title compound as a solid, Y=16%. ¹H NMR (400 MHz, DMSO-d₆) δ 14.3 (br. s, 1H), 10.48-10.35 (m, 1H), 10.32-9.99 (m, 1H), 8.96-8.74 (m, 1H), 8.26-8.24 (m, 1H), 7.84-7.78 (m, 1H), 7.77-7.72 (m, 1H), 7.19-7.15 (m, 2H), 7.07-7.04 (d, J=7 Hz, 1H), 5.10 (d, J=6 Hz, 2H), 2.09 (s, 3H). LCMS (ESI): m/z: [M+H]⁺=362.2.

### Compound 76. 3-Methyl-2-(6-((((1-methylpyrrolidin-2-yl)methyl)amino)methyl)pyridazin-3-yl)-5-(trifluoromethyl)phenol dihydrochloride

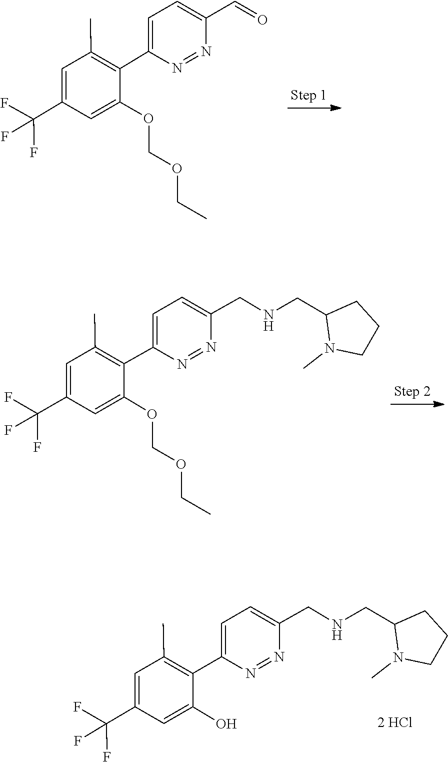 [SMILES: CCOCOc1cc(C(F)(F)F)cc(C)c1-c1ccc(C=O)nn1 ; CCOCOc1cc(C(F)(F)F)cc(C)c1-c1ccc(CNCC2CCCN2C)nn1 ; Cc1cc(C(F)(F)F)cc(O)c1-c1ccc(CNCC2CCCN2C)nn1]

### Step 1. 1-(6-(2-(Ethoxymethoxy)-6-methyl-4-(trifluoromethyl)phenyl)pyridazin-3-yl)-N-((1-methylpyrrolidin-2-yl)methyl)methanamine

To a solution of 6-[2-(ethoxymethoxy)-6-methyl-4-(trifluoromethyl)phenyl]pyridazine-3-carbaldehyde (Intermediate C4, 209 mg, 613 mol) in DCE (1.5 mL) was added AcOH (45.6 L, 797 mol) and (1-methylpyrrolidin-2-yl)methanamine (70.0 mg, 613 mol) at 0° C. under N₂ atmosphere. The mixture was stirred at 0° C. for 1 h then NaBH(OAc)₃ (156 mg, 736 mol) was added at 0° C. The mixture was stirred at 25° C. for 3 h then diluted with H₂O (10 mL), and extracted with EtOAc (3×5 mL). The combined organic phases were concentrated under reduced pressure to give the title compound as a yellow solid.

### Step 2. 3-Methyl-2-(6-((((1-methylpyrrolidin-2-yl)methyl)amino)methyl)pyridazin-3-yl)-5-(trifluoromethyl)phenol dihydrochloride

A mixture of 1-[6-[2-(ethoxymethoxy)-6-methyl-4-(trifluoromethyl)phenyl]pyridazin-3-yl]-N-[(1-methylpyrrolidin-2-yl)methyl]methanamine (0.26 g, 593 mol) and 4 M HCl in EtOAc (3.5 mL) was stirred at 25° C. for 2 h then concentrated under reduced pressure. The residue was purified by prep-HPLC (column: Phenomenex Luna C18 100×40 mm, 5 μm; mobile phase: [(A) H₂O (0.04% HCl)—(B) ACN]; gradient: 5-30% B, 8 min) and lyophilised to give the title compound as a white solid, Y=14%. ¹H NMR (400 MHz, DMSO-d₆) δ 11.12 (br. s, 1H), 10.48 (s, 1H), 9.98 (br. s, 2H), 7.98 (d, J=9 Hz, 1H), 7.87 (d, J=9 Hz, 1H), 7.20-7.17 (m, 2H), 4.68 (s, 2H), 3.70-3.48 (m, 4H), 3.21-3.07 (m, 1H), 2.91 (s, 3H), 2.49-2.33 (m, 1H), 2.12 (s, 3H), 2.10-1.84 (m, 3H). LCMS (ESI): m/z: [M+H]⁺=381.3.

### Compound 77. 3-Methyl-2-[6-({[(1-methylpyrrolidin-3-yl)methyl]amino}methyl)pyridazin-3-yl]-5-(trifluoromethyl)phenol dihydrochloride

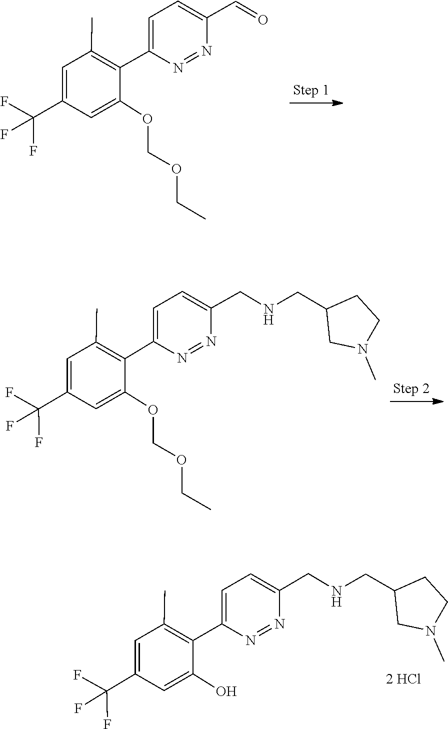 [SMILES: CCOCOc1cc(C(F)(F)F)cc(C)c1-c1ccc(C=O)nn1 ; CCOCOc1cc(C(F)(F)F)cc(C)c1-c1ccc(CNCC2CCN(C)C2)nn1 ; Cc1cc(C(F)(F)F)cc(O)c1-c1ccc(CNCC2CCN(C)C2)nn1]

Synthesised in an analogous way to Compound 76 using (1-methylpyrrolidin-3-yl)methanamine. ¹H NMR (400 MHz, DMSO-d₆) δ 11.27-11.00 (m, 1H), 10.59-10.46 (m, 1H), 9.93-9.66 (m, 2H), 7.99 (d, J=8 Hz, 1H), 7.85 (d, J=8 Hz, 1H), 7.19 (s, 2H), 4.64-4.57 (m, 2H), 3.80-3.48 (m, 2H), 3.35-3.20 (m, 2H), 3.17-2.94 (m, 2H), 2.92-2.75 (m, 4H), 2.35-2.09 (m, 4H), 2.03-1.74 (m, 1H). ¹H NMR (400 MHz, DMSO-d₆+D₂O) δ 7.86 (d, J=8 Hz, 1H), 7.80 (d, J=8 Hz, 1H), 7.18 (s, 1H), 7.11 (s, 1H), 4.58 (s, 2H), 3.78-3.43 (m, 2H), 3.28-2.94 (m, 4H), 2.88-2.77 (m, 4H), 2.36-2.15 (m, 1H), 2.07 (s, 3H), 1.98-1.67 (m, 1H). LCMS (ESI): m/z: [M+H]⁺=381.3.

### Compound 78. 3-Methyl-2-(6-{[(pyridin-2-yl)amino]methyl}pyridazin-3-yl)-5-(trifluoromethyl)phenol

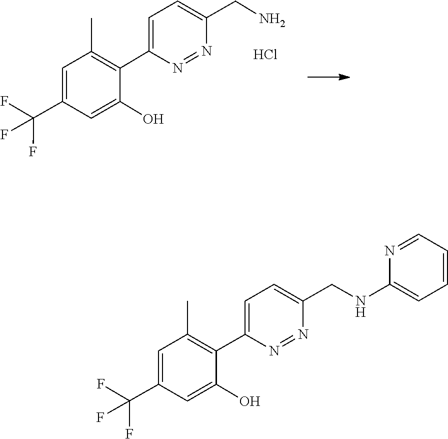 [SMILES: Cc1cc(C(F)(F)F)cc(O)c1-c1ccc(CN)nn1 ; Cl ; Cc1cc(C(F)(F)F)cc(O)c1-c1ccc(CNc2ccccn2)nn1]

To a solution of 2-[6-(aminomethyl)pyridazin-3-yl]-3-methyl-5-(trifluoromethyl)phenol hydrochloride (Intermediate C6. 80 mg, 250 mol) in DMSO (2 mL) were added 2-fluoropyridine (121 mg, 1.25 mmol), K₂CO₃ (208 mg, 1.50 mmol) and KI (4.2 mg, 25 mol) at 25° C. The mixture was stirred at 120° C. for 12 h under N₂. The reaction mixture was purified by prep-HPLC (column: Waters Xbridge Prep OBD C18 150×40 mm×10 μm; mobile phase: [H₂O (10 mM NH₄HCO₃)—ACN]; gradient: 20-50% B over 8.0 min) and lyophilised to give the title compound as a yellow oil, Y=36%. ¹H NMR (400 MHz, DMSO-d₆) δ 8.15-8.05 (m, 1H), 7.78-7.71 (m, 2H), 7.66 (s, 1H), 7.57 (d, J=9 Hz, 1H), 7.45 (s, 1H), 7.14-7.01 (m, 1H), 6.83 (d, J=9 Hz, 1H), 3.99 (s, 2H), 2.18 (s, 3H). ¹H NMR (400 MHz, DMSO-d₆+D₂O) δ 8.10-8.05 (m, 1H), 7.76-7.68 (m, 2H), 7.63 (s, 1H), 7.56 (d, J=9 Hz, 1H), 7.40 (s, H), 7.08-7.03 (m, 1H), 6.80 (d, J=9 Hz, 1H), 3.95 (s, 2H), 2.16 (s, 3H). LCMS (ESI): m/z: [M+H]⁺=361.2.

### Compound 79. 3-Methyl-2-(6-{[(pyridin-3-yl)amino]methyl}pyridazin-3-yl)-5-(trifluoromethyl)phenol

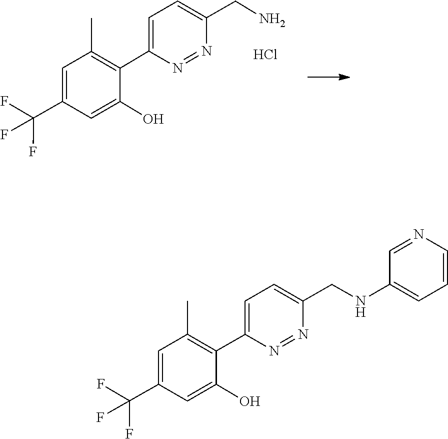 [SMILES: Cc1cc(C(F)(F)F)cc(O)c1-c1ccc(CN)nn1 ; Cl ; Cc1cc(C(F)(F)F)cc(O)c1-c1ccc(CNc2cccnc2)nn1]

To a solution of 2-[6-(aminomethyl)pyridazin-3-yl]-3-methyl-5-(trifluoromethyl)phenol hydrochloride (Intermediate C6, 80 mg, 225 mol) and 3-bromopyridine (71 mg, 449 mol) in DMSO (1.6 mL) were added Cu (1.4 mg, 22 mol) and caesium acetate (216 mg, 1.12 mmol) at 25° C. The mixture was stirred at 90° C. for 2 h under N₂ atmosphere. The reaction mixture was purified by prep-HPLC (column: Phenomenex Luna C18 100×40 mm×5 um; mobile phase: [H2O (0.04% HCl)—ACN]; gradient: 5-40% B over 8.0 min) and lyophilised to give the title compound as a yellow solid, Y=31%. 1H NMR (400 MHz, DMSO-d6) δ 10.40 (s, 1H), 8.25 (d, J=2 Hz, 1H), 8.09 (d, J=5 Hz, 1H), 8.02-7.70 (m, 5H), 7.16 (d, J=8 Hz, 2H), 4.83 (s, 2H), 2.08 (s, 3H). 1H NMR (400 MHz, DMSO-d6+D2O) δ 8.18 (d, J=2 Hz, 1H), 8.03 (d, J=5 Hz, 1H), 7.85-7.66 (m, 4H), 7.19-7.06 (m, 2H), 4.79 (s, 2H), 2.05 (s, 3H). LCMS (ESI): m/z: [M+H]+=361.2.

### Compound 80. 1-(1-{6-[2-Hydroxy-6-methyl-4-(trifluoromethyl)phenyl]pyridazin-3-yl}ethyl)pyrrolidin-2-one

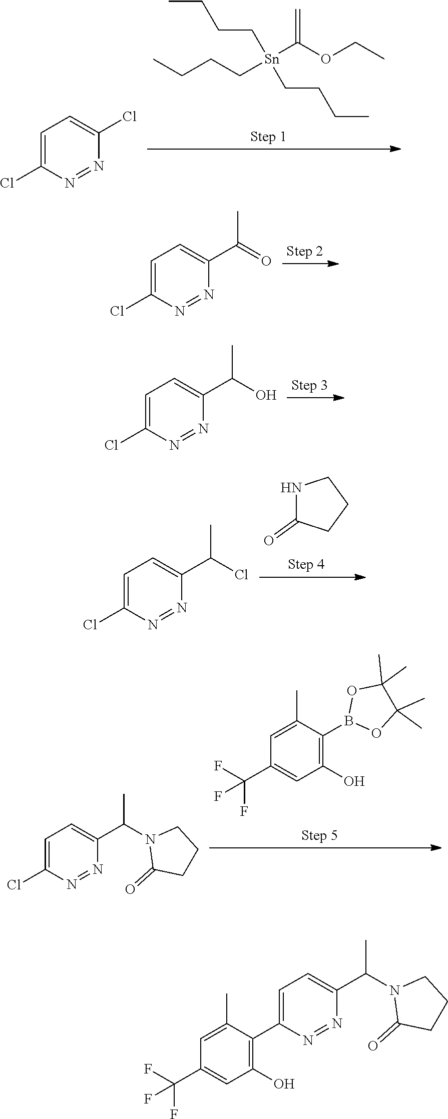

### Step 1. 1-(6-Chloropyridazin-3-yl)ethanone

To a solution of 3,6-dichloropyridazine (20 g, 134 mmol) in DMF (200 mL) were added PdCl₂(PPh₃)₂ (2.83 g, 4.03 mmol), tributyl(1-ethoxy vinyl)stannane (47.6 mL, 141 mmol) and TEA (20.54 mL, 148 mmol) at 25° C. under N₂ atmosphere. The mixture was stirred at 100° C. for 5 h under N₂ atmosphere, then concentrated under vacuum. The resulting residue was dissolved in THE (150 mL) and aqueous HCl (2 M in H₂O, 100 mL), and the resulting mixture stirred at 25° C. for 2 h. The mixture was diluted with saturated KF aqueous solution (100 mL) and extracted with EtOAc (3×200 mL). The combined organic layers were washed with brine (200 mL), dried over Na₂SO₄, filtered and concentrated under reduced pressure. The residue was purified by prep-HPLC (column: Waters Xbridge BEH C18 250×70 mm×10 μm; mobile phase: [H₂O (10 mM NH₄HCO₃)—ACN]; gradient: 1-30% B over 20 min) and lyophilised to give the title product as a yellow solid, Y=71%. ¹H NMR (400 MHz, CDCl₃) δ 8.11 (d, J=9 Hz, 1H), 7.67 (d, J=9 Hz, 1H), 2.88 (s, 3H).

### Step 2. 1-(6-Chloropyridazin-3-yl)ethanol

To a solution of 1-(6-chloropyridazin-3-yl)ethanone (5.0 g, 31.9 mmol) in MeOH (50 mL) was added NaBH₄ (1.21 g, 31.9 mmol, 1 eq) at 25° C. under N₂ atmosphere. The mixture was stirred at 25° C. under N₂ atmosphere for 3 h. The reaction mixture was quenched by addition of H₂O (50 mL) at 0° C. and extracted with DCM (3×50 mL). The combined organic layers were washed with brine (30 mL), dried over Na₂SO₄, filtered and concentrated under reduced pressure to give a residue. The residue was purified by column chromatography (SiO₂, petroleum ether/ethyl acetate=10/1 to 1/1, R_{f}=0.18) to give the title compound as a yellow oil, Y=53%.

### Step 3. 3-Chloro-6-(I-chloroethyl)pyridazine

To a solution of 1-(6-chloropyridazin-3-yl)ethanol (1.0 g, 6.31 mmol) in DCM (10 mL) at 0° C. was added SOCl₂ (1.83 mL, 25.2 mmol). The mixture was stirred at 25° C. for 2 h. The reaction mixture was quenched by addition of H₂O (10 mL) at 0° C., and the resulting mixture was extracted with DCM (3×10 mL). The combined organic layers were washed with brine (20 mL), dried over Na₂SO₄, filtered and concentrated under reduced pressure to give a residue. The residue was purified by column chromatography (SiO₂, petroleum ether/ethyl acetate=3/1 to 1/1, R_{f}=0.5) to give the title compound as a white solid, Y=61%. ¹H NMR (400 MHz, CDCl₃) δ 7.75 (d, J=9 Hz, 1H), 7.58 (d, J=9 Hz, 1H), 5.41 (q, J=7 Hz, 1H), 1.93 (d, J=7 Hz, 3H).

### Step 4. 1-[1-(6-Chloropyridazin-3-yl)ethyl]pyrrolidin-2-one

To a solution of pyrrolidin-2-one (0.50 g, 5.88 mmol) in DMF (20 mL) was added NaH (60% in mineral oil, 235 mg, 5.88 mmol) at 0° C. under N₂ atmosphere. The mixture was stirred at 0° C. under N₂ atmosphere for 0.5 h. The mixture was treated with 3-chloro-6-(1-chloroethyl)pyridazine (1.04 g, 5.87 mmol) at 0° C. under N₂ atmosphere. The resulting mixture was stirred at 25° C. for 2 h. The reaction mixture was quenched by addition of H₂O (10 mL) at 0° C., and extracted with EtOAc (3×10 mL). The combined organic layers were washed with brine (20 mL), dried over Na₂SO₄, filtered and concentrated under reduced pressure to give a residue. The residue was purified by prep-HPLC (column: Waters Xbridge Prep OBD C_{18 150}×40 mm×10 μm; mobile phase: [H₂O (10 mM NH₄HCO₃)—ACN]; gradient: 1-30% B over 8.0 min) and lyophilised to give the title compound as a white solid, Y=29%. ¹H NMR (400 MHz, DMSO-d₆) δ 7.89 (d, J=9 Hz, 1H), 7.68 (d, J=9 Hz, 1H), 5.40 (q, J=7 Hz, 1H), 3.49-3.38 (m, 1H), 3.24-3.16 (m, 1H), 2.31-2.19 (m, 2H), 1.99-1.89 (m, 2H), 1.56 (d, J=7 Hz, 3H).

### Step 5. 1-(1-{6-[2-Hydroxy-6-methyl-4-(trifluoromethyl)phenyl]pyridazin-3-yl}ethyl)pyrrolidin-2-one

To a solution of 1-[1-(6-chloropyridazin-3-yl)ethyl]pyrrolidin-2-one (100 mg, 443 mol) in dioxane (1.5 mL) and H₂O (0.5 mL) were added 3-methyl-2-(4,4,5,5-tetramethyl-1,3,2-dioxaborolan-2-yl)-5-(trifluoromethyl)phenol (Intermediate B1, 134 mg, 443 mol), Na₂CO₃ (141 mg, 1.33 mmol) and Pd(PPh₃)₄ (26 mg, 22 mol) at 25° C. The reaction mixture was degassed and purged with N₂ 3 times. The resulting mixture was stirred at 80° C. for 2 h under N₂ atmosphere. The reaction mixture was treated with H₂O (2 mL) and the resulting mixture extracted with EtOAc (3×2 mL). The combined organic layers were washed with brine (3 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [H₂O (10 mM NH₄HCO₃)—ACN]; gradient: 15-55% B over 8.0 min) and lyophilised to give the title compound as a white solid, Y=31%. ¹H NMR (400 MHz, DMSO-d₆) δ 10.20 (br. s, 1H), 7.72-7.60 (m, 2H), 7.16 (s, 1H), 7.10 (s, 1H), 5.47 (q, J=7 Hz, 1H), 3.53-3.47 (m, 1H), 3.29-3.23 (m, 1H), 2.35-2.29 (m, 2H), 2.08 (s, 3H), 2.02-1.93 (m, 2H), 1.63 (d, J=7 Hz, 3H). ¹H NMR (400 MHz, DMSO-d₆+D₂O) δ 7.68-7.61 (m, 2H), 7.14 (s, 1H), 7.08 (s, 1H), 5.43 (q, J=7 Hz, 1H), 3.53-3.49 (m, 1H), 3.29-3.22 (m, 1H), 2.34-2.29 (m, 2H), 2.05 (s, 3H), 2.00-1.91 (m, 2H), 1.60 (d, J=7 Hz, 3H). LCMS (ESI): m/z: [M+H]⁺=366.2.

### Compound 81. 1-(2-{6-[2-Hydroxy-6-methyl-4-(trifluoromethyl)phenyl]pyridazin-3-yl}propan-2-yl)pyrrolidin-2-one

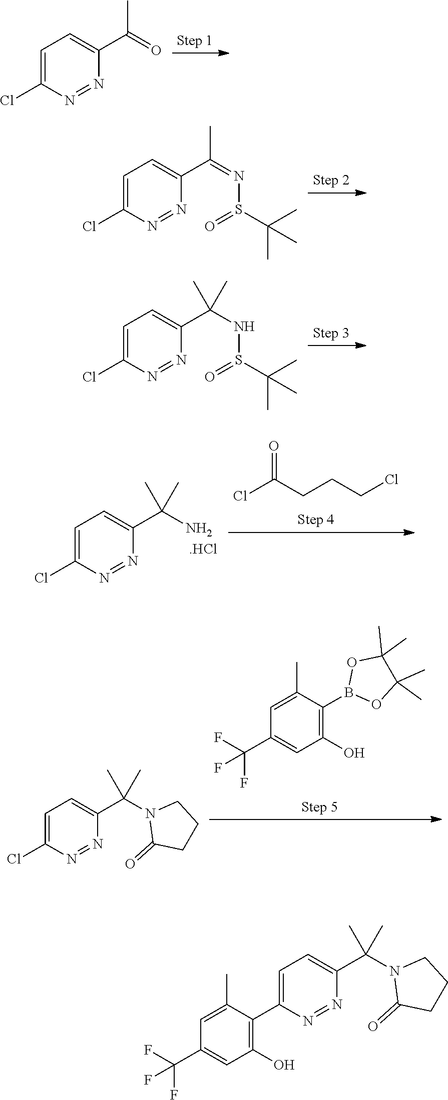

### Step 1. N-[1-(6-Chloropyridazin-3-yl)ethylidene]-2-methylpropane-2-sulfinamide

To a solution of 1-(6-chloropyridazin-3-yl)ethanone (for synthesis see Compound 80)(6.1 g, 39.0 mmol) in THE (50 mL) were added Ti(OEt)₄ (17.8 g, 77.9 mmol) and 2-methylpropane-2-sulfinamide (5.20 g, 42.9 mmol) at 25° C. The mixture was stirred at 60° C. for 1 h. The reaction mixture was quenched by addition of H₂O (50 mL) at 25° C. and extracted with EtOAc (3×60 mL). The combined organic layers were washed with brine (60 mL), dried over Na₂SO₄, filtered and concentrated under reduced pressure to give a residue. The residue was purified by column chromatography (SiO₂, petroleum ether/ethyl acetate=10/1 to 3/1, R_{f}═0.4) to give the title compound as a white solid, Y=44%. ¹H NMR (400 MHz, DMSO-d₆) δ 8.23 (d, J=9 Hz, 1H), 8.02 (d, J=9 Hz, 1H), 2.86 (s, 3H), 1.25 (s, 9H).

### Step 2. N-[1-(6-Chloropyridazin-3-yl)-1-methyl-ethyl]-2-methyl-propane-2-sulfinamide

To a solution of N-[1-(6-chloropyridazin-3-yl)ethylidene]-2-methylpropane-2-sulfinamide (1.0 g, 3.85 mmol) in THE (5 mL) at −20° C. under N₂ atmosphere was added methylmagnesium bromide (3 M in diethyl ether, 5.13 mL, 15.4 mmol). The mixture was stirred at 0° C. for 2 h. The reaction mixture was quenched by addition of H₂O (10 mL) at 0° C. and extracted with EtOAc (3×10 mL). The combined organic layers were washed with brine (10 mL), dried over Na₂SO₄, filtered and concentrated under reduced pressure to give a residue. The residue was purified by column chromatography (SiO₂, petroleum ether/ethyl acetate=10/1 to 3/1, R_{f}=0.1) to give the title compound as a yellow solid, Y=47%. ¹H NMR (400 MHz, DMSO-d₆) δ 8.03 (d, J=9 Hz, 1H), 7.91 (d, J=9 Hz, 1H), 5.79 (s, 1H), 1.65 (d, J=9 Hz, 6H), 1.13 (s, 9H).

### Step 3. 2-(6-Chloropyridazin-3-yl)propan-2-amine hydrochloride

A mixture of N-[1-(6-chloropyridazin-3-yl)-1-methyl-ethyl]-2-methyl-propane-2-sulfinamide (0.40 g, 1.45 mmol) and 4 M HCl in EtOAc (4 mL) was stirred at 25° C. for 1 h. The reaction mixture was concentrated under reduced pressure to give the title compound as a yellow solid, Y=99%.

### Step 4. 1-[1-(6-Chloropyridazin-3-yl)-1-methyl-ethyl]pyrrolidin-2-one

To a solution of 2-(6-chloropyridazin-3-yl)propan-2-amine hydrochloride (0.40 g, 1.92 mmol) in DCM (5 mL) at 0° C. were added NaOH (92 mg, 2.31 mmol) and 4-chlorobutanoyl chloride (215 μL, 1.92 mmol). The mixture was stirred at 0° C. for 1 h. The reaction mixture was treated with H₂O (3 mL) and the resulting mixture extracted with DCM (3×3 mL). The combined organic layers were washed with brine (3 mL), dried over Na₂SO₄, filtered and concentrated under reduced pressure to give a residue. The residue was dissolved in DMF (5 mL) at 0° C. under N₂ and treated with NaH (60% in mineral oil, 154 mg, 3.84 mmol). The mixture was stirred at 25° C. under N₂ for 1 h. The reaction mixture was quenched with H₂O (5 mL) at 0° C. and extracted with EtOAc (3×5 mL). The combined organic layers were washed with brine (5 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [H₂O (10 mM NH₄HCO₃)—ACN]; gradient: 1-30% B over 8.0 min) and lyophilised to give the title compound as a white solid, Y=54%. ¹H NMR (400 MHz, DMSO-d₆) δ 7.82-7.76 (m, 1H), 7.75-7.66 (m, 1H), 3.66 (t, J=7 Hz, 2H), 2.24-2.12 (m, 2H), 2.03-1.95 (m, 2H), 1.62 (s, 6H).

### Step 5. 1-(2-{6-[2-Hydroxy-6-methyl-4-(trifluoromethyl)phenyl]pyridazin-3-yl}propan-2-yl)pyrrolidin-2-one

To a solution of 1-[1-(6-chloropyridazin-3-yl)-1-methyl-ethyl]pyrrolidin-2-one (70 mg, 292 mol) in dioxane (2.1 mL) and H₂O (0.7 mL) at 25° C. under N₂ were added 3-methyl-2-(4,4,5,5-tetramethyl-1,3,2-dioxaborolan-2-yl)-5-(trifluoromethyl)phenol (Intermediate B1, 88 mg, 292 mol), Na₂CO₃ (93 mg, 876 mol) and Pd(PPh₃)₄ (17 mg, 14.6 mol). The mixture was stirred at 80° C. for 2 h under N₂ atmosphere. The reaction mixture was treated with H₂O (2 mL) and the resulting mixture extracted with EtOAc (3×2 mL). The combined organic layers were washed with brine (3 mL), dried over Na₂SO₄, filtered and concentrated under reduced pressure to give a residue. The residue was purified by prep-HPLC (column: Waters Xbridge Prep OBD C18 150×40 mm×10 μm; mobile phase: [H₂O (10 mM NH₄HCO₃)—ACN]; gradient: 15-55% B over 8.0 min) and lyophilised to give the title compound as a white solid, Y=45%. ¹H NMR (400 MHz, DMSO-d₆) δ 10.28 (s, 1H), 7.67 (d, J=9 Hz, 1H), 7.58 (d, J=9 Hz, 1H), 7.16 (s, 1H), 7.12 (s, 1H), 3.75-3.65 (m, 2H), 2.29-2.20 (m, 2H), 2.08 (s, 3H), 2.06-1.97 (m, 2H), 1.71 (s, 6H). ¹H NMR (400 MHz, DMSO-d₆+D₂O) δ 7.67 (d, J=9 Hz, 1H), 7.55 (d, J=9 Hz, 1H), 7.14 (s, 1H), 7.08 (s, 1H), 3.75-3.65 (m, 2H), 2.25-2.21 (m, 2H), 2.04 (s, 3H), 2.00-1.97 (m, 2H), 1.67 (s, 6H). LCMS (ESI): m/z: [M+H]⁺=380.3.

### Compound 82. 3-Methyl-2-(6-{[(1-methyl-1H-imidazol-2-yl)amino]methyl}pyridazin-3-yl)-5-(trifluoromethyl)phenol hydrochloride

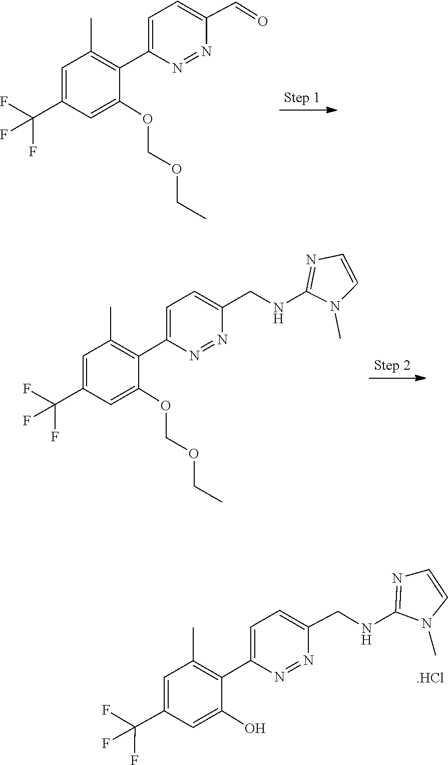 [SMILES: CCOCOc1cc(C(F)(F)F)cc(C)c1-c1ccc(C=O)nn1 ; CCOCOc1cc(C(F)(F)F)cc(C)c1-c1ccc(CNc2nccn2C)nn1 ; Cc1cc(C(F)(F)F)cc(O)c1-c1ccc(CNc2nccn2C)nn1 ; ".HCl"]

Synthesised in an analogous way to Compound 76 using 1-methylimidazol-2-amine. ¹H NMR (400 MHz, DMSO-d₆) δ 12.75 (s, 1H), 10.46 (s, 1H), 8.75 (t, J=6 Hz, 1H), 7.84 (d, J=8 Hz, 1H), 7.76 (d, J=8 Hz, 1H), 7.17 (s, 2H), 7.12-7.03 (m, 2H), 4.93 (d, J=6 Hz, 2H), 3.55 (s, 3H), 2.09 (s, 3H). ¹H NMR (400 MHz, DMSO-d₆+D₂O) δ 8.72 (t, J=6 Hz, 0.1H), 7.83 (d, J=8 Hz, 1H), 7.75 (d, J=8 Hz, 1H), 7.17-7.14 (m, 2H), 7.10-7.05 (m, 2H), 4.90 (s, 2H), 3.54 (s, 3H), 2.08 (s, 3H). LCMS (ESI): m/z: [M+H]⁺=364.2.

### Compound 83. 3-Methyl-2-(6-{[(5-methyl-1,3-thiazol-2-yl)amino]methyl}pyridazin-3-yl)-5-(trifluoromethyl)phenol

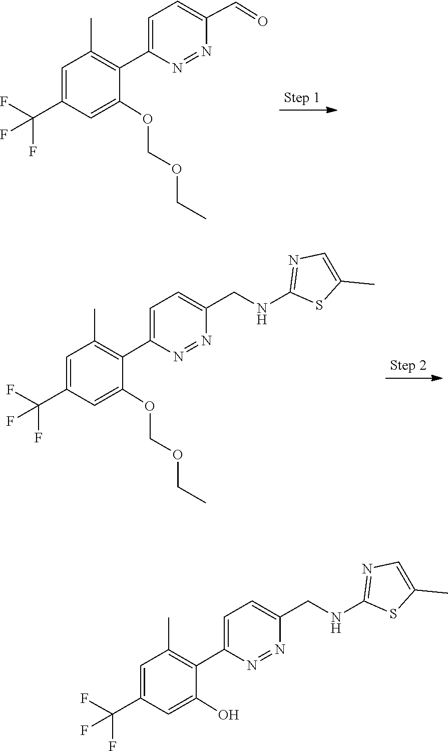 [SMILES: CCOCOc1cc(C(F)(F)F)cc(C)c1-c1ccc(C=O)nn1 ; CCOCOc1cc(C(F)(F)F)cc(C)c1-c1ccc(CNc2ncc(C)s2)nn1 ; Cc1cnc(NCc2ccc(-c3c(C)cc(C(F)(F)F)cc3O)nn2)s1]

Synthesised in an analogous way to Compound 76 using 5-methylthiazol-2-amine. ¹H NMR (400 MHz, DMSO-d₆) δ 10.87-10.16 (m, 2H), 7.84 (d, J=8 Hz, 1H), 7.78 (d, J=8 Hz, 1H), 7.25-7.17 (m, 3H), 5.03 (s, 2H), 2.27 (s, 3H), 2.09 (s, 3H). ¹H NMR (400 MHz, DMSO-d₆+D₂O) δ 7.83 (d, J=8 Hz, 1H), 7.78 (d, J=8 Hz, 1H), 7.20-7.17 (m, 2H), 7.14 (s, 1H), 5.00 (s, 2H), 2.26 (s, 3H), 2.08 (s, 3H). LCMS (ESI): m/z: [M+H]⁺=381.1.

### Compound 84. 3-Methyl-2-(6-{[(3-methyl-1,2-thiazol-5-yl)amino]methyl}pyridazin-3-yl)-5-(trifluoromethyl)phenol

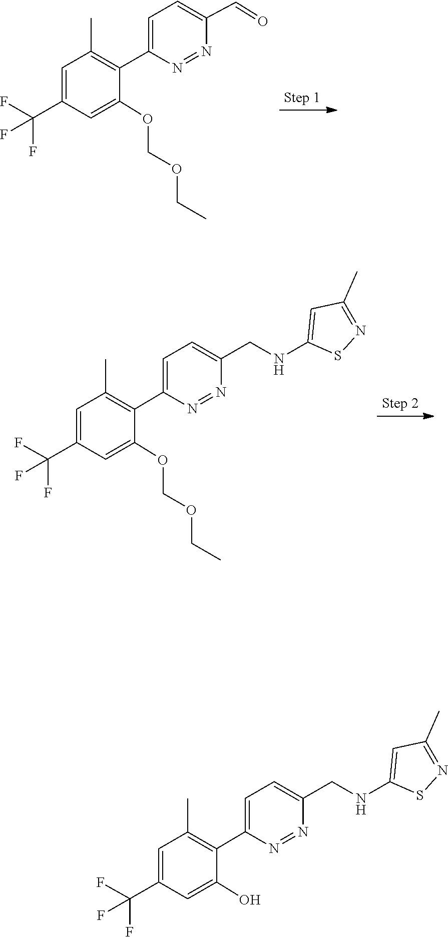 [SMILES: CCOCOc1cc(C(F)(F)F)cc(C)c1-c1ccc(C=O)nn1 ; CCOCOc1cc(C(F)(F)F)cc(C)c1-c1ccc(CNc2cc(C)ns2)nn1 ; Cc1cc(NCc2ccc(-c3c(C)cc(C(F)(F)F)cc3O)nn2)sn1]

Synthesised in an analogous way to Compound 76 using 3-methylisothiazol-5-amine. ¹H NMR (400 MHz, DMSO-d₆) δ 10.47 (br. s, 1H), 7.78 (s, 2H), 7.17 (s, 2H), 6.30 (s, 1H), 4.77 (s, 2H), 2.26 (s, 3H), 2.09 (s, 3H). ¹H NMR (400 MHz, DMSO-d₆+D₂O) δ 7.83-7.71 (m, 2H), 7.16-7.12 (m, 2H), 6.29 (s, 0.1H), 4.75 (s, 2H), 2.25 (s, 3H), 2.07 (s, 3H). LC-MS (ESI): m/z: [M+H]⁺=381.3.

### Compound 85. 2-(6-{[(Oxan-4-yl)amino]methyl}pyridazin-3-yl)-3,5-bis(trifluoromethyl)phenol

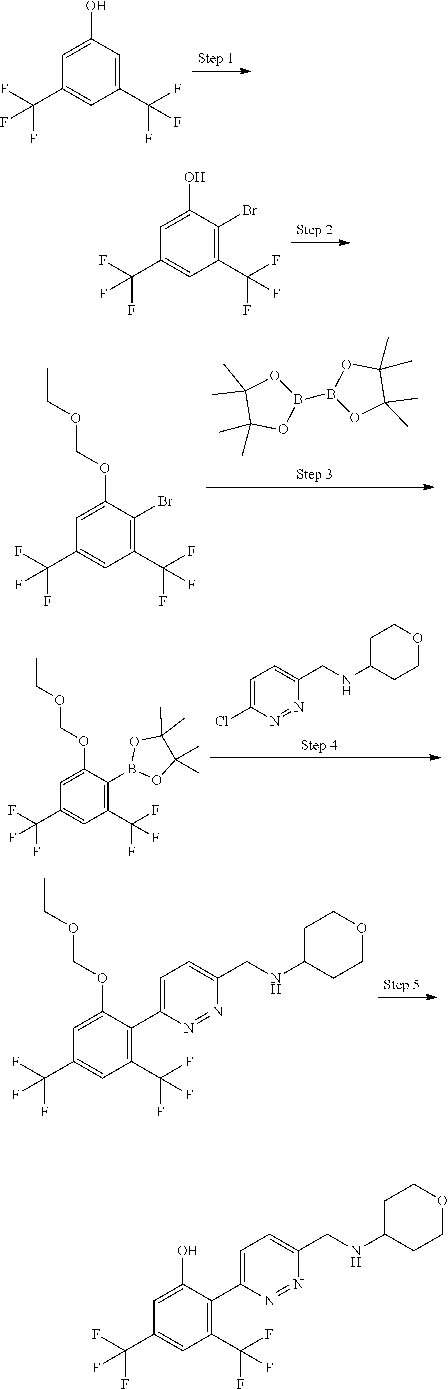

### Step 1. 2-Bromo-3,5-bis(trifiuoromethyl)phenol

A mixture of 3,5-bis(trifluoromethyl)phenol (3.0 g, 13.0 mmol), NBS (2.09 g, 11.7 mmol) and triflic acid (2.93 g, 19.6 mmol) was stirred at 25° C. for 3 h. The reaction mixture was diluted with H₂O (40 mL) and the resulting mixture extracted with EtOAc (3×40 mL). The combined organic layers were washed with brine (3×40 mL), dried over Na₂SO₄, filtered and concentrated under reduced pressure to give a residue. The residue was purified by prep-IPLC (column: Phenomenex Luna C18 250 mm×100 mm×10 μm; mobile phase: [H₂O (10 M N H₄HCO₃)—ACN]; gradient: 0-30% B over 20 m in) and lyophilised to give the title compound as a yellow oil, Y=50%. ¹H NMR (400 MHz, DMSO-d₆) δ 11.79 (s, 1H), 7.54-7.41 (in, 2H).

### Step 2. 2-Bromo-1-(ethoxymethoxy)-3,5-bis(trifluoromethyl)benzene

To a solution of 2-bromo-3,5-bis(trifluoromethyl)phenol (5.40 g, 17.5 mmol) in ACN (54 mL) was added Cs₂CO₃ (11.4 g, 35.0 mmol) and chloromethoxyethane (2.11 mL, 22.7 mmol) at 25° C. The mixture was stirred at 25° C. for 12 h. The reaction mixture was filtered and the filtrate concentrated under reduced pressure to give a residue. The residue was purified by prep-HPLC (column: Welch Xtimate C18 250×100 mm×10 μm; mobile phase: [H₂O (10 mM NH₄HCO₃)—ACN]; gradient: 50-85% B over 20 min) and lyophilised to give the title compound as a yellow oil, Y=450%. ¹H NMR (400 MHz, DMSO-d₆) δ 7.81 (s, 1H), 7.72 (s, 1H), 5.55 (s, 2H), 3.78-3.65 (m, 2H), 1.21-1.05 (in, 3H).

### Step 3. 2-[2-(Ethoxymethoxy)-4,6-bis(trifluoromethyl)phenyl]-4,4,5,5-tetramethyl-1,3,2-dioxaborolane

To a solution of 2-bromo-1-(ethoxymethoxy)-3,5-bis(trifluoromethyl)benzene (5.0 g, 13.6 mmol) in dioxane (70 mL) at 25° C. under N₂ atmosphere were added 4,4,5,5-tetramethyl-2-(4,4,5,5-tetramethyl-1,3,2-dioxaborolan-2-yl)-1,3,2-dioxaborolane (5.19 g, 20.4 mmol), Pd(PPh₃)₂Cl₂ (478 mg, 681 mol) and potassium acetate (5.35 g, 54.5 mmol). The mixture was stirred at 105° C. for 12 h under N₂ atmosphere. The reaction mixture was diluted with H₂O (50 mL) and the resulting mixture extracted with EtOAc (3×50 mL). The combined organic layers were washed with brine (3×50 mL), dried over Na₂SO₄, filtered and concentrated under reduced pressure to give a residue. The residue was purified by prep-TLC (SiO₂, petroleum ether:ethyl acetate=10:1, R_{f}=0.43) to give the title compound as a colorless oil, Y=27%.

### Step 4. N-[[6-[2-(Ethoxymethoxy)-4,6-bis(trifluoromethyl)phenyl]pyridazin-3-yl]methyl]tetrahydropyran-4-amine

To a solution of 2-[2-(ethoxymethoxy)-4,6-bis(trifluoromethyl)phenyl]-4,4,5,5-tetramethyl-1,3,2-dioxaborolane (300 mg, 724 mol) in dioxane (12 mL) and H₂O (3 mL) at 25° C. under N₂ atmosphere were added N-[(6-chloropyridazin-3-yl)methyl]tetrahydropyran-4-amine (Intermediate A1, 247 mg, 1.09 mmol), SPhos Pd G3 (85 mg, 109 mol) and K₂CO₃ (400 mg, 2.90 mmol). The mixture was stirred at reflux for 3 h. The reaction mixture was diluted with H₂O (3 mL) and the resulting mixture was extracted with EtOAc (3×3 mL). The combined organic layers were washed with brine (3 mL), dried over Na₂SO₄, filtered and concentrated under reduced pressure to give a residue. The residue was purified by prep-HPLC (column: Waters Xbridge BEH C18 100×30 mm×10 μm; mobile phase: [H₂O (10 mM NH₄HCO₃)—ACN]; gradient: 45-65% B over 8.0 min) and lyophilised to give the title compound as a yellow oil, Y=32%. ¹H NMR (400 MHz, DMSO-d₆) δ 7.91 (s, 1H), 7.89-7.86 (m, 1H), 7.83 (s, 1H), 7.80-7.76 (m, 1H), 5.34 (s, 2H), 4.10 (s, 2H), 3.92-3.75 (m, 2H), 3.45 (q, J=7 Hz, 2H), 3.29-3.24 (m, 2H), 2.69-2.61 (m, 1H), 1.85-1.75 (m, 2H), 1.39-1.22 (m, 2H), 1.02 (t, J=7 Hz, 3H).

### Step 5. 2-(6-{[(Oxan-4-yl)amino]methyl}pyridazin-3-yl)-3,5-bis(trifluoromethyl)phenol

A mixture of N-[[6-[2-(ethoxymethoxy)-4,6-bis(trifluoromethyl)phenyl]pyridazin-3-yl]methyl]tetrahydropyran-4-amine (110 mg, 229 mol) and 4 M HCl in EtOAc (4 mL) was stirred at 25° C. for 2 h. The reaction mixture was concentrated under reduced pressure to give a residue. The residue was purified by prep-HPLC (column: Waters Xbridge BEH C18 100×30 mm×10 μm; mobile phase: [H₂O (10 mM NH₄HCO₃)—ACN]; gradient: 5-60% B over 8.0 min) and lyophilised to give the title compound as a white solid, Y=46%. ¹H NMR (400 MHz, DMSO-d₆) δ 7.83 (d, J=9 Hz, 1H), 7.73 (d, J=9 Hz, 1H), 7.55 (s, 1H), 7.50 (s, 1H), 4.11 (s, 2H), 3.87-3.81 (m, 2H), 3.28-3.25 (m, 2H), 2.73-2.65 (m, 1H), 1.83-1.80 (m, 2H), 1.39-1.26 (m, 2H). ¹H NMR (400 MHz, DMSO-d₆+D₂O) δ 7.83 (d, J=9 Hz, 1H), 7.72 (d, J=9 Hz, 1H), 7.54 (s, 1H), 7.50 (s, 1H), 4.10 (s, 2H), 3.87-3.80 (m, 2H), 3.30-3.24 (m, 2H), 2.72-2.64 (m, 1H), 1.83-1.80 (m, 2H), 1.39-1.25 (m, 2H). LC-MS (ESI): m/z: [M+H]⁺=422.4.

### Compound 86. 1-(1-{6-[2-Hydroxy-6-methyl-4-(trifluoromethyl)phenyl]pyridazin-3-yl}cyclopropyl)pyrrolidin-2-one

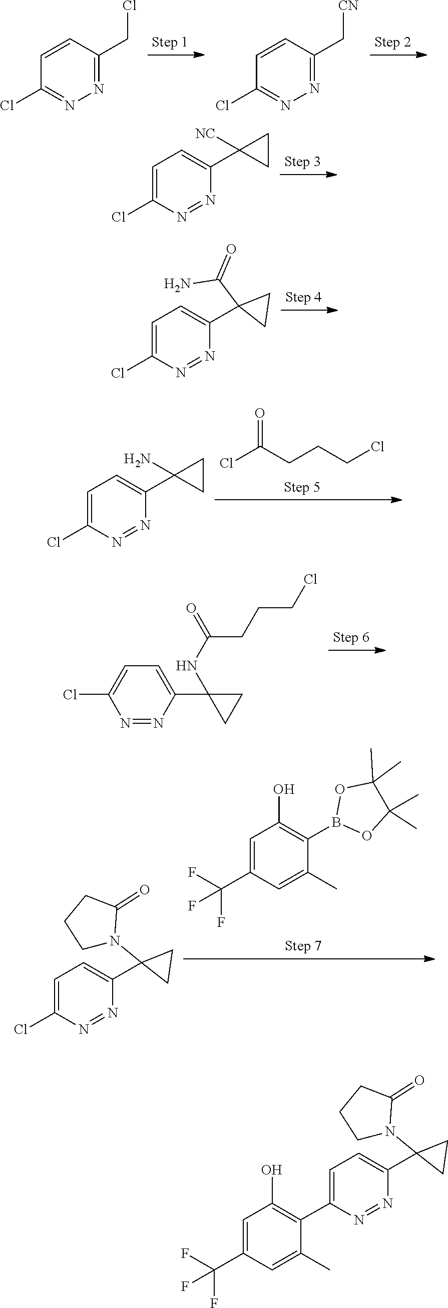

### Step 1. 2-(6-Chloropyridazin-3-yl)acetonitrile

To a solution of 3-chloro-6-(chloromethyl)pyridazine (6.5 g, 39.9 mmol) in ACN (65 mL) at 0° C. were added trimethylsilylcyanide (9.98 mL, 79.8 mmol) and 1 M TBAF (39.9 mL, 39.9 mmol). The mixture was stirred at 25° C. for 12 h. The reaction mixture was quenched by addition of saturated NH₄HCO₃ aqueous solution (70 mL) and the resulting mixture extracted with EtOAc (3×70 mL). The combined organic layers were washed with brine (2×70 mL), dried over Na₂SO₄, filtered and concentrated under reduced pressure to give a residue. The residue was purified by column chromatography (SiO₂, petroleum ether/ethyl acetate=1:1, R_{f}=0.50) to give the title compound as a white solid, Y=41%. ¹H NMR (400 MHz, chloroform-d) δ 7.68 (d, J=9 Hz, 1H), 7.62 (d, J=9 Hz, 1H), 4.20 (s, 2H).

### Step 2. 1-(6-Chloropyridazin-3-yl)cyclopropanecarbonitrile

To a solution of 2-(6-chloropyridazin-3-yl)acetonitrile (1.3 g, 8.47 mmol) in DMF (20 mL) at 0° C. under N₂ was added NaH (60% in mineral oil, 846 mg, 21.2 mmol). The mixture was stirred at 0° C. for 0.5 h under N₂, then treated with 1,2-dibromoethane (1.28 mL, 16.9 mmol) at 0° C. under N₂. The resulting mixture was stirred at 0° C. for 0.5 h under N₂. The mixture was quenched by saturated NH₄Cl aqueous solution (20 mL) and extracted with ethyl acetate (3×20 mL). The combined organic layers were washed with brine (20 mL), dried over anhydrous Na₂SO₄, filtered and concentrated under reduced pressure to give a residue. The residue was purified by column chromatography (SiO₂, petroleum ether:ethyl acetate=10:1 to 1:1, R_{f}═0.50) to give the title compound as a solid, Y=66%. ¹H NMR (400 MHz, chloroform-d) δ 7.88 (d, J=9 Hz, 1H), 7.54 (d, J=9 Hz, 1H), 2.16-2.08 (m, 2H), 1.95-1.89 (m, 2H).

### Step 3. 1-(6-chloropyridazin-3-yl)cyclopropanecarboxamide

A mixture of 1-(6-chloropyridazin-3-yl)cyclopropanecarbonitrile (1.0 g, 5.57 mmol) and conc. H₂SO₄ (20 mL) was stirred at 85° C. for 1 h. The solution was poured into saturated NaHCO₃ aqueous solution (20 mL) and the resulting mixture extracted with ethyl acetate (3×20 mL). The combined organic layers were washed with brine (20 mL), dried over anhydrous Na₂SO₄, filtered and concentrated under reduced pressure to give a residue. The residue was purified by column chromatography (SiO₂, petroleum ether:ethyl acetate=10:1 to 0:1, R_{f}=0.20) to give the title compound as a yellow solid, Y=86%. ¹H NMR (400 MHz, DMSO-d₆) δ 7.85-7.74 (m, 2H), 7.19-7.01 (m, 2H), 1.50-1.45 (m, 2H), 1.35-1.30 (m, 2H).

### Step 4. 1-(6-Chloropyridazin-3-yl)cyclopropanamine

To a solution of 1-(6-chloropyridazin-3-yl)cyclopropanecarboxamide (850 mg, 4.30 mmol) in ACN (20 mL) at 0° C. was added 10% NaClO aqueous solution (3.19 mL, 5.16 mmol) and 3 M NaOH aqueous solution (4.01 mL 12.0 mmol). The resulting mixture was stirred at 70° C. for 1 h. The solution was concentrated under reduced pressure to give a residue. The residue was purified by prep-HPLC (column: Waters Xbridge Prep OBD C18 150×40 mm×10 μm; mobile phase: [H₂O (10 mM NH₄HCO₃)—ACN]; gradient: 1-25% B over 8.0 min) and lyophilised to give the title compound as a white solid, Y=52%. ¹H NMR (400 MHz, DMSO-d₆) δ 8.02 (d, J=9 Hz, 1H), 7.78 (d, J=9 Hz, 1H), 1.33-1.27 (m, 2H), 1.15-1.08 (m, 2H).

### Step 5. 4-Chloro-N-[1-(6-chloropyridazin-3-yl)cyclopropyl]butanamide

To a solution of 1-(6-chloropyridazin-3-yl)cyclopropanamine (140 mg, 825 mol) in DCM (3 mL) at 0° C. were added NaOH (39.6 mg, 991 mol) and 4-chlorobutanoyl chloride (92.37 μL, 825 mol). The resulting mixture was stirred at 0° C. for 1 h. The solution was diluted with H₂O (3 mL) and extracted with DCM (3×3 mL). The combined organic layers were washed with brine (3 mL), dried over anhydrous Na₂SO₄, filtered and concentrated under reduced pressure to give the title compound as a yellow oil, Y=97%. ¹H NMR (400 MHz, chloroform-d) δ 7.47-7.38 (m, 2H), 6.53-6.42 (m, 1H), 3.65 (t, J=7 Hz, 2H), 2.47 (t, J=7 Hz, 2H), 2.18-2.11 (m, 2H), 1.83-1.77 (m, 2H), 1.42-1.36 (m, 2H).

### Step 6. 1-[1-(6-Chloropyridazin-3-yl)cyclopropyl]pyrrolidin-2-one

To a solution of 4-chloro-N-[1-(6-chloropyridazin-3-yl)cyclopropyl]butanamide (220 mg, 802 mol) in DMF (3 mL) at 0° C. was added NaH (60% in mineral oil, 64 mg, 1.60 mmol).

The resulting mixture was stirred at 0° C. under N₂ for 1 h. The solution was quenched by saturated NH₄Cl aqueous solution (3 mL) and extracted with ethyl acetate (3×3 mL). The combined organic layers were washed with brine (3 mL), dried over anhydrous Na₂SO₄, filtered and concentrated under reduced pressure to give the title compound as a yellow oil, Y=quantitative.

### Step 7. 1-(1-{6-[2-Hydroxy-6-methyl-4-(trifluoromethyl)phenyl]pyridazin-3-yl}cyclopropyl)pyrrolidin-2-one

To a solution of 1-[1-(6-chloropyridazin-3-yl)cyclopropyl]pyrrolidin-2-one (190 mg, 799 mol) in dioxane (3 mL) and H₂O (1 mL) at 25° C. under N₂ were added 3-methyl-2-(4,4,5,5-tetramethyl-1,3,2-dioxaborolan-2-yl)-5-(trifluoromethyl)phenol (Intermediate B1, 290 mg, 959 mol), Pd(dppf)Cl₂ (58 mg, 79.9 mol) and Na₂CO₃ (254 mg, 2.40 mmol). The resulting mixture was stirred at 85° C. for 1 h under N₂. The solution was diluted with H₂O (5 mL) and extracted with ethyl acetate (3×5 mL). The combined organic layers were washed with brine (5 mL), dried over anhydrous Na₂SO₄, filtered and concentrated under reduced pressure to give a residue. The residue was purified by prep-HPLC (column: Phenomenex Luna C18 100×40 mm×3 μm; mobile phase: [H₂O (0.04% HCl)—ACN]; gradient: 5-45% B over 30 min) and lyophilised to give the title compound as a white solid, Y=15%. ¹H NMR (400 MHz, DMSO-d₆) δ 10.48 (br. s, 1H), 7.70 (d, J=9 Hz, 1H), 7.63 (d, J=9 Hz, 1H), 7.16 (s, 2H), 3.51 (t, J=7 Hz, 2H), 2.38 (t, J=7 Hz, 2H), 2.10 (s, 3H), 2.08-2.01 (m, 2H), 1.73-1.67 (m, 2H), 1.55-1.47 (m, 2H). ¹H NMR (400 MHz, DMSO-d₆+D₂O) δ 7.72 (d, J=9 Hz, 1H), 7.64 (d, J=9 Hz, 1H), 7.17 (s, 1H), 7.13 (s, 1H), 3.50 (t, J=7 Hz, 2H), 2.37 (t, J=7 Hz, 2H), 2.09 (s, 3H), 2.07-2.00 (m, 2H), 1.71-1.65 (m, 2H), 1.54-1.49 (m, 2H). LCMS (ESI): m/z: [M+H]⁺=378.1.

### Compound 87. (5S)-5-({[6-(4-Chloro-2-hydroxy-6-methylphenyl)pyridazin-3-yl]methyl}amino)-1-methylpiperidin-2-one

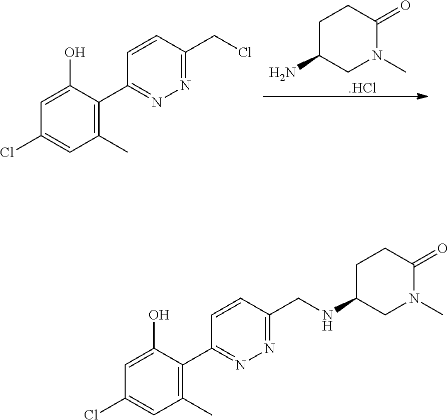

Synthesised in an analogous way to Compound 73 using (5S)-5-amino-1-methylpiperidin-2-one hydrochloride. ¹H NMR (400 MHz, DMSO-d₆) δ 9.97 (br. s, 1H), 7.76 (d, J=9 Hz, 1H), 7.61 (d, J=9 Hz, 1H), 6.87 (s, 1H), 6.85 (s, 1H), 4.18-3.98 (m, 2H), 3.44-3.40 (m, 1H), 3.13-3.06 (m, 1H), 3.02-2.93 (m, 1H), 2.76 (s, 3H), 2.73-2.64 (m, 1H), 2.36-2.29 (m, 1H), 2.22-2.11 (m, 1H), 2.01 (s, 3H), 1.98-1.87 (m, 1H), 1.74-1.63 (m, 1H). ¹H NMR (400 MHz, DMSO-d₆+D₂O) δ 7.76 (d, J=9 Hz, 1H), 7.60 (d, J=9 Hz, 1H), 6.87 (s, 1H), 6.84 (s, 1H), 4.11-4.00 (m, 2H), 3.49-3.33 (m, 1H), 3.12-3.01 (m, 1H), 3.00-2.90 (m, 1H), 2.75 (s, 3H), 2.37-2.27 (m, 1H), 2.20-2.10 (m, 1H), 1.99 (s, 3H), 1.96-1.88 (m, 1H), 1.73-1.62 (m, 1H). LCMS (ESI): m/z: [M+H]⁺=361.2.

### Compound 89. 1-{[6-(4-Chloro-2-hydroxy-6-methylphenyl)pyridazin-3-yl]methyl}pyrrolidine-2-thione

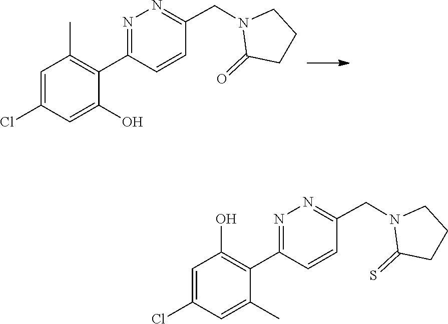 [SMILES: Cc1cc(Cl)cc(O)c1-c1ccc(CN2CCCC2=O)nn1 ; Cc1cc(Cl)cc(O)c1-c1ccc(CN2CCCC2=S)nn1]

To a solution of 1-[[6-(4-chloro-2-hydroxy-6-methyl-phenyl)pyridazin-3-yl]methyl]pyrrolidin-2-one (Compound 62, 65 mg, 205 mol) in DCM (1 mL) at 25° C. under N₂ was added Lawesson's reagent (83 mg, 205 mol). The mixture was stirred at 25° C. for 4 h.

The reaction mixture was concentrated under reduced pressure to give a residue. The residue was purified by prep-HPLC (column: Waters Xbridge BEH C18 100×30 mm×10 μm; mobile phase: [H₂O (10 mM NH₄HCO₃)—ACN]; gradient: 20-65% B over 8.0 min) and lyophilised to give the title compound as a white solid, Y=10%. ¹H NMR (400 MHz, DMSO-d₆) δ 10.00 (br. s, 1H), 7.76-7.50 (m, 2H), 6.89 (s, 1H), 6.86 (s, 1H), 5.27 (s, 2H), 3.87 (t, J=7 Hz, 2H), 2.99 (t, J=7 Hz, 2H), 2.15-1.90 (m, 5H). ¹H NMR (400 MHz, DMSO-d₆) δ 7.80-7.48 (m, 2H), 6.89 (s, 1H), 6.86 (s, 1H), 5.26 (s, 2H), 3.87 (t, J=7 Hz, 2H), 2.98 (t, J=7 Hz, 2H), 2.12-1.99 (m, 5H). LCMS (ESI): m/z: [M+H]⁺=334.1.

### Compound 90. (4S)-4-({[6-(4-Chloro-2-hydroxy-6-methylphenyl)pyridazin-3-yl]methyl}amino)-1-methylpyrrolidin-2-one

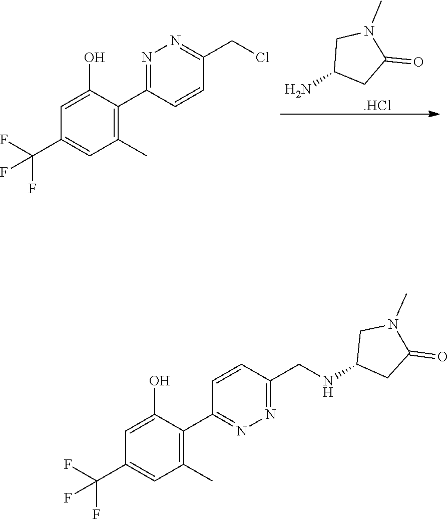

Synthesised in an analogous way to Compound 73 using (4S)-4-amino-1-methylpyrrolidin-2-one hydrochloride. ¹H NMR (400 MHz, DMSO-d₆) δ 10.06 (s, 1H), 7.74 (d, J=9 Hz, 1H), 7.61 (d, J=9 Hz, 1H), 6.88 (d, J=2 Hz, 1H), 6.85 (d, J=2 Hz, 1H), 4.07-3.92 (m, 2H), 3.55-3.48 (m, 1H), 3.46-3.35 (m, 1H), 3.20-3.10 (m, 1H), 2.91-2.79 (m, 1H), 2.70 (s, 3H), 2.48-2.43 (m, 1H), 2.15-2.05 (m, 1H), 2.01 (s, 3H). ¹H NMR (400 MHz, DMSO-d₆+D₂O) δ 7.74 (d, J=9 Hz, 1H), 7.60 (d, J=9 Hz, 1H), 6.87 (d, J=2 Hz, 1H), 6.84 (d, J=2 Hz, 1H), 4.05-3.94 (m, 2H), 3.51-3.48 (m, 1H), 3.44-3.36 (m, 1H), 3.20-3.10 (m, 1H), 2.70 (s, 3H), 2.48-2.43 (m, 1H), 2.20-2.05 (m, 1H), 1.99 (s, 3H). LCMS (ESI): m/z: [M+H]⁺=347.2.

### Compound 91. (4S)-4-({[6-(4-Chloro-2-hydroxy-6-methylphenyl)pyridazin-3-yl]methyl}(methyl)amino)-1-methylpyrrolidin-2-one

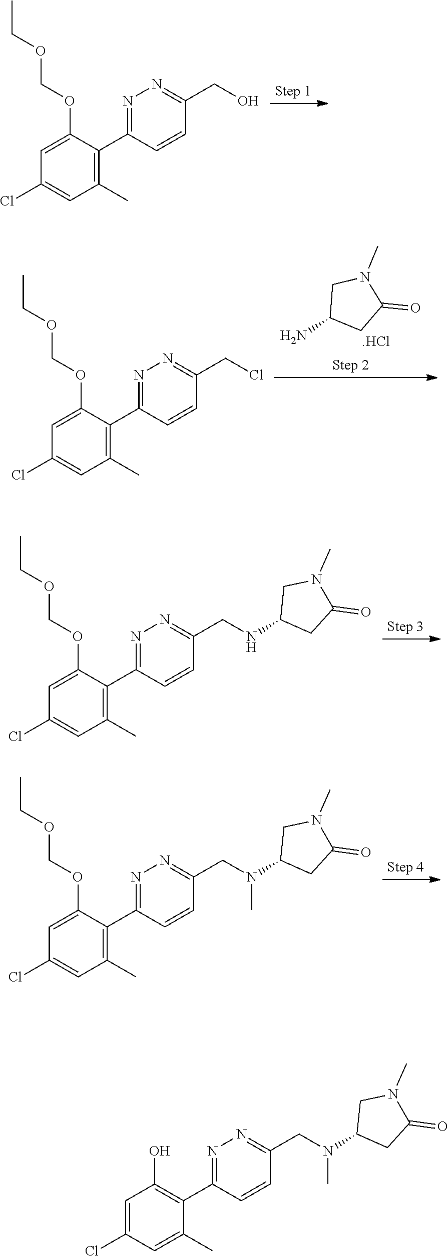

### Step 1. 3-[4-Chloro-2-(ethoxymethoxy)-6-methyl-phenyl]-6-(chloromethyl)pyridazine

To a solution of [6-[4-chloro-2-(ethoxymethoxy)-6-methyl-phenyl]pyridazin-3-yl]methanol (for synthesis see Intermediate C2)(0.70 g, 2.27 mmol) in DCM (7 mL) at 25° C. was added SOCl₂ (329 μL, 4.53 mmol). The mixture was stirred at 25° C. for 2 h under N₂. The reaction mixture was quenched by addition H₂O (7 mL) and extracted with DCM (3×7 mL). The combined organic layers were washed with brine (21 mL), dried over Na₂SO₄, filtered and concentrated under reduced pressure to give a residue. The residue was purified by column chromatography (SiO₂, petroleum ether/ethyl acetate=10/1 to 1/1, R_{f}═0.66) to give the title compound as a solid, Y=40%. ¹H NMR (400 MHz, DMSO-d₆) δ 7.93 (d, J 9 Hz, 1H), 7.75 (d, J 9 Hz, 1H), 7.19 (s, 1H), 7.13 (s, 1H), 5.17 (s, 2H), 5.07 (s, 2H), 3.45 (q, J 7 Hz, 2H), 2.02 (s, 3H), 1.03 (t, J 7 Hz, 3H).

### Step 2. (4S)-4-[[6-[4-Chloro-2-(ethoxymethoxy)-6-methyl-phenyl]pyridazin-3-yl]methylamino]-1-methyl-pyrrolidin-2-one

To a solution of (4S)-4-amino-1-methyl-pyrrolidin-2-one hydrochloride (150 mg, 1.0 mmol) in DMSO (5 mL) at 25° C. were added 3-[4-chloro-2-(ethoxymethoxy)-6-methyl-phenyl]-6-(chloromethyl) pyridazine (326 mg, 1.0 mmol) and DIPEA (347 μL 2.0 mmol). The resulting mixture was stirred at 80° C. for 2 h. The reaction mixture was purified by prep-HPLC (column: Waters Xbridge Prep OBD C18 150×40 mm×10 μm; mobile phase: [H₂O (10 mM NH₄HCO₃)—ACN]; gradient: 15-55% B over 8.0 min) and lyophilised to give the title compound as a yellow oil, Y=60%. ¹H NMR (400 MHz, DMSO-d₆) δ 7.77 (d, J=9 Hz, 1H), 7.62 (d, J=9 Hz, 1H), 7.18 (d, J=2 Hz, 1H), 7.11 (d, J=2 Hz, 1H), 5.16 (s, 2H), 4.06-4.02 (m, 2H), 3.55-3.40 (m, 4H), 3.31-3.29 (m, 1H), 3.20-3.10 (m, 1H), 2.70 (s, 3H), 2.46-2.42 (m, 1H), 2.15-2.05 (m, 1H), 2.00 (s, 3H), 1.17 (t, J=7 Hz, 3H).

### Step 3. (4S)-4-[[6-[4-Chloro-2-(ethoxymethoxy)-6-methyl-phenyl]pyridazin-3-yl]methyl-methylamino]-1-methyl-pyrrolidin-2-one

To a solution of (4S)-4-[[6-[4-chloro-2-(ethoxymethoxy)-6-methyl-phenyl]pyridazin-3-yl]methylamino]-1-methyl-pyrrolidin-2-one (200 mg, 494 mol) in DMF (3 mL) at 0° C. under N₂ was added NaH (60% in mineral oil, 40 mg, 0.99 mmol). The resulting mixture was stirred at 0° C. for 0.5 h under N₂, then treated with CH₃I (31 μL, 494 mol). The resulting mixture was stirred at 0° C. under N₂ for 2 h. The solution was quenched by saturated NH₄Cl aqueous solution (3 mL) and the resulting mixture was extracted with EtOAc (3×3 mL). The combined organic layers were washed with brine (3×3 mL), dried over anhydrous Na₂SO₄, filtered and concentrated under reduced pressure to give the title compound as a yellow oil, Y=97%.

### Step 4. (4S)-4-({[6-(4-Chloro-2-hydroxy-6-methylphenyl)pyridazin-3-yl]methyl}(methyl)amino)-1-methylpyrrolidin-2-one

A mixture of (4S)-4-[[6-[4-chloro-2-(ethoxymethoxy)-6-methyl-phenyl]pyridazin-3-yl]methyl-methyl-amino]-1-methyl-pyrrolidin-2-one (200 mg, 477 mol) and 4 M HCl in EtOAc (3 mL) was stirred at 25° C. for 1 h. The solution was concentrated under reduced pressure to give a residue. The residue was purified by prep-HPLC (column: Waters Xbridge BEH C18 100×30 mm×10 μm; mobile phase: [H₂O (10 mM NH₄HCO₃)—ACN]; gradient: 15-45% B over 8.0 min) and lyophilised to give the title compound as a yellow solid, Y=28%. ¹H NMR (400 MHz, DMSO-d₆) δ 10.02 (m, 1H), 7.73 (d, J=9 Hz, 1H), 7.63 (d, J=9 Hz, 1H), 6.88 (d, J=2 Hz, 1H), 6.85 (d, J=2 Hz, 1H), 3.89-3.76 (m, 2H), 3.60-3.50 (m, 1H), 3.43-3.38 (m, 1H), 3.31-3.29 (m, 1H), 2.73 (s, 3H), 2.49-2.42 (m, 1H), 2.35-2.27 (m, 1H), 2.11 (s, 3H), 2.01 (s, 3H). ¹H NMR (400 MHz, DMSO-d₆+D₂O) δ 7.74 (d, J=9 Hz, 1H), 7.61 (d, J=9 Hz, 1H), 6.87 (d, J=2 Hz, 1H), 6.84 (d, J=2 Hz, 1H), 3.88-3.75 (m, 2H), 3.60-3.50 (m, 1H), 3.43-3.35 (m, 1H), 3.34-3.28 (m, 1H), 2.72 (s, 3H), 2.48-2.43 (m, 1H), 2.34-2.27 (m, 1H), 2.08 (s, 3H), 1.99 (s, 3H). LCMS (ESI): m/z: [M+H]⁺=361.2.

### Compound 92. 3-Methyl-2-(6-(((pyrazin-2-ylmethyl)amino)methyl)pyridazin-3-yl)-5-(trifluoromethyl)phenol

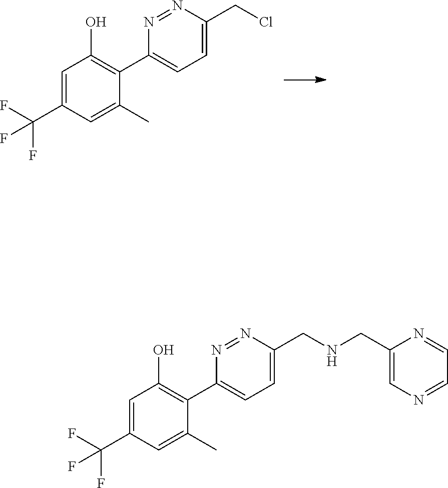 [SMILES: Cc1cc(C(F)(F)F)cc(O)c1-c1ccc(CCl)nn1 ; Cc1cc(C(F)(F)F)cc(O)c1-c1ccc(CNCc2cnccn2)nn1]

To a solution of 2-[6-(chloromethyl)pyridazin-3-yl]-3-methyl-5-(trifluoromethyl)phenol (Intermediate C1, 60 mg, 198 mol) in toluene (1 mL) was added TEA (41.4 μL, 297 mol) and pyrazin-2-ylmethanamine (32.5 mg, 297 mol) at 25° C. The mixture was stirred at 120° C. for 2 h then allowed to cool to room temperature. The reaction mixture was purified by prep-HPLC (column: Phenomenex Luna C18 100×40 mm, 5 μm; mobile phase: [(A) H₂O (0.04% HCl)—(B) ACN]; gradient: 10-40% B, 8 min) and lyophilised to give the title compound as a white solid, Y=18%. ¹H NMR (400 MHz, DMSO-d₆) δ 10.53 (br. s, 1H) 10.20 (br. s, 2H), 8.87 (d, J=1 Hz, 1H), 8.80-8.65 (m, 2H), 8.03-7.77 (m, 2H), 7.19 (s, 2H), 4.73 (s, 2H), 4.61 (s, 2H), 2.11 (s, 3H). LCMS (ESI): m/z [M+H]⁺=376.0.

The compounds in Table A were prepared by the following method.

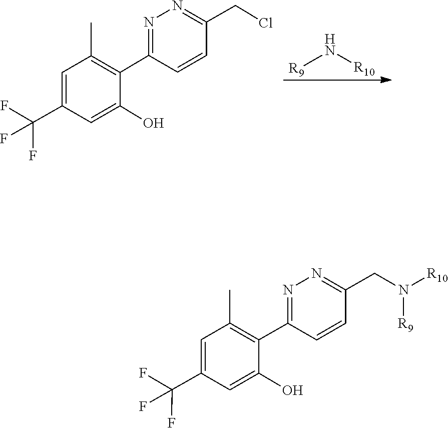

To a solution of 2-[6-(chloromethyl)pyridazin-3-yl]-3-methyl-5-(trifluoromethyl)phenol (Intermediate C, 50 mg, 165 mol) in DMSO (0.5 mL) was added the amine (2.48 mmol) and DIPEA (173 μL, 991 μmol) at 25° C. under N₂. The mixture was stirred at 120° C. for 2 h then purified directly by prep-HPLC and lyophilised to give the desired compound.

TABLE A

| Compound | | |
|---|---|---|
| No. | NMR Data | LCMS data |
| 88 | ¹H NMR (400 MHz, DMSO-d₆) δ 10.24 (br. s, 1H), 7.80 (d, | LCMS (ESI): |
| | J = 9 Hz, 1H), 7.67 (d, J = 9 Hz, 1H), 7.16 (s, 1H), 7.09 (s, | m/z: [M + H]⁺ = |
| | 1H), 4.18-4.03 (m, 2H), 3.50-3.35 (m, 1H), 3.15-3.05 (m, | 395.3. |
| | 1H), 3.02-2.95 (m, 1H), 2.76 (s, 3H), 2.74-2.66 (m, 1H), | |
| | 2.38-2.28 (m, 1H), 2.21-2.12 (m, 1H), 2.08 (s, 3H), 1.98-1.89 | |
| | (m, 1H), 1.74-1.63 (m, 1H). ¹H NMR (400 MHz, | |
| | DMSO-d₆ + D₂O) δ 7.79 (d, J = 9 Hz, 1H), 7.65 (d, J = 9 Hz, | |
| | 1H), 7.15 (s, 1H), 7.08 (s, 1H), 4.14-4.00 (m, 2H), 3.45-3.35 | |
| | (m, 1H), 3.15-3.03 (m, 1H), 3.01-2.91 (m, 1H), 2.75 | |
| | (s, 3H), 2.37-2.28 (m, 1H), 2.21-2.11 (m, 1H), 2.05 (s, | |
| | 3H), 1.97-1.87 (m, 1H), 1.73-1.62 (m, 1H). | |
| 93 | ¹H NMR (400 MHz, DMSO-d₆) δ 10.10 (br. s, 1H), 7.77 (d, | LC-MS (ESI): |
| | J = 9 Hz, 1H), 7.66 (d, J = 9 Hz, 1H), 7.16 (s, 1H), 7.09 (s, | m/z [M + H]⁺ = |
| | 1H), 3.98 (s, 2H), 2.34 (s, 3H), 2.08 (s, 3H) | 298.1. |
| 94 | ¹H NMR (400 MHz, DMSO-d₆) δ 10.27 (br. s, 1H), 7.76 (d, | LCMS (ESI): |
| | J = 9 Hz, 1H), 7.68 (d, J = 9 Hz, 1H), 7.16 (s, 1H), 7.10 (s, | m/z [M + H]⁺ = |
| | 1H), 3.77 (s, 2H), 2.24 (s, 6H), 2.08 (s, 3H) | 312.1. |
| 95 | ¹H NMR (400 MHz, DMSO-d₆) δ 10.27 (br. s, 1H), 7.80 (d, | LCMS (ESI): |
| | J = 9 Hz, 1H), 7.70 (d, J = 9 Hz, 1H), 7.16 (s, 1H), 7.10 (s, | m/z [M + H]⁺ = |
| | 1H), 4.16 (d, J = 7 Hz, 2H), 3.41-3.35 (m, 2H), 2.08 (s, 3H) | 366.0. |
| 96 | ¹H NMR (400 MHz, DMSO-d₆) δ 10.27 (br. s, 1H), 7.80 (d, | (ESI): m/z |
| | J = 9 Hz, 1H), 7.69 (d, J = 9 Hz, 1H), 7.16 (s, 1H), 7.10 (s, | [M + H]⁺ = |
| | 1H), 6.22-5.92 (m, 1H), 4.12 (d, J = 5 Hz, 2H), 3.02-2.91 | 348.0. |
| | (m, 3H), 2.08 (s, 3H) | |
| 97 | ¹H NMR (400 MHz, DMSO-d₆) δ 10.25 (br. s, 1H), 7.81 (d, | (ESI): m/z |
| | J = 9 Hz, 1H), 7.66 (d, J = 9 Hz, 1H), 7.15 (s, 1H), 7.09 (s, | [M + H]⁺ = |
| | 1H), 4.06 (s, 2H), 2.47-2.41 (m, 2H), 2.08 (s, 3H), 0.96-0.92 | 338.0. |
| | (m, 1H), 0.43-0.38 (m, 2H), 0.12-0.11 (m, 2H) | |
| 98 | ¹H NMR (400 MHz, DMSO-d₆) δ 7.79 (d, J = 9 Hz, 1H), | (ESI): m/z |
| | 7.67 (d, J = 9 Hz, 1H), 7.14 (s, 1H), 7.09 (s, 1H), 4.03 (s, | [M + H]⁺ = |
| | 2H), 2.58 (q, J = 7 Hz, 2H), 2.08 (s, 3H), 1.06 (t, J = 7 Hz, | 312.0. |
| | 3H) | |
| 99 | ¹H NMR (400 MHz, DMSO-d₆) δ 7.81 (d, J = 9 Hz, 1H), | (ESI): m/z |
| | 7.66 (d, J = 9 Hz, 1H), 7.15 (s, 1H), 7.10 (s, 1H), 4.04 (s, | [M + H]⁺ = |
| | 2H), 2.80-2.74 (m, 1H), 2.08 (s, 3H), 1.05 (d, J = 6 Hz, 6H) | 326.0. |
| 100 | ¹H NMR (400 MHz, DMSO-d₆) δ 10.19 (br. s, 1H), 7.81 (d, | (ESI): m/z |
| | J = 9 Hz, 1H), 7.65 (d, J = 9 Hz, 1H), 7.15 (s, 1H), 7.09 (s, | [M + H]⁺ = |
| | 1H), 4.06 (s, 2H), 2.44-2.43 (m, 1H), 2.07 (s, 3H), 1.96-1.82 | 366.1. |
| | (m, 2H), 1.76-1.60 (m, 2H) 1.59-1.48 (m, 1H), 1.19-1.07 | |
| | (m, 5H) | |
| 101 | ¹H NMR (400 MHz, DMSO-d₆) δ 10.25 (br. s, 1H), 7.80 (d, | (ESI): m/z: |
| | J = 9 Hz, 1H), 7.66 (d, J = 9 Hz, 1H), 7.15 (s, 1H), 7.09 (s, | [M + H]⁺ = |
| | 1H), 4.01 (s, 2H), 3.09-3.05 (m, 1H), 2.08 (s, 3H), 1.76-1.72 | 352.0. |
| | (m,2H), 1.65-1.64 (m, 2H), 1.48-1.47 (m, 2H), 1.38-1.37 | |
| | (m, 2H) | |
| 102 | ¹H NMR (400 MHz, DMSO-d₆) δ 10.25 (br. s, 1H), 7.81 (d, | (ESI): m/z |
| | J = 9 Hz, 1H), 7.67 (d, J = 9 Hz, 1H), 7.16 (s, 1H), 7.10 (s, | [M + H]+ = |
| | 1H), 4.08-4.06 (m, 2H), 3.86-3.80 (m, 1H), 3.71-3.68 (m, | 368.0. |
| | 1H), 3.30-3.27 (m, 1H), 3.08-3.03 (m, 1H), 2.54-2.52 (m, | |
| | 1H), 2.08 (s, 3H), 1.99-1.95 (m, 1H), 1.62-1.50 (m, 1H), | |
| | 1.47-1.41 (m, 1H), 1.32-1.25 (m, 1H) | |
| 103 | ¹H NMR (400 MHz, DMSO-d₆) δ 10.19 (br. s, 1H), 7.80 (d, | (ESI): m/z |
| | J = 9 Hz, 1H), 7.67 (d, J = 9 Hz, 1H), 7.16 (s, 1H), 7.10 (s, | [M + H]⁺ = |
| | 1H), 4.04-4.02 (m, 2H), 3.79-3.66 (m, 3H), 3.52-3.43 (m, | 354.0. |
| | 1H), 3.40-3.36 (m, 1H), 2.67-2.66 (m, 1H), 2.08 (s, 3H), | |
| | 2.00-1.95 (m, 1H), 1.73-1.72 (m, 1H) | |
| 104 | ¹H NMR (400 MHz, DMSO-d₆) δ 10.24 (br. s, 1H), 7.79 (d, | (ESI): m/z: |
| | J = 9 Hz, 1H), 7.68 (d, J = 9 Hz, 1H), 7.16 (s, 1H), 7.09 (s, | [M + H]⁺ = |
| | 1H), 4.70-4.60 (m, 2H), 4.28 (t, J = 7 Hz, 2H), 4.03 (s, 2H), | 354.1 |
| | 3.10-3.02 (m, 1H), 2.86-2.84 (d, J = 7 Hz, 2H), 2.08 (s, | |
| | 3H) | |
| 105 | ¹H NMR (400 MHz, DMSO-d₆) δ 10.09 (br. s, 1H), 7.80 (d, | (ESI): m/z: |
| | J = 9 Hz, 1H), 7.67 (d, J = 9 Hz, 1H), 7.21-7.13 (m, 2H), | [M + H]⁺ = |
| | 4.07 (s, 2H), 3.73-3.59 (m, 3H), 3.43-3.40 (m, 1H), 2.58-2.52 | 368.0 |
| | (m, 2H), 2.35-2.27 (m, 1H), 2.08 (s, 3H), 2.00-1.93 | |
| | (m, 1H), 1.54-1.52 (m, 1H). | |
| 106 | ¹H NMR (400 MHz, DMSO-d₆) δ 10.27 (br. s, 1H), 7.78 (d, | (ESI): m/z: |
| | J = 9 Hz, 1H), 7.66 (d, J = 9 Hz, 1H), 7.15 (s, 1H), 7.10 (s, | [M + H]⁺ = |
| | 1H), 4.04 (s, 2H), 3.88-3.85 (m, 1H), 3.34-3.30 (m, 2H), | 368.1. |
| | 2.60-2.52 (m, 2H), 2.08 (s, 3H), 1.75-1.68 (m, 1H), 1.63-1.52 | |
| | (m, 1H), 1.47-1.45 (m, 3H), 1.20-1.17 (m, 1H) | |
| 107 | ¹H NMR (400 MHz, DMSO-d₆ + D₂O) δ 7.78 (d, J = 9 Hz, | (ESI): m/z |
| | 1H), 7.66 (d, J = 9 Hz, 1H), 7.15 (s, 1H), 7.10 (s, 1H), 4.04 | [M + H]⁺ = |
| | (s, 2H), 3.88-3.85 (m, 1H), 3.34-3.30 (m, 2H), 2.60-2.52 | 382.2. |
| | (m, 2H), 2.08 (s, 3H), 1.75-1.59 (m, 1H), 1.58-1.46 (m, | |
| | 1H), 1.47-1.45 (m, 3H), 1.20-1.17 (m, 1H) | |
| 108 | ¹H NMR (400 MHz, DMSO-d₆) δ 10.25 (br. s, 1H), 7.79 (d, | (ESI): m/z: |
| | J = 9 Hz, 1H), 7.67 (d, J = 9 Hz, 1H), 7.16 (s, 1H), 7.10 (s, | [M + H]⁺ = |
| | 1H), 4.00 (s, 2H), 3.88-3.86 (m, 1H), 3.76-3.71 (m, 1H), | 382.0. |
| | 3.30-3.25 (m, 1H), 3.09-3.04 (m, 1H), 2.59-2.37 (m, 2H), | |
| | 2.08 (s, 3H), 1.97-1.81 (m, 1H), 1.77-1.62 (m, 1H), 1.61-1.37 | |
| | (m, 2H), 1.24-1.12 (m, 1H) | |
| 109 | ¹H NMR (400 MHz, DMSO-d₆) δ 9.93 (br. s, 1H), 7.79 (d, | (ESI): m/z |
| | J = 9 Hz, 1H), 7.67 (d, J = 9 Hz, 1H), 7.15 (s, 1H), 7.09 (s, | [M + H]⁺ = |
| | 1H), 4.02 (s, 2H), 3.90-3.80 (m, 2H), 3.30-3.24 (m, 2H), | 382.1 |
| | 2.46 (d, J = 6 Hz, 2H), 2.08 (s, 3H), 1.65-1.62 (m, 3H), | |
| | 1.19-1.11 (m, 2H) | |
| 110 | ¹H NMR (400 MHz, DMSO-d₆) δ 7.79 (d, J = 9 Hz, 1H), | (ESI): m/z: |
| | 7.64 (d, J = 9 Hz, 1H), 7.12 (s, 1H), 7.07 (s, 1H), 4.05 (s, | [M + H]⁺ = |
| | 2H), 2.70-2.68 (m, 2H), 2.48-2.35 (m, 2H), 2.12 (s, 3H), | 381.1 |
| | 2.07 (s, 3H), 1.90-1.78 (m, 4H), 1.37-1.26 (m, 2H) | |
| 111 | ¹H NMR (400 MHz, DMSO-d₆) δ 10.18 (br. s, 1H), 7.80 (d, | (ESI): m/z: |
| | J = 9 Hz, 1H), 7.67 (d, J = 9 Hz, 1H), 7.15 (s, 1H), 7.09 (s, | [M + H]⁺ = |
| | 1H), 4.16-4.02 (m, 2H), 3.45-3.35(m, 1H), 3.15-3.05 (m, | 395.2. |
| | 1H), 3.02-2.98 (m, 1H), 2.76 (s, 3H), 2.71-2.60 (m, 1H), | |
| | 2.38-2.28 (m, 1H), 2.21-2.12 (m, 1H), 2.08 (s, 3H), 1.97-1.92 | |
| | (m, 1H), 1.74-1.64 (m, 1H) | |
| 112 | ¹H NMR (400 MHz, DMSO-d₆) δ 10.25 (br. s, 1H), 7.79 (d, | (ESI): m/z: |
| | J = 9 Hz, 1H), 7.66 (d, J = 9 Hz, 1H), 7.16 (s, 1H), 7.09 (s, | [M + H]⁺ = |
| | 1H), 4.06-4.02 (m, 2H), 3.37-3.34 (m, 1H), 3.22-3.14 (m, | 395.1. |
| | 1H), 2.96-2.93 (m, 1H), 2.79 (s, 3H), 2.67-2.64 (m, 1H), | |
| | 2.46-2.42 (m, 1H), 2.15-2.09 (m, 1H), 2.08 (s, 3H), 2.03-1.96 | |
| | (m, 1H), 1.73-1.64 (m, 1H) | |
| 113 | ¹H NMR (400 MHz, DMSO-d₆) δ 10.24 (br. s, 1H), 7.81 (d, | (ESI): m/z: |
| | J = 9 Hz, 1H), 7.66 (d, J = 9 Hz, 1H), 7.16 (s, 1H), 7.10 (s, | [M + H]⁺ = |
| | 1H), 4.18-4.13 (m, 2H), 3.38-3.34 (m, 1H), 3.30-3.20 (m, | 381.1. |
| | 2H), 2.95-2.85 (m, 1H), 2.74 (s, 3H), 2.29-2.19 (m, 1H), | |
| | 2.08 (s, 3H), 1.73-1.68 (m, 1H). | |
| 114 | ¹H NMR (400 MHz, DMSO-d₆) δ 9.91 (br. s, 1H), 7.78 (d, | (ESI): m/z: |
| | J = 9 Hz, 1H), 7.67 (d, J = 9 Hz, 1H), 7.15 (s, 1H), 7.09 (s, | [M + H]⁺ = |
| | 1H), 4.13-4.03 (m, 2H), 3.60-3.50 (m, 1H), 3.17-3.16 (m, | 381.1. |
| | 1H), 3.20-3.10 (m, 1H), 2.88-2.87 (m, 1H), 2.71 (s, 3H), | |
| | 2.49-2.43 (m, 1H), 2.20-2.10 (m, 1H), 2.08 (s, 3H) | |
| 115 | ¹H NMR (400 MHz, DMSO-d₆) δ 9.76 (br. s, 1H), 7.78 (d, | (ESI): m/z: |
| | J = 9 Hz, 1H), 7.66 (d, J = 9 Hz, 1H), 7.15 (s, 1H), 7.09 (s, | [M + H] = |
| | 1H), 4.05 (s, 2H), 3.43 (t, J = 6 Hz, 2H), 3.25 (s, 3H), 2.72 (t, | 342.1. |
| | J = 6 Hz, 2H), 2.08 (s, 3H). | |
| 116 | ¹H NMR (400 MHz, DMSO-d₆) δ 10.21 (br. s, 1H), 7.77 (d, | (ESI): m/z: |
| | J = 9 Hz, 1H), 7.65 (d, J = 9 Hz, 1H), 7.15 (s, 1H), 7.09 (s, | [M + H]⁺ = |
| | 1H), 4.04 (s, 2H), 2.63 (t, J = 6 Hz, 2H), 2.34 (t, J = 6 Hz, | 355.1 |
| | 2H), 2.12 (s, 6H), 2.07 (s, 3H) | |
| 117 | ¹H NMR (400 MHz, DMSO-d₆) δ 10.20 (br. s, 1H), 7.80 (d, | (ESI): m/z |
| | J = 9 Hz, 1H), 7.67 (d, J = 9 Hz, 1H), 7.16 (s, 1H), 7.10 (s, | [M + H]⁺ = |
| | 1H), 4.27 (s, 1H), 4.07 (s, 2H), 2.47 (s, 2H), 2.08 (s, 3H), | 356.2. |
| | 1.12 (s, 6H) | |
| 118 | ¹H NMR (400 MHz, DMSO-d₆) δ 7.76 (d, J = 9 Hz, 1H), | (ESI): m/z |
| | 7.66 (d, J = 9 Hz, 1H), 7.15 (s, 1H), 7.09 (s, 1H), 4.02 (s, | [M + H]⁺ = |
| | 2H), 2.67 (t, J = 7 Hz, 2H), 2.07 (s, 3H), 1.57 (t, J = 7 Hz, | 370.1. |
| | 2H), 1.08 (s, 6H). | |
| 119 | ¹H NMR (400 MHz, DMSO-d₆) δ 7.76 (d, J = 9 Hz, 1H), | (ESI): m/z |
| | 7.63 (d, J = 7 Hz, 1H), 7.56 (d, J = 9 Hz, 1H), 7.10 (t, J = 8 | [M + H]⁺ = |
| | Hz, 1H), 6.92 (d, J = 5 Hz, 2H), 6.55 (d, J = 9 Hz, 1H), 6.04 | 361.0. |
| | (t, J = 6 Hz, 1H), 5.42 (s, 2H), 2.06 (s, 3H) | |
| 120 | ¹H NMR (400 MHz, DMSO-d₆) δ 10.42 (s, 1H), 9.18 (d, J = | (ESI): m/z |
| | 10 Hz, 2H), 9.01 (s, 1H), 8.41 (d, J = 8 Hz, 1H), 7.94-7.89 | [M + H]⁺ = |
| | (m, 1H), 7.85-7.78 (m, 1H), 7.17 (s, 1H), 7.14 (s, 1H), 6.87 | 362.1. |
| | (d, J = 8 Hz, 1H), 5.76 (s, 2H), 2.07 (s, 3H) | |
| 121 | ¹H NMR (400 MHz, DMSO-d₆) δ 8.36 (d, J = 8 Hz, 1H), | (ESI): m/z |
| | 8.04 (d, J = 3 Hz, 1H), 7.74 (d, J = 9 Hz, 1H), 7.61 (d, J = 9 | [M + H]⁺ = |
| | Hz, 1H), 6.69 (s, 1H), 6.64 (dd, J = 8, 3 Hz, 1H), 6.60 (s, | 362.0. |
| | 1H), 5.62 (s, 2H), 2.04 (s, 3H). | |
| 122 | ¹H NMR (400 MHz, DMSO-d₆) δ 10.31 (br. s, 1H), 7.71-7.67 | (ESI): m/z |
| | (m, 1H), 7.66-7.61 (m, 1H), 7.33 (d, J = 2 Hz, 1H), | [M + H]⁺ = |
| | 7.15 (s, 1H), 7.09 (s, 1H), 6.00 (t, J = 6 Hz, 1H), 5.48 (d, J = | 364.0. |
| | 2 Hz, 1H), 4.57 (d, J = 6 Hz, 2H), 3.59 (s, 3H), 2.08 (s, 3H) | |
| 123 | ¹H NMR (400 MHz, DMSO-d₆) δ 7.64 (d, J = 9 Hz, 1H), | (ESI): m/z |
| | 7.45 (d, J = 9 Hz, 1H), 6.92 (d, J = 3 Hz, 1H), 6.86 (d, J = | [M + H]⁺ = |
| | 3 Hz, 1H), 6.62 (s, 1H), 6.43 (s, 1H), 5.36 (s, 2H), 3.37 (s, | 364.0. |
| | 3H), 2.03 (s, 3H). | |
| 124 | ¹H NMR (400 MHz, DMSO-d₆) δ 10.26 (br. s, 1H), 8.36 (s, | (ESI): m/z: |
| | 1H), 8.26 (s, 1H), 7.77-7.72 (m, 1H), 7.71-7.66 (m, 1H), | [M + H]⁺ = |
| | 7.17 (s, 1H), 7.10 (s, 1H), 5.77 (t, J = 6 Hz, 1H), 4.46 (d, J = | 351.1. |
| | 6 Hz, 2H), 2.08 (s, 3H) | |
| 125 | ¹H NMR (400 MHz, DMSO-d₆) δ 10.33 (br. s, 1H), 7.69 (d, | ESI): m/z: |
| | J = 9 Hz, 1H), 7.54 (d, J = 9 Hz, 1H), 7.16 (s, 1H), 7.10 (s, | [M + H]⁺ = |
| | 1H), 6.66 (s, 1H), 5.12 (s, 2H), 2.08 (s, 3H), 2.01 (s, 3H). | 381.1. |
| 126 | ¹H NMR (400 MHz, DMSO-d₆) δ 10.05 (s, 1H), 7.78 (d, J = | (ESI): m/z: |
| | 9 Hz, 1H), 7.66 (d, J = 9 Hz, 1H), 7.29 (d, J = 2 Hz, 1H), | [M + H]⁺ = |
| | 7.16 (s, 1H), 7.09 (s, 1H), 6.17 (d, J = 2 Hz, 1H), 4.05 (d, J = | 378.0. |
| | 5 Hz, 2H), 3.84-3.77 (m, 5H), 3.02-2.97 (m, 1H), 2.08 (s, | |
| | 3H) | |
| 127 | ¹H NMR (400 MHz, DMSO-d₆) δ 10.21 (br. s, 1H), 7.80 (d, | (ESI): m/z |
| | J = 9 Hz, 1H), 7.65 (d, J = 9 Hz, 1H), 7.58 (d, J = 2 Hz, 1H), | [M + H]⁺ = |
| | 7.16 (s, 1H), 7.10 (s, 1H), 6.20 (d, J = 2 Hz, 1H), 4.04 (s, | 378.0. |
| | 2H), 3.78 (s, 3H), 3.69 (s, 2H), 2.90-2.75 (m, 1H), 2.08 (s, | |
| | 3H) | |
| 128 | ¹H NMR (400 MHz, DMSO-d₆) δ 9.96 (br. s, 1H), 7.80 (d, | (ESI): m/z: |
| | J = 9 Hz, 1H), 7.65 (d, J = 9 Hz, 1H), 7.59 (s, 1H), 7.36 (s, | [M + H]⁺ = |
| | 1H), 7.16 (s, 1H), 7.09 (s, 1H), 4.02 (s, 2H), 3.79 (s, 3H), | 378.0. |
| | 3.61 (s, 2H), 2.80-2.64 (m, 1H), 2.08 (s, 3H) | |
| 129 | ¹H NMR (400 MHz, DMSO-d₆) δ 10.25 (br. s, 1H), 7.84-7.71 | (ESI): m/z: |
| | (m, 1H), 7.69-7.59 (m, 1H), 7.16 (s, 1H), 7.10 (s, 1H), | [M + H]⁺ = |
| | 3.96-3.88 (m, 4H), 3.30-3.26 (m, 2H), 2.68 (m, 1H), 2.20 | 382.1. |
| | (s, 3H), 2.08 (s, 3H), 1.83-1.69 (m, 2H), 1.55 (m, 2H) | |
| 130 | ¹H NMR (400 MHz, DMSO-d₆) δ 10.21 (br. s, 1H), 7.78 (d, | ESI): m/z: |
| | J = 9 Hz, 1H), 7.68 (d, J = 9 Hz, 1H), 7.16 (s, 1H), 7.10 (s, | [M + H]⁺ = |
| | 1H), 3.85 (s, 2H), 3.65-3.57 (m, 4H), 2.48-2.45 (m, 4H), | 354.0. |
| | 2.08 (s, 3H) | |
| 131 | ¹H NMR (400 MHz, DMSO-d₆) δ 10.26 (br. s, 1H), 7.76 (d, | (ESI): m/z: |
| | J = 9 Hz, 1H), 7.66 (d, J = 9 Hz, 1H), 7.16 (s, 1H), 7.10 (s, | [M + H]⁺ = |
| | 1H), 3.83 (s, 2H), 2.49-2.41 (m, 4H), 2.40-2.25 (m, 4H), | 367.0. |
| | 2.16 (s, 3H), 2.08 (s, 3H) | |
| 132 | ¹H NMR (400 MHz, DMSO-d₆) δ 10.24 (br., 1H), 7.80 (d, | (ESI): m/z: |
| | J = 9 Hz, 1H), 7.69 (d, J = 9 Hz, 1H), 7.16 (s, 1H), 7.10 (s, | [M + H]⁺ = |
| | 1H), 3.93 (s, 2H), 3.30-3.28 (m, 2H), 3.10 (s, 2H), 2.83 (s, | 381.0. |
| | 3H), 2.76 (t, J = 6 Hz, 2H), 2.08 (s, 3H) | |
| 133 | ¹H NMR (400 MHz, DMSO-d₆) δ 10.26 (br. s, 1H), 7.80 (d, | (ESI): m/z: |
| | J = 9 Hz, 1H), 7.65 (d, J = 9 Hz, 1H), 7.16 (s, 1H), 7.10 (s, | [M + H]⁺ = |
| | 1H), 3.95 (s, 2H), 2.56-2.52 (m, 4H), 2.08 (s, 3H), 1.81-1.66 | 338.0. |
| | (m, 4H) | |
| 135 | ¹H NMR (400 MHz, DMSO-d₆) δ 10.26 (br. s, 1H), 7.79 (d, | ESI): m/z: |
| | J = 9 Hz, 1H), 7.65 (d, J = 9 Hz, 1H, 7.15 (s, 1H), 7.09 (s, | [M + H]⁺ = |
| | 1H), 3.79 (s, 2H), 2.42 (s, 4H), 2.07 (s, 3H), 1.60-1.50 (m, | 352.1. |
| | 4H), 1.45-1.35 (m, 2H) | |
| 136 | ¹H NMR (400 MHz, DMSO-d₆) δ10.01 (br. s, 1H), 7.80 (d, | (ESI): m/z: |
| | J = 9 Hz, 1H), 7.65 (d, J = 9 Hz, 1H), 7.15 (s, 1H), 7.09 (s, | [M + H]⁺ = |
| | 1H), 4.07 (s, 2H), 2.75-2.64 (m, 2H), 2.59-2.51 (m, 3H), | 384.0. |
| | 2.23-2.10 (m, 2H), 2.08 (s, 3H), 1.55-1.45 (m, 2H) | |
| 137 | ¹H NMR (400 MHz, DMSO-d₆) δ 10.26 (br. s, 1H), 7.83 (d, | (ESI): m/z: |
| | J = 9 Hz, 1H), 7.68 (d, J = 9 Hz, 1H), 7.16 (s, 1H), 7.10 (s, | [M + H]⁺ = |
| | 1H), 4.06 (d, J = 7 Hz, 2H), 3.20-3.15 (m, 2H), 3.09-2.97 | 416.1. |
| | (m, 2H), 2.90-2.80 (m, 1H), 2.79-2.70 (m, 1H), 2.18-2.05 | |
| | (m, 5H), 2.02-1.88 (m, 2H) | |
| 138 | ¹H NMR (400 MHz, DMSO-d₆) δ10.25 (br. s, 1H), 7.83 (d, | (ESI): m/z: |
| | J = 9 Hz, 1H), 7.66 (d, J = 9 Hz, 1H), 7.43-7.29 (m, 4H), | [M + H]⁺ = |
| | 7.28-7.21 (m, 1H), 7.15 (s, 1H), 7.09 (s, 1H), 4.03 (s, 2H), | 374.1. |
| | 3.78 (s, 2H), 3.00 (s, 1H), 2.08 (s, 3H) | |
| 139 | ¹H NMR (400 MHz, DMSO-d₆) δ 10.26 (br. s, 1H), 7.80 (d, | ESI): m/z: |
| | J = 9 Hz, 1H), 7.65 (d, J = 9 Hz, 1H), 7.16 (s, 1H), 7.09 (s, | [M + H]⁺ = |
| | 1H), 4.08 (s, 2H), 3.90-3.77 (m, 1H), 3.40-3.33 (m, 1H), | 382.1 |
| | 3.31-3.25 (m, 1H), 2.68-2.61 (m, 1H), 2.07 (s, 3H), 1.93-1.77 | |
| | (m, 2H), 1.25-1.14 (m, 1H), 1.09 (d, J = 6 Hz, 3H), | |
| | 1.00-0.87 (m, 1H) | |
| 140 | ¹H NMR (400 MHz, DMSO-d₆) δ 10.30 (br. s, 1H), 7.78 (d, | (ESI): m/z: |
| | J = 9 Hz, 1H), 7.64 (d, J = 9 Hz, 1H), 7.15 (s, 1H), 7.09 (s, | [M + H]⁺ = |
| | 1H), 4.04 (s, 2H), 3.21 (s, 3H), 3.13-3.02 (m, 1H), 2.45-2.35 | 396.1. |
| | (m, 1H), 2.07 (s, 3H), 1.98-1.90 (m, 4H), 1.18-1.00 | |
| | (m, 4H) | |
| 141 | ¹H NMR (400 MHz, DMSO-d₆) δ 10.23 (br. s, 1H), 7.81 (d, | (ESI): m/z: |
| | J = 9 Hz, 1H), 7.68 (d, J = 9 Hz, 1H), 7.40 (d, J = 5 Hz, 1H), | [M + H]⁺ = |
| | 7.16 (s, 1H), 7.10 (s, 1H), 7.02-6.96 (m, 2H), 4.06 (s, 2H), | 380.1. |
| | 3.98 (s, 2H), 3.16 (s, 1H), 2.08 (s, 3H) | |
| 142 | ¹H NMR (400 MHz, DMSO-d₆) δ 10.27 (br. s, 1H), 7.81 (d, | (ESI): m/z: |
| | J = 9 Hz, 1H), 7.66 (d, J = 9 Hz, 1H), 7.15 (s, 1H), 7.09 (s, | [M + H]⁺ = |
| | 1H), 4.21-4.02 (m, 3H), 3.80-3.70 (m, 1H), 3.10-3.02 (m, | 409.1. |
| | 1H), 2.79-2.65 (m, 2H), 2.08 (s, 3H), 1.98 (s, 3H), 1.93-1.70 | |
| | (m, 2H), 1.34-1.09 (m, 2H) | |
| 143 | ¹H NMR (400 MHz, DMSO-d₆) δ 10.28 (br. s, 1H), 7.79 (d, | (ESI): m/z |
| | J = 9 Hz, 1H), 7.67 (d, J = 9 Hz, 1H), 7.17 (s, 1H), 7.10 (s, | [M + H]⁺ = |
| | 1H), 3.99 (d, J = 7 Hz, 2H), 3.27-3.15 (m, 1H), 3.10-3.00 | 374.0 |
| | (m, 1H), 2.80-2.65 (m, 2H), 2.45-2.28 (m, 2H), 2.09 (s, | |
| | 3H) | |
| 144 | ¹H NMR (400 MHz, DMSO-d₆) δ 10.28 (br. s, 1H), 7.78 (d, | (ESI): m/z |
| | J = 9 Hz, 1H), 7.66 (d, J = 9 Hz, 1H), 7.16 (s, 1H), 7.09 (s, | [M + H]⁺ = |
| | 1H), 4.04 (s, 2H), 3.60-3.50 (m, 4H), 2.67 (t, J = 6 Hz, 2H), | 397.1. |
| | 2.41 (t, J = 6 Hz, 2H), 2.35-2.25 (m, 4H), 2.07 (s, 3H) | |
| 145 | ¹H NMR (400 MHz, DMSO-d₆) δ 10.59 (br. s, 1H), 8.36 (d, | (ESI): m/z |
| | J = 6 Hz, 1H), 8.29 (s, 1H), 7.96 (d, J = 9 Hz, 1H), 7.85 (d, | [M + H]⁺ = |
| | J = 9 Hz, 1H), 7.90-7.70 (m, 1H), 7.68 (d, J = 9 Hz, 1H), | 361.0. |
| | 7.25-7.15 (m, 2H), 6.10 (s, 2H), 2.07 (s, 3H). | |
| 147 | ¹H NMR (400 MHz, DMSO-d₆) δ 10.28 (s, 1H), 7.80-7.60 | (ESI): m/z |
| | (m, 2H) 7.16 (s, 1H), 7.09-7.08 (m, 2H), 6.98 (s, 1H), 5.31 | [M + H]⁺ = |
| | (t, J = 6 Hz, 1H), 4.41 (d, J = 6 Hz, 2H), 3.66 (s, 3H), 2.08 | 364.1. |
| | (s, 3H) | |
| 150 | ¹H NMR (400 MHz, DMSO-d₆) δ 10.23 (br. s, 1H), 8.23 (s, | (ESI): m/z: |
| | 1H), 7.74-7.67 (m, 2H), 7.52 (s, 1H), 7.16 (s, 1H), 7.10 (s, | [M + H]⁺ = |
| | 1H), 6.68 (t, J = 6 Hz, 1H), 4.61 (d, J = 6 Hz, 2H), 2.08 (s, | 367.0. |
| | 3H) | |
| 152 | ¹H NMR (400 MHz, DMSO-d₆) δ 10.24 (br. s, 1H), 7.81 (d, | (ESI): m/z |
| | J = 9 Hz, 1H), 7.66 (d, J = 9 Hz, 1H), 7.16 (s, 1H), 7.09 (s, | [M + H]⁺ = |
| | 1H), 4.12 (s, 1H), 4.03 (s, 2H), 2.60-2.50 (m, 1H), 2.43-2.24 | 396.2. |
| | (m, 1H), 2.07 (s, 3H), 1.81-1.80 (m, 2H), 1.56-1.54 | |
| | (m, 2H), 1.32-1.27 (m, 4H), 1.10 (s, 3H) | |
| 153 | ¹H NMR (400 MHz, DMSO-d₆) δ 10.23 (br. s, 1H), 7.82 (d, | (ESI): m/z: |
| | J = 9 Hz, 1H), 7.66 (d, J = 9 Hz, 1H), 7.17 (s, 1H), 7.10 (s, | [M + H]⁺ = |
| | 1H), 4.07 (s, 2H), 3.96 (s, 1H), 2.33-2.26 (m, 2H), 2.08 (s, | 396.1. |
| | 3H), 1.62-1.47 (m, 6H), 1.28-1.24 (m, 2H), 1.08 (s, 3H) | |
| 154 | ¹H NMR (400 MHz, DMSO-d₆) δ 7.96-7.80 (m, 2H), 6.88-6.40 | (ESI): m/z: |
| | (m, 2H), 4.70 (s, 2H), 3.86-3.74 (m, 2H), 3.29-2.98 | [M + H]⁺ = |
| | (m, 5H), 2.21-2.10 (m, 4H), 1.99-1.73 (m, 4H) | 393.1. |
| 21 | ¹H NMR (400 MHz, DMSO-d₆) δ 10.00 (br. s, 1H), 7.79 (d, | (ESI): m/z: |
| | J = 9 Hz, 1H), 7.67 (d, J = 9 Hz, 1H), 7.15 (s, 1H), 7.09 (s, | [M + H]⁺ = |
| | 1H), 4.58 (t, J = 7 Hz, 2H), 4.30 (t, J = 7 Hz, 2H), 3.98-3.93 | 340.1. |
| | (m, 3H), 2.08 (s, 3H) | |
| 22 | ¹H NMR (400 MHz, DMSO-d₆) δ 9.93 (br. s, 1H), 7.81 (d, | (ESI): m/z |
| | J = 9 Hz, 1H), 7.66 (d, J = 9 Hz, 1H), 7.15 (s, 1H), 7.09 (s, | [M + H]⁺ = |
| | 1H), 4.06-3.99 (m, 2H), 2.81-2.77 (m, 1H), 2.56-2.54 (m, | 381.1. |
| | 2H), 2.11 (s, 3H), 2.03 (s, 3H), 1.85-1.79 (m, 2H), 1.78-1.69 | |
| | (m, 1H), 1.58-1.68 (m, 1H), 1.48-1.41 (m, 1H), 1.06-1.00 | |
| | (m, 1H) | |
| 23 | ¹H NMR (400 MHz, DMSO-d₆) δ 10.47 (s, 1H), 9.66 (s, | (ESI): m/z: |
| | 2H), 7.85 (d, J = 9 Hz, 1H), 7.77 (d, J = 9 Hz, 1H), 7.17 (s, | [M + H]⁺ = |
| | 2H), 5.29 (s, 2H), 4.47 (s, 2H), 3.21 (s, 3H), 2.08 (s, 3H) | 380.1. |
| 24 | ¹H NMR (400 MHz, DMSO-d₆) δ 10.53 (s, 1H), 10.00 (s, | (ESI): m/z |
| | 2H), 8.02-7.94 (m, 1H), 7.86 (d, J = 9 Hz, 1H), 7.19 (s, | [M + H]⁺ = |
| | 2H), 4.67 (s, 2H), 3.96-3.30 (m, 5H), 2.40-2.21 (m, 2H), | 395.1. |
| | 2.11 (s, 3H), 2.02-1.92 (m, 3H) | |
| 25 | ¹H NMR (400 MHz, DMSO-d₆) δ 8.48 (br. s, 1H), 8.25 (d, | (ESI): m/z |
| | J = 7 Hz, 2H), 7.81 (d, J = 9 Hz, 1H), 7.56 (d, J = 9 Hz, 1H), | [M + H]⁺ = |
| | 6.78 (d, J = 7 Hz, 2H), 6.33 (s, 1H), 6.13 (s, 1H), 5.63 (s, | 361.1. |
| | 2H), 2.01 (s, 3H) | |
| 27 | ¹H NMR (400 MHz, DMSO-d₆) δ 10.30 (br. s, 1H), 7.85-7.75 | (ESI): m/z |
| | (m, 1H), 7.75-7.70 (m, 1H), 7.20-7.06 (m, 2H), 7.05 | [M + H]⁺ = |
| | (d, J = 2 Hz, 1H), 6.35 (t, J = 6 Hz, 1H), 5.32 (d, J = 2 Hz, | 364.0. |
| | 1H), 4.55 (d, J = 6 Hz, 2H), 3.60 (s, 3H), 2.07 (s, 3H) | |
| 28 | ¹H NMR (400 MHz, DMSO-d₆) δ 10.27 (br. s, 1H), 8.79 (d, | (ESI): m/z |
| | J = 2 Hz, 1H), 7.72-7.64 (m, 2H), 7.15 (s, 1H), 7.09 (s, 1H), | [M + H]⁺ = |
| | 6.89 (t, J = 6 Hz, 1H), 5.96 (d, J = 2 Hz, 1H), 4.65 (d, J = 6 | 367.0. |
| | Hz, 2H), 2.08 (s, 3H) | |

The compounds in Table B were prepared by the following method.

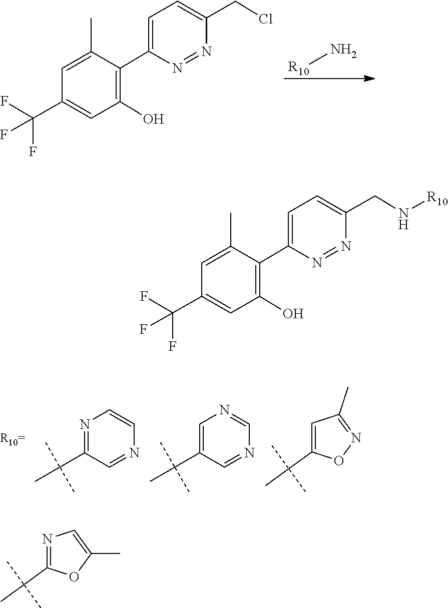

To a solution of 2-[6-(chloromethyl)pyridazin-3-yl]-3-methyl-5-(trifluoromethyl)phenol (Intermediate C1, 70 mg, 231 mol) and amine (1.39 mmol) in THE (0.3 mL) under nitrogen was added LiHMDS solution (1 M in THF, 463 μL, 463 mol) at 0° C. The mixture was stirred at 25° C. for 1 h then quenched by addition of saturated aqueous NH₄Cl (1 mL) at 0° C. and extracted with EtOAc (3×1 mL). The combined organic layers were washed with brine (1 mL), dried over Na₂SO₄, filtered and concentrated under reduced pressure. The residue was purified by prep-HPLC (column: Waters Xbridge Prep OBD C18 150×40 mm, 10 um; mobile phase: [(A) H₂O (10 mM NH₄HCO₃)—(B) ACN]; gradient: 25-50% B, 8 mins) and lyophilised to give the desired product.

TABLE B

| Compound | | |
|---|---|---|
| No. | NMR Data | LCMS Data |
| 26 | ¹H NMR (400 MHz, DMSO-d₆) δ 10.28 (br. s, 1H), | (ESI): m/z: |
| | 8.43 (s, 1H), 8.24 (s, 2H), 7.77-7.67 (m, 2H), 7.16 | [M + H]⁺ = |
| | (s, 1H), 7.10 (s, 1H), 6.95 (t, J = 6 Hz, 1H), 4.72 (d, | 362.1 |
| | J = 6 Hz, 2H), 2.08 (s, 3H) | |
| 146 | ¹H NMR (400 MHz, DMSO-d₆) δ 10.23 (br. s, 1H), | (ESI): m/z |
| | 8.11 (d, J = 1 Hz, 1H), 7.95 (dd, J = 2 Hz, 1H), 7.86 | [M + H]⁺ = |
| | (t, J = 6 Hz, 1H), 7.73 (d, J = 2 Hz, 1H), 7.69-7.59 | 362.1. |
| | (m, 2H), 7.16 (s, 1H), 7.10 (s, 1H), 4.85 (d, J = 4 Hz, | |
| | 2H), 2.08 (s, 3H) | |
| 148 | ¹H NMR (400 MHz, DMSO-d₆) δ 10.74 (br. s, 1H), | (ESI): m/z |
| | 7.74-7.61 (dm, 2H), 7.13 (d, 2H), 6.62 (s, 1H), 5.68 | [M + H]⁺ = |
| | (br. s, 1H), 4.98 (s, 2H), 2.09 (s, 3H), 2.00 (s, 3H) | 365.0. |
| 149 | ¹H NMR (400 MHz, DMSO-d₆) δ 10.13 (br. s, 1H), | (ESI): m/z |
| | 7.86 (t, J = 6 Hz, 1H), 7.74-7.66 (m, 2H), 7.17 (s, | [M + H]⁺ = |
| | 1H), 7.10 (s, 1H), 5.00 (s, 1H), 4.63 (d, J = 6 Hz, | 365.1. |
| | 2H), 2.08 (s, 3H), 2.02 (s, 3H) | |

The compounds in Table C were prepared by the following method.

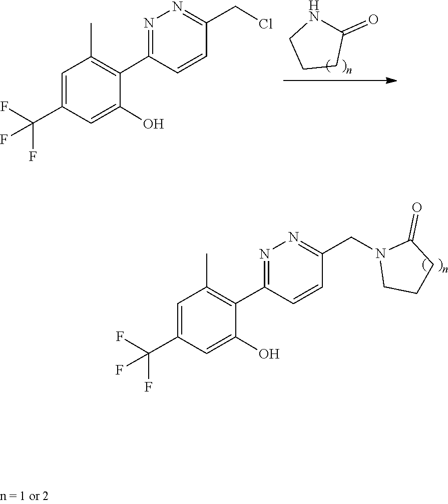

To a solution of the lactam (1.16 mmol) in DMF (2 mL) was added LiHMDS (1 M in THF, 1.16 mL, 1.16 mmol) at 0° C. under N₂. The mixture was stirred at 0° C. for 30 mi then 2-[6-(chloromethyl)pyridazin-3-yl]-3-methyl-5-(trifluoromethyl)phenol (Intermediate C1, 70 mg, 231 μmol) was added. The mixture was stirred at 25° C. for 8 h then the reaction mixture was diluted with H₂O (2 mL) and extracted with EtOAc (3×2 mL). The combined organic layers were washed with brine (2 mL), dried over anhydrous Na₂SO₄, filtered and concentrated under reduced pressure. The crude material was purified by prep-HPLC (column: Waters Xbridge BEH C18 100×30 mm, 10 am; mobile phase: [(A) H₂O (10 mM NH₄HCO₃)—ACN]; Gradient: 10-50% B, 8 mins) and lyophilised to give the desired product.

TABLE C

| Compound | | |
|---|---|---|
| No. | NMR data | LCMS |
| 134 | ¹H NMR (400 MHz, DMSO-d₆) δ 10.25 (s, 1H), | (ESI): m/z: |
| | 7.72-7.66 (m, 1H), 7.64-7.57 (m, 1H), 7.17 (s, 1H), 7.10 | [M + H]⁺ = |
| | (s, 1H), 4.73 (s, 2H), 3.44 (t, J = 7 Hz, 2H), | 352.1. |
| | 2.38-2.31 (m, 2H), 2.08 (s, 3H), 2.05-1.96 (m, 2H) | |
| 151 | ¹H NMR (400 MHz, DMSO-d₆) δ 10.44 (br. s, 1H), | (ESI): m/z: |
| | 7.75 (d, J = 9 Hz 1H), 7.65 (d, J = 9 Hz, 1H), 7.17 (s, | [M + H]⁺ = |
| | 1H), 7.15 (s, 1H), 4.83 (s, 2H), 3.42 (t, J = 5 Hz, 2H), | 366.1. |
| | 2.34 (t, J = 6 Hz, 2H), 2.10 (s, 3H), 1.80-1.79 (m, | |
| | 4H | |

### Example 2. Biological Activity of the Compounds of the Present Disclosure

The biological activity of the compounds of the present disclosure was determined utilising the assay described herein.

### PBMC IC₅₀ Determination Assay

The compounds of the present disclosure were tested for their inhibitory activity against IL-1β release upon NLRP3 activation in peripheral blood mononuclear cells (PBMC).

Protocol A. PBMC were isolated from buffy coats by density gradient centrifugation on Histopaque-1077 (Sigma, cat no. 10771). Isolated cells were seeded into the wells of a 96-well plate and incubated for 3 h with lipopolysaccharide (LPS). Following medium exchange, the compounds of the present disclosure were added (a single compound per well) and the cells were incubated for 30 min. Next, the cells were stimulated either with ATP (5 mM) or nigericin (10 μM) for 1 h and the cell culture media from the wells were collected for further analysis.

The release of IL-1β into the media was determined by a quantitative detection of IL-1β in the media using an IL-1β enzyme-linked immunosorbent assay (ELISA) Ready-SET-Go!, eBioscience cat. No. 88-7261-88. Briefly, in a first step, high affinity binding plates (Corning, Costar 9018 or NUNC Maxisorp Cat No. 44-2404) were coated overnight at 4° C. with specific capture antibody included in the kit (anti-human IL-1β ref. 14-7018-68). Subsequently, plates were blocked with blocking buffer for 1 h at room temperature (rt) and after washing with a buffer (PBS with 0.05% Tween-20) incubated with protein standard and culture media. After 2 h of incubation at rt, plates were washed and incubated with biotinylated detection antibody included in the kit (anti-human IL-1β Biotin ref. 33-7110-68) for 1 h at rt. Plates were washed and incubated with HRP-streptavidin for 30 min at rt and washed again. The signal was developed after addition of 3,3′,5,5′-tetramethylbenzidine-peroxidase (TMB) until colour appeared and the reaction was stopped by 2 M H₂SO₄. A microplate spectrophotometer (BioTek) was used to detect signals with 450 nm. The detection range of IL-1β ELISA was 2-150 ng/mL.

Protocol B. PBMC were isolated from buffy coats by density gradient centrifugation on Histopaque-1077 (Sigma, cat no. 10771). Isolated cells were seeded into the wells (280,000 cells/well) of a 96-well plate and incubated for 3 h with lipopolysaccharide (LPS, 1 μg/mL diluted 1000× from a 1 mg/mL stock solution). The compounds of the present disclosure were added (a single compound per well) and the cells were incubated for 30 min. Next, the cells were stimulated with ATP (5 mM final concentration diluted 20× from a 100 mM stock solution) for 1 h and the cell culture media from the wells were collected for further analysis.

The release of IL-1β into the media was determined by quantitative detection of IL-1β in the media using HTRF®, CisBio cat. No. 62HIL1BPEH. Briefly, cell culture supernatant were dispensed directly into the assay plate containing antibodies labelled with the HTRF® donor and acceptor. A microplate spectrophotometer (BMG) was used to detect signals at 655 nm and 620 nm. The detection range of IL-1β HTRF® was 39-6500 μg/mL.

The determination of the IC₅₀ values was preformed using the Graph Pad Prism software and the measured IC₅₀ values of compounds of the present disclosure are shown in Table D below (“+++++” means <0.5 μM; “++++” means ≥0.5 μM and <1 μM; “+++” means ≥1 and <3 μM; “++” means ≥3 and <10 μM; “+” means ≥10 μM). ND means not determined. These results show that the compounds of the present disclosure are capable of inhibiting IL-1β release upon inflammasome activation.

TABLE D

| Compound No. | THP-1 IC₅₀ | PBMC IC₅₀ |
|---|---|---|
| 1 | ++++ | ++++ |
| 2 | ++++ | ++++ |
| 3 | ++++ | ++++ |
| 4 | +++ | +++ |
| 5 | ++++ | ++++ |
| 6 | ++++ | ++++ |
| 7 | ++ | ++ |
| 8 | ++ | +++ |
| 9 | + | + |
| 10 | +++ | +++ |
| 11 | +++ | +++ |
| 12 | +++++ | +++++ |
| 13 | ++ | ++ |
| 14 | +++ | +++ |
| 15 | + | + |
| 16 | +++ | + |
| 17 | +++ | ++ |
| 18 | +++ | ++++ |
| 19 | +++ | +++ |
| 20 | ++ | +++ |
| 21 | ++++ | ++++ |
| 22 | +++ | +++ |
| 23 | ++ | ++ |
| 24 | ++++ | +++++ |
| 25 | ++ | ++ |
| 26 | +++ | ++++ |
| 27 | +++ | ++++ |
| 28 | ++++ | ++++ |
| 29 | +++++ | +++++ |
| 30 | ++ | ++ |
| 31 | +++ | +++++ |
| 32 | ++++ | +++++ |
| 33 | +++++ | +++++ |
| 34 | +++ | +++ |
| 35 | ++++ | ++++ |
| 36 | +++ | ++++ |
| 37 | +++ | ++++ |
| 38 | ++++ | +++++ |
| 39 | +++ | ++++ |
| 40 | +++ | ++++ |
| 41 | +++ | ++++ |
| 42 | ++ | ++ |
| 43 | ++++ | +++++ |
| 44 | ++++ | +++++ |
| 45 | +++ | +++ |
| 46 | +++ | +++ |
| 47 | ++ | +++ |
| 48 | +++ | +++ |
| 49 | +++++ | +++++ |
| 50 | ++ | +++ |
| 51 | +++ | ++++ |
| 52 | ++ | ++ |
| 53 | +++++ | +++++ |
| 54 | ++++ | +++++ |
| 55 | ++++ | ++++ |
| 56 | +++ | ++++ |
| 57 | ++ | +++ |
| 58 | +++ | +++ |
| 59 | ++++ | +++ |
| 60 | +++ | +++ |
| 61 | +++ | +++ |
| 62 | +++++ | +++++ |
| 63 | ++++ | +++++ |
| 64 | +++++ | +++++ |
| 65 | ++++ | +++++ |
| 66 | +++ | ++++ |
| 67 | +++++ | +++++ |
| 68 | ++++ | ++++ |
| 69 | ++ | ++ |
| 70 | +++ | +++ |
| 71 | +++ | ++++ |
| 72 | +++ | ++++ |
| 73 | +++++ | +++++ |
| 74 | +++ | +++ |
| 75 | +++++ | ++++ |
| 76 | +++ | +++ |
| 77 | +++ | +++ |
| 78 | + | + |
| 79 | ++++ | ++++ |
| 80 | +++ | +++++ |
| 81 | +++ | +++ |
| 82 | +++ | ++++ |
| 83 | +++ | ++++ |
| 84 | ++++ | ++++ |
| 85 | +++ | +++ |
| 86 | ++++ | ++++ |
| 87 | +++++ | +++++ |
| 88 | +++++ | +++++ |
| 89 | +++++ | +++++ |
| 90 | ++++ | +++++ |
| 91 | +++ | +++ |
| 92 | ++ | +++ |
| 93 | +++ | +++ |
| 94 | +++ | +++ |
| 95 | +++++ | +++++ |
| 96 | ++++ | +++++ |
| 97 | +++ | ++++ |
| 98 | ++++ | +++++ |
| 99 | ++++ | +++++ |
| 100 | +++ | +++ |
| 101 | ++++ | +++++ |
| 102 | +++ | ++++ |
| 103 | +++++ | +++++ |
| 104 | +++ | +++ |
| 105 | ++ | +++ |
| 106 | ++ | +++ |
| 107 | ++ | ++ |
| 108 | ++ | ++ |
| 109 | ++ | ++ |
| 110 | +++ | +++ |
| 111 | +++++ | +++++ |
| 112 | +++ | ++++ |
| 113 | +++ | ++++ |
| 114 | ++++ | +++++ |
| 115 | +++ | +++ |
| 116 | ++ | ++ |
| 117 | +++ | ++++ |
| 118 | ++ | +++ |
| 119 | ++++ | ++++ |
| 120 | ++ | + |
| 121 | ++ | + |
| 122 | ++++ | +++++ |
| 123 | ++++ | +++ |
| 124 | +++++ | +++++ |
| 125 | +++ | +++ |
| 126 | +++ | +++ |
| 127 | +++ | ++++ |
| 128 | +++ | +++ |
| 129 | +++ | +++ |
| 130 | ++++ | ++++ |
| 131 | ++ | +++ |
| 132 | +++ | +++ |
| 133 | ++++ | +++++ |
| 134 | +++++ | +++++ |
| 135 | +++++ | +++++ |
| 136 | ++++ | ++++ |
| 137 | +++ | +++ |
| 138 | +++ | ++++ |
| 139 | +++ | ++++ |
| 140 | ++++ | +++++ |
| 141 | +++ | +++++ |
| 142 | ++++ | +++++ |
| 143 | ND | +++++ |
| 144 | ++ | +++ |
| 145 | +++ | ++ |
| 146 | ++++ | ++++ |
| 147 | ++++ | +++++ |
| 148 | +++ | ND |
| 149 | +++ | +++ |
| 150 | +++++ | +++++ |
| 151 | +++++ | +++++ |
| 152 | +++ | +++ |
| 153 | +++ | +++ |
| 154 | + | + |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I): 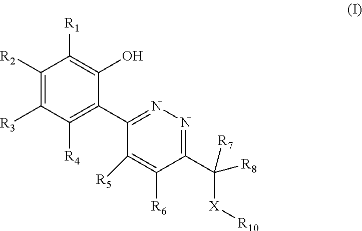
or a prodrug, solvate, or pharmaceutically acceptable salt thereof, wherein:
X is NR₉, O, S, S(O), or SO₂;
each R₁, R₂, R₃, and R₄ are independently H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{a};
R^{a} is halo, —CN, —OH, C₁-C₆ alkyl, C₂-C₆ alkenyl, C₂-C₆ alkynyl, C₁-C₆ haloalkyl, or C₁-C₆ alkoxyl;
each R₅ and R₆ are independently H, halo, —CN, —OH, —NO₂, —NH₂, —NH(C₁-C₆ alkyl), —N(C₁-C₆ alkyl)₂, C₁-C₃ alkyl, C₂-C₄ alkenyl, C₂-C₄ alkynyl, —C₁-C₃ alkoxyl, or C₁-C₃ haloalkyl;
each R₇ and R₅ are independently H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, or C₁-C₆ haloalkyl,
or R₇ and R₈, together with the atoms to which they are attached, form C₃-C₇ cycloalkyl;
R₉ is H, C₁-C₆ alkyl, C₂-C₆ alkenyl, C₂-C₆ alkynyl, or C₁-C₆ haloalkyl;
R₁₀ is H, halo, —CN, —OH, —NO₂, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ alkoxyl, C₃-C₆ cycloalkyl, C₆-C₁₀ aryl, 5- to 6-membered heteroaryl, or 3- to 10-membered heterocyclyl, wherein the alkyl, alkenyl, alkynyl, alkoxyl, cycloalkyl, aryl, heteroaryl, or heterocyclyl is optionally substituted with one or more R^{b}; and
R^{b} is halo, oxo, =NH, ═S, —CN, —OH, —NH₂, —NH(C₁-C₆ alkyl), —C(O)(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxyl, C₃-C₆ cycloalkyl, C₆-C₁₀ aryl, 5- to 6-membered heteroaryl, or 3- to 10-membered heterocyclyl, wherein the cycloalkyl, aryl, heteroaryl, or heterocyclyl is optionally substituted with one or more C₁-C₆ alkyl, C₂-C₆ alkenyl, C₂-C₆ alkynyl, C₁-C₆ haloalkyl, or C₁-C₆ alkoxyl, or
R₉ and R₁₀, together with the atoms to which they are attached, form 5- to 10-membered heteroaryl or 3- to 10-membered heterocyclyl, wherein the heteroaryl or heterocyclyl is optionally substituted with one or more R^{b}.

2. The compound of claim 1, wherein X is NR₉.

3. The compound of claim 1, wherein X is O.

4. The compound of claim 1, wherein X is NH.

5. The compound of claim 1, wherein R₁ is H or C₁-C₆ alkyl.

6. The compound of claim 1, wherein R₁ is H or —CH₃.

7. The compound of claim 1, wherein R₂ is halo, —NO₂, CN, C₁-C₆ alkyl, C₁-C₆ alkoxyl, or C₃-C₆ cycloalkyl, wherein the alkyl, alkoxyl, or cycloalkyl is optionally substituted with one or more R^{a}.

8. The compound of claim 1, wherein R₂ is Cl, F, Br, CN, —NO₂, —CF₃, —OCF₂H, —OCF₃, —CH₃, or cyclopropyl.

9. The compound of claim 1, wherein R₂ is Cl, F, Br, —NO₂, —CF₃, —OCF₂H, —OCF₃, or cyclopropyl.

10. The compound of claim 1, wherein R₂ is Cl, F, Br, —CF₃, —OCF₂H, —OCF₃, or cyclopropyl.

11. The compound of claim 1, wherein R₃ is H or C₁-C₆ alkyl.

12. The compound of claim 1, wherein R₃ is H or —CH₃.

13. The compound of claim 1, wherein R₄ is H, halo, or C₁-C₆ alkyl.

14. The compound of claim 1, wherein R₄ is H, F, Cl, or —CH₃.

15. The compound of claim 1, wherein R₅ is H or C₁-C₃ alkyl.

16. The compound of claim 1, wherein R₅ is H or —CH₃.

17. The compound of claim 1, wherein R₆ is H.

18. The compound of claim 1, wherein R₇ is H or C₁-C₆ alkyl, or R₇ and R₈, together with the atoms to which they are attached, form C₃-C₇ cycloalkyl.

19. The compound of claim 1, wherein R₇ is H or —CH₃.

20. The compound of claim 1, wherein R₈ is H or C₁-C₆ alkyl, or R₇ and R₈, together with the atoms to which they are attached, form C₃-C₇ cycloalkyl.

21.-64. (canceled)
